(19) United States
(12) Patent Application Publication
(10) Pub. No.: US 2010/0279936 A1
(43) Pub. Date: Nov. 4, 2010

(54) SUBSTITUTED ACETYLENIC COMPOUNDS USEFUL FOR THE TREATMENT OF DISEASES

(75) Inventors: Xifu Liang, Glostrup (DK); Thomas Høyer, Charlottenlund (DK); Jef Fensholdt, Stenlose (DK); Sophie Elisabeth Havez, Valby (DK); Bjarne Nørremark, Stenlose (DK)

Correspondence Address: BIRCH STEWART KOLASCH & BIRCH, PO BOX 747, FALLS CHURCH, VA, 22040-0747, US

(73) Assignee: LEO PHARMA A/S, Ballerup (DK)

(21) Appl. No.: 12/376,417

(22) PCT Filed: Aug. 16, 2007
(86) PCT No.: PCT/DK07/00375

§ 371 Date: Jun. 24, 2009

Related U.S. Application Data
(60) Provisional application No. 60/838,380, filed on Aug. 18, 2006.

(51) Int. Cl. A61K 38/23

(52) U.S. Cl. 514/11.9

(57) ABSTRACT

The invention relates to novel compounds according to formula Ia and Ib; (Formula Ia and Ib) wherein A represents substituted or unsubstituted C₁₋₁₀heteroaryl, C₆₋₁₄aryl or C₆₋₁₀heterocycloalkylaryl; R₁ is C₁₋₆alkyl, C₂₋₆alkenyl, C₂₋₆alkynyl, C₁₋₆hydroxyalkyl, C₁₋₆haloalkyl, C₁₋₆amino, C₃₋₆ cycloalkyl, or C₁₋₆heterocycloalkyl, each of which are optionally substituted; X represents —CR₃R₄—(CR₅R₆)ₙ—(CR₇═CR₈)ₘ—(C₆₋₁₄aryl)ᵣ-(C₁₋₁₀heteroaryl)ₛ-(CR₉R₁₀)ₚ—(CR₁₁═CR₁₂)_{q}, R₂ represents C₁₋₆alkyl, C₂₋₆alkenyl, C₂₋₆alkynyl, C₁₋₆hydroxyalkyl, C₁₋₆haloalkyl, C₁₋₆amino, C₁₋₁₂alkylsilyl, C₆₋₃₀alkylarylsilyl, C₁₋₁₀heteroaryl, C₆₋₁₄aryl, C₁₋₁₀heterocycloalkyl, C₁₋₁₀heterocycloalkenyl, C₁₋₈cycloalkyl, C₁₋₁₈cycloalkenyl, each of which is optionally substituted, or R₂ represents hydrogen, carboxy, or hydroxy; or a pharmaceutically acceptable salt, solvate, or ester thereof; to processes for the preparation thereof, to said compounds for use in therapy, to pharmaceutical compositions comprising said compounds, wherein said compounds being useful, e.g. in the treatment of diseases associated with disturbances of CaSR activity, such as hyperparathyroidism.

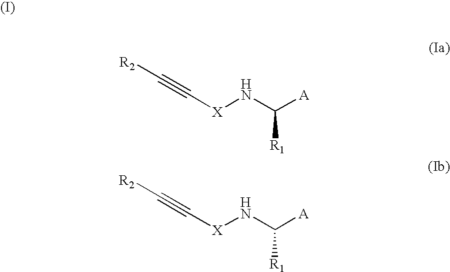

### FIELD OF THE INVENTION

This invention relates to novel substituted acetylenic compounds and derivatives thereof, processes for the preparation thereof, to said compounds for use in therapy, to pharmaceutical compositions comprising said compounds, to methods of treating diseases with said compounds, and to the use of said compounds in the manufacture of medicaments.

### BACKGROUND OF THE INVENTION

The calcium-sensing receptor (CaSR) is a G-protein-coupled receptor (GPCR) that signals through the activation of phospholipase C, increasing levels of inositol 1,4,5-triphosphate and cytosolic calcium. The CaSR belongs to the subfamily C of the GPCR superfamily, which also includes receptors for glutamate, gamma aminobutyric acid (GABA), pheromones and odorants that all possess a very large extracellular domain. This domain is highly negatively charged and is involved in binding of calcium and other positively charged molecules. The CaSR is found in the parathyroid glands but has also been identified in the brain, intestine, pituitary, thyroid glands, bone tissue and kidneys. In the parathyroid glands, the CaSR is activated by small increases in extracellular ionized calcium, which inhibits parathyroid hormone (PTH) release from within the stored intracellular granules [Brown, E. M. Calcium-Sensing Receptor. Primer of the Metabolic Bone Diseases and Disorders of Mineral Metabolism Fifth Edition, 2003 by American Society for Bone and Mineral Research, Chapter 17, p. 111; Drueke, T. E. Nephrol Dial Transplant (2004) 19, v20-v26].

In addition to endogenous ligands, small molecule allosteric activators of the CaSR (“calcimimetics”) have been developed [Urena, P.; Frazao, J. M. Calcimimetic agents: Review and perspectives. Kidney International (2003), 63, pp. s91-s96; Soudijn, W. et al. Allosteric modulation of G protein-coupled receptors: perspectives and recent developments. DDT (2004), 9, 752-758].

The binding site of known calcimimetics is believed to be located in the seven-trans-membrane domain of the receptor [Petrel, C. et al. Journal of Biological Chemistry (2004), 279, 18990-18997].

Calcimimetics have already been shown to be commercially useful for the treatment of hyperparathyroidism (HPT): The calcimimetic compound Cinacalcet® [Balfour, J. A. B. et al. Drugs (2005) 65(2), 271-281; Linberg et. al. J. Am. Soc. Nephrol (2005), 16, 800-807, Clinical Therapeutics (2005), 27(11), 1725-1751] has recently been launched for the treatment of secondary HPT in chronic kidney disease patients on dialysis and for the treatment of primary HPT in patients with parathyroid carcinoma. Thus, proof of concept for activators of calcium sensing receptor (CaSR) in humans has been achieved and the clinical relevance is already well established.

In chronic kidney disease hypocalcemia results from a disturbance in renal phosphorus handling and decreased formation of 1,25(OH)-2-VitD. In response, the PTH secretion is increased resulting in a condition referred to as secondary HPT. Primary HPT is a hypercalcemic disorder that results from excessive secretion of PTH usually caused by parathyroid adenoma or primary parathyroid hyperplasia.

Other calcimimetic compounds were for example described in WO02/059102, WO98/001417, WO05/065050, WO03/099814, WO03/099776, WO02/059102, WO00/21910, WO01/34562, WO01/090069, WO97/41090, U.S. Pat. No. 6,001,884, WO96/12697, EP1203761, WO95/11221, WO93/04373, EP1281702, WO02/12181, WO04/56365, WO04/069793, WO04/094362, US2004242602, WO04/106280, WO04/106295, WO04/106296, WO05/068433, and WO05/115975.

The calcimimetic activity corresponds to the ability to produce or induce biological responses observed through variations in the concentration of extracellular calcium ions (Ca²⁺)ₑ and extracellular magnesium ions (Mg²⁺)ₑ.

(Ca²⁺)ₑ and (Mg²⁺)ₑ ions play a major role in the body since they regulate calcium homeostasis on which the vital functions of the body depend. Thus, hypo- and hypercalcemia, that is to say conditions in which (Ca²⁺)ₑ ions are below or above the mean threshold, have a major effect on many functions, such as cardiac, renal or intestinal functions. They deeply affect the central nervous system (Chattopadhyay et al. Endocr. Review, 1998).

CaSRs are proteins which are sensitive to (Ca²⁺)ₑ and (Mg²⁺)ₑ ions, and are present in the parathyroid and thyroid glands, the kidney, the intestine, the lungs, bone cells, the brain, the spinal cord, the pituitary gland, the stomach and keratinocytes (Brown et al, Nature, 1993; Ruat et al, Proc. Natl. Acad. Sci., USA, 1995; Brown et al, Ann. Rev. Med., 1998). These proteins are encoded by a single gene isolated from various animal species. They belong to the family of G protein-coupled receptors with seven transmembrane domains, and exhibit structural homologies with metabotropic glutamate receptors, GABA receptors, and hypothetical pheromone and taste receptors. Activating or inhibitory mutations of the genes in humans are responsible for extremely serious genetic diseases which cause hypocalcemia or hypercalcemia (Pollack et al, Cell, 1993; Pollack et al, Nature Genetic, 1994; Brown et al, Ann. Rev. Med., 1998).

The functions associated with the expression of these proteins in tissues are not yet all known and are the subject of a very great deal of research activity, particularly with regard to the CaSRs present in the parathyroid and thyroid glands, the kidney, the intestine, the spinal cord, the brain and bone cells.

In the parathyroid gland, the CaSRs modulate the secretion of parathyroid hormone (PTH), which is the main regulator of calcium homeostasis: an increase in (Ca²⁺)ₑ ions in the serum will activate the CaSRs present on the cells of the parathyroid gland and decrease secretion of the PTH hormone.

The complementary DNA encoding rat CaSR has been isolated from a rat striatum cDNA library (Ruat et al, Proc. Natl. Acad. Sci., 1995). This receptor is identical, in terms of its amino acid sequence, to that expressed in the other tissues. Transfected Chinese hamster ovary (CHO) cells expressing rat CaSR(CHO(CaSR)) have been characterized and the chemical signals (second messengers) induced by activation of this receptor have been analyzed. Thus, a biochemical test for measuring the accumulation of tritiated inositol phosphates, [³H]IPs, in response to activation of the receptor has been developed (Ruat et al, J. Biol. Chem., 1996; Ferry et al, Biochem. Biophys. Res. Common., 1997).

It has been shown that Ca²⁺ and Mg²⁺ ions, but also Ba²⁺ ions, within millimolar concentration ranges, stimulate CaSRs. Activation of CaSRs might be induced in the brain by β-amyloid peptides, which are involved in neurodegenerative diseases such as Alzheimer's disease (Ye et al, J. Neurosci. Res. 1997).

Disturbance of CaSR activity is associated with biological disorders such as primary and secondary hyperparathyroidism, osteoporosis, cardiovascular, gastrointestinal, endocrine and neurodegenerative diseases, or certain cancers in which (Ca²⁺)ₑ ions are abnormally high.

Secondary hyperparathyroidism is observed in chronic renal failure and is characterized by hyperplasia of the parathyroid glands and an increase in circulating PTH. The renal failure is also accompanied by renal osteodystrophy, e.g. osteitis fibrosa, osteomalacia, adynamic bone disease, or osteoporosis. The disorders are characterized by either high or low bone turnover.

Osteoporosis is a multifactor disease which depends in particular on age and sex. While menopausal women are very greatly affected, osteoporosis is increasingly proving to be a problem in elderly men, and, for the moment, no really satisfactory treatments exist. Its social cost may become even heavier in the years to come, particularly in our European society where life expectancy is becoming longer. Osteoporosis is currently treated with estrogens, calcitonin or biphosphonates which prevent bone resorption without stimulating bone growth. More recent data demonstrate that intermittent increases in PTH or in derivatives thereof are effective in the treatment of osteoporosis and make it possible to remodel bone by stimulating bone formation (Whitfield et al., 1999). This new therapeutic approach for treatment of osteoporosis appears to be very advantageous, although major problems are associated with the use of PTH hormone, such as the route of injection, but also the appearance of tumors, observed recently during clinical trials in humans. Intermittent secretion of endogenous PTH can be obtained by blocking the calcium sensing receptor. The blocking of PTH secretion with CaSR agonists may be followed by a rapid increase in PTH (rebound effect), which is then beneficial in the treatment of osteoporosis.

### SUMMARY OF THE INVENTION

It has surprisingly been found that substituted acetylenic compounds of the present invention are modulators, e.g. activators or agonists of the human calcium sensing receptor (CaSR) and may thus be useful in the treatment or prophylaxis of a number of diseases or physiological disorders involving modulation of CaSR activity.

The substituted acetylenic compounds of the present invention may for example be useful in the treatment of complications associated with chronic kidney disease, such as hyperparathyroidism, e.g. primary and/or secondary hyperparathyroidism, or tertiary hyperparathyroidism. Other complications associated with chronic kidney disease are anemia, cardiovascular diseases, and the compounds of the present invention are also believed to have a beneficial effect on these diseases. The substituted acetylenic compounds of the present invention may furthermore be useful for promoting osteogenesis and treating or preventing osteoporosis, such as steroid induced, senile and post menopausal osteoporosis; osteomalacia and related bone disorders, or for the prevention of bone loss post renal transplantation, or in rescue therapy pre-parathyroidectomy.

It is presently believed that the substituted acetylenic compounds of the present invention may have advantageous pharmacokinetic or pharmacodynamic properties, such as oral bioavailability, in comparison to known structurally related compounds.

Accordingly, the present invention relates to a compound of general formula Ia or Ib

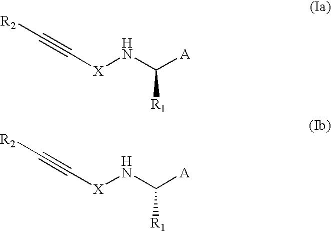

wherein
A represents C₁₋₁₀heteroaryl, C₆₋₁₄aryl or C₆₋₁₀heterocycloalkylaryl, each of which are optionally substituted with one or more, same or different substituents selected from the group consisting of halogen, hydroxy, mercapto, trifluoromethyl, cyano, carboxy, CONH₂, nitro, oxo, —S(O)₂NH₂, C₁₋₄alkyl, C₂₋₄alkenyl, C₂₋₄alkynyl, C₁₋₄hydroxyalkyl, C₁₋₆haloalkyl, C₁₋₄alkoxy, C₁₋₄alkoxycarbonyl, C₁₋₄alkylcarbonyloxy, C₁₋₄alkoxycarbonyloxy, C₁₋₄alkoxysulfonyloxy, C₁₋₄alkoxycarbamoyl, C₁₋₄aminocarbonyl, C₁₋₄alkylthio, C₃₋₆cycloalkyl, C₃₋₆cycloalkenyl, C₁₋₆amino, —NH₂, C₁₋₆imino, C₁₋₄aminosulfonyl, C₁₋₄aminocarbonyloxy, C₁₋₄alkylsulfonylamino, C₁₋₄alkoxyimino, C₁₋₄alkylcarbonylamino, C₁₋₄alkylsulfonyl, C₃₋₆heterocycloalkyl, C₃₋₆heterocycloalkenyl, C₁₋₄aminocarbonyloxy, C₁₋₁₀heteroaryl or C₆₋₁₄aryl,
wherein said C₁₋₄alkyl, C₂₋₄alkenyl, C₂₋₄alkynyl, C₁₋₄hydroxyalkyl, C₁₋₆haloalkyl, C₁₋₄alkoxy, C₁₋₄alkoxycarbonyl, C₁₋₄alkylcarbonyloxy, C₁₋₄alkoxycarbonyloxy, C₁₋₄alkoxysulfonyloxy, C₁₋₄alkoxycarbamoyl, C₁₋₄aminocarbonyl, C₁₋₄alkylthio, C₃₋₆cycloalkyl, C₃₋₆cycloalkenyl, C₁₋₆amino, C₁₋₆imino, C₁₋₄aminosulfonyl, C₁₋₄aminocarbonyloxy, C₁₋₄alkylsulfonylamino, C₁₋₄alkoxyimino, C₁₋₄alkylcarbonylamino, C₁₋₄alkylsulfonyl, C₃₋₆heterocycloalkyl, C₃₋₆heterocycloalkenyl, C₁₋₄aminocarbonyloxy, C₁₋₁₀heteroaryl or C₆₋₁₄aryl,
are optionally further substituted with one or more, same or different substituents selected from the group consisting of halogen, hydroxy, —NH₂, mercapto, trifluoromethyl, cyano, carboxy, CONH₂, nitro, oxo, —S(O)₂NH₂, C₁₋₄alkyl, C₁₋₆haloalkyl, C₁₋₃alkoxy or C₁₋₃hydroxyalkyl;
R₁ is C₁₋₆alkyl, C₂₋₆alkenyl, C₂₋₆alkynyl, C₁₋₆hydroxyalkyl, C₁₋₆haloalkyl, C₁₋₆amino, C₃₋₆cycloalkyl, or C₁₋₆heterocycloalkyl,
each of which are optionally substituted with one or more, same or different substituents selected from the group consisting of halogen, hydroxy, mercapto, trifluoromethyl, cyano, carboxy, CONH₂, nitro, oxo, C₁₋₃alkyl, C₂₋₄alkenyl, C₁₋₃hydroxyalkyl, C₁₋₃haloalkyl, C₁₋₄alkoxy, C₁₋₄alkoxycarbonyl, C₁₋₄amino, or —NH₂;
X represents —CR₃R₄—(CR₅R₆)ₙ—(CR₇═CR₈)ₘ—(C₆₋₁₄aryl)ᵣ-(C₁₋₁₀heteroaryl)ₛ-(CR₉R₁₀)ₚ—(CR₁₁═CR₁₂)_{q},
wherein n, m, p, q, r and s independently of each other is an integer of 0, 1, 2, 3, 4, 5, 6, 7, 8, 9, or 10,
R₃, R₄, R₅, R₆, R₇, R₉, R₉, R₁₀, R₁₁, R₁₂ independently of each other represent hydrogen, halogen, hydroxy, NH₂, C₁₋₆alkyl, C₁₋₆alkoxy, C₂₋₆alkenyl, C₂₋₆alkynyl, C₁₋₆hydroxyalkyl, C₁₋₆haloalkyl, C₃₋₆heterocycloalkyl, or C₃₋₆cycloalkyl,
the last eight of which are optionally substituted with one or more, same or different substituents selected from the group consisting of halogen, hydroxy, mercapto, trifluoromethyl, cyano, carboxy, CONH₂, nitro, oxo, C₁₋₃alkyl, C₂₋₄alkenyl, C₁₋₃hydroxyalkyl, C₁₋₃haloalkyl, C₁₋₄alkoxy, C₁₋₄alkoxycarbonyl, C₁₋₄amino, or —NH₂;
C₆₋₁₄aryl, C₁₋₁₀heteroaryl are optionally substituted with one or more, same or different substituents selected from the group consisting of halogen, hydroxy, mercapto, trifluoromethyl, cyano, carboxy, CONH₂, nitro, oxo, C₁₋₃alkyl, C₂₋₄alkenyl, C₁₋₃hydroxyalkyl, C₁₋₃haloalkyl, C₁₋₄alkoxy, C₁₋₄alkoxycarbonyl, C₁₋₄amino, or —NH₂;
R₂ represents C₁₋₆alkyl, C₂₋₆alkenyl, C₂₋₆alkynyl, C₁₋₆hydroxyalkyl, C₁₋₆haloalkyl, C₁₋₆amino, C₁₋₁₂alkylsilyl, C₆₋₃₀alkylarylsilyl, C₁₋₁₀heteroaryl, C₆₋₁₄aryl, C₁₋₁₀heterocycloalkyl, C₁₋₁₀heterocycloalkenyl, C₁₋₈cycloalkyl, C₁₋₁₈cycloalkenyl,
each of which is optionally substituted with one or more, same or different substituents selected from the group consisting of
halogen, hydroxy, —NH₂, mercapto, trifluoromethyl, cyano, carboxy, CONH₂, nitro, oxo, —S(O)₂NH₂, —OSi(CH(CH₃)₂)₃, C₁₋₄alkyl, C₁₋₆alkylsilyl, C₂₋₄alkenyl, C₂₋₄alkynyl, C₁₋₄hydroxyalkyl, C₁₋₆haloalkyl, C₁₋₄alkoxy, C₁₋₄alkoxycarbonyl, C₁₋₆ureido, C₁₋₆thioureido, C₁₋₄alkylcarbonyloxy, C₁₋₄alkoxycarbonyloxy, C₁₋₄alkoxysulfonyloxy, C₁₋₄aminocarbonyl, C₁₋₄alkylthio, C₃₋₆cycloalkyl, C₃₋₆cycloalkenyl, C₁₋₁₀amino, C₁₋₆imino, C₁₋₄aminosulfonyl, C₁₋₄aminocarbonyloxy, C₁₋₄alkylsulfonylamino, C₆₋₁₀arylsulfonylamino, C₆₋₁₀arylamino, C₁₋₆heterocycloalkylamino, C₁₋₆heterocycloalkylcarbonyl, C₆₋₁₀arylcarbonylamino, C₁₋₄alkoxyimino, C₁₋₄alkylcarbonylamino, C₁₋₄alkenylcarbonylamino, C₃₋₆cycloalkenylcarbonylamino, C₃₋₆cycloalkylcarbonylamino, C₁₋₄alkoxycarbonylamino, C₁₋₁₀heterocycloalkylcarbonylamino, C₁₋₄alkylsulfonyl, C₆₋₁₄aryl, C₁₋₆heteroaryl, C₁₋₁₀heterocycloalkylaryl, C₁₋₆heterocycloalkyl, or C₂₋₆heterocycloalkenyl,
wherein said C₁₋₄alkyl, C₁₋₆alkylsilyl, C₂₋₄alkenyl, C₂₋₄alkynyl, C₁₋₄hydroxyalkyl, C₁₋₆haloalkyl, C₁₋₄alkoxy, C₁₋₄alkoxycarbonyl, C₁₋₆ureido, C₁₋₆thioureido, C₁₋₄alkylcarbonyloxy, C₁₋₄alkoxycarbonyloxy, C₁₋₄alkoxysulfonyloxy, C₁₋₄aminocarbonyl, C₆₋₁₀arylcarbonylamino, C₁₋₄alkylthio, C₃₋₆cycloalkyl, C₃₋₆cycloalkenyl, C₁₋₆amino, C₆₋₁₀arylamino, C₁₋₆heterocycloalkylamino, C₁₋₆heterocycloalkylcarbonyl, C₁₋₆imino, C₁₋₄aminosulfonyl, C₁₋₄aminocarbonyloxy, C₁₋₄alkylsulfonylamino, C₆₋₁₀arylsulfonylamino, C₁₋₄alkoxyimino, C₁₋₄alkylcarbonylamino, C₁₋₁₀heterocycloalkylcarbonylamino, C₁₋₄alkylsulfonyl, C₆₋₁₄aryl, C₁₋₆heteroaryl, C₁₋₆heterocycloalkyl, or C₂₋₆heterocycloalkenyl,
are optionally further substituted with one or more, same or different substituents selected from the group consisting of halogen, hydroxy, —NH₂, mercapto, trifluoromethyl, cyano, carboxy, CONH₂, nitro, oxo, —S(O)₂NH₂, C₁₋₄alkyl, C₁₋₆alkylsilyl, C₂₋₄alkenyl, C₂₋₄alkynyl, C₁₋₄hydroxyalkyl, C₁₋₆haloalkyl, C₁₋₄alkoxy, C₁₋₄alkoxycarbonyl, C₁₋₆ureido, C₁₋₆thioureido, C₁₋₄alkylcarbonyloxy, C₁₋₄alkoxycarbonyloxy, C₁₋₄alkoxysulfonyloxy, C₁₋₄alkoxycarbamoyl, C₁₋₄aminocarbonyl, C₁₋₄alkylthio, C₃₋₆cycloalkyl, C₃₋₆cycloalkenyl, C₁₋₆amino C₁₋₆imino, C₁₋₄aminosulfonyl, C₁₋₄aminocarbonyloxy, C₁₋₄alkylsulfonylamino, C₆₋₁₀arylsulfonylamino, C₁₋₄alkoxyimino, C₁₋₄alkylcarbonylamino, C₁₋₄alkylsulfonyl, C₆₋₁₄aryl, C₁₋₆heteroaryl, C₁₋₁₀heterocycloalkylaryl, C₁₋₆heterocycloalkyl, or C₂₋₆heterocycloalkenyl,
wherein said C₁₋₄alkyl, C₁₋₆alkylsilyl, C₂₋₄alkenyl, C₂₋₄alkynyl, C₁₋₄hydroxyalkyl, C₁₋₆haloalkyl, C₁₋₄alkoxy, C₁₋₄alkoxycarbonyl, C₁₋₆ureido, C₁₋₆thioureido, C₁₋₄alkylcarbonyloxy, C₁₋₄alkoxycarbonyloxy, C₁₋₄alkoxysulfonyloxy, C₁₋₄alkoxycarbamoyl, C₁₋₄aminocarbonyl, C₁₋₄alkylthio, C₃₋₆cycloalkyl, C₃₋₆cycloalkenyl, C₁₋₁₀amino, C₁₋₆imino, C₁₋₄aminosulfonyl, C₁₋₄aminocarbonyloxy, C₁₋₄alkylsulfonylamino, C₁₋₄alkoxyimino, C₁₋₄alkylcarbonylamino, C₆₋₁₀arylsulfonylamino, C₁₋₄alkylsulfonyl, C₆₋₁₄aryl, C₁₋₆heteroaryl, C₁₋₁₀heterocycloalkylaryl, C₁₋₆heterocycloalkyl, or C₂₋₆heterocycloalkenyl,
are optionally further substituted with one or more, same or different substituents selected from the group consisting of
halogen, hydroxy, —NH₂, mercapto, trifluoromethyl, cyano, carboxy, CONH₂, nitro, oxo, —S(O)₂NH₂, C₁₋₄hydroxyalkyl, C₁₋₃alkoxy, benzyl or C₁₋₄alkoxycarbonyl.
or R₂ represents hydrogen, carboxy, or hydroxy;
or a pharmaceutically acceptable salt, solvate, or ester thereof.

In another aspect, the invention relates to the use of a compound of general formula Ia or Ib as defined above for the manufacture of a medicament for the prophylaxis, treatment or amelioration of physiological disorders or diseases associated with disturbances of CaSR activity, such as hyperparathyroidism.

In yet another aspect, the invention relates to a pharmaceutical composition comprising a compound of formula Ia or Ib or a pharmaceutically acceptable salt, solvate, or ester thereof together with a pharmaceutically acceptable excipient or vehicle.

In a further aspect, the invention relates to a method of preventing, treating or ameliorating parathyroid carcinoma, parathyroid adenoma, primary parathyroid hyperplasia, cardiac, renal or intestinal disfunctions, diseases of the central nervous system, chronic renal failure, chronic kidney disease, primary hyperparathyroidism, secondary hyperparathyroidism, tertiary hyperparathyroidism, anemia, cardiovascular diseases, renal osteodystrophy, osteitis fibrosa, adynamic bone disease, osteoporosis, steroid induced osteoporosis, senile osteoporosis, post menopausal osteoporosis, osteomalacia and related bone disorders, bone loss post renal transplantation, cardiovascular diseases, gastrointestinal diseases, endocrine and neurodegenerative diseases, cancer, neurodegenerative diseases, Alzheimer's disease, anemia, hypercalcemia, or renal bone diseases, the method comprising administering to a patient in need thereof an effective amount of a compound of general formula Ia or Ib as defined above, optionally in combination or as supplement with an active vitamin-D sterol or vitamin-D derivative, such as 1-α-hydroxycholecalciferol, ergocalciferol, cholecalciferol, 25-hydroxycholecalciferol, 1-α-25-dihydroxycholecalciferol, or in combination or as supplement with phosphate binders, estrogens, calcitonin or biphosphonates.

In a still further aspect the present invention relates to a compound of formula Ia or Ib as defined herein for use as a medicament in therapy.

### DETAILED DESCRIPTION OF THE INVENTION

### Definitions

The term “heteroaryl” is intended to include radicals of heterocyclic aromatic rings, comprising 1-4 heteroatoms (selected from O, S and N) and 1-10 carbon atoms, such as 1-3 heteroatoms and 1-6 carbon atoms, such as 1-2 heteroatoms and 1-5 carbon atoms, such as 1-2 heteroatoms and 2-4 carbon atoms, in particular 5- or 6-membered rings with 1-4 heteroatoms or 1-2 heteroatoms selected from O, S and N, or such as 1-4 heteroatoms and 6-10 carbon atoms in particular 9-membered rings with 1-2 heteroatoms, e.g. pyridyl, tetrazolyl, thiazolyl, imidazolyl, pyrazolyl, oxazolyl, oxadiazolyl, thiophenyl, 1,2,4-triazolyl, isoxazolyl, pyrrolidinyl, thienyl, pyrazinyl, pyrimidinyl, [1,2,3]triazolyl, quinolyl, indazolyl, isothiazolyl, benzo[b]thiophene or indolyl.

The term “cycloalkyl” is intended to indicate a saturated cycloalkane radical or ring, comprising 2-12 carbon atoms, such as 3-6 carbon atoms, such as 4-5 carbon atoms, e.g. cyclopropyl, cyclobutyl, cyclopentyl, and cyclohexyl.

The term “heterocycloalkylaryl” is intended to include radicals of heterocycloalkyl rings, in particular 5- or 6-membered rings, comprising 1-5 carbon atoms and 1-4 hetero atoms (selected form O, S and N), such as 1-4 carbon atoms and 1-3 hetero atoms, preferably 2-3 carbon atoms and 1-2 hetero atoms selected from O, S, or N, the heterocycloalkyl ring being fused with one or more aromatic carbocyclic rings comprising 6-20 carbon atoms, such as 6-14 carbon atoms, preferably 6-10 carbon atoms, in particular 5-, 6- or 10 membered rings, such as phenyl or naphthyl, e.g. benzodioxol.

The term “aryl” is intended to indicate a radical of aromatic carbocyclic rings comprising 6-20 carbon atoms, such as 6-14 carbon atoms, preferably 6-10 carbon atoms, in particular 5- or 6-membered rings, optionally fused carbocyclic rings with at least one aromatic ring, such as phenyl, naphthyl, e.g. 1-naphthyl, indenyl and indanyl, tetrahydro-naphthalene.

The term “halogen” is intended to indicate a substituent from the 7^{th} main group of the periodic table, preferably fluoro, chloro and bromo.

In the present context, the term “alkyl” is intended to indicate the radical obtained when one hydrogen atom is removed from a hydrocarbon. Said alkyl comprises 1-6, preferably 1-4, such as 2-3, carbon atoms. The term includes the subclasses normal alkyl (n-alkyl), secondary and tertiary alkyl, such as methyl, ethyl, n-propyl, isopropyl, n-butyl, isobutyl, sec.-butyl, tert.-butyl, pentyl, isopentyl, hexyl and isohexyl.

The term “alkenyl” is intended to indicate a mono-, di-, or triunsaturated hydrocarbon radical comprising 2-6 carbon atoms, in particular 2-4 carbon atoms, such as 2-3 carbon atoms, e.g. ethenyl, allyl, propenyl, butenyl, pentenyl, or hexenyl.

The term “alkynyl” is intended to indicate an hydrocarbon radical comprising 1-4 C—C triple bonds, e.g. 2 or 3 triple bonds, and 2-6 carbon atoms, the alkane chain typically comprising 2-5 carbon atoms, in particular 2-4 carbon atoms, such as 2-3 carbon atoms, e.g. ethynyl, propynyl, butynyl, pentynyl, or hexynyl.

The term “hydroxyalkyl” is intended to indicate an alkyl radical as defined above, wherein one, two, three or more hydrogen atoms are replaced by hydroxy.

The term “haloalkyl” is intended to indicate an alkyl radical as defined above, wherein one, two, three or more hydrogen atoms are replaced by halogen, same or different, such as bromo, iodo, chloro and/or fluoro.

The term “alkoxy” is intended to indicate a radical of the formula —OR, wherein R is alkyl or alkenyl as indicated above, e.g. methoxy, ethoxy, n-propoxy, isopropoxy, butoxy, etc.

The term “alkoxycarbonyl” is intended to indicate a radical of the formula —C(O)—O—R, wherein R is alkyl as indicated above, e.g. methoxycarbonyl, ethoxycarbonyl, n-propoxycarbonyl, isopropoxycarbonyl, etc.

The term “alkylcarbonyloxy” is intended to indicate a radical of the formula —O—C(O)—R, wherein R is alkyl as indicated above, e.g. methylcarbonyloxy, or ethylcarbonyloxy.

The term “alkoxycarbonyloxy” is intended to indicate a radical of the formula —O—C(O)—O—R, wherein R is alkyl as indicated above.

The term “alkoxysulfonyloxy” is intended to represent a radical of the formula —O—S(O)₂—O—R, wherein R is alkyl as indicated above.

The term “alkoxycarbamoyl” intended to indicate a radical of the formula —C(O)NR′—O—R, wherein R′ is hydrogen or alkyl as indicated above, and R is alkyl as indicated above.

The term “amino” is intended to indicate a radical of the formula —NRR′, wherein R and R′ independently represent hydrogen, alkyl, alkenyl, or cycloalkyl, as indicated above, e.g. —NH₂, dimethylamino, methylamino, diethylamino, cyclohexylamino, tert-butylamino, ethylmethylamino, or ethylamino.

The term “arylamino” is intended to indicate a radical of the formula —NRR′, wherein R represents hydrogen or alkyl and R′ represents aryl as indicated above, e.g. indalylamino, tetrahydro-naphtaleneamino.

The term “heterocycloalkylamino” is intended to indicate a radical of the formula —NRR′, wherein R represents hydrogen or alkyl and R′ represents heterocycloalkyl as indicated below e.g. piperidinylamino.

The term “heterocycloalkylcarbonyl” is intended to indicate a radical of the formula —C(O)—R, wherein R is heterocycloalkyl as indicated below, e.g. piperidylcarbonyl, morpholinylcarbonyl, piperazinylcarbonyl, pyrrolidinylcarbonyl or piperidiocarbonyl.

The term “aminocarbonyl” is intended to indicate a radical of the formula —C(O)—NR′₂, wherein each R′ is independently hydrogen, alkyl, or alkenyl as indicated above, e.g. carbamoyl, methylaminocarbonyl, ethylaminocarbonyl, propylaminocarbonyl, or butylaminocarbonyl.

The term “alkylthio” is intended to indicate a radical of the formula —S—R, wherein R is alkyl as indicated above.

The term “cycloalkenyl” is intended to indicate mono-, or di-unsaturated non-aromatic cyclic hydrocarbons radicals, comprising 2-10 carbon atoms, such as 3-6 carbon atoms, such as 4-5 carbon atoms, e.g. cyclopropenyl, cyclobutenyl, cyclopentenyl, or cyclohexenyl.

The term “imino” is intended to indicate a radical of the formula ═N—R, wherein R represents hydrogen or alkyl as indicated above.

The term “aminosulfonyl” is intended to indicate a radical of the formula —S(O)₂—NR₂, wherein each R independently represents hydrogen, or alkyl as indicated above.

The term “aminocarbonyloxy” is intended to indicate a radical of the formula —O—C(O)—NRR′, wherein R and R′ independently represent hydrogen or alkyl as indicated above, e.g. dimethylcarbamoyloxy.

The term “alkoxycarbonylamino” is intended to indicate a radical of the formula —NR′—C(O)—O—R, wherein R′ is hydrogen or alkyl as indicated above, and R is alkyl as indicated above, e.g. aminocarbonyl-tert-butoxy.

The term “alkylsulfonylamino” is intended to indicate a radical of the formula —NR′—S(O)₂—R, wherein R is alkyl as indicated above, and R′ is hydrogen or alkyl as indicated above, e.g. methylsulfonylamino, ethylsulfonylamino, propylsulfonylamino or butylsulfonylamino.

The term “arylsulfonylamino” is intended to indicate a radical of the formula —NR′—S(O)₂—R, wherein R is aryl as indicated above, and R′ is hydrogen or alkyl as indicated above, e.g. phenylsulfonylamino.

The term “alkoxyimino” intended to indicate a radical of the formula ═N—O—R, wherein R is hydrogen or alkyl as indicated above, e.g. methoxyimino.

The term “alkylcarbonylamino” is intended to indicate a radical of the formula —NR′—C(O)—R, wherein R′ is hydrogen or alkyl as indicated above, and R is alkyl, as indicated above, e.g. acetamino, acetylamine, propylcarbonylamino, propenylcarbonylamino, methylethylcarbonylamino, butylcarbonylamino, dimethylpropylcarbonylamino, ethylpropylcarbonylamino.

The term “arylcarbonylamino” is intended to indicate a radical of the formula —NR′—C(O)—R, wherein R′ is hydrogen or alkyl as indicated above, and R is aryl as indicated above e.g. phenyl.

The term “alkenylcarbonylamino” is intended to indicate a radical of the formula —NR′—C(O)—R, wherein R′ is hydrogen or alkyl as indicated above, and R is alkenyl as indicated above, e.g. propenylcarbonylamino.

The term “cycloalkylcarbonylamino” is intended to indicate a radical of the formula —NR′—C(O)—R, wherein R′ is hydrogen or alkyl as indicated above, and R is cycloalkyl as indicated above, e.g. cyclopropylcarbonylamino.

The term “cycloalkenylcarbonylamino” is intended to indicate a radical of the formula —NR′—C(O)—R, wherein R′ is hydrogen or alkyl as indicated above, and R is cycloalkenyl as indicated above, e.g. cyclohexenylcarbonylamino.

The term “heterocycloalkylcarbonylamino” is intended to indicate a radical of the formula —NR′—C(O)—R, wherein R′ is hydrogen or alkyl as indicated above, and R is heterocycloalkyl as indicated below, e.g. piperidinylcarbonylamino.

The term “alkylsulfonyl” is intended to indicate a radical of the formula —SO₂—R, wherein R is alkyl as indicated above.

The term “heterocycloalkyl” is intended to indicate a cycloalkyl radical as defined above, in particular 5- or 6-membered rings, including polycyclic radicals, comprising 1-4 heteroatoms, preferably 1-3 heteroatoms, selected from O, N, or S, e.g. tetrahydropyranyl, morpholinyl, imidazolidinyl, dioxolanyl, piperidyl, piperazinyl, pyrrolidinyl, piperidino or piperidinyl.

The term “heterocycloalkenyl” is intended to indicate a cycloalkenyl radical as defined above, including polycyclic radicals, comprising 1-4 heteroatoms, preferably 1-3 heteroatoms, selected from O, N, or S, e.g. 1,6-dihydropyridinyl, 4,5-dihydro-1H-[1,2,4]-triazolyl, 4,5-dihydro-oxazolyl, 1-H-pyrazolyl, or 4,5-dihydro-isoxazolyl.

The term “alkylsilyl” is intended to indicate a radical of the formula —SiRR″R′″, wherein R, R′, and R′″ independently represent hydrogen or alkyl as indicated above, such as tert-butyldimethylsilyl, trimethylsilyl, triethylsilyl, triisopropylsilyl, or tert-butyldiphenylsilyl.

The term “alkylarylsilyl” is intended to indicate a radical of the formula —SiRR″R′″, wherein R, R′, and R′″ independently represent hydrogen, alkyl, or aryl as indicated above, such diphenylmethylsilyl.

The term “ureido” is intended to indicate a radical of the formula “—NR′—C(O)—NH—R, wherein R′ is hydrogen or alkyl as indicated above, and R is hydrogen, alkyl, alkenyl, alkynyl, cycloalkyl or aryl as indicated above, e.g. —NH—C(O)—NH₂, methylureido, ethylureido, tert-butylureido, cyclohexylureido, methylthioureido, isopropylureido, n-propylureido or phenylureido.

The term “thioureido” is intended to indicate a radical of the formula “—NR′—C(S)—NH—R, wherein R′ is hydrogen or alkyl as indicated above, and R is hydrogen, alkyl, or cycloalkyl as indicated above, e.g. —NH—C(S)—NH₂.

The term “alkoxysulfonyloxy” is intended to represent a radical of the formula —O—S(O)₂—O—R, wherein R is alkyl as indicated above.

The term “pharmaceutically acceptable salt” is intended to indicate salts prepared by reacting a compound of formula I with a suitable inorganic or organic acid, such as hydrochloric, hydrobromic, hydroiodic, sulfuric, nitric, phosphoric, formic, acetic, 2,2-dichloroacetic, adipic, ascorbic, L-aspartic, L-glutamic, galactaric, lactic, maleic, L-malic, phthalic, citric, propionic, benzoic, glutaric, gluconic, D-glucuronic, methanesulfonic, salicylic, succinic, malonic, tartaric, benzenesulfonic, ethane-1,2-disulfonic, 2-hydroxy ethanesulfonic acid, toluenesulfonic, sulfamic or fumaric acid. Pharmaceutically acceptable salts of compounds of formula I may also be prepared by reaction with a suitable base such as sodium hydroxide, potassium hydroxide, magnesium hydroxide, calcium hydroxide, ammonia, or suitable non-toxic amines, such as lower alkylamines, for example triethylamine, hydroxy-lower alkylamines, for example 2-hydroxyethylamine, bis-(2-hydroxyethyl)-amine, cycloalkylamines, for example dicyclohexylamine, or benzylamines, for example N,N′-dibenzylethylene-diamine, and dibenzylamine, or L-arginine or L-lysine.

The term “solvate” is intended to indicate a species formed by interaction between a compound, e.g. a compound of formula I, and a solvent, e.g. alcohol, glycerol or water, wherein said species are in a solid form. When water is the solvent, said species is referred to as a hydrate.

The term “pharmaceutically acceptable ester” is intended to indicate easily hydrolysable esters, i.e. in vivo hydrolysable esters of the compounds of formula I such as alkanoyloxyalkyl, aralkanoyloxyalkyl, aroyloxyalkyl, e.g. acetoxymethyl, pivaloyloxymethyl, benzoyloxymethyl esters and the corresponding 1′-oxyethyl derivatives, or alkoxycarbonyloxyalkyl esters, e.g. methoxycarbonyloxymethyl esters and ethoxycarbonyloxymethyl esters and the corresponding 1′-oxyethyl derivatives, or lactonyl esters, e.g. phthalidyl esters, or dialkylaminoalkyl esters, e.g. dimethylaminoethyl esters. Such esters may be prepared by conventional methods known to persons skilled in the art, such as method disclosed in GB patent No. 1 490 852 incorporated herein by reference.

The term compound I, or a compound of formula I, includes both compound Ia and Ib.

When X is defined as —CH₂-phenylene-, the alkynyl radical attached to the phenylene ring may be in ortho, meta or para position. When X is defined as —CH₂-thienyl- the alkynyl radical is preferably attached to the thienyl ring in the 4 or 5 position. When X is defined as —CH₂-pyridyl- the alkynyl radical is preferably attached to the pyridyl ring is the 2 or 6 position.

Compounds of formula Ia or Ib may comprise asymmetrically substituted (chiral) carbon atoms and carbon-carbon double bonds which may give rise to the existence of isomeric forms, e.g. enantiomers, diastereomers and geometric isomers. The present invention includes all such isomers, either in pure form or as mixtures thereof. Pure stereoisomeric forms of the compounds and the intermediates of this invention may be obtained by the application of procedures known in the art. Diastereomers may be separated by physical separation methods such as selective crystallization and chromatographic techniques, e.g. liquid chromatography using chiral stationary phases. Enantiomers may be separated from each other by the selective crystallization of their diastereomeric salts with optically active acids. Alternatively, enantiomers may be separated by chromatographic techniques using chiral stationary phases. Said pure stereoisomeric forms may also be derived from the corresponding pure stereoisomeric forms of the appropriate starting materials, provided that the reaction occurs stereoselectively or stereospecifically. Preferably, if a specific stereoisomer is desired, said compound will be synthesized by stereoselective or stereospecific methods of preparation. These methods will advantageously employ chirally pure starting materials. Likewise, pure geometric isomers may be obtained from the corresponding pure geometric isomers of the appropriate starting materials. A mixture of geometric isomers will typically exhibit different physical properties, and they may thus be separated by standard chromatographic techniques well-known in the art.

The present invention includes further to prodrugs of compounds of general formula I, that means derivatives such esters, ethers, complexes or other derivatives which undergo a biotransformation in vivo before exhibiting their pharmacological effects.

The compounds of formula I, may be obtained in crystalline form either directly by concentration from an organic solvent or by crystallisation or recrystallisation from an organic solvent or mixture of said solvent and a cosolvent that may be organic or inorganic, such as water. The crystals may be isolated in essentially solvent-free form or as a solvate, such as a hydrate. The invention covers all crystalline modifications and forms and also mixtures thereof.

### Embodiments

In one embodiment of the present invention, X represents cis —CH═CH—CH₂—, trans —CH═CH—CH₂—, —CH₂—, —CH₂—CH₂—, —CH₂—CH₂—CH₂— or —CH₂—CH₂—CH₂—CH₂— optionally substituted with trifluoromethyl.

In another embodiment of the present invention X represents -phenylene-CH₂—, -thienyl-CH₂— or -pyridyl-CH₂—

wherein the phenylene, thienyl and pyridyl rings are optionally further substituted with one or more substituents selected from hydroxy or halogen.

In yet another embodiment of the present invention X represents -phenylene-CH₂—, -thienyl-CH₂— or -pyridyl-CH₂—, wherein the phenylene, thienyl and pyridyl rings are optionally further substituted with one or more substituents selected from hydroxyl or halogen,

and wherein the ethynylene group defined in formula Ia or Ib is attached to the phenylene ring in ortho-, meta- or para-position.

In yet another embodiment of the present invention, A represents 1-naphthyl, 2-naphthyl or phenyl, each of which are optionally substituted as defined above the substitution of C₆₋₁₄aryl representing A.

In yet another embodiment of the present invention, R₁ is methyl, ethyl, n-propyl, optionally substituted with halogen or hydroxy.

In yet another embodiment of the present invention, R₂ represents hydrogen, carboxy, C₁₋₆alkyl, C₂₋₆alkenyl, C₃₋₈cylcoalkyl, C₆₋₁₀aryl, C₁₋₃hydroxyalkyl or C₁₋₃alkylsilyl, the last six of which are optionally substituted with one or more, same or different substituents selected from the group consisting of NH₂, hydroxy, trifluoromethyl, C₁₋₃haloalkyl, fluoro, chloro, bromo, C₁₋₃alkylamino, phenyl, C₁₋₃alkylsilyl, OSi(CH(CH₃)₂)₃, C₁₋₃alkoxy, C₁₋₃alkyl, cyano, C₁₋₃hydroxyalkyl or C₃₋₆heterocycloalkyl, the latter further substituted with fluoro or methoxy.

In yet another embodiment of the present invention, R₂ represents hydrogen, methyl, tert-butyl, cyclopropyl, aminopropyl, aminoethyl, hydroxymethyl, hydroxyethylvinyl, methylaminoethyl, dimethylaminoethyl, trifluoromethylphenyl, dimethylhydroxyethyl, trifluoromethylvinylene, hydroxylpropyl, hydroxyisopropyl, hydroxypropenyl, bromophenyl, aminophenyl, hydroxybutyl, phenyl, trimethylsilyl, hydroxyethylpropyl, hydroxymethylpropyl, phenylhydroxymethyl, trimethylsilylmethyl, methoxyphenyl, difluorophenyl, tolyl, hydroxyphenyl, chlorophenyl, cyanophenyl, cyanopropyl, fluorophenyl, hydroxymethylphenyl, hydroxyphenylmethyl, trimethylsilyl, methoxymethyl, methoxyisopropyl, hydroxyethyl, carboxy, fluorooxacyclohexanephenyl or methoxyoxacyclohexanephenylene or dimethylaminophenylene.

In yet another embodiment of the present invention, R₂ represents C₁₋₄alkyl substituted with one or more, same or different substituents selected from C₁₋₄alkyl, C₁₋₆amino, C₁₋₄alkylcarbonylamino, C₁₋₄alkenylcarbonylamino, C₃₋₆cycloalkenylcarbonylamino, C₃₋₆cycloalkylcarbonylamino, C₆₋₁₀arylcarbonylamino, C₁₋₄alkoxycarbonylamino, C₁₋₁₀heterocycloalkylcarbonylamino, C₁₋₄alkylsulfonylamino, C₆₋₁₀arylsulfonylamino or C₁₋₆ureido, which are optionally substituted with one or more, same or different substituents selected from oxo, halogen, CF₃—, C₁₋₄alkyl, C₂₋₄alkynyl, C₆₋₁₀aryl, C₁₋₄alkoxy, C₁₋₃haloalkyl, C₁₋₆heterocycloalkyl, trifluoromethylphenylene or trifluoromethylbenzyl.

In yet another embodiment of the present invention, R₂ represents —CH₂—CH₂—, —CH₂— or —CH₂—C(CH₃)₂— substituted with formylamino, dimethylamino, diethylamino, dipropylamino, propenylcarbonylamino, butynecarbonylamino, benzylmethylamino, propylcarbonylamino, cyclohexenylcarbonylamino, cyclopropylcarbonylamino, dimethylpropylcarbonylamino, ethylpropylcarbonylamino, methoxymethylcarbonylamino, methylpiperidylcarbonylamino, isopropylcarbonylamino, dihydroxyphenylcarbonylamino, methylphenylsulfonylamino, ethylsulfonylamino, propylsulfonylamino, butylsulfonylamino, methoxyphenylsulfonylamino, trifluoromethylchlorophenylsulfonylamino, ethylureylene, isopropylureylene, cyclohexylreylene, phenylureylene, propylureylene, di-trifluoromethylphenylureylene, dimethoxyphenylureylene, trifluoromethylphenylureylene, di-trifluoromethylbenzylamino or tert-butoxycarbonylamino.

In yet another embodiment of the present invention, R₂ represents C₁₋₄alkyl, substituted with one or more same or different substituents selected from hydrogen or C₁₋₃alkyl;

and wherein C₁₋₄alkyl is further substituted with one or more same or different substituents selected from oxo, methoxy, carboxy, C₁₋₄alkyl, C₁₋₆alkoxycarbonyl or C₃₋₆heterocycloalkyl which are optionally substituted with one or more, same or different substituents selected from halogen, C₁₋₄alkyl, C₁₋₄alkoxy, C₁₋₃haloalkyl or C₆₋₁₀aryl.

In yet another embodiment of the present invention, R₂ represents —CR₁₃R₁₄—(CH₂)ᵤ—,

wherein
u is an integer of 0, 1,
R₁₃, R₁₄ independently of each other represents hydrogen or methyl;
and wherein —CR₁₃R₁₄— is substituted with carboxy or C₁₋₆alkoxycarbonyl which are optionally substituted with one or more, same or different substituents selected from methyl, C₁₋₄alkyl, C₁₋₄alkoxy, C₁₋₃haloalkyl or C₆₋₁₀aryl.

In yet another embodiment of the present invention, R₂ represents phenyl-CH₂—O—C(O)—C(CH₃)₂—, HO—C(O)—C(CH₃)₂— or R₁₅—O—C(O)—C(CH₃)₂—CH₂—.

In yet another embodiment of the present invention, R₂ represents R₁₅—O—C(O)—(C(CH₃)₂)ₜ-phenylene- or R₁₅—O—C(O)-phenylene-,

wherein t is an integer of 0, 1,
R₁₅ represent hydrogen or methyl.
and the phenylene ring is optionally substituted with methoxy.

In yet another embodiment of the present invention, A represents 1-naphthyl or phenyl,

wherein X represents —CH₂—, cis —CH═CH—CH₂— or trans —CH═CH—CH₂—,
R₁ is methyl.

In yet another embodiment of the present invention, A represents 1-naphthyl or phenyl; X represents -phenylene-CH₂—, the phenylene being optionally substituted with halogen or hydroxyl; R₁ is methyl.

In yet another embodiment of the present invention, R₂ represents —CR₁₃R₁₄—C(O)—NR₁₆R₁₇, wherein R₁₃, R₁₄ independently of each other are as defined above; and wherein R₁₆, R₁₇ independently represents hydrogen, C₁₋₃alkyl, C₃₋₆heterocycloalkyl or C₆₋₁₀aryl substituted with one or more, same or different substituents selected from hydroxy, C₁₋₃alkyl, C₁₋₃alkoxy, C₁₋₄hydroxyalkyl, C₃₋₆heterocycloalkyl, C₁₋₁₀heterocycloalkylaryl, C₁₋₁₀heteroaryl, C₆₋₁₀aryl, wherein said C₁₋₃alkyl, C₁₋₃alkoxy, C₁₋₄hydroxyalkyl, C₃₋₆heterocycloalkyl, C₁₋₁₀heteroaryl or C₆₋₁₀aryl, is optionally further substituted with methyl, tert-butyl, C₆₋₁₀aryl, halogen, methoxy, C₁₋₄alkoxycarbonyl or trifluoromethyl,

or R₁₆ and R₁₇ together with the N-atom to which they are attached form a C₁₋₆heterocycloalkyl ring optionally substituted with hydroxyl, C₁₋₃alkyl or phenyl the last two substituents optionally substituted with fluoro or methoxy.

In yet another embodiment of the present invention, A represents 1-naphthyl,

wherein X represents —CH═CH—CH₂—,
R₁ is methyl.

In yet another embodiment of the present invention, R₂ represents

### —C(CH₃)₂—C(O)—NR₁₈R₁₉,

wherein R₁₈ represents hydrogen or methyl,
and wherein R₁₉ represents pyridylmethylene, morpholinopropyl, diphenylmethylene, diphenylethylene, phenylpiperidinoethylene, hydroxyethylene, dimethyloxazolylmethylene, methoxycarbonylbenzyl, benzodioxolmethylene, trifluoromethylphenylethyl, methoxyphenylethyl, difluorobenzyl, tert-butylbenzyl, bromobenzyl phenylhydroxypropyl, diphenylhydroxyethyl, hydroxyindanyl, benzylpiperidyl, chlorobenzyl, phenylethylene, phenylethyl, benzyl, pyrrolidyloxodimethylethyl or ethoxyethylene.

In yet another embodiment of the present invention, R₂ represents

—C(O)—C(CH₃)₂— substituted with pyrrolidinyl, hydroxypyrrolidinyl, methoxyethylpiperazinyl or hydroxypiperidino.

In yet another embodiment, the present invention relates to compounds of formula Ia,

wherein X represents —CH═CH—CH₂—, —CH₂—, —CH₂—CH₂—, or —CH₂—CH₂—CH₂—;
wherein A represents 1-naphthyl or phenyl, optionally substituted with hydroxy, halogen or methoxy;
R₁ is methyl;
and wherein R₂ is as defined above.

In yet another embodiment, the present invention relates to compounds of formula Ia wherein X represents -phenylene-CH₂—, -thienyl-CH₂—, -pyridyl-CH₂— wherein the phenylene, thienyl and pyridyl rings are optionally substituted with one or more substituents selected from hydroxy, fluoro or bromo;

wherein A represents 1-naphthyl or phenyl optionally substituted with methoxy.
R₁ is methyl
and R₂ is as defined above.

In yet another embodiment of the present invention A represents indolyl, benzothienyl, benzodioxol, methylphenylpyrazolyl,

R₁ is methyl;
X is cis or trans —CH═CH—CH₂— and
R₂ is tert-butyl.

Specific examples of compounds of formula I may be selected from the group consisting of
(R)-But-2-ynyl-(1-naphthalen-1-yl-ethyl)-amine,
(R)-(1-Naphthalen-1-yl-ethyl)-pent-4-ynyl-amine (compound 102),
(E)/(R)-(6,6-Dimethyl-hept-2-en-4-ynyl)-(1-naphthalen-1-yl-ethyl)-amine (compound 103),
(Z)/(R)-(6,6-Dimethyl-hept-2-en-4-ynyl)-(1-naphthalen-1-yl-ethyl)-amine (compound 104),
(E)/(R)-(5-Cyclopropyl-pent-2-en-4-ynyl)-(1-naphthalen-1-yl-ethyl)-amine (compound 105),
(E)/(R)-6-Methyl-(1-naphthalen-1-yl-ethyl)-hept-2-en-4-yne-1,6-diamine (compound 106),
(E)/(R)-6-(1-Naphthalen-1-yl-ethylamino)-hex-4-en-2-yn-1-ol (compound 107),
(E)/(R)-2-Methyl-7-(1-naphthalen-1-yl-ethylamino)-hept-5-en-3-yn-2-ol (compound 108),
(E)/(3R/S)-3-Methyl-8-[(R)-1-naphthalen-1-yl-ethylamino]-oct-6-en-4-yn-3-ol (compound 109),
(E)/(1R/S)-6-[(R)-1-naphthalen-1-yl-ethylamino]-1-phenyl-hex-4-en-2-yn-1-ol (compound 110),
(Z)/(R)-6-(1-Naphthalen-1-yl-ethylamino)-hex-4-en-2-ynoic acid (compound 111),
(E)/(R)-(1-Naphthalen-1-yl-ethyl)-(5-trimethylsilanyl-pent-2-en-4-ynyl)-amine (compound 112),
(Z)/(R)-(1-Naphthalen-1-yl-ethyl)-(5-trimethylsilanyl-pent-2-en-4-ynyl)-amine (compound 113),
(E)/(R)-(1-Naphthalen-1-yl-ethyl)-(6-trimethylsilanyl-hex-2-en-4-ynyl)-amine (compound 114),
(E)/(R)-(1-Naphthalen-1-yl-ethyl)-(5-phenyl-pent-2-en-4-ynyl)-amine (compound 115),
(E)/(R)-(1-Naphthalen-1-yl-ethyl)-(5-p-tolyl-pent-2-en-4-ynyl)-amine (compound 116),
(E)/(R)-(1-Naphthalen-1-yl-ethyl)-[5-(4-trifluoromethyl-phenyl)-pent-2-en-4-ynyl]-amine (compound 117),
(E)/(R)-(1-Naphthalen-1-yl-ethyl)-[5-(3-trifluoromethyl-phenyl)-pent-2-en-4-ynyl]-amine (compound 118),
(E)/(R)-[5-(2-Fluoro-phenyl)-pent-2-en-4-ynyl]-(1-naphthalen-1-yl-ethyl)-amine (compound 119),
(E)/(R)-[5-(2,4-Difluoro-phenyl)-pent-2-en-4-ynyl]-(1-naphthalen-1-yl-ethyl)-amine (compound 120),
(E)/(R)-[5-(4-Chloro-phenyl)-pent-2-en-4-ynyl]-(1-naphthalen-1-yl-ethyl)-amine (compound 121),
(E)/(R)-[5-(4-Bromo-phenyl)-pent-2-en-4-ynyl]-(1-naphthalen-1-yl-ethyl)-amine (compound 122),
(E)/(R)-4-[5-(1-Naphthalen-1-yl-ethylamino)-pent-3-en-1-ynyl]-benzonitrile (compound 123),
(E)/(R)-4-[5-(1-Naphthalen-1-yl-ethylamino)-pent-3-en-1-ynyl]-aniline (compound 124),
(E)/(R)-2-[5-(1-Naphthalen-1-yl-ethylamino)-pent-3-en-1-ynyl]-aniline (compound 125),
(E)/(R)-Dimethyl-{4-[5-(1-naphthalen-1-yl-ethylamino)-pent-3-en-1-ynyl]}-aniline (compound 126),
(E)/(R)-3-[5-(1-Naphthalen-1-yl-ethylamino)-pent-3-en-1-ynyl]-phenol (compound 127),
(E)/(R)-[5-(3-Methoxy-phenyl)-pent-2-en-4-ynyl]-(1-naphthalen-1-yl-ethyl)-amine (compound 128),
(E)/(R)-{4-[5-(1-Naphthalen-1-yl-ethylamino)-pent-3-en-1-ynyl]-phenyl}-methanol (compound 129),
(E)/(R)-(6,6-Dimethyl-hept-2-en-4-ynyl)-[1-(3-methoxy-phenyl)-ethyl]-amine, and
(Z)/(R)-(6,6-Dimethyl-hept-2-en-4-ynyl)-[1-(3-methoxy-phenyl)-ethyl]-amine,
   or a pharmaceutically acceptable salt, solvate, or ester thereof, such as
(R)-But-2-ynyl-(1-naphthalen-1-yl-ethyl)-amine hydrochloride (compound 101),
(E)/(R)-(6,6-Dimethyl-hept-2-en-4-ynyl)-[1-(3-methoxy-phenyl)-ethyl]-amine hydrochloride (compound 130),
(Z)/(R)-(6,6-Dimethyl-hept-2-en-4-ynyl)-[1-(3-methoxy-phenyl)-ethyl]amine hydrochloride (compound 131),
(R)-(1-Naphthalen-1-yl-ethyl)-prop-2-ynyl-amine (compound 132),
(R)-[1-(3-Methoxy-phenyl)-ethyl]-prop-2-ynyl-amine (compound 133),
(R)-But-3-ynyl-(1-naphthalen-1-yl-ethyl)-amine (compound 134),
(R)-Hex-5-ynyl-(1-naphthalen-1-yl-ethyl)-amine (compound 135),
(E)/(R)-(6,6-Dimethyl-hept-2-en-4-ynyl)-[1-(4-fluoro-3-methoxy-phenyl)-ethyl]-amine (compound 136),
(Z)/(R)-(6,6-Dimethyl-hept-2-en-4-ynyl)-[1-(4-fluoro-3-methoxy-phenyl)-ethyl]-amine (compound 137),
(E)/(R)-(6,6-Dimethyl-hept-2-en-4-ynyl)-[1-(1H-indol-7-yl)-ethyl]-amine (compound 138),
(Z)/(R)-(6,6-Dimethyl-hept-2-en-4-ynyl)-[1-(1H-indol-7-yl)-ethyl]-amine (compound 139),
(E)/(R)-(6,6-Dimethyl-hept-2-en-4-ynyl)-[1-(1H-indol-4-yl)-ethyl]-amine (compound 140),
(Z)/(R)-(6,6-Dimethyl-hept-2-en-4-ynyl)-[1-(1H-indol-4-yl)-ethyl]-amine (compound 141),
(E)/(R)-(1-Benzo[1,3]dioxol-5-yl-ethyl)-(6,6-dimethyl-hept-2-en-4-ynyl)-amine (compound 142),
(Z)/(R)-(1-Benzo[1,3]dioxol-5-yl-ethyl)-(6,6-dimethyl-hept-2-en-4-ynyl)-amine (compound 143),
(E)/(R)-(1-Benzo[b]thiophen-3-yl-ethyl)-(6,6-dimethyl-hept-2-en-4-ynyl)-amine (compound 144),
(E)/(R)-(1-Benzo[b]thiophen-3-yl-ethyl)-(6,6-dimethyl-hept-2-en-4-ynyl)-amine (compound 145),
(E)/(R)-(6,6-Dimethyl-hept-2-en-4-ynyl)-[1-(1-methyl-5-phenyl-1H-pyrazol-3-yl)-ethyl]amine (compound 146),
(Z)/(R)-(6,6-Dimethyl-hept-2-en-4-ynyl)-[1-(1-methyl-5-phenyl-1H-pyrazol-3-yl)-ethyl]-amine (compound 147),
(Z)/(R)-2-Methyl-7-(1-naphthalen-1-yl-ethylamino)-hept-5-en-3-yn-2-ol (compound 148),
(E)/(R)-9-(1-Naphthalen-1-yl-ethylamino)-non-7-en-5-yn-1-ol (compound 149),
(E)/(R)-9-(1-Naphthalen-1-yl-ethylamino)-non-7-en-5-ynenitrile (compound 150),
(E)/(R)-{2-[5-(1-Naphthalen-1-yl-ethylamino)-pent-3-en-1-ynyl]-phenyl}-methanol (compound 151),
(E,E)/((R)-8-(1-Naphthalen-1-yl-ethylamino)-octa-2,6-dien-4-yn-1-ol (compound 152),
(E)/(R)-3-Ethyl-8-(1-naphthalen-1-yl-ethylamino)-oct-6-en-4-yn-3-ol (compound 153),
(E)/(R)-(6-Methoxy-hex-2-en-4-ynyl)-(1-naphthalen-1-yl-ethyl)-amine hydrochloride (compound 154),
(E)/(R)-(6-Methoxy-6-methyl-hept-2-en-4-ynyl)-(1-naphthalen-1-yl-ethyl)-amine hydrochloride (compound 155),
(E)/(R)-7-(1-Naphthalen-1-yl-ethylamino)-hept-5-en-3-yn-1-ol hydrochloride (compound 156),
(E)/(R)-(6,6-Dimethyl-7-triisopropylsilanyloxy-hept-2-en-4-ynyl)-(1-naphthalen-1-yl-ethyl)-amine (compound 157),
(E)/(R)-2,2-Dimethyl-7-(1-naphthalen-1-yl-ethylamino)-hept-5-en-3-yn-1-ol hydrochloride (compound 158),
(E)/(2R/S)-7-[(R)-1-Naphthalen-1-yl-ethylamino]-hept-5-en-3-yn-2-ol hydrochloride (compound 159),
(E)/(R)-8-(1-Naphthalen-1-yl-ethylamino)-oct-6-en-4-yn-1-ol hydrochloride (compound 160),
(E)/(R)—N*6*,N*6*-Dimethyl-N*1*-(1-naphthalen-1-yl-ethyl)-hex-2-en-4-yne-1,6-diamine dihydrochloride (compound 161),
(E)/(R)—N*6*-Benzyl-N*6*-methyl-N*1*-(1-naphthalen-1-yl-ethyl)-hex-2-en-4-yne-1,6-diamine dihydrochloride (compound 162),
(E)/(R)—N*6*,N*6*-Diethyl-N*1*-(1-naphthalen-1-yl-ethyl)-hex-2-en-4-yne-1,6-diamine dihydrochloride (compound 163),
(E)/(R)—N*1*-(1-Naphthalen-1-yl-ethyl)-N*6*,N*6*-dipropyl-hex-2-en-4-yne-1,6-diamine dihydrochloride (compound 164),
(E)/(R)-(4-{5-[1-(3-Methoxy-phenyl)-ethylamino]-pent-3-en-1-ynyl}-phenyl)-methanol (compound 165),
(E)/(R)-7-[1-(3-Methoxy-phenyl)-ethylamino]-2-methyl-hept-5-en-3-yn-2-ol (compound 166),
(E)/(4R/S)-9-[(R)-1-(3-Methoxy-phenyl)-ethylamino]-non-7-en-5-yn-4-ol (compound 167),
(E)/(R)-Benzyl 2,2-dimethyl-7-(1-naphthalen-1-yl-ethylamino)-hept-5-en-3-ynoate (compound 168),
(E)/(R)-Methyl 2,2-dimethyl-8-(1-naphthalen-1-yl-ethylamino)-oct-6-en-4-ynoate (compound 169),
(Z)/(R)Benzyl 2,2-dimethyl-7-(1-naphthalen-1-yl-ethylamino)-hept-5-en-3-ynoate (compound 170),
(E)/(R)-tert-Butyl [6-(1-naphthalen-1-yl-ethylamino)-hex-4-en-2-ynyl]-carbamate (compound 171),
(R)-(1-Naphthalen-1-yl-ethyl)-[3-(3-trifluoromethyl-phenyl)-prop-2-ynyl]-amine hydrochloride (compound 172),
(R)-(1-Naphthalen-1-yl-ethyl)-[3-(4-trifluoromethyl-phenyl)-prop-2-ynyl]-amine hydrochloride (compound 173),
(R)-Methyl 2-methyl-2-{4-[3-(1-naphthalen-1-yl-ethylamino)-prop-1-ynyl]-phenyl}-propanoate hydrochloride (compound 174),
(R)-[1-(3-Methoxy-phenyl)-ethyl]-{3-[3-(4-methoxy-tetrahydro-pyran-4-yl)-phenyl]-prop-2-ynyl}-amine (compound 175),
(R)-[1-(3-Methoxy-phenyl)-ethyl]-{3-[3-(4-fluoro-tetrahydro-pyran-4-yl)-phenyl]-prop-2-ynyl}-amine (compound 176),
(R)-(1-Naphthalen-1-yl-ethyl)-(3-trimethylsilanylethynyl-benzyl)-amine (compound 177),
(R)-(1-Phenyl-ethyl)-(3-trimethylsilanylethynyl-benzyl)-amine (compound 178),
(R)-(1-Naphthalen-1-yl-ethyl)-(4-trimethylsilanylethynyl-benzyl)-amine (compound 179),
(R)-(1-Phenyl-ethyl)-(4-trimethylsilanylethynyl-benzyl)-amine (compound 180),
(R)-3-{3-[(1-Naphthalen-1-yl-ethylamino)-methyl]-phenyl}-prop-2-yn-1-ol (compound 181),
(R)-2-Methyl-4-{3-[(1-naphthalen-1-yl-ethylamino)-methyl]-phenyl}-but-3-yn-2-ol (compound 182),
(R)-2-Methyl-4-{2-[(1-phenyl-ethylamino)-methyl]-phenyl}-but-3-yn-2-ol (compound 183),
(R)-1,1-Dimethyl-3-{3-[(1-naphthalen-1-yl-ethylamino)-methyl]-phenyl}-prop-2-ynylamine (compound 184),
(R)-(1-Phenyl-ethyl)-(4-phenylethynyl-benzyl)-amine (compound 185),
(R)-(1-Naphthalen-1-yl-ethyl)-(4-phenylethynyl-benzyl)-amine (compound 186),
(R)-3-{4-[(1-Naphthalen-1-yl-ethylamino)-methyl]-phenyl}-prop-2-yn-1-ol (compound 187),
(R)-3-{4-[(1-Phenyl-ethylamino)-methyl]-phenyl}-prop-2-yn-1-ol (compound 188),
(R)-4-{4-[(1-Naphthalen-1-yl-ethylamino)-methyl]-phenyl}-but-3-yn-1-ol (compound 189),
(R)-4-{4-[(1-Phenyl-ethylamino)-methyl]-phenyl}-but-3-yn-1-ol (compound 190),
(R)-(1-Naphthalen-1-yl-ethyl)-(3-phenylethynyl-benzyl)-amine (compound 191),
(R)-(1-Phenyl-ethyl)-(3-phenylethynyl-benzyl)-amine (compound 192),
(R)-Benzyl 2,2-dimethyl-4-{3-[(1-phenyl-ethylamino)-methyl]-phenyl}-but-3-ynoate (compound 193),
(R)-Benzyl 4-{4-fluoro-3-[(1-phenyl-ethylamino)-methyl]-phenyl}-2,2-dimethyl-but-3-ynoate (compound 194),
(R)-Benzyl 2,2-dimethyl-4-{3-[(1-naphthalen-1-yl-ethylamino)-methyl]-phenyl}-but-3-ynoate (compound 195),
(R)-2-Methyl-4-{3-[(1-phenyl-ethylamino)-methyl]-phenyl}-but-3-yn-2-ol (compound 196),
(R)-4-{4-Fluoro-3-[(1-phenyl-ethylamino)-methyl]-phenyl}-2-methyl-but-3-yn-2-ol (compound 197),
(R)-4-{4-Fluoro-3-[(1-naphthalen-1-yl-ethylamino)-methyl]-phenyl}-2-methyl-but-3-yn-2-ol (compound 198),
(R)-2-Methyl-4-{6-[(1-naphthalen-1-yl-ethylamino)-methyl]-pyridin-2-yl}-but-3-yn-2-ol (compound 199),
(R)-(3-{3-[(1-Phenyl-ethylamino)-methyl]-phenyl}-prop-2-ynyl)-bis-(4-trifluoromethyl-benzyl)-amine (compound 200),
(R)-Diethyl-(3-{3-[(1-naphthalen-1-yl-ethylamino)-methyl]-phenyl}-prop-2-ynyl)-amine (compound 201),
(R)-Benzyl 2,2-dimethyl-4-{4-[(1-naphthalen-1-yl-ethylamino)-methyl]-phenyl}-but-3-ynoate (compound 202),
(R)-Benzyl 4-(4-{[1-(3-methoxy-phenyl)-ethylamino]-methyl}-phenyl)-2,2-dimethyl-but-3-ynoate (compound 203),
(R)-(4-Ethynyl-benzyl)-(1-naphthalen-1-yl-ethyl)-amine (compound 204),
(R)-(4-Ethynyl-benzyl)-(1-phenyl-ethyl)-amine (compound 205),
(R)-2-(3-Hydroxy-3-methyl-but-1-ynyl)-5-[(1-naphthalen-1-yl-ethylamino)-methyl]-phenol (compound 206),
(R)-2-(3-Diethylamino-prop-1-ynyl)-5-[(1-naphthalen-1-yl-ethylamino)-methyl]-phenol (compound 207),
(R)-Benzyl 4-{2-Hydroxy-4-[(1-naphthalen-1-yl-ethylamino)-methyl]-phenyl}-2,2-dimethyl-but-3-ynoate (compound 208),
(R)-(2-Ethynyl-benzyl)-(1-naphthalen-1-yl-ethyl)-amine (compound 209),
(R)-(2-Ethynyl-benzyl)-(1-phenyl-ethyl)-amine (compound 210),
(R)-2-Methyl-4-{5-[(1-naphthalen-1-yl-ethylamino)-methyl]-thiophen-2-yl}-but-3-yn-2-ol (compound 211),
(R)-(1-Naphthalen-1-yl-ethyl)-(4-phenylethynyl-thiophen-2-ylmethyl)-amine (compound 212),
(R)-(1-Naphthalen-1-yl-ethyl)-(5-phenylethynyl-thiophen-2-ylmethyl)-amine (compound 213),
(R)-3-{5-[(1-Naphthalen-1-yl-ethylamino)-methyl]-thiophen-3-yl}-prop-2-yn-1-ol (compound 214),
(R)-3-{5-[(1-Phenyl-ethylamino)-methyl]-thiophen-3-yl}-prop-2-yn-1-ol (compound 215),
(R)-(1-Phenyl-ethyl)-(5-phenylethynyl-thiophen-2-ylmethyl)-amine (compound 216),
(R)-(1-Phenyl-ethyl)-(4-phenylethynyl-thiophen-2-ylmethyl)-amine (compound 217),
(E)/(R)-2-Ethyl-N-[6-(1-naphthalen-1-yl-ethylamino)-hex-4-en-2-ynyl]-butyramide (compound 218),
(E)/(R)-1-Methyl-piperidine-4-carboxylic acid [6-(1-naphthalen-1-yl-ethylamino)-hex-4-en-2-ynyl]-amide (compound 219),
(E)/(R)-3,3-Dimethyl-N-[6-(1-naphthalen-1-yl-ethylamino)-hex-4-en-2-ynyl]-butyramide (compound 220),
(E)/(R)—N-[7-(1-Naphthalen-1-yl-ethylamino)-hept-5-en-3-ynyl]-butyramide (compound 221),
(E)/(R)-3,3-Dimethyl-N-[7-(1-naphthalen-1-yl-ethylamino)-hept-5-en-3-ynyl]-butyramide (compound 222),
(E)/(R)-2-Ethyl-N-[7-(1-naphthalen-1-yl-ethylamino)-hept-5-en-3-ynyl]-butyramide (compound 223),
(E)/(R)-2-Methoxy-N-[7-(1-naphthalen-1-yl-ethylamino)-hept-5-en-3-ynyl]-acetamide (compound 224),
(E)/(R)-1-Methyl-piperidine-4-carboxylic acid [7-(1-naphthalen-1-yl-ethylamino)-hept-5-en-3-ynyl]-amide (compound 225),
(E)-/(R)—N-[7-(1-Naphthalen-1-yl-ethylamino)-hept-5-en-3-ynyl]-formamide (compound 226),
(R)—N-[7-(1-Naphthalen-1-yl-ethylamino)-hept-5-en-3-ynyl]isobutyramide (compound 227),
(E)/(R)-But-2-enoic acid [2,2-dimethyl-7-(1-naphthalen-1-yl-ethylamino)-hept-5-en-3-ynyl]-amide (compound 228),
(E)/(R)-Cyclohex-3-enecarboxylic acid [2,2-dimethyl-7-(1-naphthalen-1-yl-ethylamino)-hept-5-en-3-ynyl]-amide hydrogen hexafluorophosphate (compound 229),
(E)/(R)-Pent-4-ynoic acid [2,2-dimethyl-7-(1-naphthalen-1-yl-ethylamino)-hept-5-en-3-ynyl]-amide hydrogen hexafluorophosphate (compound 230),
(E)/(R)—N-[2,2-Dimethyl-7-(1-naphthalen-1-yl-ethylamino)-hept-5-en-3-ynyl]-2,4-dihydroxy-benzamide (compound 231),
(E)/(R)-Cyclopropanecarboxylic acid [2,2-dimethyl-7-(1-naphthalen-1-yl-ethylamino)-hept-5-en-3-ynyl]-amide hydrogen hexafluorophosphate (compound 232),
(E)/(R)-Ethanesulfonic acid [6-(1-naphthalen-1-yl-ethylamino)-hex-4-en-2-ynyl]-amide (compound 233),
(E)/(R)-Propane-1-sulfonic acid [6-(1-naphthalen-1-yl-ethylamino)-hex-4-en-2-ynyl]-amide (compound 234),
(E)/(R)-Butane-1-sulfonic acid [6-(1-naphthalen-1-yl-ethylamino)-hex-4-en-2-ynyl]-amide (compound 235),
(E)/(R)-4-Methyl-N-[7-(1-naphthalen-1-yl-ethylamino)-hept-5-en-3-ynyl]-benzenesulfonamide (compound 236),
(E)/(R)-2-Chloro-N-[7-(1-naphthalen-1-yl-ethylamino)-hept-5-en-3-ynyl]-5-trifluoromethyl-benzenesulfonamide (compound 237),
(E)/(R)-4-Methoxy-N-[7-(1-naphthalen-1-yl-ethylamino)-hept-5-en-3-ynyl]-benzenesulfonamide (compound 238),
(E)/(R)-Ethanesulfonic acid [7-(1-naphthalen-1-yl-ethylamino)-hept-5-en-3-ynyl]-amide (compound 239),
(E)/(R)-Propane-1-sulfonic acid [7-(1-naphthalen-1-yl-ethylamino)-hept-5-en-3-ynyl]-amide (compound 240),
(E)/(R)-Butane-1-sulfonic acid [7-(1-naphthalen-1-yl-ethylamino)-hept-5-en-3-ynyl]-amide (compound 241),
(E)/(R)-1-Ethyl-3-[6-(1-naphthalen-1-yl-ethylamino)-hex-4-en-2-ynyl]urea (compound 242),
(E)/(R)-1-(2,6-Dimethoxy-phenyl)-3-[6-(1-naphthalen-1-yl-ethylamino)-hex-4-en-2-ynyl]-urea (compound 243),
(E)/(R)-1-Ethyl-3-[7-(1-naphthalen-1-yl-ethylamino)-hept-5-en-3-ynyl]urea (compound 244),
(E)/(R)-1-[7-(1-Naphthalen-1-yl-ethylamino)-hept-5-en-3-ynyl]-3-propyl-urea (compound 245),
(E)/(R)-1-Isopropyl-3-[7-(1-naphthalen-1-yl-ethylamino)-hept-5-en-3-ynyl]-urea (compound 246),
(E)/(R)-1-Cyclohexyl-3-[7-(1-naphthalen-1-yl-ethylamino)-hept-5-en-3-ynyl]-urea (compound 247),
(E)/(R)-1-[7-(1-Naphthalen-1-yl-ethylamino)-hept-5-en-3-ynyl]-3-phenyl-urea (compound 248),
(E)/(R)-1-(3,5-Bis-trifluoromethyl-phenyl)-3-[7-(1-naphthalen-1-yl-ethylamino)-hept-5-en-3-ynyl]-urea (compound 249),
(E)/(R)-1-[7-(1-Naphthalen-1-yl-ethylamino)-hept-5-en-3-ynyl]-3-(3-trifluoromethyl-phenyl)-urea (compound 250),
(E)/(R)-1-(2,4-Dimethoxy-phenyl)-3-[7-(1-naphthalen-1-yl-ethylamino)-hept-5-en-3-ynyl]-urea (compound 251),
(E)/(R)-2,2-Dimethyl-1-morpholin-4-yl-7-(1-naphthalen-1-yl-ethylamino)-hept-5-en-3-yn-1-one hydrochloride (compound 252),
(E)/(R)-2,2-Dimethyl-1-(4-methyl-piperazin-1-yl)-7-(1-naphthalen-1-yl-ethylamino)-hept-5-en-3-yn-1-one dihydrochloride (compound 253),
(E)/(R)-2,2-Dimethyl-7-(1-naphthalen-1-yl-ethylamino)-hept-5-en-3-ynoic acid (pyridin-3-ylmethyl)-amide (compound 254),
(E)/(R)-2,2-Dimethyl-7-(1-naphthalen-1-yl-ethylamino)-hept-5-en-3-ynoic acid (pyridin-4-ylmethyl)-amide (compound 255),
(E)/(R)-2,2-Dimethyl-7-(1-naphthalen-1-yl-ethylamino)-hept-5-en-3-ynoic acid (3-morpholin-4-yl-propyl)-amide (compound 256),
(E)/(R)-2,2-Dimethyl-7-(1-naphthalen-1-yl-ethylamino)-hept-5-en-3-ynoic acid benzhydryl-amide (compound 257),
(E)/(R)-2,2-Dimethyl-7-(1-naphthalen-1-yl-ethylamino)-hept-5-en-3-ynoic acid (3,3-diphenyl-propyl)-amide (compound 258),
(E)-1-[(R/S)]-3-Hydroxy-pyrrolidin-1-yl)-2,2-dimethyl-7-[(R)1-naphthalen-1-yl-ethylamino]-hept-5-en-3-yn-1-on (compound 259),
(E)/(R)-1-[4-(2-Methoxy-ethyl)-piperazin-1-yl]-2,2-dimethyl-7-(1-naphthalen-1-yl-ethylamino)-hept-5-en-3-yn-1-one (compound 260),
(E)-2,2-Dimethyl-7-[(R)-1-naphthalen-1-yl-ethylamino)-hept-5-en-3-ynoic acid [(R/S)-2-phenyl-2-piperidin-1-yl-ethyl]-amide (compound 261),
(E)/(R)-2,2-Dimethyl-7-(1-naphthalen-1-yl-ethylamino)-hept-5-en-3-ynoic acid (2-hydroxy-ethyl)-amide (compound 262),
(E)/(R)-1-(4-Hydroxy-piperidin-1-yl)-2,2-dimethyl-7-(1-naphthalen-1-yl-ethylamino)-hept-5-en-3-yn-1-one (compound 263),
(E)/(R)-2,2-Dimethyl-7-(1-naphthalen-1-yl-ethylamino)-hept-5-en-3-ynoic acid (2,5-dimethyl-oxazol-4-ylmethyl)-amide (compound 264),
(E)/(R)-3-{[2,2-Dimethyl-7-(1-naphthalen-1-yl-ethylamino)-hept-5-en-3-ynoylamino]-methyl}-benzoic acid methyl ester (compound 265),
(E)/(R)-2,2-Dimethyl-7-(1-naphthalen-1-yl-ethylamino)-hept-5-en-3-ynoic acid (benzo[1,3]dioxol-5-ylmethyl)-amide (compound 266),
(E)-2,2-Dimethyl-7-[(R)-1-naphthalen-1-yl-ethylamino)-hept-5-en-3-ynoic acid {1-[(S)-3-trifluoromethyl-phenyl)-ethyl}-amide (compound 267),
(E)-2,2-Dimethyl-7-[(R)-1-naphthalen-1-yl-ethylamino)-hept-5-en-3-ynoic acid {1-[(S)-3-methoxy-phenyl)-ethyl}-amide (compound 268),
(E)/(R)-2,2-Dimethyl-7-(1-naphthalen-1-yl-ethylamino)-hept-5-en-3-ynoic acid 3,5-difluoro-benzylamide (compound 269),
(E)/(R)-2,2-Dimethyl-7-(1-naphthalen-1-yl-ethylamino)-hept-5-en-3-ynoic acid 4-t-butyl-benzylamide (compound 270),
(E)/(R)-2,2-Dimethyl-7-(1-naphthalen-1-yl-ethylamino)-hept-5-en-3-ynoic acid 3-bromo-benzylamide (compound 271),
(E)-2,2-Dimethyl-7-[(R)-1-naphthalen-1-yl-ethylamino]-hept-5-en-3-ynoic acid [(R/S)-3-hydroxy-1-phenyl-propyl)-amide (compound 272),
(E)-2,2-Dimethyl-7-[(R)-1-naphthalen-1-yl-ethylamino)-hept-5-en-3-ynoic acid [(1R,2R)-2-hydroxy-1,2-diphenyl-ethyl]-amide (compound 273),
(E)-2,2-Dimethyl-7-[(R)-1-naphthalen-1-yl-ethylamino)-hept-5-en-3-ynoic acid [(1S,2S)-2-hydroxy-1,2-diphenyl-ethyl]-amide (compound 274),
(E)-2,2-Dimethyl-7-[(R)-1-naphthalen-1-yl-ethylamino]-hept-5-en-3-ynoic acid [(1S,2R)-2-hydroxy-indan-1-yl]amide (compound 275),
(E)/(R)-2,2-Dimethyl-7-(1-naphthalen-1-yl-ethylamino)-hept-5-en-3-ynoic acid (1-benzyl-piperidin-4-yl)-amide (compound 276),
(E)/(R)-2,2-Dimethyl-7-(1-naphthalen-1-yl-ethylamino)-hept-5-en-3-ynoic acid 4-chloro-benzylamide (compound 277),
(E)/(R)-2,2-Dimethyl-7-(1-naphthalen-1-yl-ethylamino)-hept-5-en-3-ynoic acid phenethyl-amide (Compound 278),
(E)-2,2-Dimethyl-7-[(R)-1-naphthalen-1-yl-ethylamino]-hept-5-en-3-ynoic acid [(R/S)-1-phenyl-ethyl)-amide (Compound 279),
(E)/(R)-2,2-Dimethyl-7-(1-naphthalen-1-yl-ethylamino)-hept-5-en-3-ynoic acid benzylamide (compound 280),
(E)/(R)-2,2-Dimethyl-7-(1-naphthalen-1-yl-ethylamino)-1-pyrrolidin-1-yl-hept-5-en-3-yn-1-one (compound 281),
(E)/(R)-2,2-Dimethyl-7-(1-naphthalen-1-yl-ethylamino)-hept-5-en-3-ynoic acid (2-ethoxy-ethyl)-amide (compound 282),
(E)/(R)-2,2-Dimethyl-7-(1-naphthalen-1-yl-ethylamino)-hept-5-en-3-ynoic acid hydrochloride (compound 283),
(E)/(R)-2,2-Dimethyl-8-(1-naphthalen-1-yl-ethylamino)-oct-6-en-4-ynoic acid hydrochloride (compound 284),
(Z)/(R)-2,2-Dimethyl-7-(1-naphthalen-1-yl-ethylamino)-hept-5-en-3-ynoic acid hydrochloride (compound 285),
(R)-2-Methyl-2-{4-[3-(1-naphthalen-1-yl-ethylamino)-prop-1-ynyl]-phenyl}-propionic acid hydrochloride (compound 286),
(R)-2,2-Dimethyl-4-{3-[(1-naphthalen-1-yl-ethylamino)-methyl]-phenyl}-but-3-ynoic acid (compound 287),
(E)/(R)-6,6-Dimethyl-N*1*-(1-naphthalen-1-yl-ethyl)-hept-2-en-4-yne-1,7-diamine (compound 288),
(E)/(R)—N*1*-(1-naphthalen-1-yl-ethyl)-hept-2-en-4-yne-1,7-diamine (compound 289),
[(R)-1-Naphthalen-1-yl-ethyl]-[(R/S)-1-trifluoromethyl-but-3-ynyl]-amine (compound 290),
[(R/S)-8,8,8-Trifluoro-3-methylene]-7-[(R)-1-naphthalen-1-yl-ethylamino]-oct-4-yn-1-ol (compound 291),
2-{[(R/S)-5,5,5-Trifluoro]-4-[(R)-1-naphthalen-1-yl-ethylamino]-pent-1-ynyl}-phenylamine (compound 292),
4-Methoxy-3-{[(R/S)-5,5,5-trifluoro]-4-[(R)-1-naphthalen-1-yl-ethylamino]-pent-1-ynyl}-benzoic acid methyl ester (compound 293) or
(E)/(R)-(1-Naphthalen-1-yl-ethyl)-(6,6,6-trifluoro-hex-4-en-2-ynyl)-amine hydrochloride (compound 294).

### Methods of Preparation

The compounds of the present invention may be prepared in a number of ways well known to those skilled in the art of organic synthesis. The compounds of the present invention may be synthesised using the methods outlined below, together with methods known in the art of synthetic organic chemistry, or variations thereof as appreciated by those skilled in the art. Preferred methods include, but are not limited to, those described below.

The compounds of formula Ia and Ib may be prepared using the reactions and techniques described in this section. The reactions are performed in solvents that are appropriate with respect to the reagents and materials employed and that are suitable for the transformations being effected. Also, in the synthetic methods described below, it is to be understood that all proposed reaction conditions, including choice of solvent, reaction atmosphere, reaction temperature, duration of experiment and work-up procedures, are chosen to be conditions of standard for that reaction, which should be readily recognised by one skilled in the art. It is understood by one skilled in the art of organic synthesis, that the functionality present on various positions of the molecules used as the starting compounds or intermediates in the syntheses, must be compatible with the reagents and reactions proposed. Not all compounds of formula I falling into a given class may be compatible with some of the reaction conditions required in some of the methods described. Such restrictions of substituents or functional groups which are compatible with the reaction conditions will be readily apparent to one skilled in the art and alternative methods can be used.

Compounds of general formula I may be prepared by methods known in the art, where such a method is shown in Scheme I.

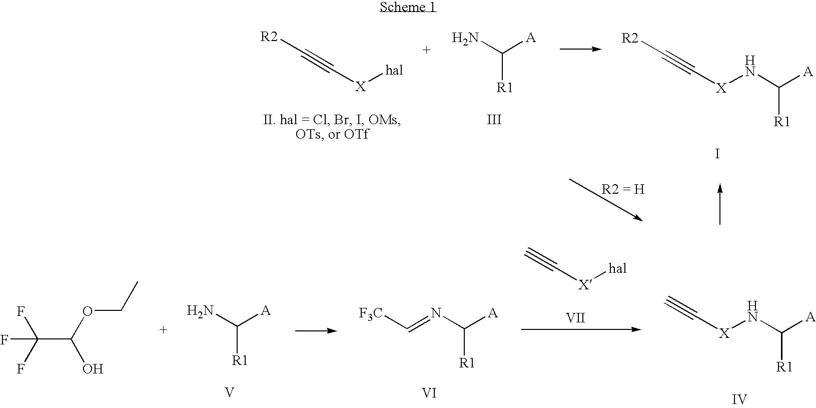

A, R1, R2, and X are defined as for general formula (I).

Hal represents a leaving group such as chloride, bromide, iodide, mesylate, tosylate, or triflate.

General formula (I) may be generated by treatment of formula (III) with formula (II) in the presence of a base, such as NEt₃, NaH, NaOH, KOH, or carbonates in an appropriate solvent such as DMF, DMSO, CH₃CN at e.g. ambient or higher temperature.

Where R2 is hydrogen, formula (IV) may first be generated.

Alternatively, IV may be formed by treatment of formula (V) with trifluoroacetaldehyde ethyl hemiacetal in a suitable solvent such as toluene at 110° C., followed by Barbier-type allylation of formula (VI) with formula (VII) in the presence of activated zinc (Zn*) at room temperature in DMF, or at reflux in THF (Gong, Y.; Kato, K. Tetrahedron: Asymmetry 2001, 12, 2121; Magueur, G.; Legros, J.; Meyer, F.; Ourévitch, M.; Crousse, B.; Bonnet-Delpon, D. Eur. J. Org. Chem. 2005, 1258). IV may secondly be converted to I by Sonogashira coupling with an alkenyl or (het)aryl halide in the presence 0f a Pd⁰/Cu^{I} catalyst system and a base such Et₂NH, Et₃N, or piperidine in a suitable solvent such as THF (Sonogashira, K.; Tohda, Y.; Hagihara, N. Tetrahedron Lett. 1975, 16, 4467; For reviews, see: Sonogashira, K. in Comprehensive Organic Synthesis, Trost, B. M., Fleming, I., Eds, Pergamon Press: New York, 1991, Vol. 3, p. 521; Rossi, R.; Carpita, A.; Bellina, F. Org. Prep. Proceed. Int. 1995, 27, 129; Truji, J. Palladium Reagents and Catalysts, Wiley: Chichester, UK, 1995, p. 168; Nicolaou, K. C.; Sorensen, E. J. Classics in Total Synthesis, VCH: Weinheim, Germany, 1996, p. 582; Sonogashira, K. in Metal-Catalyzed Cross-coupling Reactions, Diederich, F.; Stang, P. J., Eds, Wiley-VCH: Weinheim, Germany, 1998, p. 203; For synthesis of enynes, see: Alami, M; Ferri, F; Gaslain, Y. Tetrahedron Letters 1996, 37, 57).

An alternative route of preparation of compounds of general formula (I) is outlined in Scheme 2.

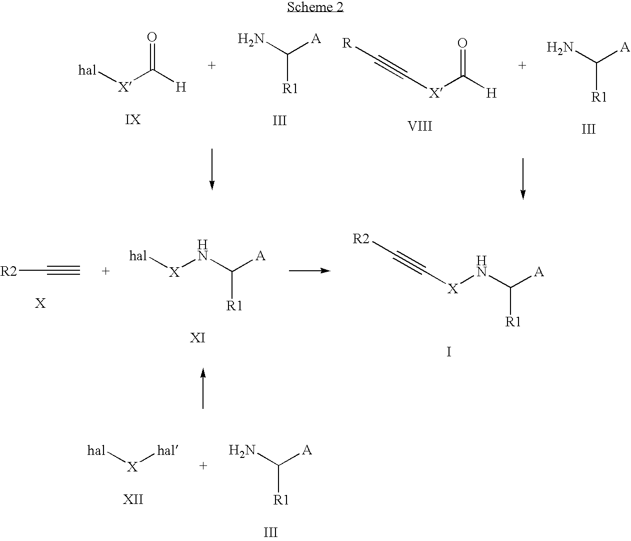

General formula (I) may be formed by treatment of III with formula (VIII) in the presence of a reducing agent such as NaBH₄, NaBH₃CN, and NaB(OAc)₃H.

Furthermore, general formula (I) may be generated by a two step synthesis. Formula XI may first be generated. Thus, alkylation of III by an allyl halide or a (het)arylmethyl halide IX as described for Scheme 1 gives a secondary amine XI.

Alternatively, XI may be formed by treatment of III with formula (XII) in the presence of a reducing agent. Secondly, Sonogashira coupling between formula (X) and XI furnishes I.

Scheme 3 depicts the synthesis of a compound of formula (I), which contains an amide function.

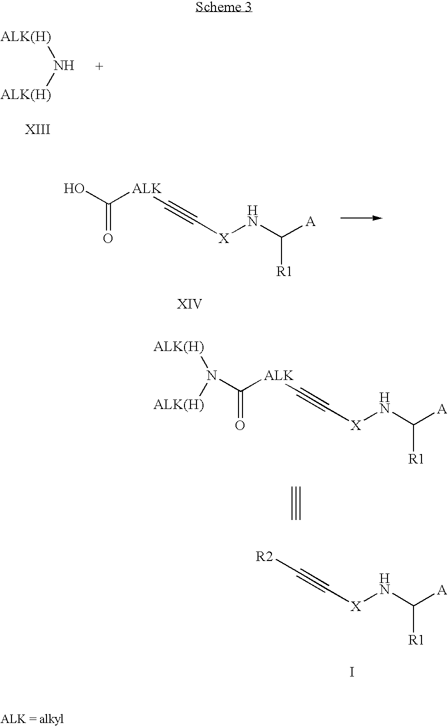

The conversion of formula (XIV) to I may be achieved by methods known in peptide chemistry; for example, the reaction may be performed using a coupling reagent such as DCC, HATU, and, pentafluorophenyl diphenyl-phosphate in a suitable solvent such as DCM, acetonitrile, and DMF.

Scheme 4 depicts the synthesis of a compound of formula (I), which contains a reversed amide, sulphonamide or urea function.

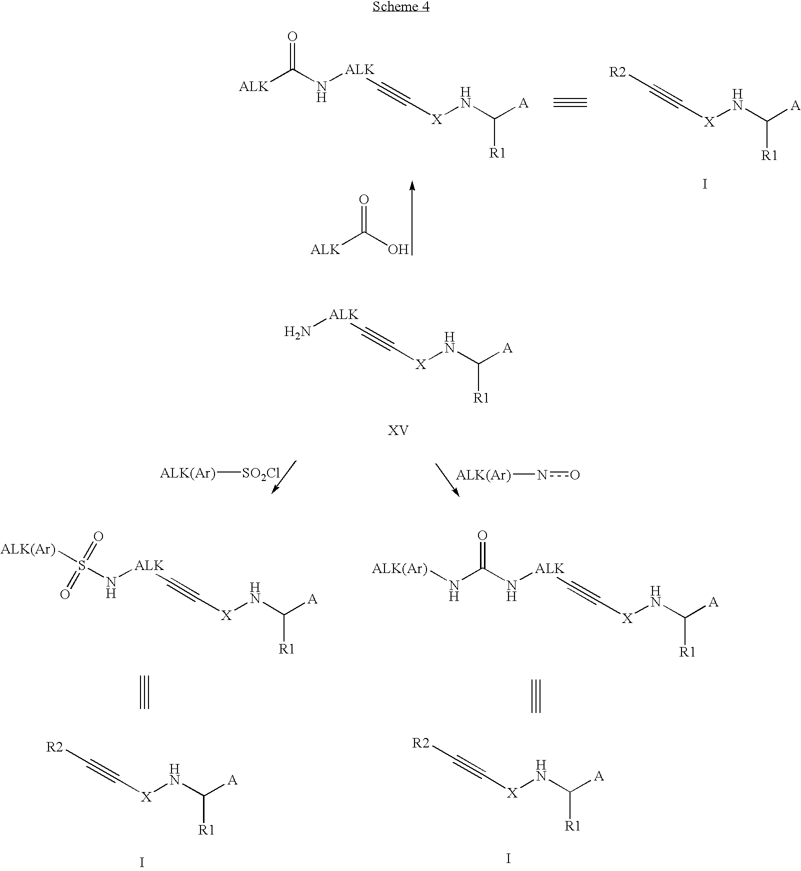

General formula (I), which contains a reversed amide function may be generated as describe for Scheme 3, while treatment of formula (XV) with a sulfonyl chloride or an aryl/alkyl isocyanate in the presence of a base such as Et₃N or DIPEA furnishes general formula (I), which contains a urea or sulphonamide function.

### Pharmaceutical Compositions

For use in therapy, compounds of the present invention are typically in the form of a pharmaceutical composition. The invention therefore relates to a pharmaceutical composition comprising a compound of formula I, optionally together with one or more other therapeutically active compound(s), together with a pharmaceutically acceptable excipient or vehicle. The excipient must be “acceptable” in the sense of being compatible with the other ingredients of the composition and not deleterious to the recipient thereof.

Conveniently, the active ingredient comprises from 0.05-99.9% by weight of the formulation.

Pharmaceutical compositions of the invention may be in unit dosage form such as tablets, pills, capsules, powders, granules, elixirs, syrups, emulsions, ampoules, suppositories or parenteral solutions or suspensions; for oral, parenteral, ophthalmic, transdermal, intra-articular, topical, pulmonal, nasal, buccal or rectal administration or in any other manner appropriate for the formulation of compounds used in nephrology and in accordance with accepted practices such as those disclosed in Remington: The The Science and Practice of Pharmacy, 21^{st} ed., 2000, Lippincott Williams & Wilkins. In the composition of the invention, the active component may be present in an amount of from about 0.01 to about 99%, such as 0.1% to about 10% by weight of the composition.

For oral administration in the form of a tablet or capsule, a compound of formula I may suitably be combined with an oral, non-toxic, pharmaceutically acceptable carrier such as ethanol, glycerol, water or the like. Furthermore, suitable binders, lubricants, disintegrating agents, flavouring agents and colourants may be added to the mixture, as appropriate. Suitable binders include, e.g., lactose, glucose, starch, gelatin, acacia gum, tragacanth gum, sodium alginate, carboxymethylcellulose, polyethylene glycol, waxes or the like. Lubricants include, e.g., sodium oleate, sodium stearate, magnesium stearate, sodium benzoate, sodium acetate, sodium chloride or the like. Disintegrating agents include, e.g., starch, methyl cellulose, agar, bentonite, xanthan gum or the like.

Additional excipients for capsules include macrogols or lipids.

For the preparation of solid compositions such as tablets, the active compound of formula I is mixed with one or more excipients, such as the ones described above, and other pharmaceutical diluents such as water to make a solid preformulation composition containing a homogenous mixture of a compound of formula I. The term “homogenous” is understood to mean that the compound of formula I is dispersed evenly throughout the composition so that the composition may readily be subdivided into equally effective unit dosage forms such as tablets or capsules. The preformulation composition may then be subdivided into unit dosage forms containing from about 0.05 to about 1000 mg, in particular from about 0.1 to about 500 mg, e.g. 10-200 mg, such as 30-180 mg, such as 20-50 mg of the active compound of the invention.

In the form of a dosage unit, the compound may be administered one or more times a day at appropriate intervals, always depending, however, on the condition of the patient, and in accordance with the prescription made by the medical practitioner. Conveniently, a dosage unit of a formulation contain between 0.1 mg and 1000 mg, preferably between 1 mg and 100 mg, such as 5-50 mg of a compound of formula Ia or Ib.

A suitable dosage of the compound of the invention will depend, inter alia, on the age and condition of the patient, the severity of the disease to be treated and other factors well known to the practising physician. The compound may be administered either orally, parenterally or topically according to different dosing schedules, e.g. daily or with weekly intervals. In general a single dose will be in the range from 0.01 to 400 mg/kg body weight. The compound may be administered as a bolus (i.e. the entire daily doses is administered at once) or in divided doses two or more times a day.

If the treatment involves administration of another therapeutically active compound it is recommended to consult Goodman & Gilman's The Pharmacological Basis of Therapeutics, 9^{th} Ed., J. G. Hardman and L. E. Limbird (Eds.), McGraw-Hill 1995, for useful dosages of said compounds. The administration of a compound of the present invention with one or more other active compounds may be either concomitantly or sequentially.

Liquid formulations for either oral or parenteral administration of the compound of the invention include, e.g., aqueous solutions, syrups, aqueous or oil suspensions and emulsion with edible oils such as cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose or polyvinylpyrolidone.

For parenteral administration, e.g. intramuscular, intraperitoneal, subcutaneous or intravenous injection or infusion, the pharmaceutical composition preferably comprises a compound of formula I dissolved or solubilised in an appropriate, pharmaceutically acceptable solvent. For parenteral administration, the composition of the invention may include a sterile aqueous or non-aqueous solvent, in particular water, isotonic saline, isotonic glucose solution, buffer solution or other solvent conventionally used for parenteral administration of therapeutically active substances. The composition may be sterilised by, for instance, filtration through a bacteria-retaining filter, addition of a sterilising agent to the composition, irradiation of the composition, or heating the composition. Alternatively, the compound of the invention may be provided as a sterile, solid preparation, e.g. a freeze-dried powder, which is dissolved in sterile solvent immediately prior to use.

The composition intended for parenteral administration may additionally comprise conventional additives such as stabilisers, buffers or preservatives, e.g. antioxidants such as methylhydroxybenzoate or the like.

Compositions for rectal administration may be in the form of a suppository incorporating the active ingredient and a carrier such as cocoa butter, or in the form of an enema.

Compositions suitable for intra-articular administration may be in the form of a sterile aqueous preparation of the active ingredient which may be in microcrystalline form, for example, in the form of an aqueous microcrystalline suspension. Liposomal formulations or biodegradable polymer systems may also be used to present the active ingredient for both intra-articular and ophthalmic administration.

Compositions suitable for topical administration, including ophthalmic treatment, include liquid or semi-liquid preparations such as liniments, lotions, gels, applicants, oil-in-water or water-in-oil emulsions such as creams, ointments or pastes; or solutions or suspensions such as drops. For topical administration, the compound of formula I may typically be present in an amount of from 0.01 to 20% by weight of the composition, such as 0.1% to about 10%, but may also be present in an amount of up to about 50% of the composition.

Compositions for ophthalmic treatment may preferably additionally contain a cyclodextrin.

Compositions suitable for administration to the nasal or buccal cavity or for inhalation include powder, self-propelling and spray formulations, such as aerosols and atomizers. Such compositions may comprise a compound of formula I in an amount of 0.01-20%, e.g. 2%, by weight of the composition.

The composition may additionally comprise one or more other active components conventionally used in the treatment of physiological disorders or diseases associated with disturbances of CaSR activity, such as hyperparathyroidism.

A suitable dosage of the compound of the invention will depend, inter alia, on the age and condition of the patient, the severity of the disease to be treated and other factors well known to the practising physician. The compound may be administered either orally, parenterally or topically according to different dosing schedules, e.g. daily or with weekly intervals. In general a single dose will be in the range from 0.01 to 400 mg/kg body weight. The compound may be administered as a bolus (i.e. the entire daily doses is administered at once) or in divided doses two or more times a day.

### Pharmacological Methods

The calcium sensing receptor (CaSR) and its use in identifying or screening for calcimimetic compounds has been described in EP637237, EP1296142, EP1100826, EP1,335,978, and EP1,594446.

In vitro and vivo methods for testing the compounds of the present invention are well established and may be found in the references listed above, or e.g. in Journal of Biological Chemistry (2004), 279(8), 7254-7263 or in U.S. Pat. No. 5,858,684 and references cited therein.

### Biological Assay for Analysis of In Vitro Activity

The assay investigates a compound's functional ability to act as a biological positive modulator on the human CaSR. Activation of the receptor expressed on CHO-K1 cells is detected through the G alpha q pathway, the activation of phospholipase C and the accumulation of intracellular inositol phosphate (IP) as described earlier [Sandrine Ferry, Bruno Chatel, Robert H. Dodd, Christine Lair, Danielle Gully, Jean-Pierre Maffrand, and Martial Ruat. Effects of Divalent Cations and of a Calcimimetic on Adrenocorticotropic Hormone Release in Pituitary Tumor Cells. BIOCHEMICAL AND BIOPHYSICAL RESEARCH COMMUNICATIONS 238, 866-873 (1997)]. The human CaSR is stably expressed on a CHO-K1 cell clone, stimulated with a basal level of calcium and challenged with the tested compound. The level of IP1 is determined using the IP-One htrf kit (Cisbio, France). CHO-K1 cells not transfected with the CaSR fail to elicit an IP1 response upon calcium and/or compound stimulation.

### Cloning of the Human CaSR Gene

The ORF coding for the human CaSR (genebank: NM_{—}000388) was acquired from Invitrogen Corp, USA and subsequently cloned into the mammalian expression vector pCDA3.1.

### Generation of Cell Line Expressing CaSR

CHO-K1 cells were transfected using Lipofectamine according to manufacturer's protocol (400.000 cells/well were seeded in a 6-well plate and transfected after 24 hours using 2 μg DNA and 5 μl lipofectamine). After another 24 hours the cells were detached, seeded and subjected to 1 mg/ml of G-418. Following 7 days growth single clones were picked, the CaSR expression evaluated using the 5C10 antibody against CaSR, the clones with the highest expression were selected and tested for functional response. The preferred clone was continuously cultured according to standard procedures described in ATCC (American Type Culture Collection) protocols for CHO-K1 with the addition of 500 μg/ml G-418.

### Functional Whole Cell Assay

On the assay day cells were harvested and resuspended to 13*10⁶ cells/ml in stimulation buffer (containing: Hepes 10 mM, MgCl₂ 0.5 mM, KCl 4.2 mM, NaCl 146 mM, glucose 5.5 mM, LiCl 50 mM at pH 7.4). Five μl cell solution were pipetted into a well (white 384-well plate, Perkin Elmer Optiplate) followed by 5 μl compound diluted in a Ca²⁺-containing (to the final concentration of 2 mM) buffer. After compound stimulation for 1 hour at 37° C. 10 ul of IP-One assay reagents were added and incubated for another 1 hour at room temperature. Finally the plate was read using a Perkin Elmer EnVision, according to protocol supplied by the IP-One assay kit manufacturer. The FRET ratio was calculated by dividing the 665 nm emission signal with that of the 615 nm.

Testing data of compounds of the present invention indicate that compounds of the present invention are potent modulators of CaSR, thus making them potentially useful in the treatment of diseases related to kidneys or bones.

As described above, the compounds described in the present invention are modulators of CaSR activity. The CaSR can be found in the parathyroid gland, the thyroid, bone cells, the stomach, the lung, the kidney, pituitary gland, the brain, the hypothalamus, the olfactory areas or the hippocampus. Compounds according to the present invention may preferably be more selective, in their use, with respect to the receptors of the parathyroid compared with those of the thyroid gland.

The compounds according to the invention, and the pharmaceutical compositions comprising them, may be used as a medicinal product, in particular for the treatment of physiological disorders or diseases associated with disturbances of CaSR activity. Even more particularly, these physiological disorders or diseases of the type including primary or secondary hyperparathyroidism, osteoporosis, cardiovascular, gastrointestinal, endocrine or neurodegenerative diseases or certain cancers in which (Ca²⁺)ₑ ions are abnormally high. The secondary hyperparathyroidism is more particularly observed in chronic renal failure.

Screening for P450 2D6 Inhibition The assay rapidly screen for potential inhibitors of human P450 2D6 catalytic activity, by using recombinant human P450 2D6. The IC50 determination is performed in duplicate at eight concentrations.

Incubations were conducted in 96 well microtiter plates based on a method described by BD Biosciences. To the first well in each row, a NADPH regenerating system and test compound was added. In the second well and all remaining wells, NADPH regenerating system and acetonitrile (final concentration of 2%) was added. The final assay concentration of the NADPH regenerating system was 8.2 μM NADP⁺, 0.41 mM glucose-6-phosphate, 0.41 mM magnesium chloride hexahydrate and 0.4 U/ml glucose-6-phosphate dehydrogenase and 0.01 mg/mL control insect cell membrane protein. The test compound solution was serially diluted 1:3 through the eighth wells. The final concentration of the test compounds were in the range 100 μM to 45.7 nM in the eight rows. Wells 9 and 10 contained no test compound (only NADPH regenerating system and enzyme/substrate mix) and wells 11 and 12 were used as controls for background fluorescence (enzyme and substrate were added after the reaction was terminated). The plate was then pre-incubated at 37° C. for 10 min, and the reaction was initiated by the addition of pre-warmed enzyme/substrate mix. The assay concentration of the enzyme/substrate mix was 100 mM potassium phosphate, pH 7.4, 1.5 pmol recombinant human P450 CYP2D6 and 1.5 μM of the fluorescent substrate 3-[2-(N,N diethyl-N-methylamino)ethyl]-7-methoxy-4-methylcoumarin (AMMC). The assay was conducted in duplicate in a final volume of 200 μL per well. Reactions were terminated after 30 min by addition of a 4:1, acetonitrile:0.5 M Tris base solution. Quinidine was used as positive control, 0.5 μM as highest concentration. Fluorescence per well was measured using a fluorescence plate reader (excitation: 390 nm, emission: 460 nm). The IC50 values were calculated.

Testing data of compounds of the present invention indicate that compounds of the present invention show low or no inhibition towards human P450 2D6 (pIC50-value below 6).

The invention is described in further detail in the following non-limiting examples which are not in any way intended to limit the scope of the invention as claimed.

### EXAMPLES

### General

All the starting materials used are commercially available, unless otherwise described.

For ¹H nuclear magnetic resonance (NMR) spectra (300 MHz) and ¹³C NMR (75.6 MHz) chemical shift values (δ) (in ppm) are quoted, unless otherwise specified; for deuteriochloroform solutions relative to internal tetramethylsilane (δ=0.00) or chloroform (δ=7.26) or deuteriochloroform (δ=76.81 for ¹³C NMR) standard. The value of a multiplet, either defined (doublet (d), triplet (t), quartet (q)) or not (m) at the approximate mid point is given unless a range is quoted. All organic solvents used were anhydrous.

For some of the compounds, only LC/MS data are given. Two methods for LC/MS analysis are used:

### Method A

Analytical HPLC/MS was performed on a Dionex APS-system with a P680A analytical pump and a Thermo MSQ Plus mass spectrometer. Column: Waters XTerra C-18, 150 mm×4.6 mm, 5 μm; solvent system: A=water (0.1% formic acid) and B=acetonitrile (0.1% formic acid); flow rate=1.0 mL/min; method (10 min): Linear gradient method going from 10% B to 100% in 6.6 minutes and staying at 100% B for another 1.5 minutes.

### Method B

Analytical UPLC/MS was performed on a Waters Acquity UPLC system with a Waters LCT Premier XE mass spectrometer. Column: Waters Acquity UPLC HSS T3 1.8 5 μm; solvent system: A=water (0.1% formic acid) and B=acetonitrile (0.1% formic acid); Method: 95% A and 5% B with a flow rate=0.350 mL/min for 0.5 minutes; Linear gradient going from 95% A and 5% B to 5% A and 95% B in 2.5 minutes and staying at this level for another 1.5 minutes with a flow rate=0.350 mL/min; Linear gradient going from 5% A and 95% B to 95% A and 5% B in 1 minutes with a flow rate=0.700 mL/min and then decreasing the flow rate to 0.35 mL/min during 0.8 minutes.

Flash chromatography was performed on silica gel. Appropriate mixtures of ethyl acetate, dichloromethane, methanol, and petroleum ether (40-60) were used as eluents unless otherwise noted.

HPLC purifications of the crude products were performed by using Waters LC-MS system [column: Waters X Terra C18, 5 μm or Luna C18 100 Å 5μ; Size: 250×10.00 mm (Phenomenex)]; Sample Manager: Waters 2767; Pump: Waters 2525; Single Quadrupole: Waters ZQ; PDA-detector: Waters 2996).

The following abbreviations have been used:
aq. aqueous
DAST (Diethylamino)sulphur trifluoride
1,2-DCE 1,2-Dichloroethane
DCM Dichloromethane
DIPEA Diisopropylethylamine
DMAP 4-Dimethylaminopyridine
DMF N,N-Dimethylformamide
DMSO Dimethylsulphoxide
Et₂O Diethyl ether
EtOAc Ethyl acetate
h Hour(s)
HATU O-(7-Azabenzotriazol-1-yl)-N,N,N′,N′-tetramethyluronium hexafluorophosphate
HPLC High performance liquid chromatography
min Minutes
NMR Nuclear magnetic resonance
PE Petroleum ether
rt Room temperature
TEA Triethylamine
TFA Trifluoroacetic acid
THF Tetrahydrofuran

### General Procedure 1 (GP1):

Alkylation of Amines with Alkylhalides

A mixture of alkyl halide (1 mmol), 1-aryl-ethylamine (1 mmol), and K₂CO₃ (2 mmol) in DMF (1 mL) was stirred at rt for 5 h. The reaction mixture was then poured into brine and extracted with Et₂O. The combined organic phases were washed with brine, dried over MgSO₄, and concentrated in vacuo. The crude was purified by chromatography.

### General Procedure 2 (GP2):

### a) Sonogashira Reaction

To a mixture of an alkenyl bromide or aryl iodide (1 mmol), (Ph₃P)₂PdCl₂ (35 mg, 0.05 mmol), and CuI (19 mg, 0.1 mmol) in diethyl amine (3 mL) was added a terminal alkyne (3.5 mmol) at rt. After being stirred for 3 h at this temperature, the reaction mixture was concentrated in vacuo together with silica gel. The crude was purified by chromatography.

### b) Sonogashira Reaction and HPLC Purification

To an alkenyl bromide (0.15 mmol) and an alkyne (0.3 mmol) in Et₂NH (1 mL) were added CuI (5 mg, 0.015 mmol) and (Ph₃P)₂PdCl₂ (5 mg, 0.007 mmol) at rt. After being stirred for 3.5 h at this temperature, the reaction mixture was treated with PL-BuSH MP-resin 100A from Polymer Laboratories, Ltd. UK (60 mg) to remove Pd. The mixture was then filtered. The filtrate was concentrated in vacuo. The crude product was dissolved in DMSO (0.3 mL) and subjected to HPLC purification [Column: XTerra 150×19.0 mm, 5 μM (Waters)]. 0.1% aq solution of HCO₂H (A)/0.1% CH₃CN solution of HCO₂H (B) were used as eluent.

Table for Gradient

| | flow rate | |
|---|---|---|
| time (min) | (mL/min) | % of B |
| 0.0 | 6 | 35 |
| 1.0 | 6 | 35 |
| 6.0 | 6 | 100 |
| 8.0 | 6 | 100 |
| 10.0 | 6 | 0 |

### c) Sonogashira Reaction and HPLC Purification

To a solution of an aryl halide (0.1 mmol) and a terminal alkyne (0.5 mmol) in Et₂NH (0.5 mmol) were added CuI (5 mg) and Pd(Ph₃P)₂Cl₂ (5 mg) at rt. The reaction mixture was shaken overnight at this temperature and concentrated in vacuo. The residue was taken up in 1,2-DCE and insoluble substances were filtered off. To the filtrate was added QuadraSil™ MTU (Reaxa Ltd, Manchester, UK). After being shaken for 15 min, the mixture was filtered. The filtrate was concentrated in vacuo. The residue was redissolved in DMSO (0.3 mL) and subjected to HPLC purification (For conditions, see GP3a).

### General Procedure 3 (GP3):

### Preparation of Amide

a)

A solution of a carboxylic acid (0.1 mmol) and pentafluorophenyl diphenyl-phosphate (0.12 mmol) in DMF (0.25 mL) was shaken for 1 h at rt. A solution of an amine (0.1 mmol) and DIPEA (DMF, 0.25 mL) was added. The reaction mixture was shaken at the same temperature overnight and then subjected to HPLC purification [Column: Luna C18 100 Å 5μ; Size: 250×10.00 mm (Phenomenex)]. 0.1% aq solution of HCO₂H (A)/0.1% CH₃CN solution of HCO₂H (B) was used as eluent (see Table for Gradient below).

Table for Gradient

| time (min)* | % of B |
|---|---|
| 0.0 | 10 |
| 0.2 | 10 |
| 6.0 | 100 |
| 8.0 | 100 |
| 8.1 | 10 |
| 10.0 | 10 |

Flow rate: 18 mL/min

b)

To a solution of a carboxylic acid (0.1 mmol) and HATU (0.12 mmol) in DMF (0.25 mL) was added a solution of an amine (0.1 mmol) in DMF (0.1 mL). The reaction mixture was shaken at the same temperature overnight and then subjected to HPLC purification purification (Conditions: see GP3a).

### General Procedure 4 (GP4):

### Preparation of Sulphonamide

To a solution of an amine (0.1 mmol) and DIPEA (0.3 mmol) in DMF (0.5 mL) was added a sulphonyl chloride (0.1 mmol) in DMF (0.5 mL) at rt. The reaction solution was diluted with 1,2-DCE and shaken overnight. The reaction mixture was filtered and concentrated in vacuo. The residue was redissolved in DMF (0.3 mL) and subjected to HPLC purification (Conditions: see GP3a).

### General Procedure 5 (GP5):

### Preparation of Urea

To a solution of an amine (0.1 mmol) and DIPEA (0.3 mmol) in DMF (0.5 mL) was added an isocyanate (0.1 mmol) in DMF (0.5 mL) at rt. The reaction solution was diluted with 1,2-DCE and shaken overnight. The reaction mixture was filtered and concentrated in vacuo. The residue was redissolved in DMF (0.3 mL) and subjected to HPLC purification (Conditions: see GP3).

### General Procedure 6 (GP6):

### Reductive Amination

To a solution of an amine (1 equiv) in 1,2-DCE (5 mL/mmol) was added an aldehyde (1.1 equiv) at rt. After the reaction solution was stirred at this temperature for 0.5 h, NaBH(OAc)₃ (1.4 equiv) was added. The obtained mixture was stirred overnight and filtered. The filtrate was concentrated in vacuo. The residue was purified by chromatography, giving the desired compound.

### General Procedure 7 (GP7):

### Preparation of Sulfinimine

To a mixture of an aldehyde (1.0 mmol) and (S)-(−)-2-methyl-2-propanesulfinamide (1.1 mmol) in 1,2-DCE (10 mL) at rt. Anhydrous CuSO₄ (3 mmol) was added and the reaction mixture was stirred at this temperature for 20 h (TLC control). Heating was necessary in some cases. After the reaction was complete, the reaction mixture was concentrated in vacuo together silica gel. The residue was purified by chromatography.

### General Procedure 8 (GP8):

### Preparation of Sulfinamine

To a solution of (S)-(−)-sulfinimine (1 mmol) was dissolved in dry DCM (10 mL) was added methylmagnesium bromide (1.3 mmol) dropwise at −40° C. under argon atmosphere.

After being stirred at this temperature for 0.5 h, the reaction solution was gradually warmed to rt and stirred (TLC control). After completion of the reaction, sat. aq. NH₄Cl was added. The mixture was diluted with DCM and washed with water and brine. The organic phase was dried over MgSO₄ and concentrated in vacuo.

The residue was purified by flash chromatography (heptane/EtOAc 1:0→0:1), giving the title compound.

### General Procedure 9 (GP9):

### Preparation of Free Amine

To a solution of a substituted sulfinamide (1.0 mmol) in methanol (4 ml) was added 4 N HCl in 1,4-dioxane (4 mL) at rt. The obtained reaction solution was stirred at this temperature (TLC control) and then taken up in EtOAc/0.1M aq HCl. To the aqueous phase was added 1 N NaOH until pH 10. The aqueous phase was extracted with EtOAc. The organic phases were dried over MgSO₄ and concentrated in vacuo, giving the desired free amine.

TABLE 1
Exemplified compounds of general formula Ia
Ia

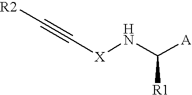

| Compound | Example | Structure |
|---|---|---|
| 101 | 1 | 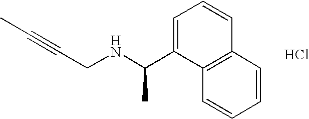 |
| 102 | 2 | 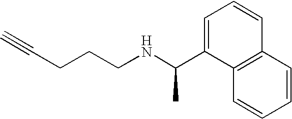 |
| 103 | 3 | 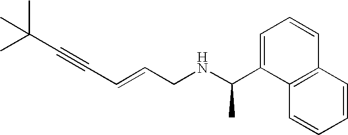 |
| 104 | 3 | 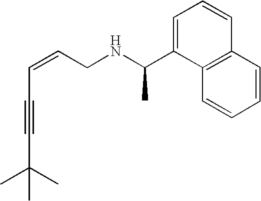 |
| 105 | 4 | 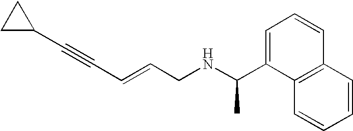 |
| 106 | 5 | 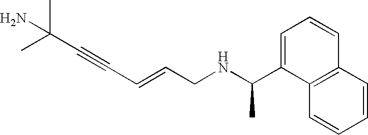 |
| 107 | 6 | 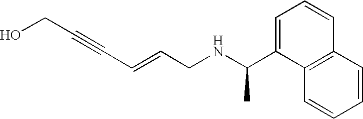 |
| 108 | 7 | 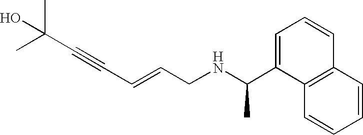 |
| 109 | 8 | 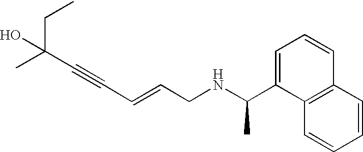 |
| 110 | 9 | 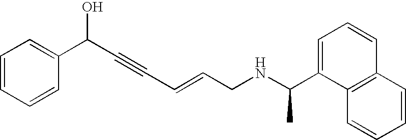 |
| 111 | 10 | 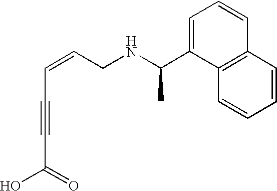 |
| 112 | 11 | 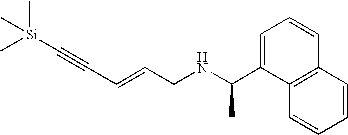 |
| 113 | 12 | 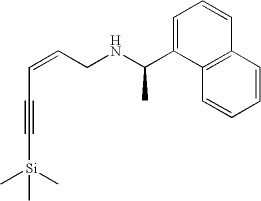 |
| 114 | 13 | 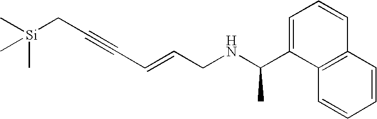 |
| 115 | 14 | 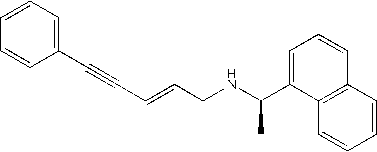 |
| 116 | 15 | 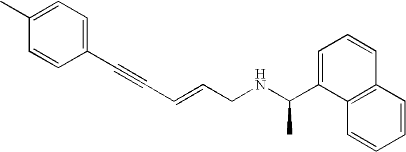 |
| 117 | 16 | 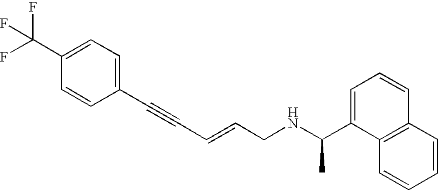 |
| 118 | 17 | 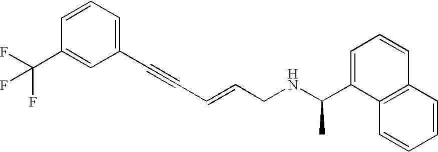 |
| 119 | 18 | 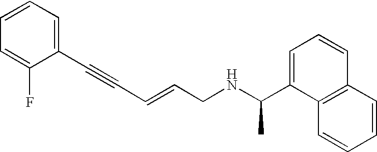 |
| 120 | 19 | 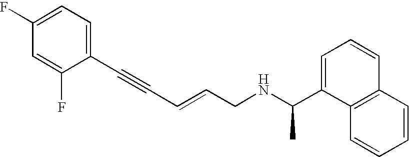 |
| 121 | 20 | 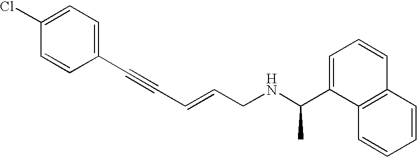 |
| 122 | 21 | 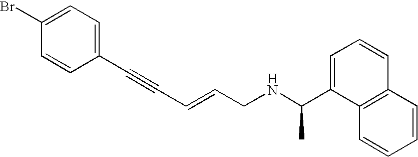 |
| 123 | 22 | 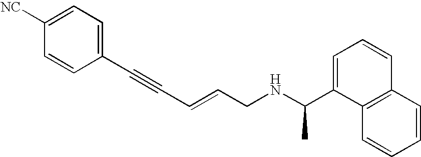 |
| 124 | 23 | 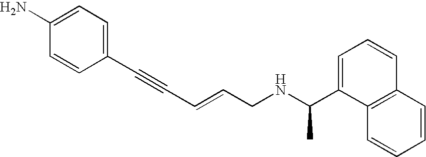 |
| 125 | 24 | 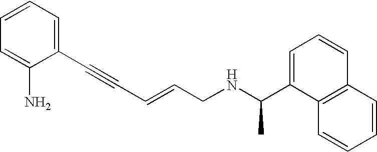 |
| 126 | 25 | 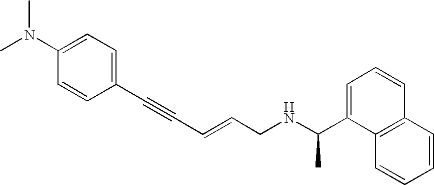 |
| 127 | 26 | 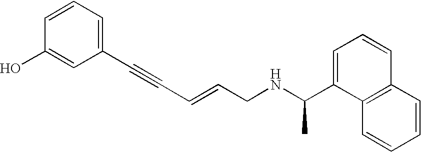 |
| 128 | 27 | 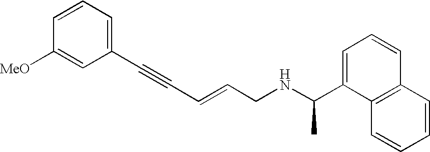 |
| 129 | 28 | 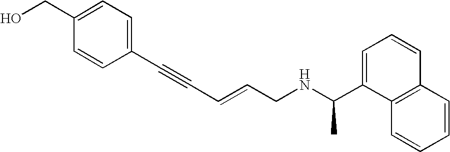 |
| 130 | 29 | 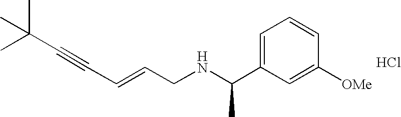 |
| 131 | 29 | 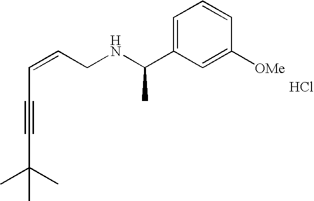 |
| 132 | 30 | 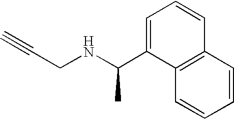 |
| 133 | 30 | 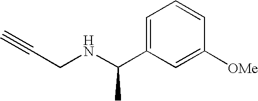 |
| 134 | 31 | 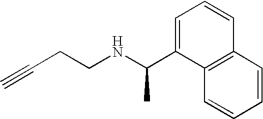 |
| 135 | 32 | 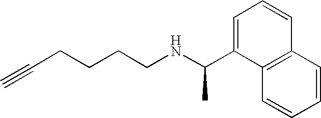 |
| 136 | 33 | 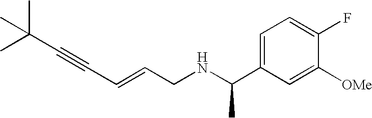 |
| 137 | 33 | 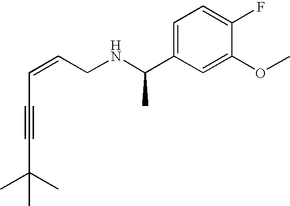 |
| 138 | 34 | 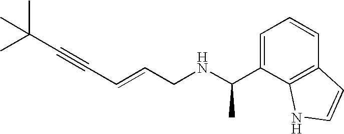 |
| 139 | 34 | 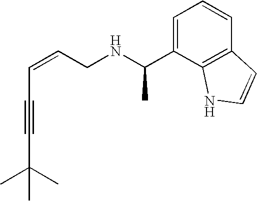 |
| 140 | 35 | 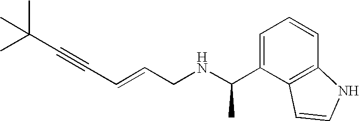 |
| 141 | 35 | 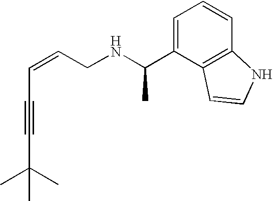 |
| 142 | 36 | 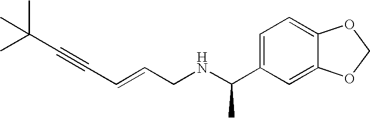 |
| 143 | 36 | 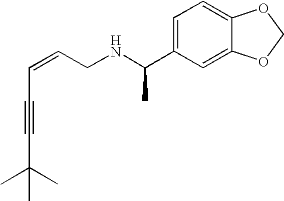 |
| 144 | 37 | 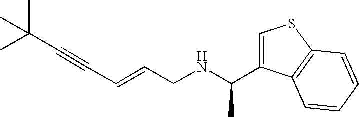 |
| 145 | 37 | 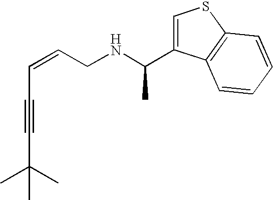 |
| 146 | 38 | 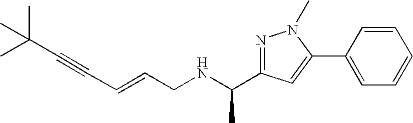 |
| 147 | 38 | 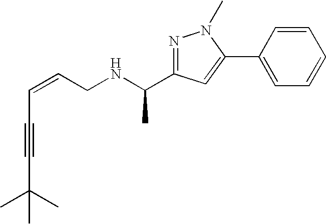 |
| 148 | 39 | 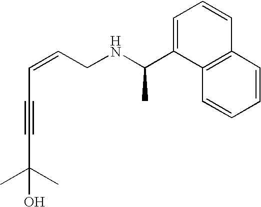 |
| 149 | 40 | 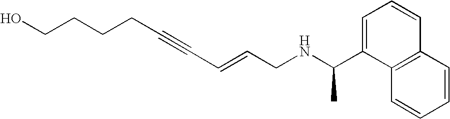 |
| 150 | 41 | 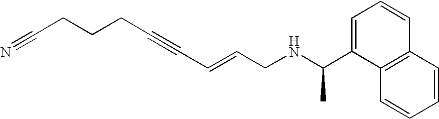 |
| 151 | 42 | 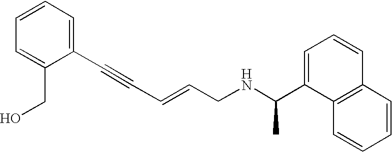 |
| 152 | 43 | 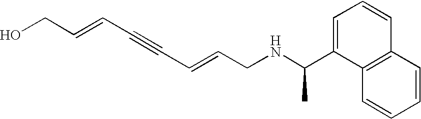 |
| 153 | 44 | 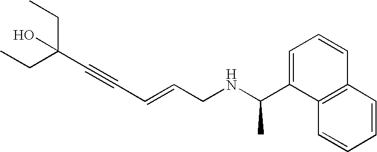 |
| 154 | 45 | 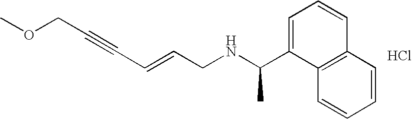 |
| 155 | 46 | 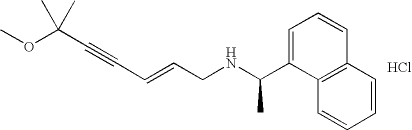 |
| 156 | 47 | 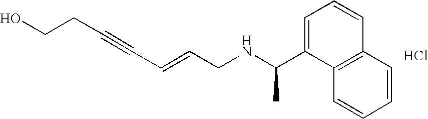 |
| 157 | 48 | 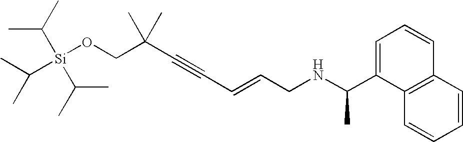 |
| 158 | 49 | 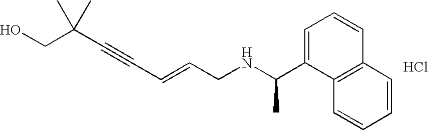 |
| 159 | 50 | 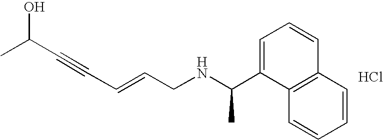 |
| 160 | 51 | 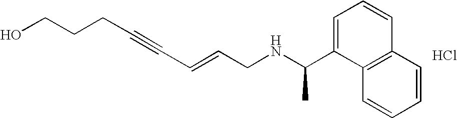 |
| 161 | 52 | 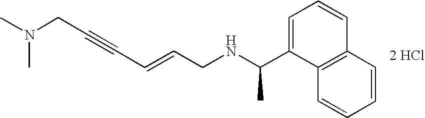 |
| 162 | 53 | 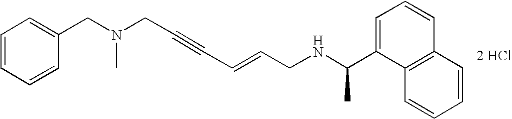 |
| 163 | 54 | 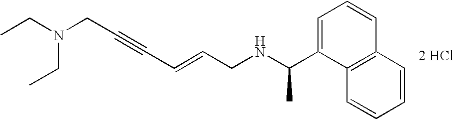 |
| 164 | 55 | 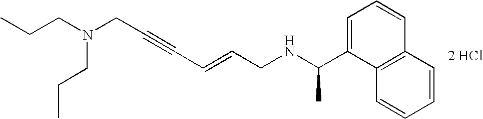 |
| 165 | 56 | 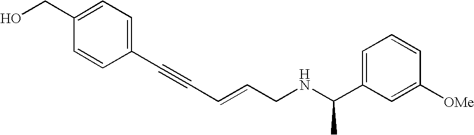 |
| 166 | 57 | 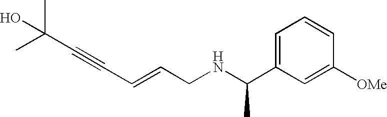 |
| 167 | 58 | 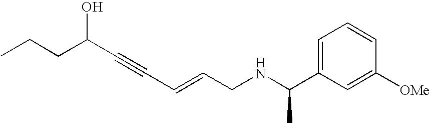 |
| 168 | 59 | 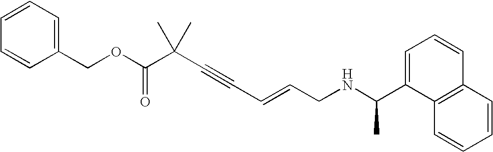 |
| 169 | 60 | 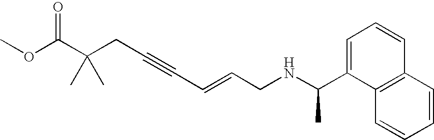 |
| 170 | 61 | 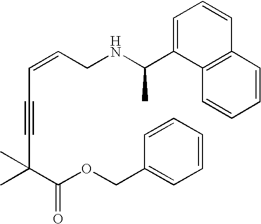 |
| 171 | 62 | 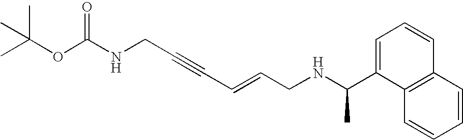 |
| 172 | 63 | 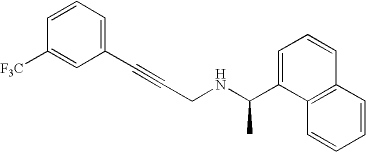 |
| 173 | 64 | 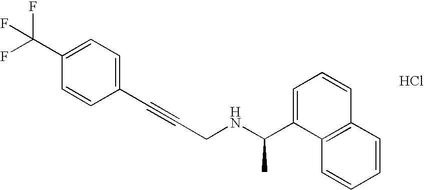 |
| 174 | 65 | 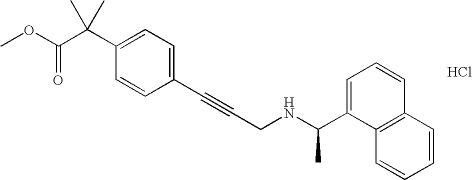 |
| 175 | 66 | 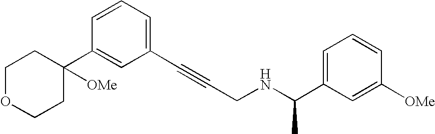 |
| 176 | 67 | 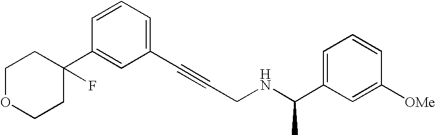 |
| 177 | 68 | 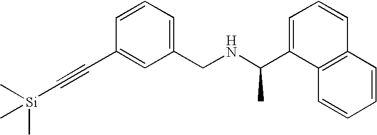 |
| 178 | 69 | 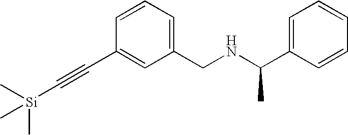 |
| 179 | 70 | 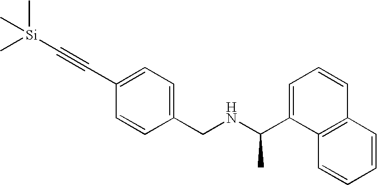 |
| 180 | 71 | 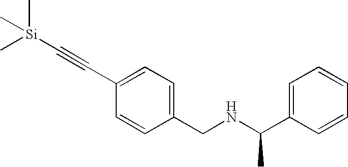 |
| 181 | 72 | 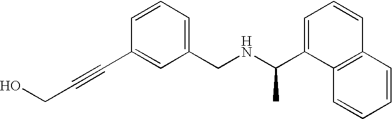 |
| 182 | 73 | 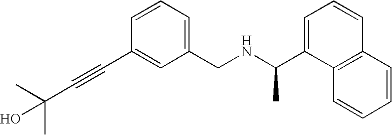 |
| 183 | 74 | 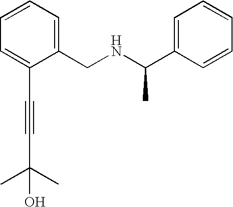 |
| 184 | 75 | 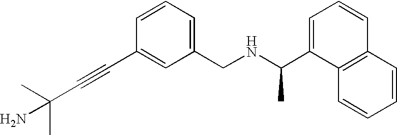 |
| 185 | 76 | 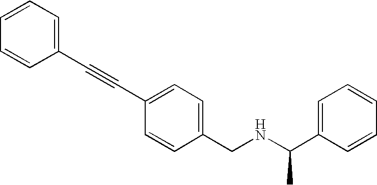 |
| 186 | 77 | 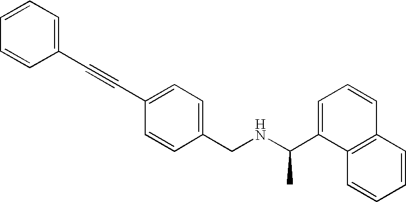 |
| 187 | 78 | 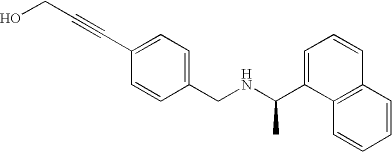 |
| 188 | 79 | 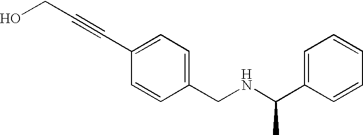 |
| 189 | 80 | 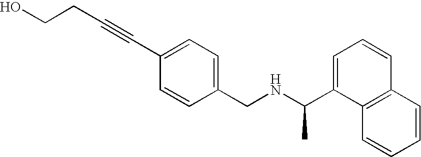 |
| 190 | 81 | 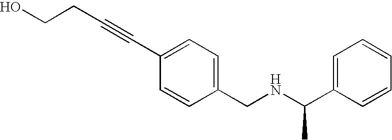 |
| 191 | 82 | 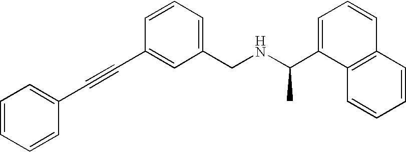 |
| 192 | 83 | 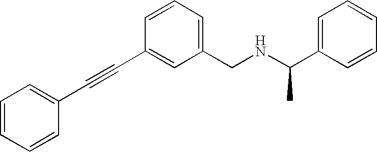 |
| 193 | 84 | 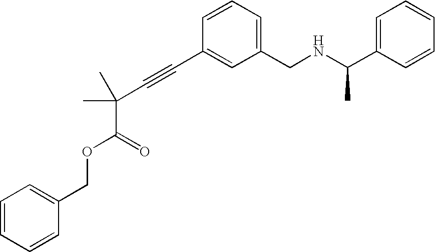 |
| 194 | 85 | 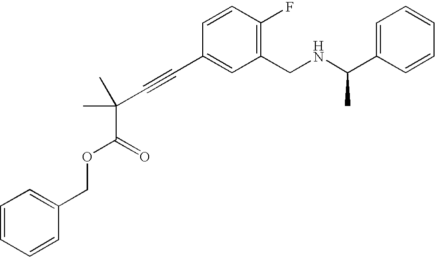 |
| 195 | 86 | 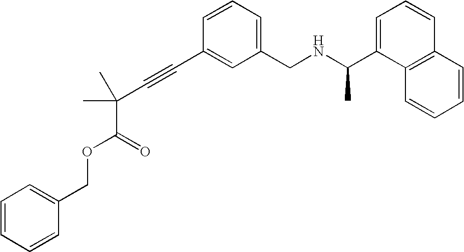 |
| 196 | 87 | 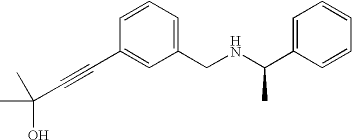 |
| 197 | 88 | 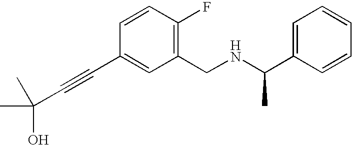 |
| 198 | 89 | 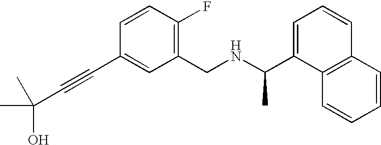 |
| 199 | 90 | 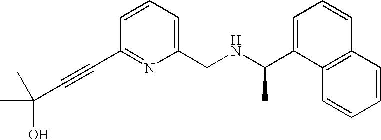 |
| 200 | 91 | 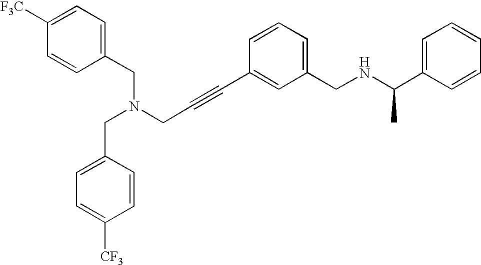 |
| 201 | 92 | 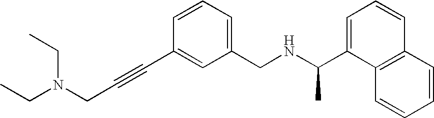 |
| 202 | 93 | 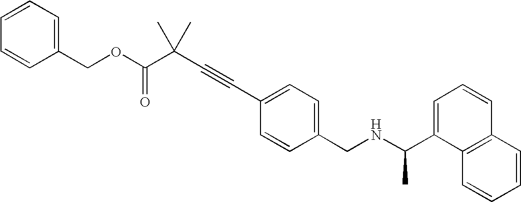 |
| 203 | 94 | 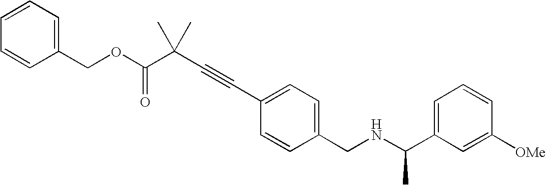 |
| 204 | 95 | 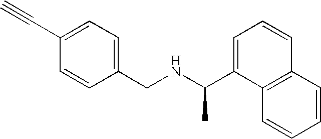 |
| 205 | 96 | 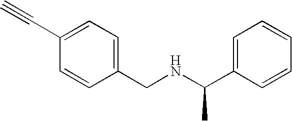 |
| 206 | 97 | 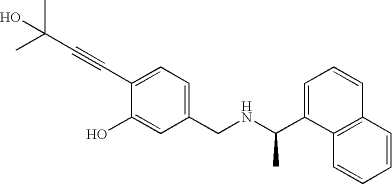 |
| 207 | 98 | 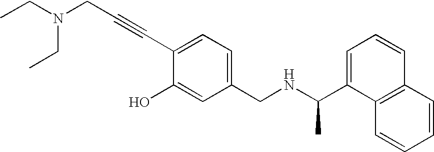 |
| 208 | 99 | 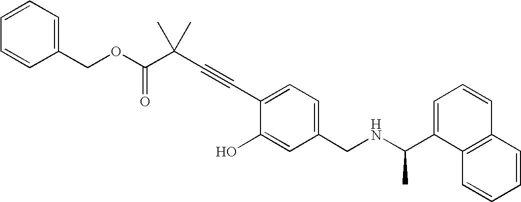 |
| 209 | 100 | 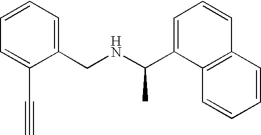 |
| 210 | 101 | 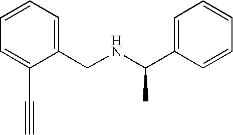 |
| 211 | 102 | 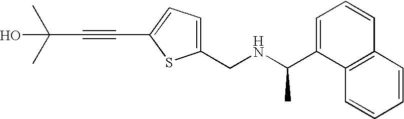 |
| 212 | 103 | 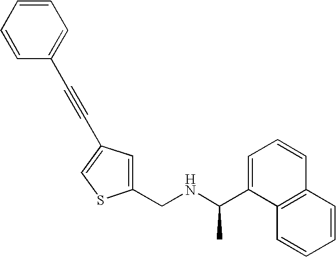 |
| 213 | 104 | 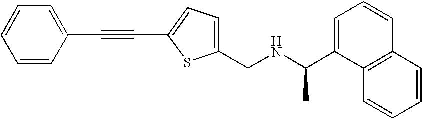 |
| 214 | 105 | 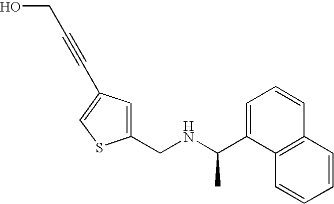 |
| 215 | 106 | 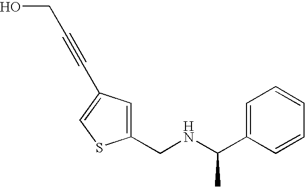 |
| 216 | 107 | 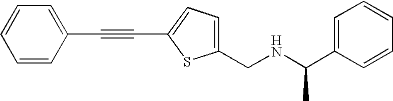 |
| 217 | 108 | 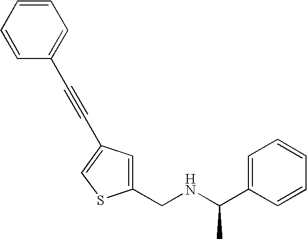 |
| 218 | 109 | 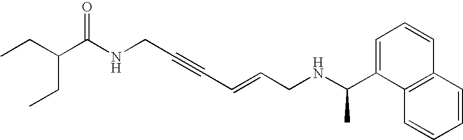 |
| 219 | 110 | 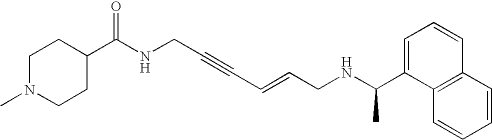 |
| 220 | 111 | 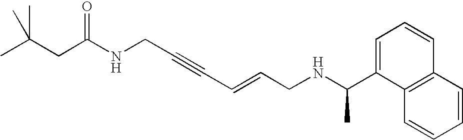 |
| 221 | 112 | 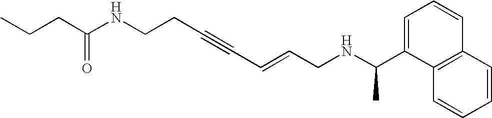 |
| 222 | 113 | 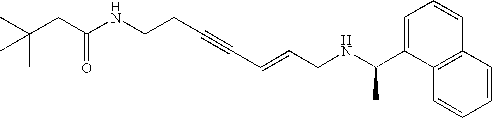 |
| 223 | 114 | 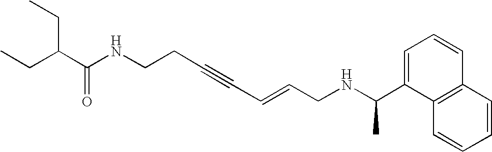 |
| 224 | 115 | 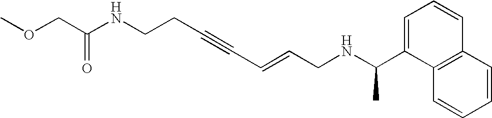 |
| 225 | 116 | 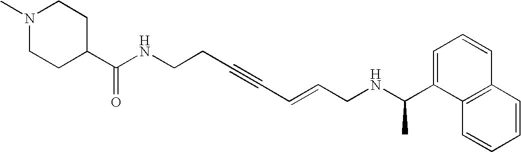 |
| 226 | 117 | 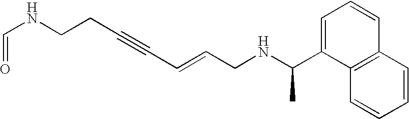 |
| 227 | 118 | 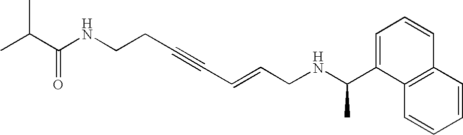 |
| 228 | 119 | 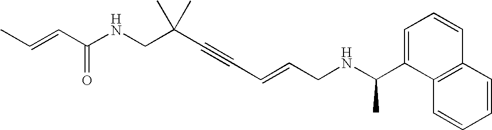 |
| 229 | 120 | 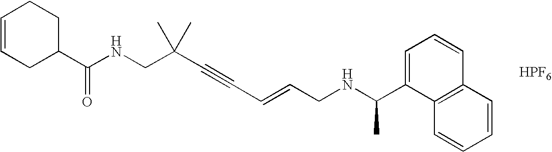 |
| 230 | 121 | 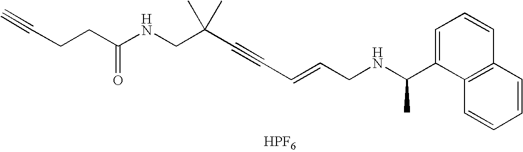 |
| 231 | 122 | 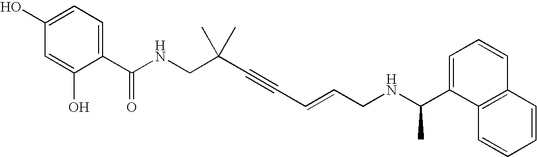 |
| 232 | 123 | 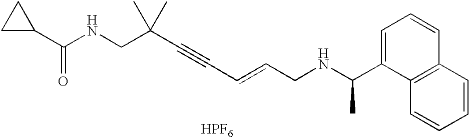 |
| 233 | 124 | 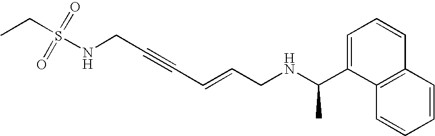 |
| 234 | 125 | 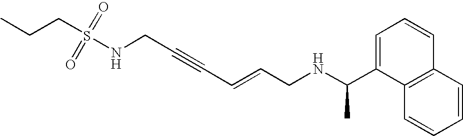 |
| 235 | 126 | 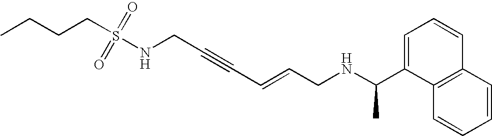 |
| 236 | 127 | 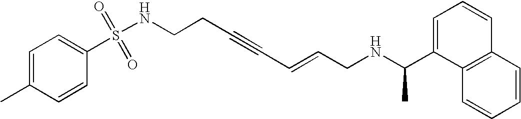 |
| 237 | 128 | 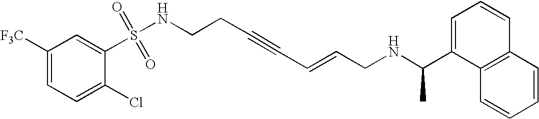 |
| 238 | 129 | 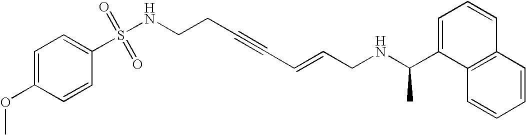 |
| 239 | 130 | 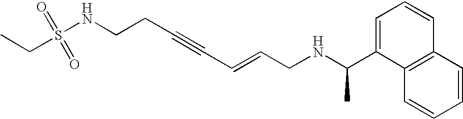 |
| 240 | 131 | 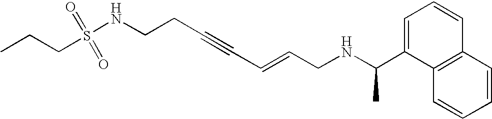 |
| 241 | 132 | 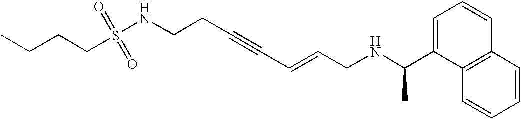 |
| 242 | 133 | 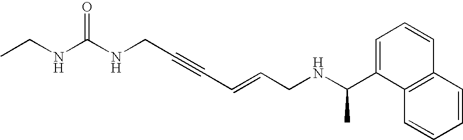 |
| 243 | 134 | 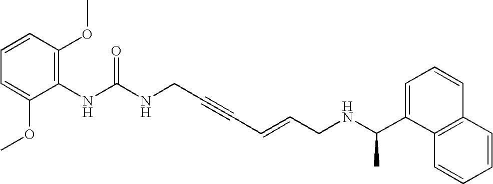 |
| 244 | 135 | 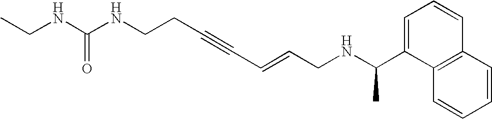 |
| 245 | 136 | 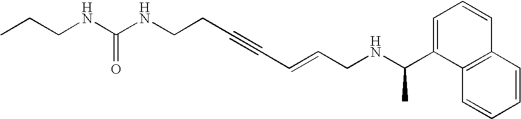 |
| 246 | 137 | 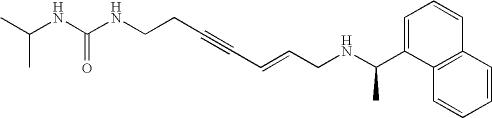 |
| 247 | 138 | 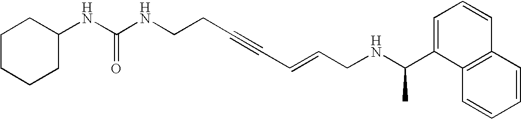 |
| 248 | 139 | 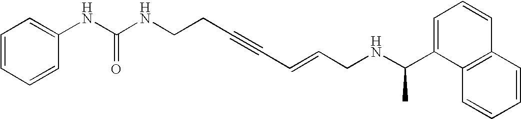 |
| 249 | 140 | 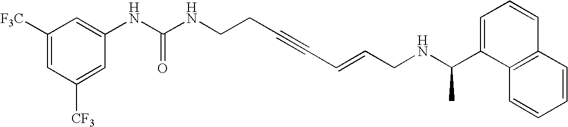 |
| 250 | 141 | 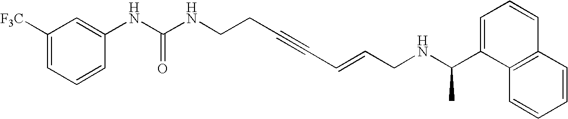 |
| 251 | 142 | 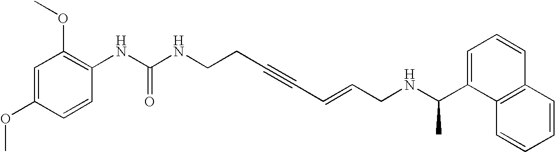 |
| 252 | 143 | 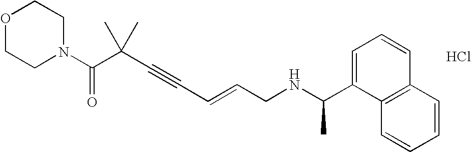 |
| 253 | 144 | 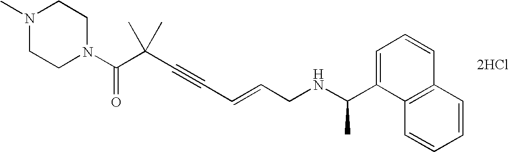 |
| 254 | 145 | 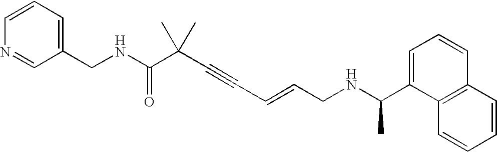 |
| 255 | 146 | 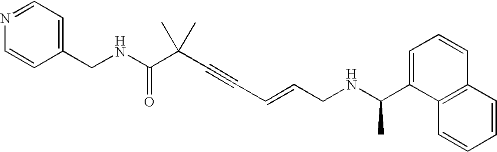 |
| 256 | 147 | 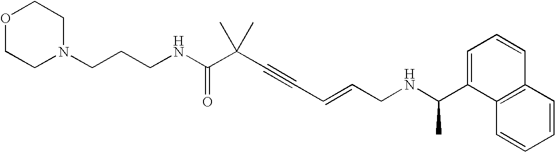 |
| 257 | 148 | 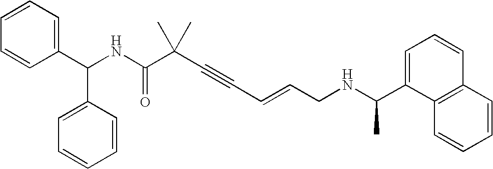 |
| 258 | 149 | 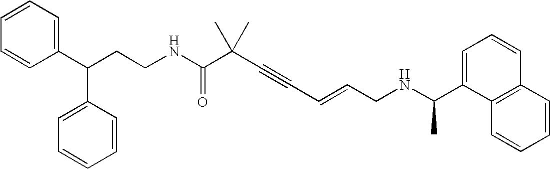 |
| 259 | 150 | 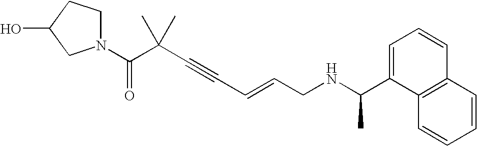 |
| 260 | 151 | 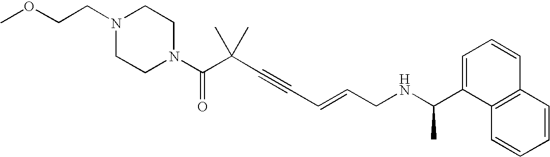 |
| 261 | 152 | 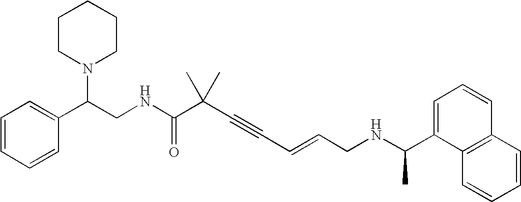 |
| 262 | 153 | 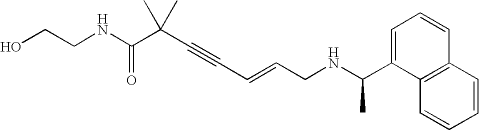 |
| 263 | 154 | 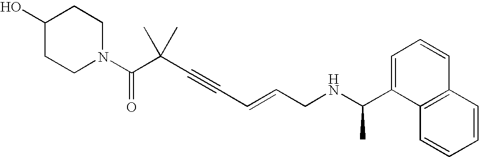 |
| 264 | 155 | 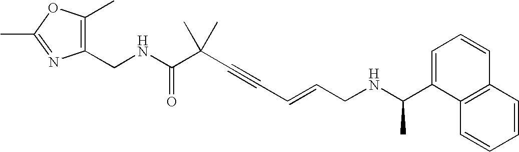 |
| 265 | 156 | 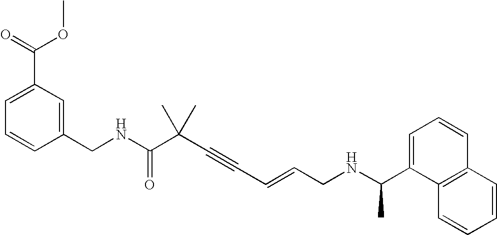 |
| 266 | 157 | 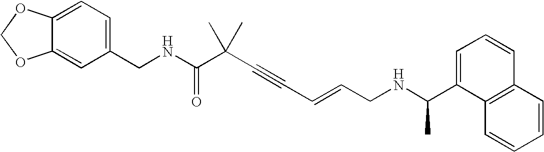 |
| 267 | 158 | 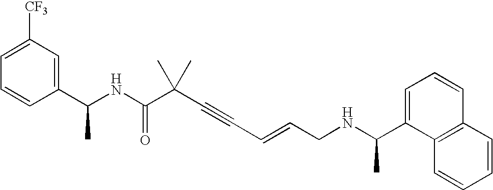 |
| 268 | 159 | 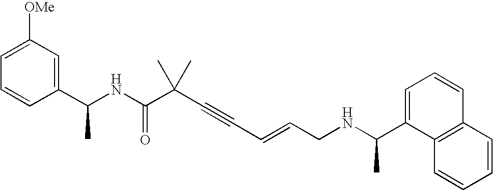 |
| 269 | 160 | 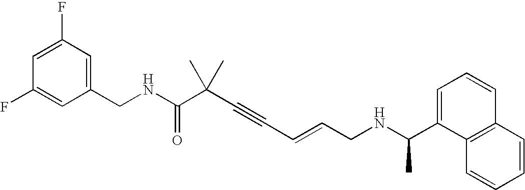 |
| 270 | 161 | 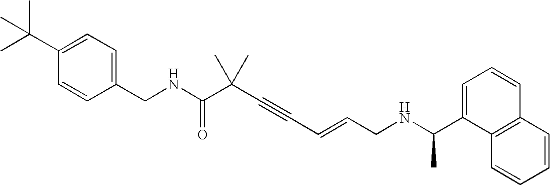 |
| 271 | 162 | 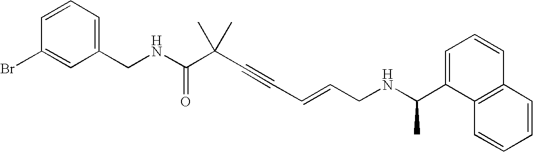 |
| 272 | 163 | 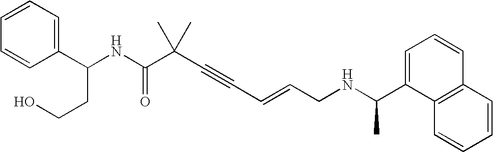 |
| 273 | 164 | 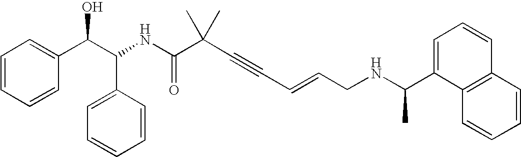 |
| 274 | 165 | 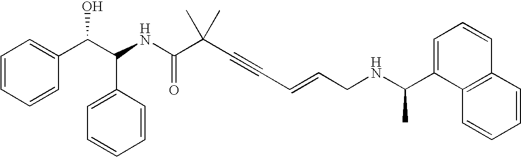 |
| 275 | 166 | 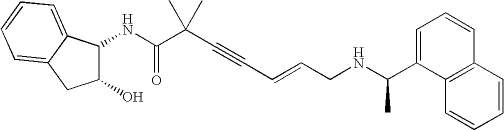 |
| 276 | 167 | 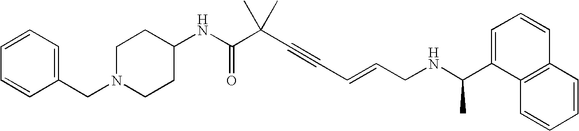 |
| 277 | 168 | 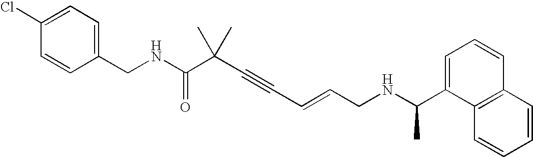 |
| 278 | 169 | 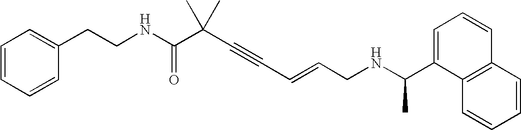 |
| 279 | 170 | 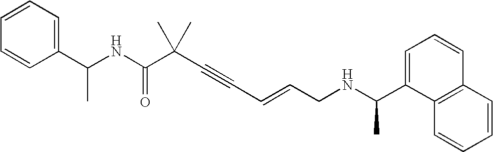 |
| 280 | 171 | 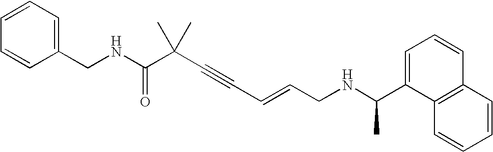 |
| 281 | 172 | 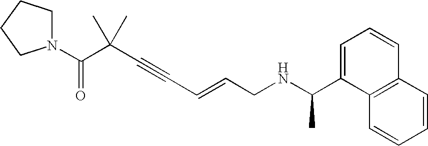 |
| 282 | 173 | 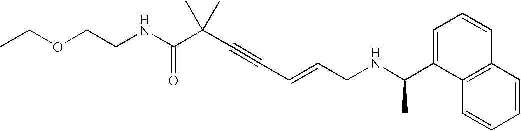 |
| 283 | 174 | 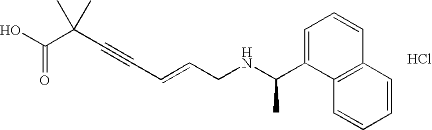 |
| 284 | 175 | 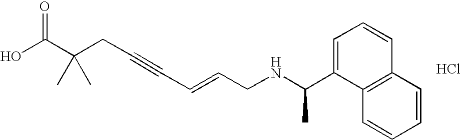 |
| 285 | 176 | 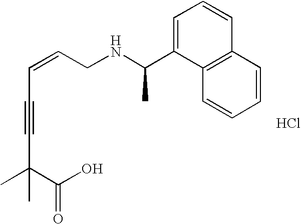 |
| 286 | 177 | 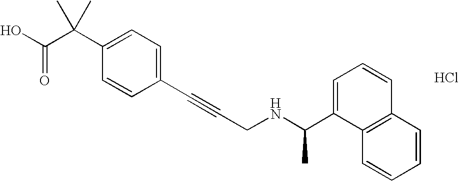 |
| 287 | 178 | 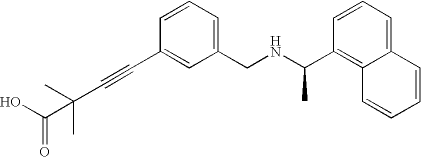 |
| 288 | 179 | 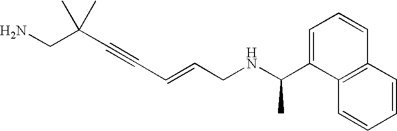 |
| 289 | 180 | 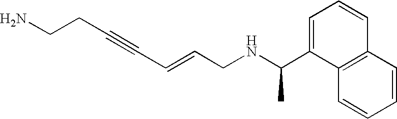 |
| 290 | 181 | 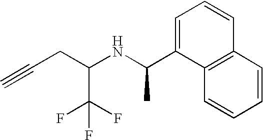 |
| 291 | 182 | 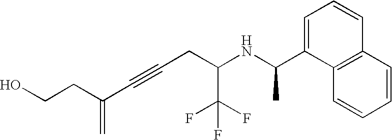 |
| 292 | 183 | 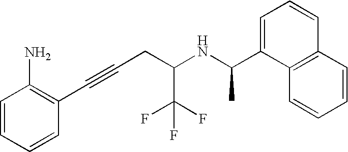 |
| 293 | 184 | 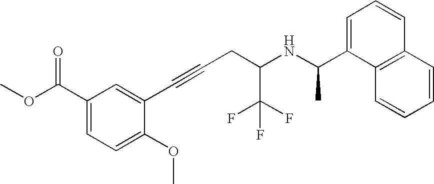 |
| 294 | 185 | 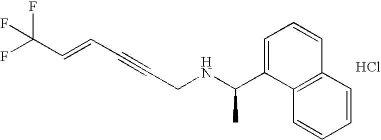 |

### Example 1

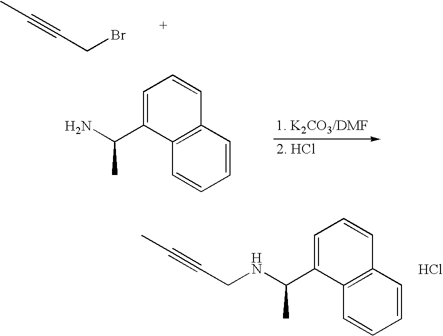

### (R)-But-2-ynyl-(1-naphthalen-1-yl-ethyl)-amine hydrochloride (Compound 101)

1-Bromo-but-2-yne (0.66 g, 5.0 mmol) and (R)-1-naphthalen-1-yl-ethylamine (2.0 g, 11.7 mmol) were treated as described in General procedure 1. The crude was purified by chromatography (PE/EtOAc 5:1), giving a free amine. The free amine was redissolved in MeOH. To this solution was added aqueous HCl (1N) until pH2 and the product was crystallized out. Filtration and wash of the crystals with small amount of Et₂O gave the title compound as a white solid.

¹³C NMR (DMSO-d₆): δ=129.3, 127.2, 126.5, 125.9, 124.9, 122.9, 85.2, 70.8, 51.2, 34.8, 20.2, 3.6.

### Example 2

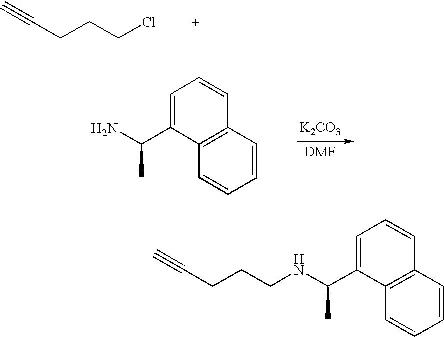

### (R)-(1-Naphthalen-1-yl-ethyl)-pent-4-ynyl-amine (Compound 102)

(R)-1-naphthalen-1-yl-ethylamine (4.1 g, 24.0 mmol) and 5-chloro-pent-1-yne (2.1 g, 20.0 mmol) were dissolved in DMF (25 mL). To this solution were added K₂CO₃ (3.9 g, 28 mmol) and NaI (1.0 g) at room temperature. The reaction mixture was stirred at 50° C. for 48 h and then poured into H₂O. The obtained mixture was extracted with EtOAc. The combined organic phases were dried over MgSO₄ and concentrated in vacuo. The residue was purified by chromatography (PE/EtOAc 1:1→PE/EtOAc 0:1), giving the title compound as an oil.

¹³C NMR (DMSO-d₆): δ=130.6, 129.4, 129.3, 127.2, 126.5, 125.9, 124.9, 122.9, 85.2, 70.8, 51.2, 34.8, 20.2, 3.6.

### Example 3

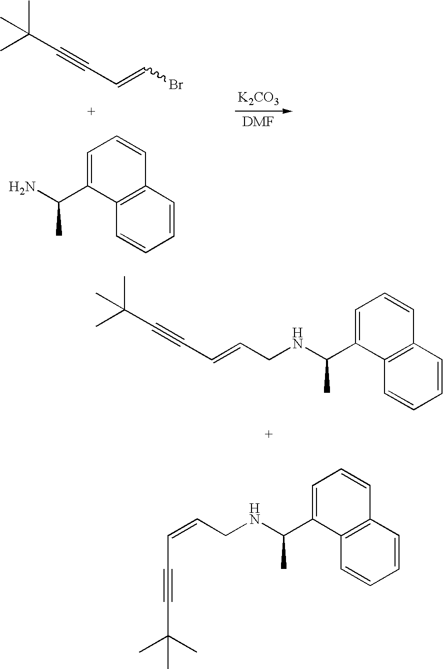

### (E)/(R)-(6,6-Dimethyl-hept-2-en-4-ynyl)-(1-naphthalen-1-yl-ethyl)-amine (Compound 103) and (Z)/(R)-(6,6-Dimethyl-hept-2-en-4-ynyl)-(1-naphthalen-1-yl-ethyl)-amine (Compound 104)

(E/Z)-1-bromo-6,6-dimethyl-hept-2-en-4-yne (374 mg, 2.0 mmol) and (R)-1-naphthalen-1-yl-ethylamine (342 mg, 2.0 mmol) were treated as described in General procedure 1. The crude was purified by chromatography (PE/EtOAc 6:1), giving compound 103 and compound 104 as colorless oils.

### Compound 103:

¹³C NMR (CDCl₃): δ=140.9, 140.5, 134.0, 131.3, 129.0, 127.2, 125.7, 125.7, 125.3, 123.0, 122.7, 111.3, 98.4, 77.2, 52.9, 49.3, 31.0, 27.9, 23.6.

### Compound 104:

¹³C NMR (CDCl₃): δ=141.1, 139.8, 134.0, 131.4, 128.9, 127.2, 125.7, 125.7, 125.3, 123.0, 122.7, 111.4, 103.8, 75.2, 52.7, 46.6, 30.9, 28.0, 23.4.

### Example 4

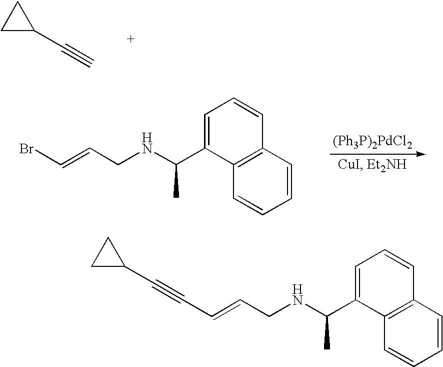

### (E)/(R)-(5-Cyclopropyl-pent-2-en-4-ynyl)-(1-naphthalen-1-yl-ethyl)-amine (Compound 105)

(E)/(R)-(3-Bromo-allyl)-(1-naphthalen-1-yl-ethyl)-amine (compound 401) from preparation 1 (290 mg, 1.0 mmol) and ethynyl-cyclopropane (70% in toluene, 3.0 mL, 3.5 mmol) were treated as described in General procedure 2. The crude was purified by chromatography (DCM/EtOAc 10:1), giving the title compound as an oil.

¹³C NMR (CDCl₃): δ=140.8, 140.7, 133.9, 131.2, 128.8, 127.1, 125.6, 125.6, 125.2, 122.8, 122.6, 111.1, 93.1, 73.9, 52.7, 49.1, 23.5, 8.4.

### Example 5

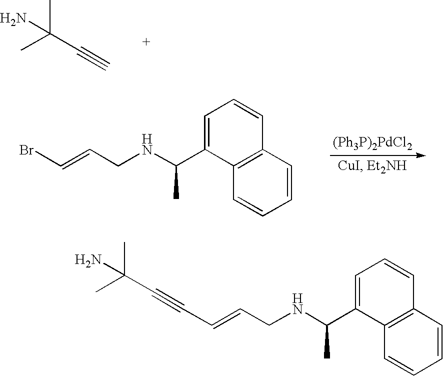

### (E)/(R)-6-Methyl-(1-naphthalen-1-yl-ethyl)-hept-2-en-4-yne-1,6-diamine (Compound 106)

(E)/(R)-(3-Bromo-allyl)-(1-naphthalen-1-yl-ethyl)-amine (compound 401) from preparation 1 (290 mg, 1.0 mmol) and 1,1-dimethyl-prop-2-ynylamine (291 mg, 3.5 mmol) were treated as described in General procedure 2. Chromatography (MeOH/EtOAc 1:2) gave the title compound as a solid.

¹³C NMR (DMSO-d₆): δ=142.7, 141.4, 133.5, 130.9, 128.7, 126.8, 125.7, 125.7, 125.3, 123.0, 122.8, 109.5, 78.9, 52.5, 48.5, 45.7, 31.0, 23.7.

### Example 6

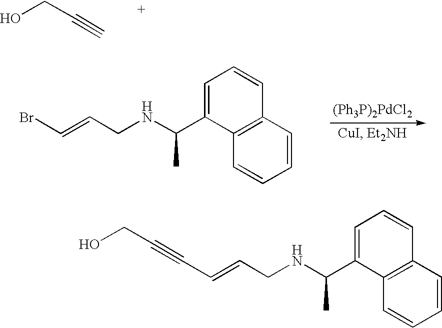

### (E)/(R)-6-(1-Naphthalen-1-yl-ethylamino)-hex-4-en-2-yn-1-ol (Compound 107)

(E)/(R)-(3-Bromo-allyl)-(1-naphthalen-1-yl-ethyl)-amine (compound 401) from preparation 1 (290 mg, 1.0 mmol) and 2-propyn-1-ol (0.2 mL, 3.5 mmol) were treated as described in General procedure 2. Chromatography (EtOAc) gave the title compound as an oil.

¹³C NMR (DMSO-d₆): δ=143.1, 141.4, 133.5, 130.9, 128.7, 126.8, 125.8, 125.7, 125.3, 123.0, 122.8, 109.4, 89.3, 82.4, 52.4, 49.4, 48.5, 23.7.

### Example 7

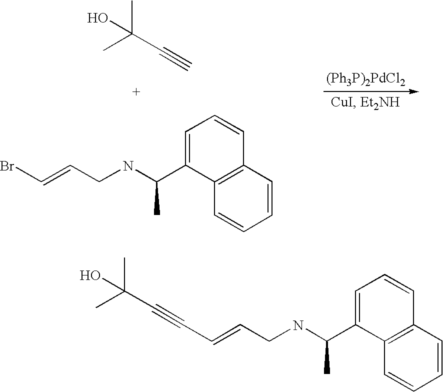

### (E)/(R)-2-Methyl-7-(1-naphthalen-1-yl-ethylamino)-hept-5-en-3-yn-2-ol (Compound 108)

(E)/(R)-(3-Bromo-allyl)-(1-naphthalen-1-yl-ethyl)-amine (compound 401) from preparation 1 (44 mg, 0.15 mmol) and 2-methyl-but-3-yn-2-ol (25 mg, 0.3 mmol) were treated as described in General procedure 2. Chromatography (PE/EtOAc 3:1→1:3) gave the title compound.

¹H NMR (CDCl₃): δ=8.17 (d, 1H), 7.87 (m, 1H), 7.74 (d, 1H), 7.64 (d, 1H), 7.53-7.42 (m, 3H), 6.21 (dt, 1H), 5.63 (d, 1H), 4.65 (q, 1H), 3.23 (m, 2H), 1.53 (s, 6H), 1.48 (d, 3H).

### Example 8

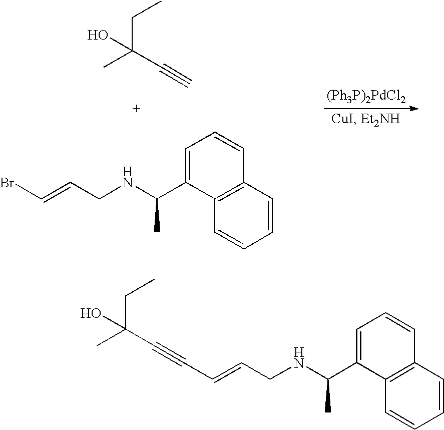

### (E)/(3R/S)-3-Methyl-8-[(R)-1-naphthalen-1-yl-ethylamino]-oct-6-en-4-yn-3-ol (Compound 109)

(E)/(R)-(3-Bromo-allyl)-(1-naphthalen-1-yl-ethyl)-amine (compound 401) from preparation 1 (44 mg, 0.15 mmol) and (R/S)-3-methyl-pent-1-yn-3-ol (29 mg, 0.3 mmol) were treated as described in General procedure 3, giving the title compound.

¹H NMR (DMSO-d₆): δ=8.23 (d, 1H), 7.93 (d, 1H), 7.81 (d, 1H), 7.69 (br, 1H), 7.58-7.47 (m, 3H), 6.38/6.08 (br, 1H), 5.70 (d, 1H), 4.73 (br, 1H), 3.25 (br, 2H), 1.56 (m, 2H), 1.32 (s, 3H), 0.93 (t, 3H).

### Example 9

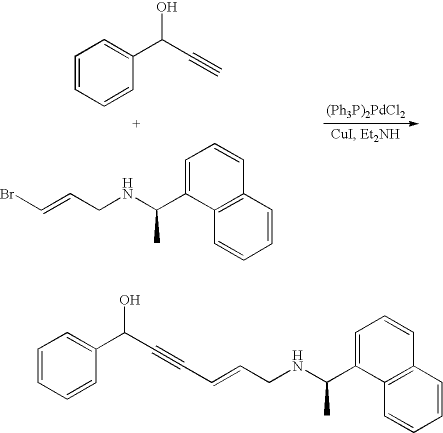

### (E)/(1R/S)-6-[(R)-1-Naphthalen-1-yl-ethylamino]-1-phenyl-hex-4-en-2-yn-1-ol (Compound 110)

(E)/(R)-(3-Bromo-allyl)-(1-naphthalen-1-yl-ethyl)-amine (compound 401) from preparation 1 (44 mg, 0.15 mmol) and (R/S)-1-phenyl-prop-2-yn-1-ol (40 mg, 0.3 mmol) were treated as described in General procedure 3, giving the title compound.

¹H NMR (DMSO-d₅): δ=8.22 (d, 1H), 7.93 (d, 1H), 7.80 (d, 1H), 7.69 (d, 1H), 7.55-7.47 (m, 3H), 7.46 (d, 2H), 7.36 (t, 2H), 7.29 (t, 1H), 6.17 (br, 1H), 5.74 (d, 1H), 5.48 (s, 1H), 4.70 (br, 1H), 3.23 (br, 2H), 1.40 (br, 3H).

### Example 10

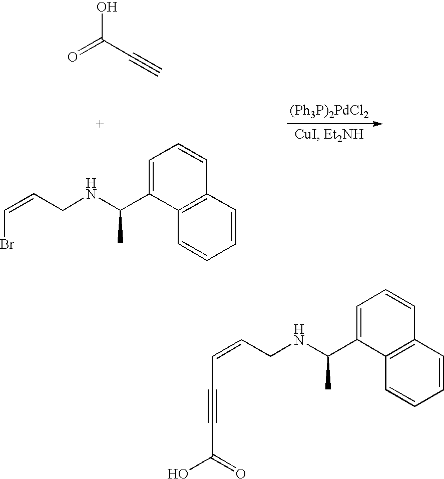

### (Z)/(R)-6-(1-Naphthalen-1-yl-ethylamino)-hex-4-en-2-ynoic acid (Compound 111)

(Z)/(R)-(3-Bromo-allyl)-(1-naphthalen-1-yl-ethyl)-amine (compound 402) from preparation 1 (44 mg, 0.15 mmol) and propynoic acid (21 mg, 0.3 mmol) were treated as described in General procedure 3, giving the title compound.

¹H NMR (CDCl₃): δ=8.16 (d, 1H), 7.87 (d, 1H), 7.77 (d, 1H), 7.73 (d, 1H), 7.57-7.44 (m, 3H), 6.34 (m, 1H), 6.25 (d, 1H), 4.76 (q, 1H), 3.42 (dd, 2H), 1.58 (d, 3H).

### Example 11

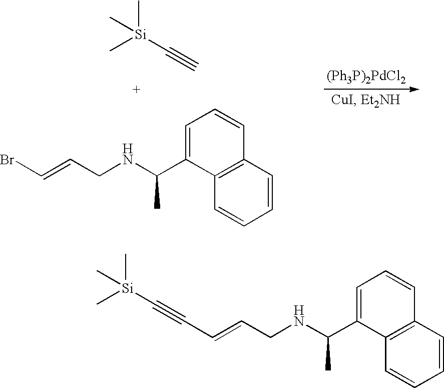

### (E)/(R)-(1-Naphthalen-1-yl-ethyl)-(5-trimethylsilanyl-pent-2-en-4-ynyl)-amine (Compound 112)

(E)/(R)-(3-Bromo-allyl)-(1-naphthalen-1-yl-ethyl)amine (compound 401) from preparation 1 (290 mg, 1.0 mmol) and ethynyl-trimethyl-silane (344 mg, 3.5 mmol) were treated as described in General procedure 2. Chromatography (PE/EtOAc 6:1) gave the title compound as an oil.

¹³C NMR (DMSO-d₆): δ=145.1, 141.4, 133.5, 130.9, 128.7, 126.7, 125.7, 125.7, 125.3, 123.0, 122.7, 109.4, 104.4, 93.4, 52.5, 48.5, 23.7, −0.1.

### Example 12

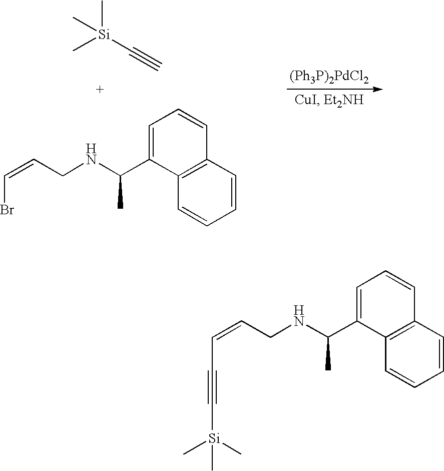

### (Z)/(R)-(1-Naphthalen-1-yl-ethyl)-(5-trimethylsilanyl-pent-2-en-4-ynyl)-amine (Compound 113)

(Z)/(R)-(3-Bromo-allyl)-(1-naphthalen-1-yl-ethyl)-amine (compound 402) from preparation 1 (290 mg, 1.0 mmol) and ethynyl-trimethyl-silane (344 mg, 3.5 mmol) were treated as described in General procedure 2. Chromatography (PE/EtOAc 6:1) gave the title compound as an oil.

¹³C NMR (DMSO-d₆): δ=144.7, 141.4, 133.6, 131.0, 128.7, 126.7, 125.7, 125.6, 125.2, 123.0, 122.8, 109.5, 101.9, 98.9, 52.3, 46.4, 23.6, −0.3.

### Example 13

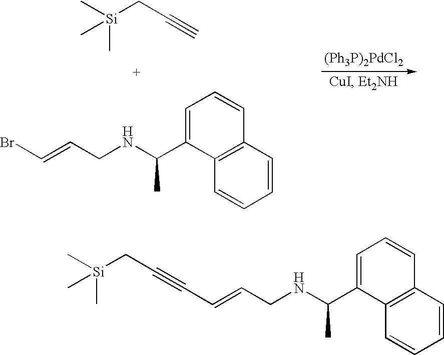

### (E)/(R)-(1-Naphthalen-1-yl-ethyl)-(6-trimethylsilanyl-hex-2-en-4-ynyl)-amine (Compound 114)

(E)/(R)-(3-Bromo-allyl)-(1-naphthalen-1-yl-ethyl)-amine (compound 401) from preparation 1 (44 mg, 0.15 mmol) and Trimethyl-prop-2-ynyl-silane (34 mg, 0.3 mmol) were treated as described in General procedure 3, giving the title compound.

¹H NMR (DMSO-d₆): δ=8.22 (d, 1H), 7.92 (d, 1H), 7.80 (d, 1H), 7.69 (d, 1H), 7.55-7.47 (m, 3H), 5.97 (dt, 1H), 5.60 (d, 1H), 4.64 (br, 1H), 3.14 (br, 2H), 1.64 (d, 2H), 1.39 (d, 3H), 0.07 (s, 9H).

### Example 14

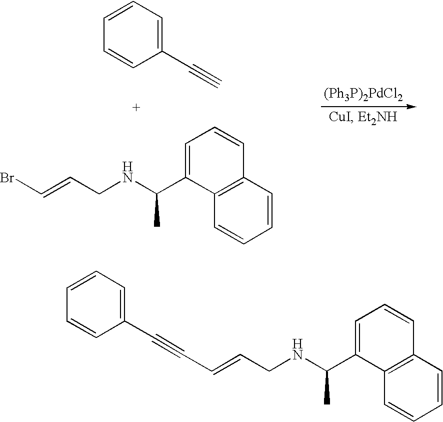

### (E)/(R)-(1-Naphthalen-1-yl-ethyl)-(5-phenyl-pent-2-en-4-ynyl)-amine (Compound 115)

(E)/(R)-(3-Bromo-allyl)-(1-naphthalen-1-yl-ethyl)-amine (compound 401) from preparation 1 (44 mg, 0.15 mmol) and ethynyl benzene (31 mg, 0.3 mmol) were treated as described in General procedure 3, giving the title compound.

¹H NMR (DMSO-d₆): δ=8.20 (d, 1H), 7.94 (d, 1H), 7.82 (d, 1H), 7.72 (d, 1H), 7.57-7.48 (m, 3H), 7.44 (m, 2H), 7.38 (m, 3H), 6.29 (dt, 1H), 5.94 (d, 1H), 4.68 (br, 1H), 3.24 (br, 2H), 1.41 (d, 3H)

### Example 15

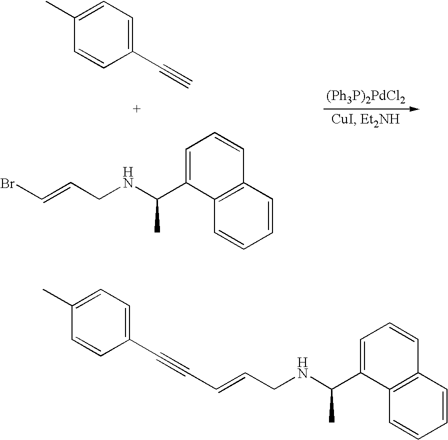

### (E)/(R)-(1-Naphthalen-1-yl-ethyl)-(5-p-tolyl-pent-2-en-4-ynyl)-amine (Compound 116)

(E)/(R)-(3-Bromo-allyl)-(1-naphthalen-1-yl-ethyl)-amine (compound 401) from preparation 1 (44 mg, 0.15 mmol) and 1-ethynyl-4-methyl-benzene (35 mg, 0.3 mmol) were treated as described in General procedure 3, giving the title compound.

¹H NMR (DMSO-d₆): δ=8.25 (d, 1H), 7.95 (d, 1H), 7.84 (d, 1H), 7.72 (d, 1H), 7.55-7.49 (m, 3H), 7.33 (d, 2H), 7.19 (d, 2H), 6.25 (dt, 1H), 5.95 (d, 1H), 4.76 (br, 1H), 3.35 (br, 2H), 2.31 (s, 3H), 1.45 (d, 3H).

### Example 16

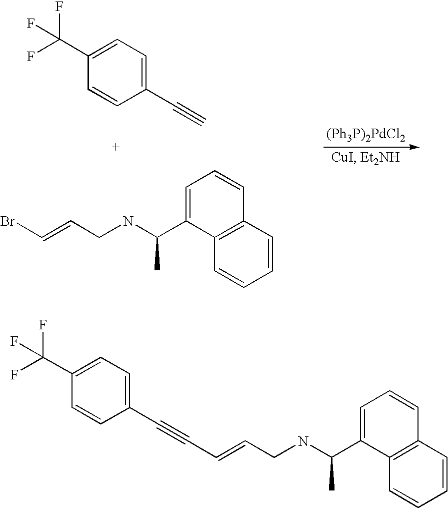

### (E)/(R)-(1-Naphthalen-1-yl-ethyl)-[5-(4-trifluoromethyl-phenyl)-pent-2-en-4-ynyl]-amine (Compound 117)

(E)/(R)-(3-Bromo-allyl)-(1-naphthalen-1-yl-ethyl)-amine (compound 401) from preparation 1 (290 mg, 1 mmol) and 1-ethynyl-4-trifluoromethyl-benzene (0.37 mL, 3.5 mmol) were treated as described in General procedure 2. Chromatography (PE/EtOAc 4:1) gave the title compound as an oil.

¹³C NMR (DMSO-d₆): δ=145.8, 141.4, 133.6, 131.9, 131.0, 128.7, 128.3, 127.0, 126.8, 125.8, 125.7, 125.5, 125.3, 123.1, 122.8, 122.2, 108.7, 91.1, 87.3, 52.6, 48.6, 23.8.

### Example 17

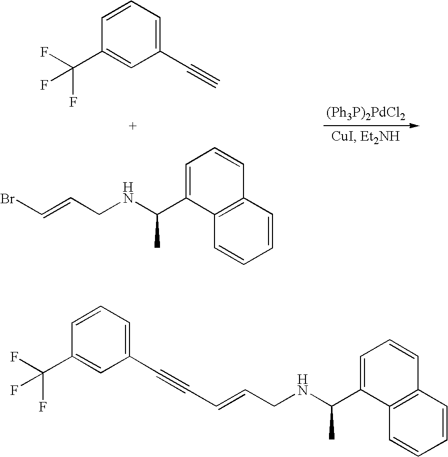

### (E)/(R)-(1-Naphthalen-1-yl-ethyl)-[5-(3-trifluoromethyl-phenyl)-pent-2-en-4-ynyl]-amine (Compound 118)

(E)/(R)-(3-Bromo-allyl)-(1-naphthalen-1-yl-ethyl)-amine (compound 401) from preparation 1 (44 mg, 0.15 mmol) and 1-ethynyl-3-trifluoromethyl-benzene (51 mg, 0.3 mmol) were treated as described in General procedure 3, giving the title compound.

¹H NMR (DMSO-d₆): δ=8.25 (d, 1H), 7.93 (d, 1H), 7.81 (d, 1H), 7.77 (s, 1H), 7.74 (d, 1H), 7.71 (d, 1H), 7.58-7.48 (m, 5H), 6.38 (dt, 1H), 5.95 (d, 1H), 4.63 (q, 1H), 3.21 (br, 2H), 1.41 (d, 3H).

### Example 18

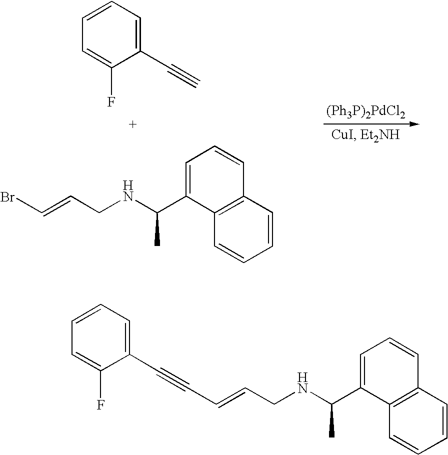

### (E)/(R)-[5-(2-Fluoro-phenyl)-bent-2-en-4-ynyl]-(1-naphthalen-1-yl-ethyl)-amine ((Compound 119)

(E)/(R)-(3-Bromo-allyl)-(1-naphthalen-1-yl-ethyl)-amine (compound 401) from preparation 1 (44 mg, 0.15 mmol) and 1-ethynyl-2-fluoro-benzene (36 mg, 0.3 mmol) were treated as described in General procedure 3, giving the title compound.

¹H NMR (DMSO-d₆): δ=8.25 (d, 1H), 7.94 (d, 1H), 7.81 (d, 1H), 7.72 (d, 1H), 7.56-7.48 (m, 4H), 7.43 (m, 1H), 7.29 (t, 1H), 7.22 (t, 1H), 6.34 (dt, 1H), 5.97 (d, 1H), 4.66 (br, 1H), 3.23 (br, 2H), 1.41 (d, 3H).

### Example 19

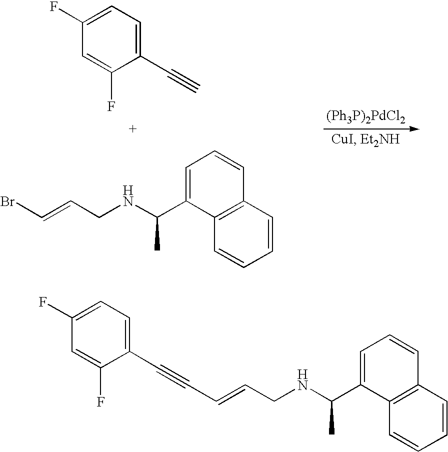

### (E)/(R)-[5-(2,4-Difluoro-phenyl)-pent-2-en-4-ynyl]-(1-naphthalen-1-yl-ethyl)-amine (Compound 120)

(E)/(R)-(3-Bromo-allyl)-(1-naphthalen-1-yl-ethyl)-amine (compound 401) from preparation 1 (44 mg, 0.15 mmol) and 1-ethynyl-2,4-difluoro-benzene (41 mg, 0.3 mmol) were treated as described in General procedure 3, giving the title compound.

¹H NMR (DMSO-d₆): δ=8.25 (d, 1H), 7.94 (d, 1H), 7.81 (d, 1H), 7.71 (d, 1H), 7.58 (m, 1H), 7.55-7.48 (m, 3H), 7.39 (dt, 1H), 7.14 (dt, 1H), 6.34 (dt, 1H), 5.97 (d, 1H), 4.65 (br, 1H), 3.23 (br, 2H), 1.41 (d, 3H).

### Example 20

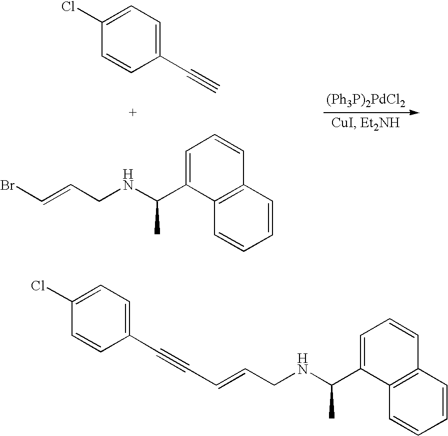

### (E)/(R)-[5-(4-Chloro-phenyl)-pent-2-en-4-ynyl]-(1-naphthalen-1-yl-ethyl)-amine (Compound 121)

(E)/(R)-(3-Bromo-allyl)-(1-naphthalen-1-yl-ethyl)-amine (compound 401) from preparation 1 (44 mg, 0.15 mmol) and 1-chloro-4-ethynyl-benzene (41 mg, 0.3 mmol) were treated as described in General procedure 3, giving the title compound.

¹H NMR (DMSO-d₆): δ=8.25 (d, 1H), 7.95 (d, 2H), 7.85 (d, 1H), 7.72 (d, 1H), 7.58-7.49 (m, 3H), 7.46 (s, 4H), 6.31 (dt, 1H), 5.98 (d, 1H), 4.78 (br, 1H), 3.30 (br, 2H), 1.46 (d, 3H).

### Example 21

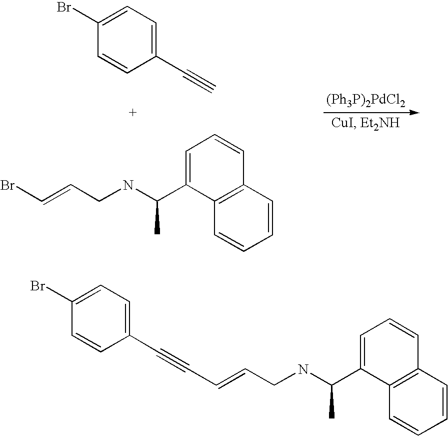

### (E)/(R)-[5-(4-Bromo-phenyl)-pent-2-en-4-ynyl]-(1-naphthalen-1-yl-ethyl)-amine (Compound 122)

To a mixture of (E)/(R)-(3-Bromo-allyl)-(1-naphthalen-1-yl-ethyl)-amine (compound 401) (44 mg, 0.15 mmol) from preparation 1 and 1-bromo-4-ethynyl-benzene (54 mg, 0.3 mmol) treated as described in General procedure 3, giving the title compound.

¹H NMR (CDCl₃): δ=8.19 (d, 1H), 7.87 (dd, 1H), 7.75 (d, 1H), 7.65 (d, 1H), 7.55-7.38 (m, 5H), 7.31-7.21 (m, 2H), 6.33 (dt, 1H), 5.83 (d, 1H), 4.68 (q, 1H), 3.29 (m, 2H), 1.53 (br, 1H), 1.50 (d, 3H).

### Example 22

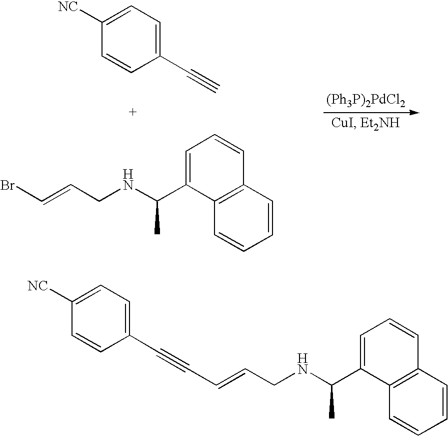

### (E)/(R)-4-[5-(1-Naphthalen-1-yl-ethylamino)-pent-3-en-1-ynyl]-benzonitrile (Compound 123)

(E)/(R)-(3-Bromo-allyl)-(1-naphthalen-1-yl-ethyl)-amine (compound 401) from preparation 1 (44 mg, 0.15 mmol) and 4-ethynyl-benzonitrile (38 mg, 0.3 mmol) were treated as described in General procedure 3, giving the title compound.

¹H NMR (DMSO-d₆): δ=8.25 (d, 1H), 7.94 (d, 1H), 7.85 (d, 2H), 7.83 (d, 1H), 7.71 (d, 1H), 7.62 (d, 2H), 7.57-7.49 (m, 3H), 6.40 (dt, 1H), 6.00 (d, 1H), 4.70 (br, 1H), 3.29 (br, 2H), 1.42 (d, 3H).

### Example 23

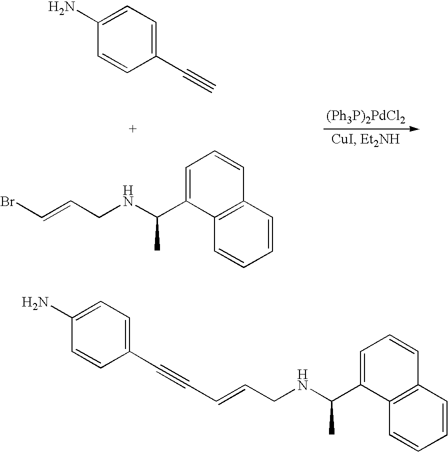

### (E)/(R)-4-[5-(1-Naphthalen-1-yl-ethylamino)-pent-3-en-1-ynyl]-aniline (Compound 124)

(E)/(R)-(3-Bromo-allyl)-(1-naphthalen-1-yl-ethyl)-amine (compound 401) from preparation 1 (44 mg, 0.15 mmol) and 4-ethynyl-aniline (35 mg, 0.3 mmol) were treated as described in General Procedure 2. Chromatography (PE/EtOAc 1:0→1:1) gave the title compound.

¹H NMR (CDCl₃): δ=8.20 (d, 1H), 7.86 (dd, 1H), 7.75 (d, 1H), 7.66 (d, 1H), 7.56-7.41 (m, 3H), 7.23 (d, 2H), 6.85 (d, 2H), 6.24 (dt, 1H), 5.82 (d, 1H), 4.68 (q, 1H), 3.76 (br, 2H), 3.27 (m, 2H), 1.55 (br, 1H), 1.50 (d, 3H).

### Example 24

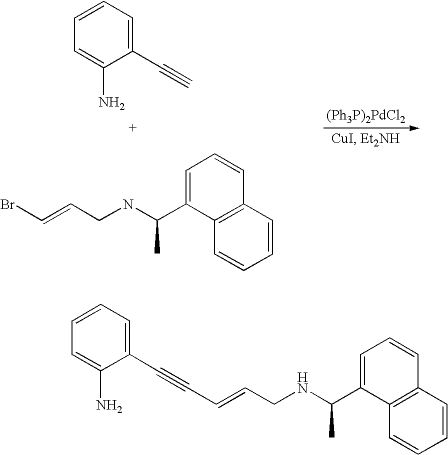

### (E)/(R)-2-[5-(1-Naphthalen-1-yl-ethylamino)-pent-3-en-1-ynyl]-aniline (Compound 125)

(E)/(R)-(3-Bromo-allyl)-(1-naphthalen-1-yl-ethyl)-amine (compound 401) from preparation 1 (44 mg, 0.15 mmol) and 2-ethynylaniline (35 mg, 0.3 mmol) were treated as described in General procedure 3, giving the title compound.

¹H NMR (DMSO-d₆): δ=8.25 (d, 1H), 7.94 (d, 1H), 7.82 (d, 1H), 7.72 (d, 1H), 7.58-7.48 (m, 3H), 7.11 (dd, 1H), 7.04 (t, 1H), 6.69 (d, 1H), 6.49 (t, 1H), 6.28 (dt, 1H), 5.95 (d, 1H), 4.69 (q, 1H), 3.24 (br, 2H), 1.43 (d, 3H).

### Example 25

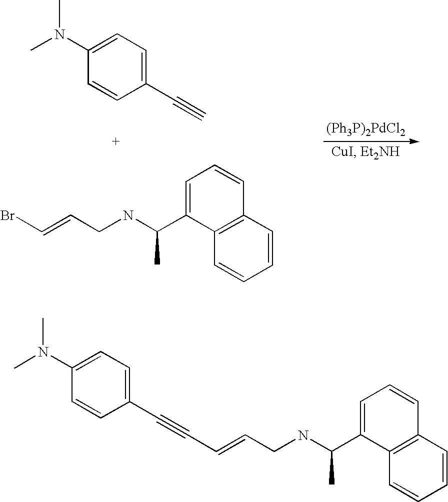

### (E)/(R)-Dimethyl-{4-[5-(1-naphthalen-1-yl-ethylamino)-pent-3-en-1-ynyl]}-aniline (Compound 126)

(E)/(R)-(3-Bromo-allyl)-(1-naphthalen-1-yl-ethyl)-amine (compound 401) from preparation 1 (44 mg, 0.15 mmol) and (4-ethynyl-phenyl)-dimethyl-amine (44 mg, 0.3 mmol) were treated as described in General procedure 2. Chromatography (PE/EtOAc 1:0→3:1) gave the title compound.

¹H NMR (CDCl₃): δ=8.20 (d, 1H), 7.86 (d, 1H), 7.75 (d, 1H), 7.67 (d, 1H), 7.47 (m, 3H), 7.30 (d, 2H), 6.61 (d, 2H), 6.23 (dt, 1H), 5.84 (d, 1H), 4.69 (q, 1H), 3.27 (m, 2H), 2.96 (s, 6H), 1.54 (br, 1H), 1.49 (d, 3H).

### Example 26

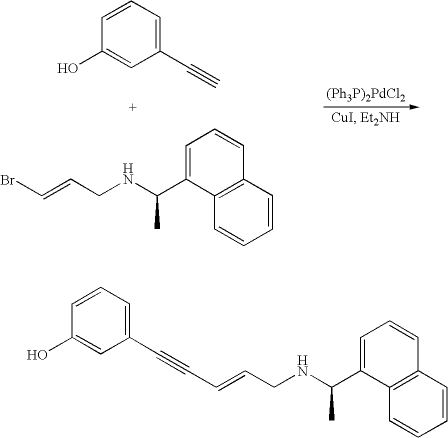

### (E)/(R)-3-[5-(1-Naphthalen-1-yl-ethylamino)-pent-3-en-1-ynyl]-phenol (Compound 127)

(E)/(R)-(3-Bromo-allyl)-(1-naphthalen-1-yl-ethyl)-amine (compound 401) from preparation 1 (44 mg, 0.15 mmol) and 3-ethynyl-phenol (35 mg, 0.3 mmol) were treated as described in General procedure 3, giving the title compound.

¹H NMR (DMSO-d₆): δ=8.25 (d, 1H), 7.94 (d, 1H), 7.82 (d, 1H), 7.72 (d, 1H), 7.57-7.48 (m, 3H), 7.16 (t, 1H), 6.85 (d, 1H), 6.78 (m, 2H), 6.27 (br, 1H), 5.92 (d, 1H), 4.71 (br, 1H), 3.30 (br, 2H), 1.42 (d, 3H)

### Example 27

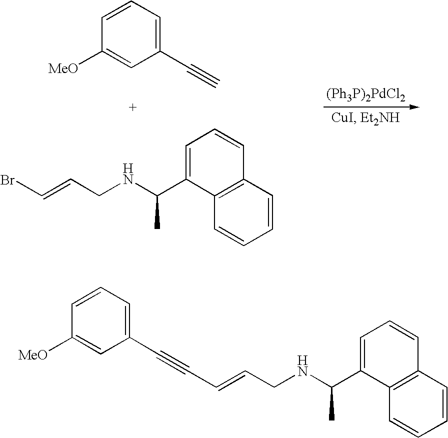

### (E)/(R)-[5-(3-Methoxy-phenyl)-pent-2-en-4-ynyl]-(1-naphthalen-1-yl-ethyl)-amine (Compound 128)

(E)/(R)-(3-Bromo-allyl)-(1-naphthalen-1-yl-ethyl)-amine (compound 401) from preparation 1 (44 mg, 0.15 mmol) and 1-ethynyl-3-methoxy-benzene (40 mg, 0.3 mmol) were treated as described in General Procedure 3, giving the title compound.

¹H NMR (DMSO-d₆): δ=8.25 (d, 1H), 7.94 (d, 1H), 7.82 (d, 1H), 7.72 (d, 1H), 7.58-7.49 (m, 3H), 7.29 (t, 1H), 7.01 (d, 1H), 6.98-6.93 (m, 2H), 6.30 (dt, 1H), 5.93 (d, 1H), 4.69 (br, 1H), 3.76 (s, 3H), 3.25 (br, 2H), 1.42 (d, 3H).

### Example 28

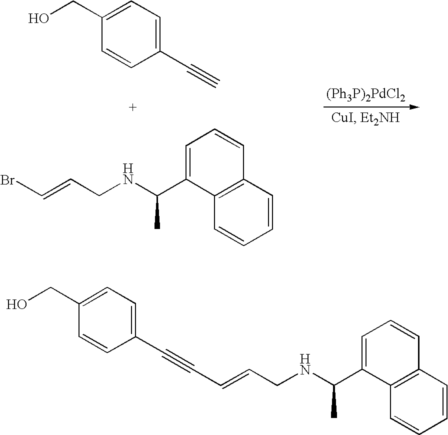

### (E)/(R)-{4-[5-(1-Naphthalen-1-yl-ethylamino)-pent-3-en-1-ynyl]-phenyl}-methanol (Compound 129)

(E)/(R)-(3-Bromo-allyl)-(1-naphthalen-1-yl-ethyl)-amine (compound 401) from preparation 1 (44 mg, 0.15 mmol) and 4-ethynyl-benzyl alcohol (40 mg, 0.3 mmol) were treated as described in General procedure 3, giving the title compound.

¹H NMR (DMSO-d₆): δ=8.25 (d, 1H), 7.94 (d, 1H), 7.82 (d, 1H), 7.71 (d, 1H), 7.57-7.47 (m, 3H), 7.39 (d, 2H), 7.32 (d, 2H), 6.28 (br, 1H), 5.93 (d, 1H), 4.70 (br, 1H), 4.50 (s, 2H), 3.26 (br, 2H), 1.42 (d, 3H).

### Example 29

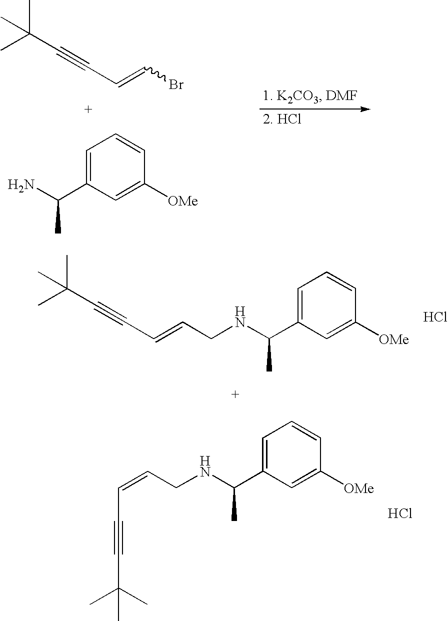

### (E)/(R)-(6,6-Dimethyl-hept-2-en-4-ynyl)-[1-(3-methoxy-phenyl)-ethyl]-amine hydrochloride (Compound 130) and (Z)/(R)-(6,6-Dimethyl-hept-2-en-4-ynyl)-[1-(3-methoxy-phenyl)-ethyl]-amine hydrochloride (Compound 131)

(E/Z)-1-bromo-6,6-dimethyl-hept-2-en-4-yne (374 mg, 2.0 mmol) and (R)-1-(3-methoxy-phenyl)-ethylamine (342 mg, 2.0 mmol) were treated as described in General procedure 1. Chromatography (PE/EtOAc 5:1) gave two products as colorless oils. The oil products were treated separately with aq. 4 N HCl/Et₂O, giving compound 130 and compound 131 as white solids.

### Compound 130:

¹³C NMR (DMSO-d₆): δ=159.6, 138.7, 132.0, 130.0, 119.9, 116.9, 114.4, 113.3, 100.7, 76.6, 56.6, 55.2, 46.0, 30.6, 27.6, 19.6.

### Compound 131:

¹³C NMR (DMSO-d₆): δ=159.6, 138.4, 131.4, 130.0, 119.9, 115.1, 114.3, 113.4, 106.0, 74.2, 56.5, 55.2, 43.5, 30.3, 27.6, 19.6.

### Example 30

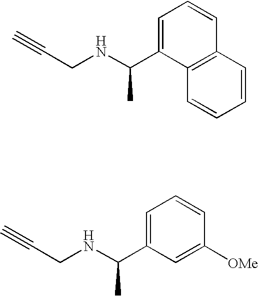

### (R)-(1-Naphthalen-1-yl-ethyl)-prop-2-ynyl-amine (Compound 132) and (R)-[1-(3-Methoxy-phenyl)-ethyl]-prop-2-ynyl-amine (Compound 133)

1-Bromo-prop-2-yne (4.8 g, 40.0 mmol), (R)-1-naphthalen-1-yl-ethylamine (3.4 g, 20.0 mmol), and (R)-1-(3-methoxy-phenyl)-ethylamine (3.0 g, 20.0 mmol) were treated as described in GP1. The residue was separated by chromatography (PE/EtOAc 3:1), giving the title compounds separately.

### Compound 132:

¹³C NMR (DMSO-d₆): δ=140.5, 133.5, 130.9, 128.6, 126.8, 125.7, 125.6, 125.2, 122.9, 82.9, 73.6, 51.2, 35.2, 23.2

### Compound 133:

¹³C NMR (DMSO-d₆): δ=159.9, 146.3, 129.5, 119.3, 112.6, 112.3, 82.3, 71.2, 56.4, 55.2, 35.9, 23.9.

### Example 31

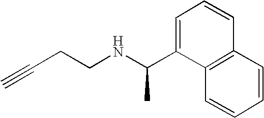

### (R)-But-3-ynyl-(1-naphthalen-1-yl-ethyl)-amine (Compound 134)

To a solution of but-3-ynyl tosylate (10.2 g, 45.0 mmol) and (R)-1-naphthalen-1-yl-ethylamine (14.7 g, 86.0 mmol) in DMF (25 mL) were added Na₂CO₃ (23.6 g, 170.8 mmol) and NaI (1.0 g). The mixture was stirred at 50° C. overnight. Aqueous work up with EtOAc, followed by chromatography in a gradient from 0 to 25% EtOAc in PE furnished the product.

¹³C NMR (CDCl₃): δ=140.9, 134.0, 131.3, 129.0, 127.2, 125.8, 125.7, 125.3, 122.9, 122.8, 82.7, 69.5, 53.2, 45.9, 23.7, 19.8.

### Example 32

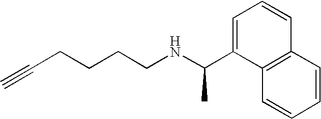

### (R)-Hex-5-ynyl-(1-naphthalen-1-yl-ethyl)-amine (Compound 135)

To a solution of 1-chloro-5-hexyne (5.0 g, 42.9 mmol) and (R)-1-naphthalen-1-yl-ethylamine (7.33 g, 42.8 mmol) in DMF (25 mL) were added Na₂CO₃ (11.8 g, 11.1 mmol) and NaI (0.3 mg). The mixture was stirred overnight at 40° C. Aqueous work up with EtOAc, followed by chromatography in a gradient from 0 to 25% EtOAc in PE furnished the product.

¹³C NMR (CDCl₃): δ=141.4, 134.0, 131.3, 129.0, 127.1, 125.7, 125.7, 125.3, 122.9, 122.6, 84.4, 68.3, 53.7, 47.4, 29.5, 26.3, 23.7, 18.3.

### Example 33

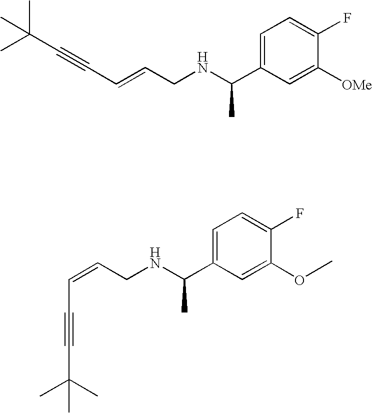

### (E)/(R)-(6,6-Dimethyl-hept-2-en-4-ynyl)-[1-(4-fluoro-3-methoxy-phenyl)-ethyl]-amine (Compound 136) and (Z)/(R)-(6,6-Dimethyl-hept-2-en-4-ynyl)-[1-(4-fluoro-3-methoxy-phenyl)-ethyl]-amine (Compound 137)

(E/Z)-1-bromo-6,6-dimethyl-hept-2-en-4-yne (1 equiv) and 1-(4-fluoro-3-methoxy-phenyl)-ethylamine (1 equiv) were treated as described in GP1. The crude was separated by chromatography (PE/EtOAc 1:0→3:1), giving the title compounds separately.

### Compound 136:

¹H NMR (DMSO-d₆): δ=7.11 (dd, J₁=8.4, J₂=1.5 Hz, 2H), 6.85 (m, 1H), 5.96 (dt, J₁=16.1 Hz, J₂=6.1 Hz, 1H), 5.57 (dt, J₁=16.1 Hz, J₂=1.5 Hz, 1H), 3.85 (s, 3H), 3.65 (q, J=6.5 Hz, 1H), 3.05-2.89 (m, 2H), 1.28 (d, 3H), 1.20 (s, 9H).

### Compound 137:

¹H NMR (DMSO-d₆): δ=7.19-7.05 (m, 2H), 6.92-6.82 (m, 1H), 5.89 (dt, J₁=10.7 Hz, J₂=6.4 Hz, 1H), 5.53 (dt, J₁=10.7 Hz, J₂=1.4 Hz, 1H), 3.83 (s, 3H), 3.77 (q, 1H), 3.22 (dt, 2H), 1.28 (d, 3H), 1.09 (s, 9H).

### Example 34

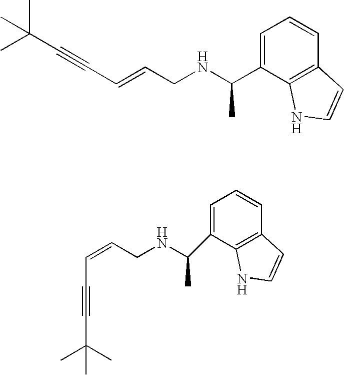

### (E)/(R)-(6,6-Dimethyl-hept-2-en-4-ynyl)-[1-(1H-indol-7-yl)-ethyl]-amine (compound 138) and (Z)/(R)-(6,6-Dimethyl-hept-2-en-4-ynyl)-[1-(1H-indol-7-yl)-ethyl]-amine (compound 139)

(E/Z)-1-bromo-6,6-dimethyl-hept-2-en-4-yne (1 equiv) and 1-(1H-indol-7-yl)-ethylamine (1 equiv) were treated as described in GP1. The crude was separated by chromatography (PE/EtOAc 1:0→3:1), giving the title compounds separately.

### Compound 138:

¹H NMR (DMSO-d₆): δ=10.81 (bs, 1NH), 7.38 (d, 1H), 7.27 (t, 1H), 7.02 (d, 1H), 6.93 (t, 1H), 6.40 (dd, 1H), 6.01 (dt, J₁=16.1 Hz, J₂=5.7 Hz, 1H), 5.58 (dt, J₁=16.1 Hz, J₂=1.5 Hz, 1H), 4.14 (q, 1H), 3.01 (d, 2H), 2.35 (bs, 1NH), 1.33 (d, 3H), 1.19 (s, 9H).

### Compound 139:

¹H NMR (DMSO-d₆): δ=10.91 (bs, 1NH), 7.41 (d, 1H), 7.29 (t, 1H), 7.09 (d, 1H), 6.96 (t, 1H), 6.41 (dd, 1H), 5.95 (dt, J₁=10.7 Hz, J₂=6.5 Hz, 1H), 5.53 (dt, J1=10.7 Hz, J2=1.5 Hz, 1H), 4.30 (q, 1H), 3.29 (d, 2H), 1.41 (d, 3H), 1.04 (s, 9H).

### Example 35

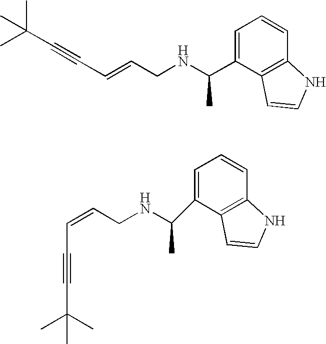

### (E)/(R)-(6,6-Dimethyl-hept-2-en-4-ynyl)-[1-(1H-indol-4-yl)-ethyl]-amine (Compound 140) and (Z)/(R)-(6,6-Dimethyl-hept-2-en-4-ynyl)-[1-(1H-indol-4-yl)-ethyl]-amine (Compound 141)

(E/Z)-1-bromo-6,6-dimethyl-hept-2-en-4-yne (1 equiv) and 1-(1H-indol-4-yl)-ethylamine (1 equiv) were treated as described in GP1. The crude was separated by chromatography (PE/EtOAc 1:0→3:1), giving the title compounds separately.

### Compound 140:

¹H NMR (DMSO-d₆): δ=7.32-7.19 (m, 2H), 7.08-6.93 (m, 2H), 6.60-6.54 (m, 1H), 5.99 (dt, J₁=15.8 Hz, J₂=5.9 Hz, 1H), 5.56 (dt, J₁=15.8 Hz, J₂=1.5 Hz, 1H), 4.09 (q, 1H), 3.09-2.94 (m, 2H), 1.33 (d, 3H), 1.19 (s, 9H).

### Compound 141:

¹H NMR (DMSO-d₆): δ=11.09 (bs, 1NH), 7.35-7.24 (m, 2H), 7.10-6.98 (m, 2H), 6.62-6.56 (m, 1H), 5.94 (dt, J₁=10.3 Hz, J₂=6.8 Hz, 1H), 5.56 (dt, J₁=10.3 Hz, J₂=1.4 Hz, 1H), 4.31 (q, 1H), 3.33 (dd, 2H), 1.44 (d, 3H), 1.05 (s, 9H).

### Example 36

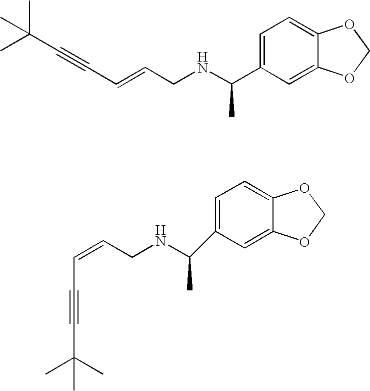

### (E)/(R)-(1-Benzo[1,3]dioxol-5-yl-ethyl)-(6,6-dimethyl-hept-2-en-4-ynyl)-amine (Compound 142) and (Z)/(R)-(1-Benzo[1,3]dioxol-5-yl-ethyl)-(6,6-dimethyl-hept-2-en-4-ynyl)-amine (compound 143)

(E/Z)-1-bromo-6,6-dimethyl-hept-2-en-4-yne (1 equiv) and 1-benzo[1,3]dioxol-5-yl-ethylamine (1 equiv) were treated as described in GP1. The crude was separated by chromatography (PE/EtOAc 1:0→3:1), giving the title compounds separately.

### Compound 142:

¹H NMR (DMSO-d₆): δ=6.90 (d, 1H), 6.81 (d, 1H), 6.73 (dd, 1H), 5.96 (dd, 2H), 5.95 (dt, J₁=16.0 Hz, J₂=6.1 Hz, 1H), 5.56 (dt, J₁=16.0 Hz, J₂=1.5 Hz, 1H), 3.60 (q, 1H), 3.04-2.86 (m, 2H), 1.19 (s, 9H), 1.18 (d, 3H).

### Compound 143:

¹H NMR (DMSO-d₆): δ=6.95 (d, 1H), 6.84 (d, 1H), 6.80 (dd, 1H), 5.97 (dd, 2H), 5.89 (dt, J₁=10.7 Hz, J₂=6.7 Hz, 1H), 5.54 (dt, J₁=10.7 Hz, J₂=1.5 Hz, 1H), 3.75 (q, 1H), 3.29-3.14 (m, 2H), 1.27 (d, 3H), 1.11 (s, 9H).

### Example 37

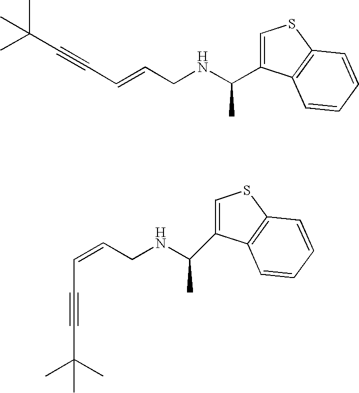

### (E)/(R)-(1-Benzo[b]thiophen-3-yl-ethyl)-(6,6-dimethyl-hept-2-en-4-ynyl)-amine Compound 144) and (E)/(R)-(1-Benzo[b]thiophen-3-yl-ethyl)-(6,6-dimethyl-hept-2-en-4-ynyl)-amine (Compound 145)

(E/Z)-1-bromo-6,6-dimethyl-hept-2-en-4-yne (1 equiv) and 1-benzo[b]thiophen-3-yl-ethylamine (1 equiv) were treated as described in GP1. The crude was separated by chromatography (PE/EtOAc 1:0→3:1), giving the title compounds separately.

### Compound 144:

¹H NMR (DMSO-d₆): δ=8.03-7.91 (m, 2H), 7.52 (s, 1H), 7.41-7.29 (m, 2H), 6.00 (dt, J₁=15.9 Hz, J₂=5.9 Hz, 1H), 5.60 (dt, J₁=15.9 Hz, J₂=1.6 Hz, 1H), 4.17 (q, 1H), 3.19-3.03 (m, 2H), 2.35 (bs, 1NH), 1.37 (d, 3H), 1.19 (s, 9H).

### Compound 145:

¹H NMR (DMSO-d₆): δ=8.02-7.90 (m, 2H), 7.56 (s, 1H), 7.42-7.30 (m, 2H), 5.93 (dt, J₁=10.7 Hz, J₂=6.7 Hz, 1H), 5.52 (bd, J=10.7 Hz, 1H), 4.24 (q, 1H), 3.33 (bd, 2H), 1.41 (d, 3H), 1.03 (s, 9H).

### Example 38

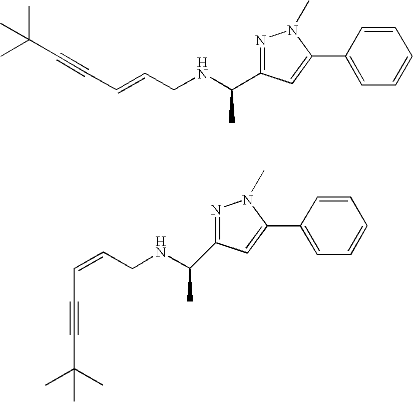

### (E)/(R)-(6,6-Dimethyl-hept-2-en-4-ynyl)-[1-(1-methyl-5-phenyl-1H-pyrazol-3-yl)-ethyl]-amine (Compound 146) and (Z)/(R)-(6,6-Dimethyl-hept-2-en-4-ynyl)-[1-(1-methyl-5-phenyl-1H-pyrazol-3-yl)-ethyl]-amine (Compound 147)

(E/Z)-1-bromo-6,6-dimethyl-hept-2-en-4-yne (1 equiv) and 1-(1-methyl-5-phenyl-1H-pyrazol-3-yl)-ethylamine (1 equiv) were treated as described in GP1. The crude was separated by chromatography (PE/EtOAc 1:0→3:1), giving the title compounds separately.

### Compound 146:

¹H NMR (DMSO-d₆): δ=7.55-7.39 (m, 5H), 6.34 (s, 1H), 5.97 (dt, J₁=16.0 Hz, J₂=6.1 Hz, 1H), 5.67 (dt, J₁=16.0 Hz, J₂=1.5 Hz, 1H), 3.87 (q, 1H), 3.80 (s, 3H), 3.27-3.11 (m, 2H), 1.33 (d, 3H), 1.20 (s, 9H).

### Compound 147:

¹H NMR (DMSO-d₆): δ=7.55-7.39 (m, 5H), 6.36 (s, 1H), 5.93 (dt, J₁=10.7 Hz, J₂=6.7 Hz, 1H), 5.57 (dt, J₁=10.7 Hz, J₂=0.9 Hz, 1H), 3.84 (q, 1H), 3.80 (s, 3H), 3.48-3.29 (m, 2H), 1.39 (d, 3H), 1.12 (s, 9H).

### Example 39

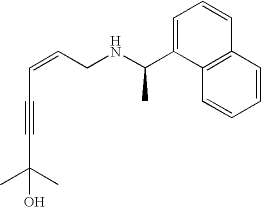

### (Z)/(R)-2-Methyl-7-(1-naphthalen-1-yl-ethylamino)-hept-5-en-3-yn-2-ol (Compound 148)

See GP2b.

¹H NMR (CDCl₃): δ=8.18 (bd, 1H), 7.89-7.84 (m, 1H), 7.75 (d, 1H), 7.68 (d, 1H), 7.55-7.43 (m, 3H), 6.01 (dt, J₁=11.0 Hz, J₂=7.0 Hz, 1H), 5.56 (dt, J₁=11.0 Hz, J₂=1.0 Hz, 1H), 4.69 (q, 1H), 3.44 (dd, 2H), 1.49 (d, 3H), 1.31 (d, 6H).

### Example 40

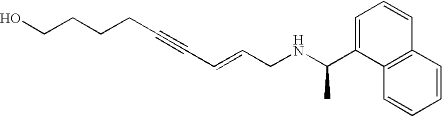

### (E)/(R)-9-(1-Naphthalen-1-yl-ethylamino)-non-7-en-5-yn-1-ol (Compound 149)

See GP2b.

¹H NMR (600 MHz, DMSO-d₆): δ=8.22 (bd, 1H), 7.93 (m, 1H), 7.81 (d, 1H), 7.70 (d, 1H), 7.56-7.47 (m, 3H), 6.04 (dt, J₁=15.9 Hz, J₂=6.1 Hz, 1H), 5.64 (dm, J=15.9 Hz, 1H), 4.66 (q, 1H), 3.40 (m, 2H), 3.16 (m, 2H), 2.30 (m, 2H), 1.49 (m, 4H), 1.41 (d, 3H).

### Example 41

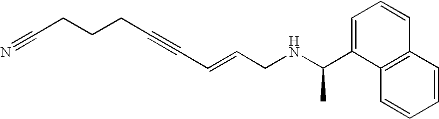

### (E)/(R)-9-(1-Naphthalen-1-yl-ethylamino)-non-7-en-5-ynenitrile (Compound 150)

See GP2b.

¹H NMR (600 MHz, DMSO-d₆): δ=8.23 (d, 1H), 7.93 (d, 1H), 7.81 (d, 1H), 7.70 (d, 1H), 7.56-7.48 (m, 3H), 6.10 (dt, J₁=15.9 Hz, J₂=6.0 Hz, 1H), 5.65 (dm, J=15.9 Hz, 1H), 4.65 (q, 1H), 3.16 (m, 2H), 2.56 (t, 2H), 2.41 (m, 2H), 1.76 (m, 2H), 1.40 (d, 3H).

### Example 41

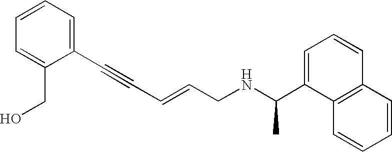

### (E)/(R)-{2-[5-(1-Naphthalen-1-yl-ethylamino)-pent-3-en-1-ynyl]-phenyl}-methanol (Compound 151)

See GP2b.

¹H NMR (600 MHz, DMSO-d₆): δ=8.25 (d, 1H), 7.94 (m, 1H), 7.82 (d, 1H), 7.72 (d, 1H), 7.58-7.47 (m, 4H), 7.40-7.36 (m, 2H), 7.24 (m, 1H), 6.30 (dt, J₁=15.9 Hz, J₂=5.9 Hz, 1H), 5.96 (dm, J=15.9 Hz, 1H), 4.67 (q, 1H), 4.62 (s, 2H), 3.23 (m, 2H), 1.42 (d, 3H).

### Example 43

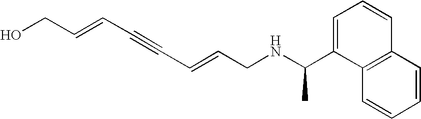

### (E,E)/((R)-8-(1-Naphthalen-1-yl-ethylamino)-octa-2,6-dien-4-yn-1-ol (Compound 152)

See GP2b.

¹H NMR (600 MHz, DMSO-d₆): δ=8.23 (bd, 1H), 7.94 (m, 1H), 7.82 (d, 1H), 7.70 (d, 1H), 7.57-7.48 (m, 3H), 6.21 (dt, J₁=15.9 Hz, J₂=4.6 Hz, 1H), 6.15 (dt, J₁=15.9 Hz, J₂=6.0 Hz, 1H), 5.82 (m, 2H), 4.68 (q, 1H), 4.03 (m, 2H), 3.21 (m, 2H), 1.42 (d, 3H).

### Example 44

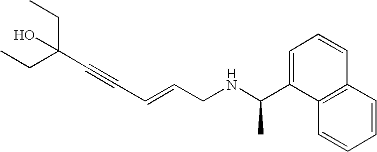

### (E)/(R)-3-Ethyl-8-(1-naphthalen-1-yl-ethylamino)-oct-6-en-4-yn-3-ol (Compound 153)

See GP2b.

¹H NMR (600 MHz, DMSO-d₆): δ=8.23 (d, 1H), 7.92 (m, 1H), 7.79 (d, 1H), 7.68 (d, 1H), 7.55-7.47 (m, 3H), 6.08 (dt, J₁=15.8 Hz, J₂=5.9 Hz, 1H), 5.67 (dm, J=15.8 Hz, 1H), 4.59 (q, 1H), 3.12 (m, 2H), 1.53 (m, 4H), 1.38 (d, 3H), 0.91 (t, 6H).

### Example 45

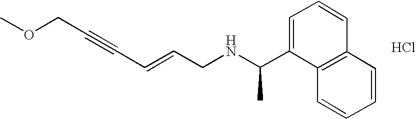

### (E)/(R)-(6-Methoxy-hex-2-en-4-ynyl)-(1-naphthalen-1-yl-ethyl)-amine hydrochloride (Compound 154)

(E)/(R)-(3-Bromo-allyl)-(1-naphthalen-1-yl-ethyl)-amine (1 mmol) and 3-methoxy-propyne (3.5 mmol) were treated as described in GP2a. Chromatography (PE/EtOAc 4:1) gave a free amine. The free amine was redissolved in Et₂O. To the solution was added HCl in 1,4-dioxane (4N) until pH2 and the product was crystallized out. Filtration and wash of the crystals with small amount of Et₂O gave the title compound as a white solid.

¹³C NMR (DMSO-d₆): δ=134.5, 134.2, 133.7, 130.5, 129.3, 127.3, 126.5, 125.9, 124.8, 122.9, 116.1, 88.2, 83.7, 59.7, 57.3, 52.1, 46.6, 20.2.

### Example 46

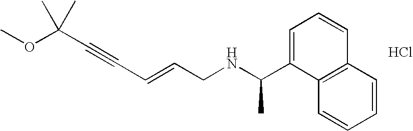

### (E)/(R)-(6-Methoxy-6-methyl-hept-2-en-4-ynyl)-(1-naphthalen-1-yl-ethyl)-amine hydrochloride (Compound 155)

(E)/(R)-(3-Bromo-allyl)-(1-naphthalen-1-yl-ethyl)-amine (1 mmol) and 3-methoxy-3-methyl-but-1-yne (3.5 mmol) were treated as described in GP2a. Chromatography (PE/EtOAc 4:1) gave a free amine. The free amine was redissolved in Et₂O. To the solution was added HCl in 1,4-dioxane (4N) until pH2 and the product was crystallized out. Filtration and wash of the crystals with small amount of Et₂O gave the title compound as a white solid.

¹³C NMR (DMSO-d₆): δ=133.8, 133.8, 133.3, 130.1, 128.9, 128.8, 126.8, 126.1, 125.5, 124.4, 122.5, 115.8, 93.3, 81.2, 70.1, 51.7, 50.9, 46.2, 27.9, 19.9.

### Example 47

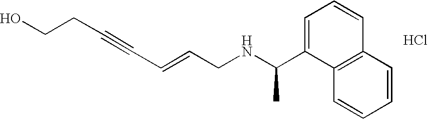

### (E)/(R)-7-(1-Naphthalen-1-yl-ethylamino)-hept-5-en-3-yn-1-ol hydrochloride (Compound 156)

(E)/(R)-(3-Bromo-allyl)-(1-naphthalen-1-yl-ethyl)-amine (1 mmol) and but-3-yn-1-ol (2 mmol) were treated as described in GP2a. Chromatography (EtOAc) gave a free amine. The free amine was redissolved in EtO. To the solution was added conc. HCl until pH2 and the product was crystallized out. Filtration and wash of the crystals with small amount of Et₂O gave the title compound as a white solid.

¹³C NMR (DMSO-d₆): δ=133.9, 133.3, 132.2, 130.1, 128.8, 126.8, 126.1, 125.5, 124.3, 122.5, 117.0, 90.9, 78.5, 59.5, 51.6, 46.3, 23.1, 19.8.

### Example 48

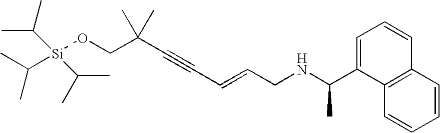

### (E)/(R)-(6,6-Dimethyl-7-triisopropylsilanyloxy-hept-2-en-4-ynyl)-(1-naphthalen-1-yl-ethyl)-amine (Compound 157)

(E)/(R)-(3-Bromo-allyl)-(1-naphthalen-1-yl-ethyl)-amine (1 equiv) and (2,2-dimethyl-but-3-ynyloxy)-triisopropyl-silane (2.0 equiv) were treated as described in GP2a.

### Example 49

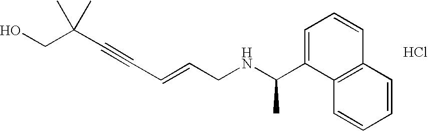

### (E)/(R)-2,2-Dimethyl-7-(1-naphthalen-1-yl-ethylamino)-hept-5-en-3-yn-1-ol hydrochloride (Compound 158)

To a solution of compound 157 (1.8 g, 3.3 mmol) in THF (20 mL) was added a 1 M solution of TBAF in THF at rt. After being stirred at this temperature for 2 days, the reaction solution was diluted with Et₂O and washed with brine/H₂O (1/1). The aqueous solution was extracted with Et₂O. The combined organic phases were dried and concentrated in vacuo. The residue was purified by chromatography (PE/EtOAc 1:1), furnishing an oil. The oil product was dissolved in Et₂O and acidified to pH 1-2 with conc. HCl. The mixture was concentrated to dryness in vacuo, giving the title compound as a white solid.

¹³C NMR (DMSO-d₆): δ=133.9, 132.0, 130.6, 129.6, 129.4, 127.2, 126.3, 126.1, 125.2, 121.7, 119.3, 98.1, 78.5, 71.4, 52.3, 46.8, 34.8, 25.2, 25.2, 21.5.

### Example 50

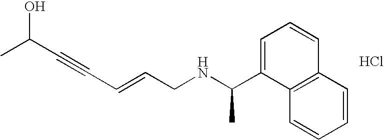

### (E)/(2R/S)-7-[(R)-1-Naphthalen-1-yl-ethylamino]-hept-5-en-3-yn-2-ol hydrochloride (Compound 159)

(E)/(R)-(3-Bromo-allyl)-(1-naphthalen-1-yl-ethyl)-amine (1 equiv) and but-3-yn-1-ol (2.0 equiv) were treated as described in GP2a. Chromatography (PE/EtOAc 1:1) gave a free amine. The free amine was redissolved in Et₂O. To the solution was added conc. HCl until pH2 and the product was crystallized out. Filtration and wash of the crystals with small amount of Et₂O gave the title compound as a solid.

¹³C NMR (DMSO-d₆): δ=133.8, 133.3, 133.0, 130.1, 128.9, 128.8, 126.8, 126.1, 125.5, 124.3, 122.5, 116.3, 95.4, 79.6, 56.4, 51.6, 46.3, 24.4, 19.7.

### Example 51

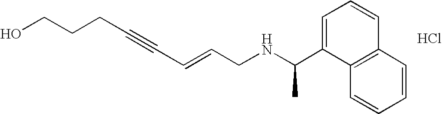

### (E)/(R)-8-(1-Naphthalen-1-yl-ethylamino)-oct-6-en-4-yn-1-ol hydrochloride (Compound 160)

(E)/(R)-(3-Bromo-allyl)-(1-naphthalen-1-yl-ethyl)-amine from preparation 1 (1 equiv) and pent-4-yn-1-ol (3.5 equiv) were treated as described in GP2a. Chromatography (EtOAc) gave a free amine. The free amine was redissolved in Et₂O. To the solution was added HCl in 1,4-dioxane (4N) until pH2 and the product was crystallized out. Filtration and wash of the crystals with small amount of Et₂O gave the title compound as a solid.

¹³C NMR (DMSO-d₆): δ=133.8, 133.3, 132.0, 130.1, 128.8, 126.8, 126.1, 125.5, 124.3, 122.5, 117.1, 92.9, 77.8, 59.2, 51.5, 46.3, 31.3, 19.7, 15.2.

### Example 52

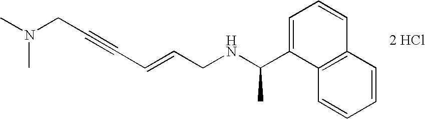

### (E)/(R)—N*6*,N*6*-Dimethyl-N*1*-(1-naphthalen-1-yl-1-ethyl)-hex-2-en-4-yne-1,6-diamine dihydrochloride (Compound 161)

(E)/(R)-(3-Bromo-allyl)-(1-naphthalen-1-yl-ethyl)-amine (1 equiv) and dimethyl-prop-2-ynyl-amine (3.5 equiv) were treated as described in GP2a. Chromatography (EtOAc) gave a free amine. The free amine was redissolved in Et₂O. To the solution was added HCl in 1,4-dioxane (4N) until pH2 and the product was crystallized out. Filtration and wash of the crystals with small amount of Et₂O gave the title compound as a solid.

¹³C NMR (DMSO-d₆): δ=136.3, 133.9, 133.4, 130.2, 129.0, 127.0, 126.2, 125.6, 124.6, 122.6, 114.7, 86.1, 80.7, 66.4, 51.9, 46.2, 45.9, 41.3, 20.0.

### Example 53

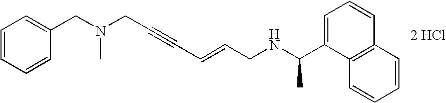

### (E)/(R)—N*6*-Benzyl-N*6*-methyl-N*1*-(1-naphthalen-1-yl-ethyl)-hex-2-en-4-yne-1,6-diamine dihydrochloride (Compound 162)

(E)/(R)-(3-Bromo-allyl)-(1-naphthalen-1-yl-ethyl)-amine (1 equiv) and benzyl-methyl-prop-2-ynyl-amine (3.5 equiv) were treated as described in GP2a. Chromatography (PE/EtOAc 1:1→0:1) gave a free amine. The free amine was redissolved in Et₂O. To the solution was added HCl in 1,4-dioxane (4N) until pH2 and the product was crystallized out. Filtration and wash of the crystals with small amount of Et₂O gave the title compound as a solid.

¹³C NMR (DMSO-d₆): δ=136.7, 134.2, 133.7, 131.6, 130.5, 130.2, 129.9, 129.3, 129.2, 127.3, 126.5, 125.9, 124.9, 122.9, 115.0, 87.0, 80.7, 57.7, 52.2, 46.5, 44.5, 39.0, 20.3.

### Example 54

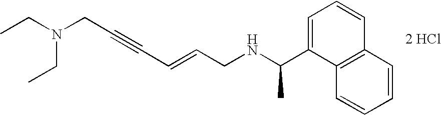

### (E)/(R)—N*6*,N*6*-Diethyl-N*1*-(1-naphthalen-1-yl-ethyl)-hex-2-en-4-yne-1,6-diamine dihydrochloride (Compound 163)

(E)/(R)-(3-Bromo-allyl)-(1-naphthalen-1-yl-ethyl)-amine (1 equiv) and diethyl-prop-2-ynyl-amine (3.5 equiv) were treated as described in GP2a. Chromatography (PE/EtOAc 1:1→0:1) gave a free amine. The free amine was redissolved in Et₂O. To the solution was added HCl in 1,4-dioxane (4N) until pH2 and the product was crystallized out. Filtration and wash of the crystals with small amount of Et₂O gave the title compound as a solid.

¹³C NMR (DMSO-d₆): δ=136.6, 134.2, 133.7, 130.5, 129.3, 127.3, 126.5, 125.9, 125.0, 122.9, 115.0, 86.3, 80.6, 52.2, 47.4, 47.2, 46.5, 40.9, 20.4.

### Example 55

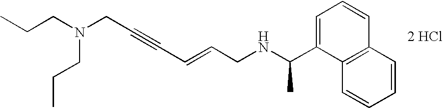

### (E)/(R)—N*1*-(1-Naphthalen-1-yl-ethyl)-N*6*,N*6*-dipropyl-hex-2-en-4-yne-1,6-diamine dihydrochloride (Compound 164)

(E)/(R)-(3-Bromo-allyl)-(1-naphthalen-1-yl-ethyl)-amine (1 equiv) and dipropyl-prop-2-ynyl-amine (3.5 equiv) were treated as described in GP2a. Chromatography (PE/EtOAc 1:1→0:1) gave a free amine. The free amine was redissolved in Et₂O. To the solution was added HCl in 1,4-dioxane (4N) until pH2 and the product was crystallized out. Filtration and wash of the crystals with small amount of Et₂O gave the title compound as a solid.

¹³C NMR (DMSO-d₆): δ=136.2, 133.8, 133.3, 130.1, 128.8, 126.8, 126.0, 125.5, 124.5, 122.5, 114.5, 86.1, 80.1, 53.8, 51.7, 46.1, 41.8, 19.9, 16.7, 10.9.

### Example 56

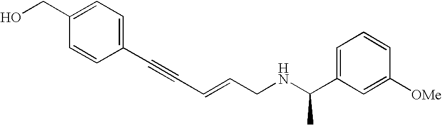

### (E)/(R)-(4-{5-[1-(3-Methoxy-phenyl)-ethylamino]-pent-3-en-1-ynyl}-phenyl)-methanol (Compound 165)

See GP2b.

¹H NMR (CDCl₃): δ=8.44 (bs, 1H), 7.40 (d, 2H), 7.34-7.27 (m, 3H), 7.03-6.94 (m, 2H), 6.89-6.84 (m, 1H), 6.30 (dt, J₁=15.6 Hz, J₂=6.9 Hz, 1H), 5.83 (bd, J=15.6 Hz, 1H), 4.68 (s, 2H), 4.02 (q, 1H), 3.83 (s, 3H), 3.43-3.21 (m, 2H), 1.55 (d, 3H).

### Example 57

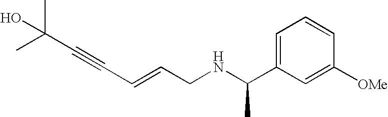

### (E)/(R)-7-[1-(3-Methoxy-phenyl)-ethylamino]-2-methyl-hept-5-en-3-yn-2-ol (Compound 166)

See GP2b.

¹H NMR (CDCl₃): δ=7.29 (t, 1H), 7.02-6.93 (m, 2H), 6.86 (dd, 1H), 6.13 (dt, J₁=15.6 Hz, J₂=6.8 Hz, 1H), 5.65 (bd, J=15.6 Hz, 1H), 4.01 (q, 1H), 3.83 (s, 3H), 3.37-3.18 (m, 2H), 1.54 (d, 3H), 1.51 (s, 6H).

### Example 58

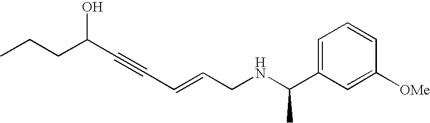

### (E)/(4R/S)-9-[(R)-1-(3-Methoxy-phenyl)-ethylamino]-non-7-en-5-yn-4-ol (Compound 167)

See GP2b.

¹H NMR (CDCl₃): δ=7.27 (t, 1H), 6.95-6.89 (m, 2H), 6.85-6.79 (m, 1H), 6.17 (dt, J₁=16.4 Hz, J₂=6.0 Hz, 1H), 5.64 (bd, J=16.4 Hz, 1H), 4.47 (dt, 1H), 3.88 (q, 1H), 3.82 (s, 3H), 3.30-3.11 (m, 2H), 1.74-1.61 (m, 2H), 1.47 (h, 2H), 1.45 (d, 3H), 0.95 (t, 3H).

### Example 59

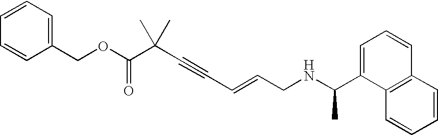

### (E)/(R)-Benzyl 2,2-dimethyl-7-(1-naphthalen-1-yl-ethylamino)-hept-5-en-3-ynoate (Compound 168)

(E)/(R)-(3-Bromo-allyl)-(1-naphthalen-1-yl-ethyl)-amine (1 equiv) and benzyl 2,2-dimethyl-but-3-ynoate (2.0 equiv) were treated as described in GP2a. Chromatography (PE/EtOAc 1:0→3:1) gave the title compound.

¹H NMR (CDCl₃): δ=8.17-8.06 (m, 1H), 7.93-7.68 (m, 3H), 7.57-7.42 (m, 3H), 7.39-7.18 (m, 5H), 6.22 (dt, J₁=15.8 Hz, J₂=6.4 Hz, 1H), 5.58 (bd, J=15.7 Hz, 1H), 5.17 (s, 2H), 4.78 (q, 1H), 3.40-3.10 (m, 2H), 1.61 (d, 3H), 1.49 (s, 6H).

### Example 60

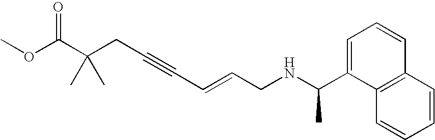

### (E)/(R)-Methyl 2,2-dimethyl-8-(1-naphthalen-1-yl-ethylamino)-oct-6-en-4-ynoate (Compound 169)

(E)/(R)-(3-Bromo-allyl)-(1-naphthalen-1-yl-ethyl)-amine (1 equiv) and methyl 2,2-dimethyl-pent-4-ynoate (2.0 equiv) were treated as described in GP2a. Chromatography (PE/EtOAc 1:0→3:1) gave the title compound.

¹³C NMR (CDCl₃): δ=177.3, 141.3, 140.9, 134.0, 131.3, 129.0, 127.3, 125.8, 125.7, 125.3, 122.9, 122.7, 111.0, 86.3, 80.9, 52.9, 52.0, 49.3, 42.5, 30.6, 24.6, 23.6.

### Example 61

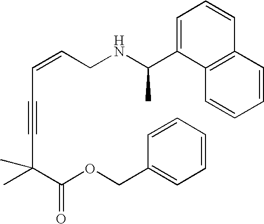

### (Z)/(R)Benzyl 2,2-dimethyl-7-(1-naphthalen-1-yl-ethylamino)-hept-5-en-3-ynoate (Compound 170)

(Z)/(R)-(3-Bromo-allyl)-(1-naphthalen-1-yl-ethyl)-amine (1 equiv) and 2,2-dimethyl-but-3-ynoic acid benzyl ester (2.0 equiv) were treated as described in GP2a. Chromatography (PE/EtOAc 1:0→3:1) gave the title compound.

¹³C NMR (CDCl₃): δ=173.4, 141.4, 140.9, 135.9, 134.0, 131.3, 128.9, 128.5, 128.1, 127.7, 127.2, 125.8, 125.7, 125.3, 123.0, 122.7, 111.6, 96.6, 78.2, 66.9, 52.8, 46.7, 38.9, 27.0, 23.4.

### Example 62

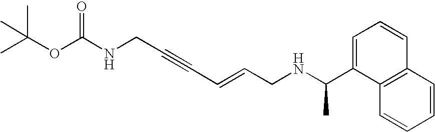

### (E)/(R)-tert-Butyl [6-(1-naphthalen-1-yl-ethylamino)-hex-4-en-2-ynyl]-carbamate (Compound 171)

(E)/(R)-(3-Bromo-allyl)-(1-naphthalen-1-yl-ethyl)-amine (1 equiv) and tert-butyl prop-2-ynyl-carbamate (2.0 equiv) were treated as described in GP2a. Chromatography (PE/EtOAc 1:0→3:1) gave the title compound.

¹³C NMR (CDCl₃): δ=155.3, 142.7, 140.8, 134.0, 131.3, 129.0, 127.3, 125.8, 125.7, 125.4, 122.9, 122.7, 110.2, 85.1, 81.4, 52.9, 49.2, 31.2, 28.4, 23.6.

### Example 63

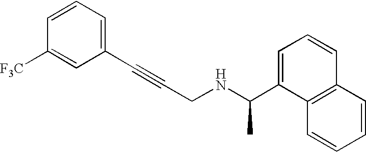

### (R)-(1-Naphthalen-1-yl-ethyl)-[3-(3-trifluoromethyl-phenyl)-prop-2-ynyl]-amine hydrochloride (Compound 172)

To a mixture of (R)-(1-naphthalen-1-yl-ethyl)-prop-2-ynyl-amine hydrochloride (1.23 g, 5.0 mmol) and 1-iodo-3-trifluoromethyl-benzene (1.35 g, 5.0 mmol) in Et₃N/THF (1:10, 10 mL) were added (Ph₃P)₂PdCl₂ (350 mg, 0.5 mmol), and CuI (48 mg, 0.25 mmol) at rt. After being stirred for 18 h at this temperature, the reaction mixture was concentrated in vacuo together with silica gel. The crude was purified by chromatography (PE/EtOAc 4:1), giving the title compound.

¹H NMR (CDCl₃): δ=8.30 (m, 1H), 7.88 (m, 1H), 7.77 (d, J=8.0 Hz, 1H), 7.71 (d, J=7.3 Hz, 1H), 7.66 (s, 1H), 7.58-7.37 (m, 6H), 4.96 (q, J=6.5 Hz, 1H), 3.70/3.54 (d, J=17.2 Hz, 2H), 1.55 (d, J=6.5 Hz, 3H).

### Example 64

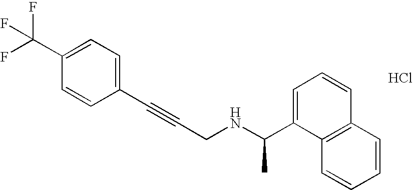

### (R)-(1-Naphthalen-1-yl-ethyl)-[3-(4-trifluoromethyl-phenyl)-prop-2-ynyl]-amine hydrochloride (Compound 173)

[1-(3-methoxy-phenyl)-ethyl]-prop-2-ynyl-amine and 1-iodo-3-trifluoromethyl-benzene were treated as described for the preparation of compound 172. The crude was purified by chromatography (PE/EtOAc 4:1), giving a free amine. This free amine was dissolved in Et₂O. To the solution was added HCl in 1,4-dioxane (4N) until pH2 and the product was crystallized out. Filtration and wash of the crystals with Et₂O gave the title compound as a solid.

¹³C NMR (DMSO-d₆): δ=133.4, 132.2, 130.2, 129.1, 129.1 (²J_{CF}=32.7 Hz), 128.9, 126.8, 126.1, 125.6, 125.6, 125.3, 124.7, 123.8 (¹J_{CF}=272.9 Hz), 122.5, 85.7, 83.6, 51.3, 34.6, 19.8.

### Example 65

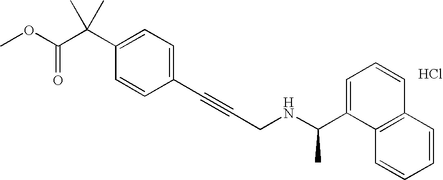

### (R)-Methyl 2-methyl-2-{4-[3-(1-naphthalen-1-yl-ethylamino)-prop-1-ynyl]-phenyl}-propanoate hydrochloride (Compound 174)

To a mixture of (R)-(1-naphthalen-1-yl-ethyl)-prop-2-ynyl-amine hydrochloride (0.98 g, 4.0 mmol) and methyl 2-(4-bromo-phenyl)-2-methyl-propanoate (1.03 g, 4.0 mmol) in Et₃N/THF (1:10, 14 mL) were added (Ph₃P)₂PdCl₂ (350 mg, 0.5 mmol), and CuI (85 mg, 0.5 mmol) at rt. After being refluxed for 18 h, the reaction mixture was concentrated in vacuo together with silica gel. The crude was purified by chromatography (PE/EtOAc 4:1), giving the title compound.

¹³C NMR (DMSO-d₆): δ=175.8, 145.8, 133.3, 131.5, 130.2, 129.1, 128.9, 126.8, 126.1, 126.0, 125.5, 124.5, 122.5, 119.3, 86.9, 80.7, 52.1, 51.1, 46.2, 34.7, 26.0, 19.6.

### Example 66

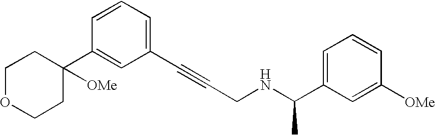

### (R)-[1-(3-Methoxy-phenyl)-ethyl]-{3-[3-(4-methoxy-tetrahydro-pyran-4-yl)-phenyl]-prop-2-ynyl}-amine (Compound 175)

[1-(3-methoxy-phenyl)-ethyl]-prop-2-ynyl-amine (189 mg, 1.0 mmol) and 4-(3-bromo-phenyl)-4-methoxy-tetrahydro-pyran (271 mg, 1.0 mmol) were treated as described for the preparation of compound 172. The crude product was purified by flash chromatography (PE/EtOAc 10:1→3:1), giving the title compound.

¹³C NMR (CDCl₃): δ=159.9, 146.4, 144.6, 130.7, 129.5, 129.4, 128.5, 125.8, 123.4, 119.3, 112.6, 112.4, 87.9, 83.4, 74.8, 63.6, 56.8, 55.2, 49.9, 36.9, 35.2, 24.0.

### Example 67

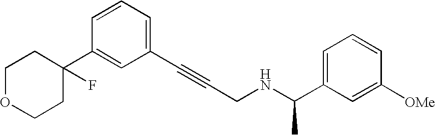

### (R)-[1-(3-Methoxy-phenyl)-ethyl]-{3-[3-(4-fluoro-tetrahydro-pyran-4-yl)-phenyl]-prop-2-ynyl}-amine (Compound 176)

[1-(3-methoxy-phenyl)-ethyl]-prop-2-ynyl-amine (189 mg, 1.0 mmol) and 4-(3-bromo-phenyl)-4-fluoro-tetrahydro-pyran (271 mg, 1.0 mmol) were treated as described for the preparation of compound 172. The crude product was purified by flash chromatography (PE/EtOAc 10:1→3:1), giving the title compound.

¹³C NMR (CDCl₃): δ=159.9, 146.3, 144.3 (²J_{CF}=21.9 Hz), 130.9, 129.5, 128.5, 127.4, 127.3, 123.7, 123.6, 119.3, 112.6, 112.4, 93.2 (¹J_{CF}=175.1 Hz), 88.1, 83.2, 63.7, 56.7, 55.2, 37.0 (²J_{CF}=23.2 Hz), 24.0.

### Example 68

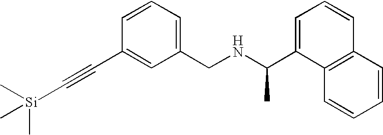

### (R)-(1-Naphthalen-1-yl-ethyl)-(3-trimethylsilanylethynyl-benzyl)-amine (Compound 177)

3-Trimethylsilanylethynyl-benzaldehyde and (R)-1-naphthalen-1-yl-ethylamine were treated as described in GP6. The crude was purified by flash chromatography (heptane/EtOAc 1:0→0:1), giving the title compound.

¹³C NMR (DMSO-d₆): δ=141.7, 141.4, 133.6, 131.1, 131.0, 129.7, 128.8, 128.8, 128.4, 126.8, 125.8, 125.8, 125.3, 123.1, 122.9, 122.0, 105.6, 93.8, 52.5, 50.4, 23.8, 0.0.

### Example 69

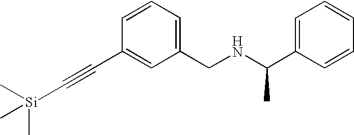

### (R)-(1-Phenyl-ethyl)-(3-trimethylsilanylethynyl-benzyl)-amine (Compound 178)

3-Trimethylsilanylethynyl-benzaldehyde and (R)-1-phenyl-ethylamine were treated as described in GP6. The crude was purified by flash chromatography (heptane/EtOAc 1:0→0:1), giving the title compound.

¹³C NMR (DMSO-d₆): δ=146.1, 141.7, 131.0, 129.7, 128.7, 128.4, 128.3, 126.6, 126.6, 122.0, 105.6, 93.8, 56.9, 50.2, 24.6, 0.0.

### Example 70

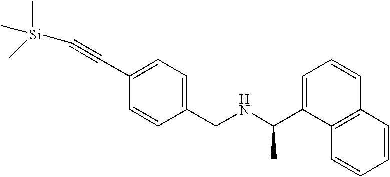

### (R)-(1-Naphthalen-1-yl-ethyl)-(4-trimethylsilanylethynyl-benzyl)-amine (Compound 179)

4-Trimethylsilanylethynyl-benzaldehyde and (R)-1-naphthalen-1-yl-ethylamine were treated as described in GP6. The crude was purified by flash chromatography (heptane/EtOAc 1:0→0:1).

¹³C NMR (DMSO-d₆): δ=142.3, 141.3, 133.6, 131.4, 131.0, 128.7, 128.1, 126.8, 125.7, 125.3, 123.0, 122.8, 120.3, 105.5, 93.5, 52.5, 50.6, 23.8, 0.0.

### Example 71

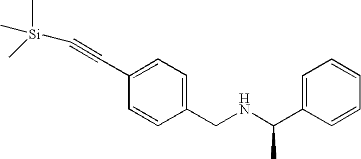

### (R)-(1-Phenyl-ethyl)-(4-trimethylsilanylethynyl-benzyl)-amine (Compound 180)

4-Trimethylsilanylethynyl-benzaldehyde and (R)-1-phenyl-ethylamine were treated as described in GP6. The crude was purified by flash chromatography (heptane/EtOAc 1:0→0:1).

¹³C NMR (DMSO-d₆): δ=146.0, 142.3, 131.4, 128.3, 128.0, 126.5, 120.2, 105.5, 93.5, 56.8, 50.4, 24.5, 0.0

### Example 72

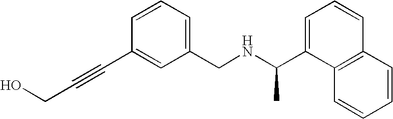

### (R)-3-{3-[(1-Naphthalen-1-yl-ethylamino)-methyl]-phenyl}-prop-2-yn-1-ol (Compound 181)

(R)-(3-Iodo-benzyl)-(1-naphthalen-1-yl-ethyl)-amine (compound 416) from preparation 15 (267 mg, 0.7 mmol) and prop-2-yn-1-ol (39 mg, 0.7 mmol) were treated as described for preparation of compound 172. The crude was purified by chromatography (heptane/EtOAc 1:0→2:3), giving the title compound.

¹³C NMR (DMSO-d₆): δ=141.5, 141.2, 133.5, 130.9, 130.6, 129.3, 128.6, 128.3, 128.0, 126.7, 125.6, 125.2, 122.9, 122.7, 122.1, 89.5, 83.7, 52.4, 50.3, 49.4, 23.6.

### Example 73

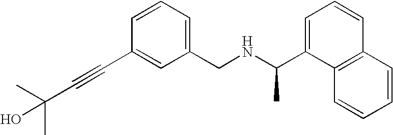

### (R)-2-Methyl-4-{3-[(1-naphthalen-1-yl-ethylamino)-methyl]-phenyl}-but-3-yn-2-ol (Compound 182)

(R)-(3-Iodo-benzyl)-(1-naphthalen-1-yl-ethyl)-amine (compound 416) from preparation 15 (267 mg, 0.7 mmol) and 2-methyl-but-3-yn-2-ol (61 mg, 0.7 mmol) were treated as described for preparation of compound 172. The crude was purified by chromatography (heptane/EtOAc 1:0→2:3), giving the title compound.

¹³C NMR (DMSO-d₆): δ=141.9, 141.7, 133.9, 131.3, 131.0, 129.6, 129.0, 128.6, 128.3, 127.1, 126.1, 125.6, 123.3, 123.2, 122.7, 96.1, 80.9, 63.9, 52.8, 50.8, 32.0, 24.1.

### Example 74

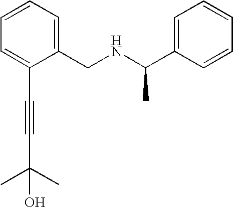

### (R)-2-Methyl-4-{2-[(1-phenyl-ethylamino)-methyl]-phenyl}-but-3-yn-2-ol (Compound 183)

To a mixture of (R)-(2-Iodo-benzyl)-(1-phenyl-ethyl)-amine (compound 422) from preparation 21 (233 mg, 0.69 mmol), CuI (5 mg, 0.028 mmol) and Pd(Ph₃P)₂Cl₂ (10 mg, 0.014 mmol) in piperidine (2 mL) was dropwise added a solution of 2-methyl-but-3-yn-2-ol (61 mg, 0.7 mmol) in piperidine (0.5 mL) at rt. After being stirred at this temperature overnight, the reaction mixture was concentrated in vacuo together with celite. The residue was purified by flash chromatography (heptane/EtOAc 1:0→2:3), giving the title compound.

¹³C NMR (DMSO-d₆): δ=145.9, 142.5, 131.3, 128.1, 128.0, 127.9, 126.4, 121.3, 100.2, 78.4, 63.6, 56.9, 48.9, 31.5, 24.2.

### Example 75

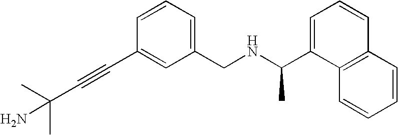

### (R)-1,1-Dimethyl-3-{3-[(1-naphthalen-1-yl-ethylamino)-methyl]-phenyl}-prop-2-ynylamine (Compound 184)

(R)-(3-Iodo-benzyl)-(1-naphthalen-1-yl-ethyl)-amine (compound 416) from preparation 15 (267 mg, 0.7 mmol) and 1,1-dimethyl-prop-2-ynylamine (60 mg, 0.7 mmol) were treated as described for preparation of compound 172. The crude was purified by chromatography (heptane/EtOAc 1:0→2:3), giving the title compound.

¹³C NMR (DMSO-d₆): δ=142.0, 141.6, 133.9, 131.3, 131.0, 129.0, 128.9, 128.8, 128.3, 127.1, 126.1, 125.6, 123.3, 123.2, 121.8, 90.8, 87.9, 59.0, 52.9, 50.7, 31.1, 24.0.

### Example 76

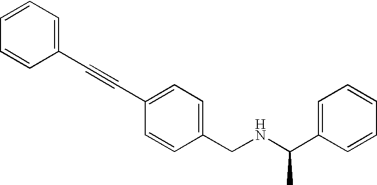

### (R)-(1-Phenyl-ethyl)-(4-phenylethynyl-benzyl)-amine (Compound 185)

4-Phenylethynyl-benzaldehyde and (R)-1-phenyl-ethylamine were treated as described in GP6. The crude was purified by flash chromatography (heptane/EtOAc 1:0→0:1).

¹³C NMR (DMSO-d₆): δ=145.9, 142.0, 131.2, 131.0, 128.6, 128.6, 128.2, 128.1, 126.5, 122.3, 120.1, 89.4, 88.7, 56.7, 50.3, 24.4.

### Example 77

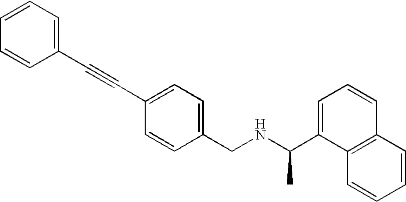

### (R)-(1-Naphthalen-1-yl-ethyl)-(4-phenylethynyl-benzyl)-amine (Compound 186)

4-Phenylethynyl-benzaldehyde and (R)-1-naphthalen-1-yl-ethylamine were treated as described in GP6. The crude was purified by flash chromatography (heptane/EtOAc 1:0→0:1).

¹³C NMR (DMSO-d₆): δ=142.0, 141.3, 133.5, 131.2, 131.0, 130.9, 128.6, 128.6, 128.1, 126.7, 125.6, 125.2, 122.9, 122.8, 122.3, 120.2, 89.4, 88.8, 52.5, 50.5, 23.7.

### Example 78

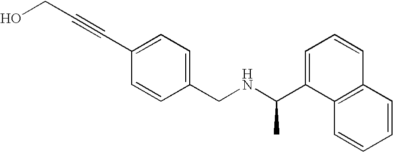

### (R)-3-{4-[(1-Naphthalen-1-yl-ethylamino)-methyl]-phenyl}-prop-2-yn-1-ol (Compound 187)

4-(3-Hydroxy-prop-1-ynyl)-benzaldehyde and (R)-1-naphthalen-1-yl-ethylamine were treated as described in GP6. The crude was purified by flash chromatography (heptane/EtOAc 1:0→0:1).

¹³C NMR (DMSO-d₆): δ=141.6, 141.2, 133.5, 130.9, 128.6, 128.0, 126.7, 125.6, 125.2, 122.9, 122.7, 120.4, 89.3, 83.6, 52.5, 50.5, 49.4, 23.7.

### Example 79

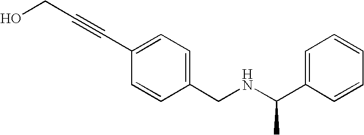

### (R)-3-{4-[(1-Phenyl-ethylamino)-methyl]-phenyl}-prop-2-yn-1-ol (Compound 188)

4-(3-Hydroxy-prop-1-ynyl)-benzaldehyde and (R)-1-phenyl-ethylamine were treated as described in GP6. The crude was purified by flash chromatography (heptane/EtOAc 1:0→0:1).

¹³C NMR (DMSO-d₆): δ=145.8, 141.4, 130.9, 128.2, 128.0, 126.5, 126.5, 120.4, 89.3, 83.6, 56.7, 50.2, 49.4, 24.3.

### Example 80

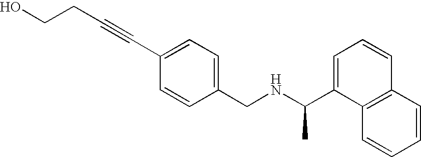

### (R)-4-{4-[(1-Naphthalen-1-yl-ethylamino)-methyl]-phenyl}-but-3-yn-1-ol (Compound 189)

4-(4-Hydroxy-but-1-ynyl)-benzaldehyde and (R)-1-naphthalen-1-yl-ethylamine were treated as described in GP6. The crude was purified by flash chromatography (heptane/EtOAc 1:0→0:1).

¹³C NMR (DMSO-d₆): δ=141.3, 141.0, 133.5, 130.9, 130.9, 128.6, 127.9, 126.7, 125.6, 125.2, 122.9, 122.7, 121.2, 87.8, 81.0, 59.7, 52.4, 50.4, 23.7, 23.2.

### Example 81

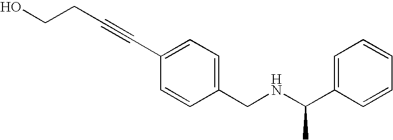

### (R)-4-{4-[(1-Phenyl-ethylamino)-methyl]-phenyl}-but-3-yn-1-ol (Compound 190)

4-(4-Hydroxy-but-1-ynyl)-benzaldehyde and (R)-1-phenyl-ethylamine were treated as described in GP6. The crude was purified by flash chromatography (heptane/EtOAc 1:0→0:1).

¹³C NMR (DMSO-d₆): δ=145.9, 140.9, 130.9, 128.1, 127.9, 126.4, 121.1, 87.8, 81.0, 59.7, 56.7, 50.2, 24.4, 23.2, 21.0.

### Example 82

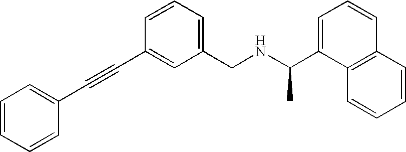

### (R)-(1-Naphthalen-1-yl-ethyl)-(3-phenylethynyl-benzyl)-amine (Compound 191)

3-Phenylethynyl-benzaldehyde and (R)-1-naphthalen-1-yl-ethylamine were treated as described in GP6. The crude was purified by flash chromatography (heptane/EtOAc 1:0→0:1).

¹³C NMR (DMSO-d₆): δ=141.7, 141.3, 133.5, 131.3, 130.9, 130.7, 129.4, 128.7, 128.4, 128.4, 126.7, 125.6, 125.2, 122.9, 122.8, 122.3, 121.9, 89.5, 89.0, 52.5, 50.4, 23.7.

### Example 83

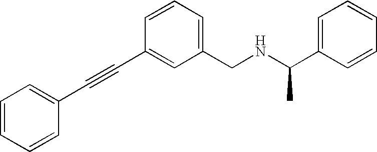

### (R)-(1-Phenyl-ethyl)-(3-phenylethynyl-benzyl)-amine (Compound 192) 3-Phenylethynyl-benzaldehyde and (R)-1-phenyl-ethylamine were treated as described in GP6. The crude was purified by flash chromatography (heptane/EtOAc 1:0→0:1).

¹³C NMR (DMSO-d₆): δ=145.6, 141.3, 131.2, 130.7, 129.5, 128.7, 128.4, 128.2, 126.6, 126.5, 122.2, 121.9, 89.4, 89.0, 56.8, 50.1, 24.2.

### Example 84

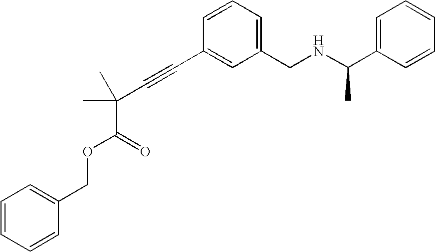

### (R)-Benzyl 2,2-dimethyl-4-{3-[(1-phenyl-ethylamino)-methyl]-phenyl}-but-3-ynoate (Compound 193)

See GP2c.

¹H NMR (600 MHz, DMSO-d₅): δ=7.42-7.2 (m, 14H), 5.20 (s, 2H), 3.73 (t, J=6.6 Hz, 1H), 3.52/3.49 (d, J=14.3 Hz, 2H), 1.51 (s, 6H), 1.28 (d, 3H).

### Example 85

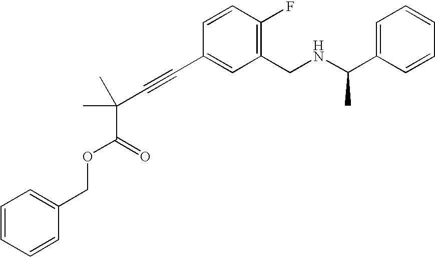

### (R)-Benzyl 4-{4-fluoro-3-[(1-phenyl-ethylamino)-methyl]-phenyl}-2,2-dimethyl-but-3-ynoate (Compound 194)

See GP2c.

LC/MS (METHOD A): (m/z) 430.4 (MH+); RT=5.39 min.

### Example 86

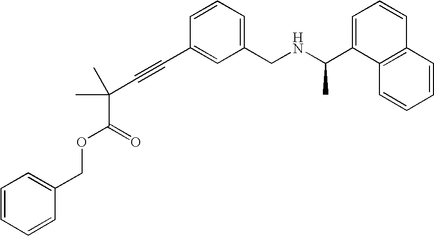

### (R)-Benzyl 2,2-dimethyl-4-{3-[(1-naphthalen-1-yl-ethylamino)-methyl]-phenyl}-but-3-ynoate (Compound 195)

To a mixture of (R)-(3-Iodo-benzyl)-(1-naphthalen-1-yl-ethyl)-amine (compound 416) from preparation 15 (134 mg, 0.35 mmol), CuI (5 mg, 0.028 mmol) and Pd(Ph₃P)₂Cl₂ (10 mg, 0.014 mmol) in Et₂NH (2 mL) was added benzyl 2,2-dimethyl-but-3-ynoate (162 mg, 0.8 mmol). The obtained reaction mixture was stirred at this temperature overnight. The reaction mixture was concentrated in vacuo together with celite. The residue was purified by flash chromatography (heptane/EtOAc 1:0→2:3), giving the title compound.

¹H NMR (600 MHz, DMSO-d₆): δ=8.16 (m, 1H), 7.93 (m, 1H), 7.81 (d, J=8.1 Hz, 1H), 7.76 (d, J=6.6 Hz, 1H), 7.53 (d, J=7.7 Hz, 1H), 7.52-7.48 (m, 2H), 7.41-7.37 (m, 2H), 7.37-7.32 (m, 3H), 7.31-7.26 (m, 3H), 7.25-7.21 (m, 1H), 5.22 (s, 2H), 4.60 (q, J=6.6 Hz, 1H), 3.63/3.57 (d, J=13.6 Hz, 2H), 1.51 (s, 6H), 1.40 (d, 3H).

### Example 87

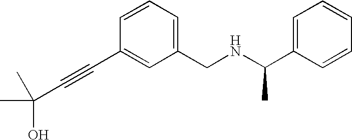

### (R)-2-Methyl-4-{3-[(1-phenyl-ethylamino)-methyl]-phenyl}-but-3-yn-2-ol (Compound 196)

See GP2c.

LC/MS (METHOD A): (m/z) 294.5 (MH+); RT=4.24 min.

### Example 88

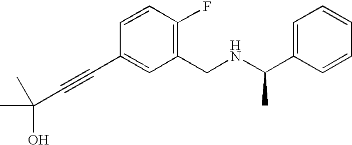

### (R)-4-{4-Fluoro-3-[(1-phenyl-ethylamino)-methyl]-phenyl}-2-methyl-but-3-yn-2-ol (Compound 197)

See GP2c.

LC/MS (METHOD A): (m/z) 312.5 (MH+); RT=4.31 min.

### Example 89

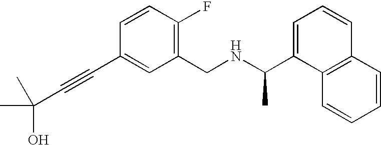

### (R)-4-{4-Fluoro-3-[(1-naphthalen-1-yl-ethylamino)-methyl]-phenyl}-2-methyl-but-3-yn-2-ol (Compound 198)

See GP2c

LC/MS (METHOD A): (m/z) 362.5 (MH+); RT=4.67 min.

### Example 90

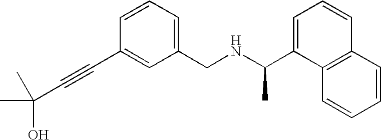

### (R)-2-Methyl-4-{6-[(1-naphthalen-1-yl-ethylamino)-methyl]-pyridin-2-yl}-but-3-yn-2-ol (Compound 199)

See GP2c.

LC/MS (METHOD A): (m/z) 345.4 (MH+); RT=4.41 min.

### Example 91

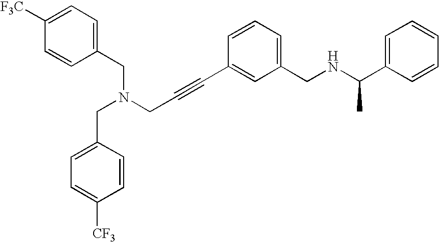

### (R)-(3-{3-[(1-Phenyl-ethylamino)-methyl]-phenyl}-prop-2-ynyl)-bis-(4-trifluoromethyl-benzyl)-amine (Compound 200)

See GP2c.

LC/MS (METHOD A): (m/z) 581.4 (MH+); RT=6.41 min.

### Example 92

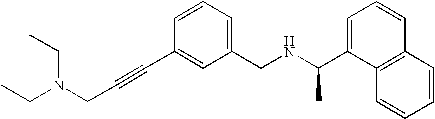

### (R)-Diethyl-(3-{3-[(1-naphthalen-1-yl-ethylamino)-methyl]-phenyl}-prop-2-ynyl)-amine (Compound 201)

See GP2c.

LC/MS (METHOD A): (m/z) 371.5 (MH+); RT=3.76 min.

### Example 93

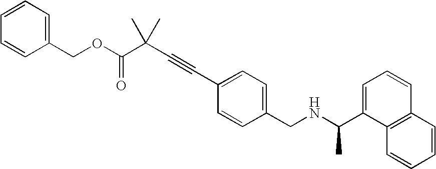

### (R)-Benzyl 2,2-dimethyl-4-{4-[(1-naphthalen-1-yl-ethylamino)-methyl]-phenyl}-but-3-ynoate (Compound 202)

See GP2c.

LC/MS (METHOD A): (m/z) 371.5 (MH+); RT=3.76 min.

### Example 94

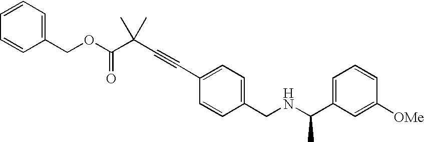

### (R)-Benzyl 4-(4-{[1-(3-methoxy-phenyl)-ethylamino]-methyl}-phenyl)-2,2-dimethyl-but-3-ynoate (Compound 203)

See GP2c.

LC/MS (METHOD A): (m/z) 442.6 (MH+); RT=5.44 min.

### Example 95

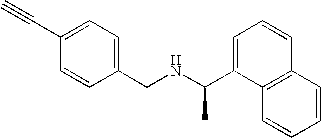

### (R)-(4-Ethynyl-benzyl)-(1-naphthalen-1-yl-ethyl)-amine (Compound 204)

2-Ethynyl-benzaldehyde and (R)-1-naphthalen-1-yl-ethylamine were treated as described in GP6. The crude was purified by flash chromatography (heptane/EtOAc 1:0→0:1).

¹³C NMR (DMSO-d₆): δ=142.6, 141.7, 133.9, 131.8, 131.3, 129.1, 128.5, 127.2, 126.1, 125.7, 123.4, 123.2, 120.2, 84.0, 80.6, 52.9, 50.9, 24.1.

### Example 96

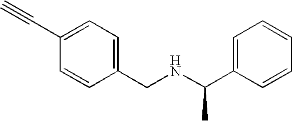

### (R)-(4-Ethynyl-benzyl)-(1-phenyl-ethyl)-amine (Compound 205)

4-Ethynyl-benzaldehyde and (R)-1-phenyl-ethylamine were treated as described in GP6. The crude was purified by flash chromatography (heptane/EtOAc 1:0→0:1).

¹³C NMR (DMSO-d₆): δ=145.9, 142.1, 131.3, 128.1, 128.0, 126.5, 126.4, 119.6, 83.5, 80.1, 56.6, 50.2, 24.4.

### Example 97

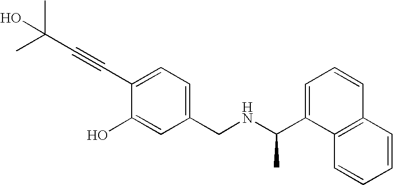

### (R)-2-(3-Hydroxy-3-methyl-but-1-ynyl)-5-[(1-naphthalen-1-yl-ethylamino)-methyl]-phenol (Compound 206)

To a mixture of (R)-(3-hydroxy-4-iodo-benzyl)-(1-naphthalen-1-yl-ethyl)-amine (compound 414) from Preparation 13 (200 mg, 0.5 mmol), CuI (5 mg, 0.028 mmol) and Pd(Ph₃P)₂Cl₂ (10 mg, 0.014 mmol) in Et₂NH (4 mL) was added 2-methyl-but-3-yn-2-ol (210 mg, 2.5 mmol). The obtained reaction mixture was stirred at this temperature overnight. The reaction mixture was concentrated in vacuo together with celite. The residue was purified by flash chromatography (heptane/EtOAc 1:0→0:1), giving the title compound.

¹³C NMR (DMSO-d₆): δ=157.8, 142.9, 141.3, 133.5, 132.3, 130.9, 128.6, 126.6, 125.7, 125.6, 125.2, 122.9, 122.6, 118.4, 114.6, 108.0, 98.4, 77.5, 63.6, 52.4, 50.5, 31.7, 23.7.

### Example 98

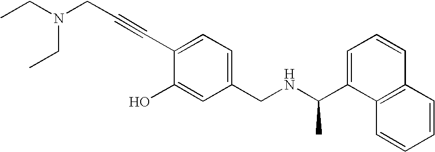

### (R)-2-(3-Diethylamino-prop-1-ynyl)-5-[(1-naphthalen-1-yl-ethylamino)-methyl]-phenol (Compound 207)

(R)-(3-hydroxy-4-iodo-benzyl)-(1-naphthalen-1-yl-ethyl)-amine (compound 414) from Preparation 13 (200 mg, 0.5 mmol) and diethyl-prop-2-ynyl-amine (0.34 mmol, 2.5 mmol) were treated as described for the preparation of compound 206. The residue was purified by flash chromatography (heptane/EtOAc 1:0→0:1), giving the title compound.

LC/MS (METHOD B): (m/z) 387.2 (MH+); RT=2.22 min.

### Example 99

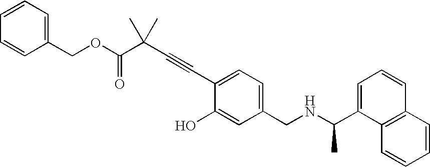

### (R)-Benzyl 4-{2-Hydroxy-4-[(1-naphthalen-1-yl-ethylamino)-methyl]-phenyl}-2,2-dimethyl-but-3-ynoate (Compound 208)

(R)-(3-hydroxy-4-iodo-benzyl)-(1-naphthalen-1-yl-ethyl)-amine (compound 414) from Preparation 13 (200 mg, 0.5 mmol) and benzyl 2,2-dimethyl-but-3-ynoate (506 mg, 2.5 mmol) were treated as described for the preparation of compound 206. The residue was purified by flash chromatography (heptane/EtOAc 1:0→1:9), giving the title compound.

¹³C NMR (DMSO-d₆): δ=172.6, 158.0, 143.1, 141.3, 136.1, 133.4, 132.5, 130.9, 128.6, 128.3, 127.8, 127.2, 126.6, 125.7, 125.6, 125.2, 122.9, 122.6, 118.4, 114.6, 107.6, 94.1, 78.6, 66.1, 52.3, 50.4, 38.2, 26.8, 23.7.

### Example 100

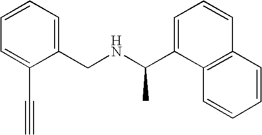

### (R)-(2-Ethynyl-benzyl)-(1-naphthalen-1-yl-ethyl)-amine (Compound 209)

2-Ethynyl-benzaldehyde and (R)-1-naphthalen-1-yl-ethylamine were treated as described in GP6. The crude was purified by flash chromatography (heptane/EtOAc 1:0→0:1).

¹³C NMR (DMSO-d₆): δ=141.0, 133.5, 132.1, 130.8, 128.7, 128.6, 128.1, 126.8, 126.6, 125.7, 125.6, 125.2, 122.9, 122.8, 120.6, 84.6, 81.5, 52.7, 49.0, 23.5.

### Example 101

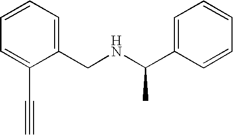

### (R)-(2-Ethynyl-benzyl)-(1-phenyl-ethyl)-amine (Compound 210)

2-Ethynyl-benzaldehyde and (R)-1-phenyl-ethylamine were treated as described in GP6. The crude was purified by flash chromatography (heptane/EtOAc 1:0→0:1).

¹³C NMR (DMSO-d₆): δ=145.9, 143.0, 132.1, 128.7, 128.1, 127.9, 126.4, 120.5, 84.6, 81.4, 56.9, 48.7, 24.4.

### Example 102

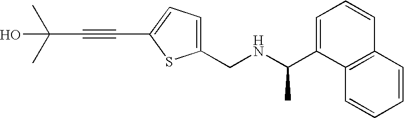

### (R)-2-Methyl-4-{5-[(1-naphthalen-1-yl-ethylamino)-methyl]-thiophen-2-yl}-but-3-yn-2-ol (Compound 211)

5-(3-Hydroxy-3-methyl-but-1-ynyl)-thiophene-2-carbaldehyde and (R)-1-naphthalen-1-yl-ethylamine were treated as described in GP6. The crude was purified by flash chromatography (heptane/EtOAc 1:0→1:2).

¹³C NMR (CDCl₃): δ 146.8, 140.5, 134.0, 131.9, 131.4, 129.0, 127.4, 125.8, 125.7, 125.4, 124.4, 123.0, 122.9, 121.3, 97.1, 75.9, 65.8, 52.7, 46.3, 31.4, 23.7.

### Example 103

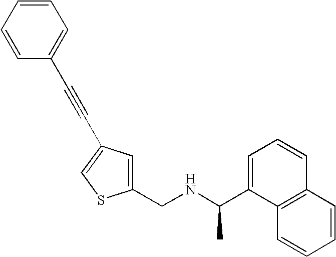

### (R)-(1-Naphthalen-1-yl-ethyl)-(4-phenylethynyl-thiophen-2-ylmethyl)-amine (Compound 212)

4-Phenylethynyl-thiophene-2-carbaldehyde and (R)-1-naphthalen-1-yl-ethylamine were treated as described in GP6. The crude was purified by flash chromatography (heptane/EtOAc 1:0→3:1).

¹³C NMR (DMSO-d₆): δ=146.3, 141.0, 133.5, 131.1, 130.8, 128.6, 128.6, 128.5, 126.8, 126.2, 125.7, 125.6, 125.3, 122.9, 122.6, 122.3, 120.2, 87.9, 85.2, 52.3, 45.3, 23.6

### Example 104

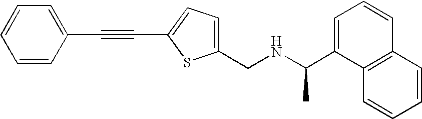

### (R)-(1-Naphthalen-1-yl-ethyl)-(5-phenylethynyl-thiophen-2-ylmethyl)-amine (Compound 213)

5-Phenylethynyl-thiophene-2-carbaldehyde and (R)-1-naphthalen-1-yl-ethylamine were treated as described in GP6. The crude was purified by flash chromatography (heptane/EtOAc 1:0→3:1).

¹³C NMR (DMSO-d₆): δ=148.8, 140.9, 133.5, 132.4, 131.0, 130.9, 128.7, 128.6, 126.8, 125.7, 125.6, 125.3, 124.5, 122.9, 122.7, 122.0, 119.9, 92.3, 83.1, 52.3, 45.7, 23.5.

### Example 105

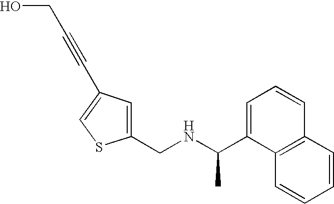

### (R)-3-{5-[(1-Naphthalen-1-yl-ethylamino)-methyl]-thiophen-3-yl}-prop-2-yn-1-ol (Compound 214)

4-(3-Hydroxy-prop-1-ynyl)-thiophene-2-carbaldehyde and (R)-1-naphthalen-1-yl-ethylamine were treated as described in GP6. The crude was purified by flash chromatography (heptane/EtOAc 1:0→1:2).

¹³C NMR (DMSO-d₆): δ=146.0, 141.0, 133.4, 130.8, 128.6, 127.9, 126.8, 126.1, 125.7, 125.6, 125.3, 122.9, 122.6, 120.4, 88.4, 79.4, 52.2, 49.3, 45.3, 23.6.

### Example 106

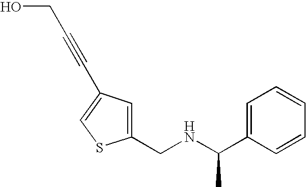

### (R)-3-{5-[(1-Phenyl-ethylamino)-methyl]-thiophen-3-yl}-prop-2-yn-1-ol (Compound 215)

4-(3-Hydroxy-prop-1-ynyl)-thiophene-2-carbaldehyde and (R)-1-phenyl-ethylamine were treated as described in GP6. The crude was purified by flash chromatography (heptane/EtOAc 1:0→1:2).

¹³C NMR (DMSO-d₆): δ=146.1, 145.5, 128.2, 127.9, 126.5, 126.4, 126.0, 120.4, 88.4, 79.4, 56.4, 49.3, 45.1, 24.1

### Example 107

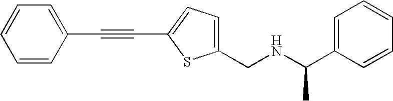

### (R)-(1-Phenyl-ethyl)-(5-phenylethynyl-thiophen-2-ylmethyl)-amine (Compound 216)

5-Phenylethynyl-thiophene-2-carbaldehyde and (R)-1-phenyl-ethylamine were treated as described in GP6. The crude was purified by flash chromatography (heptane/EtOAc 1:0→3:1).

¹³C NMR (DMSO-d₆): δ=149.0, 145.5, 132.3, 131.0, 128.7, 128.2, 126.6, 126.4, 124.3, 122.0, 119.8, 92.3, 83.1, 56.4, 45.5, 24.1.

### Example 108

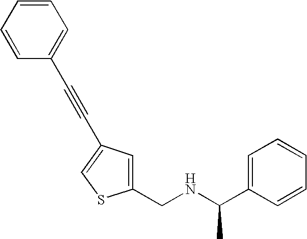

### (R)-(1-Phenyl-ethyl)-(4-phenylethynyl-thiophen-2-ylmethyl)-amine (Compound 217)

4-Phenylethynyl-thiophene-2-carbaldehyde and (R)-1-phenyl-ethylamine were treated as described in GP6. The crude was purified by flash chromatography (heptane/EtOAc 1:0→3:1).

¹³C NMR (DMSO-d₆): δ=146.3, 145.6, 131.1, 128.6, 128.5, 128.4, 128.2, 126.6, 126.4, 126.1, 122.3, 120.3, 87.9, 85.2, 56.4, 45.1, 24.1.

### Example 109

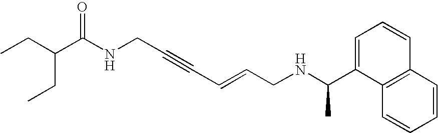

### (E)/(R)-2-Ethyl-N-[6-(1-naphthalen-1-yl-ethylamino)-hex-4-en-2-ynyl]-butyramide (Compound 218)

See GP3a.

¹H NMR (600 MHz, DMSO-d₆): δ=8.26-8.17 (m, 2H), 7.92 (m, 1H), 7.79 (d, J=8.0 Hz, 1H), 7.66 (d, J=6.1 Hz, 1H), 7.56-7.46 (m, 3H), 6.10 (dt, J₁=16.1 Hz, J₂=6.1 Hz, 1H), 5.62 (dt, J₁=16.1 Hz, J₂=1.7 Hz, 1H), 4.56 (q, 1H), 3.98 (dd, J₁=5.4, J₂=1.9 Hz, 2H), 3.10 (t, J=4.6 Hz, 2H), 1.96 (m, 1H), 1.53-1.25 (m, 2H), 1.37 (d, 3H), 0.84 (t, 3H).

### Example 110

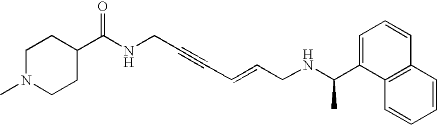

### (E)/(R)-1-Methyl-piperidine-4-carboxylic acid [6-(1-naphthalen-1-yl-ethylamino)-hex-4-en-2-ynyl]-amide (Compound 219)

See GP3a.

¹H NMR (600 MHz, DMSO-d₆): δ=8.26-8.17 (m, 2H), 7.92 (m, 1H), 7.79 (d, J=8.0 Hz, 1H), 7.67 (d, J=6.1 Hz, 1H), 7.56-7.46 (m, 3H), 6.10 (dt, J₁=16.0 Hz, J₂=5.7 Hz, 1H), 5.62 (dt, J₁=16.0 Hz, J₂=1.7 Hz, 1H), 4.56 (q, 1H), 3.96 (dd, J₁=5.4, J₂=1.9 Hz, 2H), 3.10 (m, 2H), 2.80-2.90 (m, 2H), 2.22 (s, 3H), 2.15 (m, 3H), 1.70-1.50 (m, 4H), 1.37 (d, 3H).

### Example 111

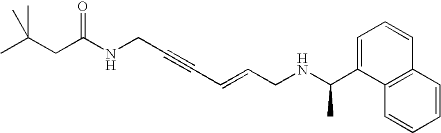

### (E)/(R)-3,3-Dimethyl-N-[6-(1-naphthalen-1-yl-ethylamino)-hex-4-en-2-ynyl]-butyramide (Compound 220)

See GP3a.

¹H NMR (600 MHz, DMSO-d₆): δ=8.26-8.10 (m, 2H), 7.92 (m, 1H), 7.79 (d, J=8.0 Hz, 1H), 7.66 (d, J=6.1 Hz, 1H), 7.56-7.45 (m, 3H), 6.10 (dt, J₁=16.1 Hz, J₂=5.7 Hz, 1H), 5.62 (dt, J₁=16.1 Hz, J₂=1.7 Hz, 1H), 4.56 (q, 1H), 3.96 (dd, J₁=5.4, J₂=1.9 Hz, 2H), 3.10 (t, J=4.6 Hz, 2H), 1.96 (s, 2H), 1.53-1.25 (m, 2H), 1.37 (d, 3H), 0.95 (t, 9H).

### Example 112

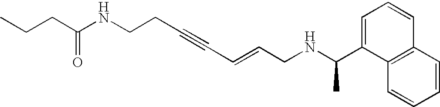

### (E)/(R)—N-[7-(1-Naphthalen-1-yl-ethylamino)-hept-5-en-3-ynyl]-butyramide (Compound 221)

See GP3a.

¹H NMR (600 MHz, DMSO-d₆): δ=8.22 (d, 1H), 7.97-7.91 (m, 1H, 1NH), 7.80 (d, 1H), 7.69 (d, 1H), 7.56-7.48 (m, 3H), 6.08 (dt, J₁=16.0 Hz, J₂=6.0 Hz, 1H), 5.62 (dt, J₁=16.0 Hz, J₂=1.7 Hz, 1H), 4.62 (q, 1H), 3.18-3.12 (m, 4H), 2.41 (dt, 2H), 2.03 (t, 2H), 1.50 (h, 2H), 1.39 (d, 3H), 0.84 (t, 3H).

### Example 113

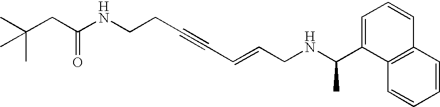

### (E)/(R)-3,3-Dimethyl-N-[7-(1-naphthalen-1-yl-ethylamino)-hept-5-en-3-ynyl]-butyramide (Compound 222)

See GP3a.

¹H NMR (600 MHz, DMSO-d₆): δ=8.22 (d, 1H), 7.93 (dd, 1H), 7.89 (t, 1NH), 7.81 (d, 1H), 7.69 (d, 1H), 7.57-7.48 (m, 3H), 6.06 (dt, J₁=16.1 Hz, J₂=5.9 Hz, 1H), 5.62 (dt, J₁=16.1 Hz, J₂=1.5 Hz, 1H), 4.63 (q, 1H), 3.19-3.10 (m, 4H), 2.44-2.39 (m, 2H), 1.94 (s, 2H), 1.40 (d, 3H), 0.97 (s, 9H).

### Example 114

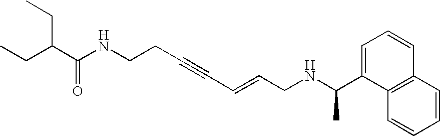

### (E)/(R)-2-Ethyl-N-[7-(1-naphthalen-1-yl-ethylamino)-hept-5-en-3-ynyl]-butyramide (Compound 223)

See GP3a.

¹H NMR (600 MHz, DMSO-d₆): δ=8.21 (bd, 1H), 7.96 (t, NH), 7.94 (m, 1H), 7.82 (d, 1H), 7.69 (d, 1H), 7.58-7.48 (m, 3H), 6.05 (dt, J₁=16.0 Hz, J₂=6.1 Hz, 1H), 5.63 (bd, J=16.0 Hz, 1H), 4.67 (m, 1H), 3.18 (q, 4H), 2.43 (m, 2H), 1.93 (m, 1H), 1.47-1.38 (m, 2H), 1.41 (d, 3H), 1.32 (m, 2H), 0.79 (t, 6H).

### Example 115

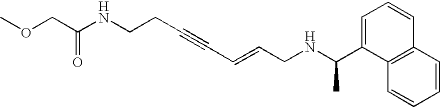

### (E)/(R)-2-Methoxy-N-[7-(1-naphthalen-1-yl-ethylamino)-hept-5-en-3-ynyl]-acetamide (Compound 224)

See GP3a.

¹H NMR (600 MHz, DMSO-d₆): δ=8.22 (bd, 1H), 7.94 (m, 1H), 7.92 (t, NH), 7.82 (d, 1H), 7.70 (d, 1H), 7.58-7.49 (m, 3H), 6.08 (dt, J₁=15.9 Hz, J₂=6.1 Hz, 1H), 5.64 (dm, J=15.9 Hz, 1H), 4.67 (q, 1H), 3.79 (s, 2H), 3.30 (s, 3H), 3.22 (q, 2H), 3.17 (m, 2H), 2.46 (m, 2H), 1.41 (d, 3H).

### Example 116

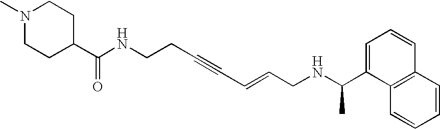

### (E)/(R)-1-Methyl-piperidine-4-carboxylic acid [7-(1-naphthalen-1-yl-ethylamino)-hept-5-en-3-ynyl]-amide (Compound 225)

See GP3a.

¹H NMR (600 MHz, DMSO-d₆): δ=8.22 (bd, 1H), 7.99 (t, NH), 7.93 (m, 1H), 7.80 (d, 1H), 7.69 (d, 1H), 7.56-7.47 (m, 3H), 6.07 (dt, J₁=16.0 Hz, J₂=6.0 Hz, 1H), 5.62 (dm, J=16.0 Hz, 1H), 4.62 (q, 1H), 3.23-3.07 (m, 4H), 2.97 (m, 2H), 2.41 (m, 2H), 2.33 (s, 3H), 2.22 (m, 2H), 2.14 (m, 1H), 1.73-1.60 (m, 4H), 1.39 (d, 3H).

### Example 117

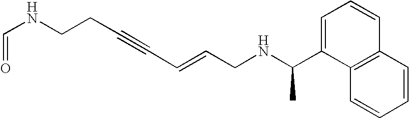

### (E)-/(R)—N-[7-(1-Naphthalen-1-yl-ethylamino)-hept-5-en-3-ynyl]-formamide (Compound 226)

See GP3a.

LC/MS (METHOD A): (m/z) 307.3 (MH+); RT=4.07 min.

### Example 118

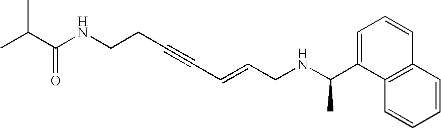

### (R)—N-[7-(1-Naphthalen-1-yl-ethylamino)-hept-5-en-3-ynyl]-isobutyramide (Compound 227)

See GP3a.

LC/MS (METHOD A): (m/z) 349.4 (MH+); RT=4.36 min.

### Example 119

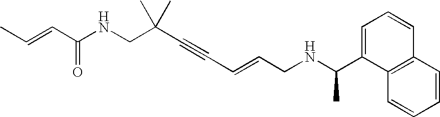

### (E)/(R)-But-2-enoic acid [2,2-dimethyl-7-(1-naphthalen-1-yl-ethylamino)-hept-5-en-3-ynyl]-amide (Compound 228)

See GP3b.

LC/MS (METHOD A): (m/z) 375.2 (MH+); RT=4.97 min.

### Example 120

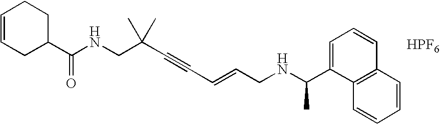

### (E)/(R)-Cyclohex-3-enecarboxylic acid [2,2-dimethyl-7-(1-naphthalen-1-yl-ethylamino)-hept-5-en-3-ynyl]-amide hydrogen hexafluorophosphate (Compound 229)

See GP3b.

LC/MS (METHOD A): (m/z) 415.2 (MH+); RT=5.27 min.

### Example 121

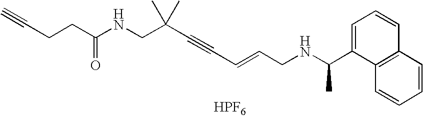

### (E)/(R)-Pent-4-ynoic acid [2,2-dimethyl-7-(1-naphthalen-1-yl-ethylamino)-hept-5-en-3-ynyl]-amide hydrogen hexafluorophosphate (Compound 230)

See GP3b.

LC/MS (METHOD A): (m/z) 387.1 (MH+); RT=4.99 min.

### Example 122

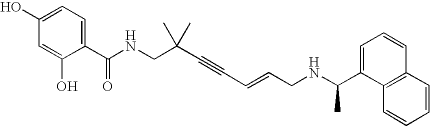

### (E)/(R)—N-[2,2-Dimethyl-7-(1-naphthalen-1-yl-ethylamino)-hept-5-en-3-ynyl]-2,4-dihydroxy-benzamide (Compound 231)

See GP3b.

LC/MS (METHOD A): (m/z) 443.2 (MH+); RT=5.21 min.

### Example 123

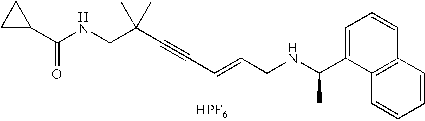

### (E)/(R)-Cyclopropanecarboxylic acid [2,2-dimethyl-7-(1-naphthalen-1-yl-ethylamino)-hept-5-en-3-ynyl]-amide hydrogen hexafluorophosphate (Compound 232)

See GP3b.

LC/MS (METHOD A): (m/z) 375.1 (MH+); RT=4.97 min.

### Example 124

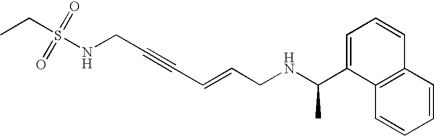

### (E)/(R)-Ethanesulfonic acid [6-(1-naphthalen-1-yl-ethylamino)-hex-4-en-2-ynyl]-amide (Compound 233)

See GP4.

¹H NMR (DMSO-d₆): δ=8.26-8.18 (m, 1H), 7.97-7.89 (m, 1H), 7.80 (d, 1H), 7.69 (d, 1H), 7.62-7.42 (m, 3H), 6.16 (dt, J₁=16.1 Hz, J₂=5.9 Hz, 1H), 5.69 (dt, J₁=16.1 Hz, J₂=1.8 Hz, 1H), 4.61 (q, 1H), 3.91 (bd, 2H), 3.16 (bd, 2H), 3.05 (q, 2H), 1.39 (d, 3H), 1.21 (t, 3H).

### Example 125

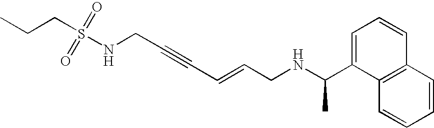

### (E)/(R)-Propane-1-sulfonic acid [6-(1-naphthalen-1-yl-ethylamino)-hex-4-en-2-ynyl]-amide (Compound 234)

See GP4.

¹H NMR (DMSO-d₆): δ=8.25-8.17 (m, 1H), 7.98-7.87 (m, 1H, 1NH), 7.80 (d, 1H), 7.68 (d, 1H), 7.59-7.44 (m, 3H), 6.16 (dt, J₁=15.6 Hz, J₂=6.0 Hz, 1H), 5.69 (dt, J₁=15.6 Hz, J₂=1.8 Hz, 1H), 4.61 (q, 1H), 3.94-3.87 (m, 2H), 3.19-3.12 (m, 2H), 3.07-2.99 (m, 2H), 1.70 (h, 2H), 1.39 (d, 3H), 0.96 (t, 3H).

### Example 126

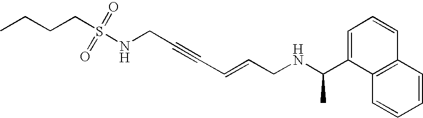

### (E)/(R)-Butane-1-sulfonic acid [6-(1-naphthalen-1-yl-ethylamino)-hex-4-en-2-ynyl]-amide (Compound 235)

See GP4.

¹H NMR (DMSO-d₆): δ=8.25-8.17 (m, 1H), 7.98-7.87 (m, 1H), 7.79 (d, 1H), 7.67 (d, 1H), 7.59-7.44 (m, 3H), 6.16 (dt, J₁=15.6 Hz, J₂=6.0 Hz, 1H), 5.69 (dt, J₁=15.6 Hz, J₂=1.8 Hz, 1H), 4.55 (q, 1H), 3.94-3.87 (m, 2H), 3.19-3.12 (m, 2H), 3.07-2.99 (m, 2H), 1.65 (m, 2H), 1.37 (d, 3H), 0.87 (t, 3H).

### Example 127

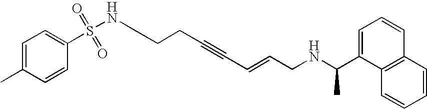

### (E)/(R)-4-Methyl-N-[7-(1-naphthalen-1-yl-ethylamino)-hept-5-en-3-ynyl]-benzenesulfonamide (Compound 236)

See GP4.

¹H NMR (600 MHz, DMSO-d₆): δ=8.22 (bd, 1H), 7.92 (m, 1H), 7.79 (d, 1H), 7.74 (t, NH), 7.70-7.65 (m, 3H), 7.55-7.45 (m, 3H), 7.39 (m, 2H), 6.06 (dt, J₁=15.6 Hz, J₂=5.9 Hz, 1H), 5.57 (dm, J=15.8 Hz, 1H), 4.57 (q, 1H), 3.09 (m, 2H), 2.84 (q, 2H), 2.39 (m, 2H), 2.37 (s, 3H), 1.37 (d, 3H).

### Example 128

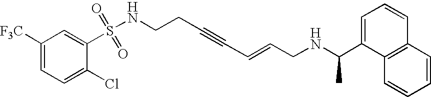

### (E)/(R)-2-Chloro-N-[7-(1-naphthalen-1-yl-ethylamino)-hept-5-en-3-ynyl]-5-trifluoromethyl-benzenesulfonamide (Compound 237)

See GP4.

¹H NMR (600 MHz, DMSO-d₆): δ=8.40 (bs, 1NH), 8.22 (d, 1H), 8.18 (d, 1H), 8.01 (dd, 1H), 7.95-7.90 (m, 2H), 7.82 (d, 1H), 7.70 (d, 1H), 7.56-7.49 (m, 3H), 6.02 (dₜ, J1=16.1 Hz, J₂=6.1 Hz, 1H), 5.55 (dt, J₁=16.1 Hz, J₂=1.6 Hz, 1H), 4.65 (q, 1H), 3.16 (bd, 2H), 3.06 (t, 2H), 2.43 (dt, 2H), 1.41 (d, 3H).

### Example 129

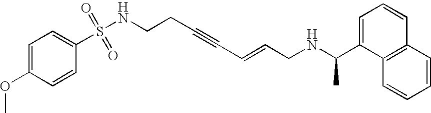

### (E)/(R)-4-Methoxy-N-[7-(1-naphthalen-1-yl-ethylamino)-hept-5-en-3-ynyl]-benzenesulfonamide (Compound 238)

See GP4.

¹H NMR (600 MHz, DMSO-d₆): δ=8.22 (d, 1H), 7.93 (dd, 1H), 7.81 (d, 1H), 7.74-7.65 (m, 4H), 7.56-7.46 (m, 4H), 7.11 (dt, 2H), 6.07 (dt, J₁=16.1 Hz, J₂=6.0 Hz, 1H), 5.61 (dt, J₁=16.1 Hz, J₂=1.8 Hz, 1H), 4.63 (q, 1H), 3.83 (s, 3H), 3.16-3.12 (bm, 2H), 2.83 (q, 2H), 2.40 (dt, 2H), 1.39 (d, 3H).

### Example 130

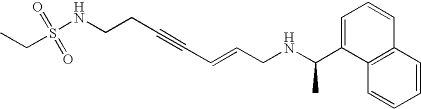

### (E)/(R)-Ethanesulfonic acid [7-(1-naphthalen-1-yl-ethylamino)-hept-5-en-3-ynyl]-amide (Compound 239)

See GP4.

¹H NMR (600 MHz, DMSO-d₆): δ=8.22 (d, 1H), 7.93 (dd, 1H), 7.81 (d, 1H), 7.74-7.65 (m, 4H), 7.56-7.46 (m, 4H), 7.11 (dt, 2H), 6.07 (dt, J₁=16.1 Hz, J₂=6.0 Hz, 1H), 5.61 (dt, J₁=16.1 Hz, J₂=1.8 Hz, 1H), 4.63 (q, 1H), 3.83 (s, 3H), 3.16-3.12 (bm, 2H), 2.83 (q, 2H), 2.40 (dt, 2H), 1.39 (d, 3H).

### Example 131

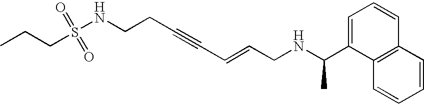

### (E)/(R)-Propane-1-sulfonic acid [7-(1-naphthalen-1-yl-ethylamino)-hept-5-en-3-ynyl]-amide (Compound 240)

See GP4.

¹H NMR (600 MHz, DMSO-d₆): δ=8.22 (d, 1H), 7.95 (d, 1H), 7.84 (d, 1H), 7.71 (d, 1H), 7.58-7.50 (m, 3H). 7.22 (t, 1NH), 6.09 (dt, J₁=15.9 Hz, J₂=6.1 Hz, 1H), 5.69 (dt, J₁=15.9 Hz, J₂=1.5 Hz, 1H), 4.74 (q, 1H), 3.24 (d, 2H), 3.06 (q, 2H), 3.02-2.97 (m, 2H), 2.48 (dt, 2H), 1.66 (h, 2H), 1.44 (d, 3H), 0.96 (t, 3H).

### Example 132

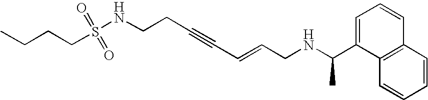

### (E)/(R)-Butane-1-sulfonic acid [7-(1-naphthalen-1-yl-ethylamino)-hept-5-en-3-ynyl]-amide (Compound 241)

See GP4.

¹H NMR (600 MHz, DMSO-d₆): δ=8.22 (d, 1H), 7.95 (d, 1H), 7.85 (d, 1H), 7.72 (d, 1H), 7.59-7.50 (m, 3H), 7.21 (t, 1NH), 6.09 (dt, J₁=15.7 Hz, J₂=6.1 Hz, 1H), 5.71 (bd, J=15.7 Hz, 1H), 4.79 (bq, 1H), 3.28 (bs, 2H), 3.06 (q, 2H), 3.03-2.98 (m, 2H), 2.49 (dt, 2H), 1.65-1.57 (m, 2H), 1.46 (d, 3H), 1.37 (h, 2H), 0.87 (t, 3H).

### Example 133

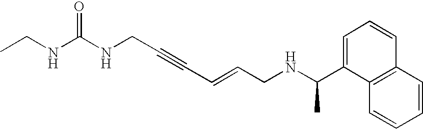

### (E)/(R)-1-Ethyl-3-[6-(1-naphthalen-1-yl-ethylamino)-hex-4-en-2-ynyl]-urea (Compound 242)

See GP5.

¹H NMR (DMSO-d₆): δ=8.27-8.17 (m, 1H), 7.95-7.87 (m, 1H), 7.78 (d, 1H), 7.77 (d, 1H), 7.59-7.44 (m, 3H), 6.10 (dt, J₁=15.7 Hz, J₂=5.4 Hz, 1H), 5.90 (t, J=5.7 Hz, 1H), 5.69 (dt, J₁=15.6 Hz, J₂=1.8 Hz, 1H), 4.55 (q, 1H), 3.94-3.87 (m, 2H), 3.19-3.12 (m, 4H), 1.36 (d, 3H), 0.80 (t, 3H).

### Example 134

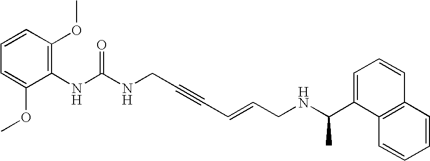

### (E)/(R)-1-(2,6-Dimethoxy-phenyl)-3-[6-(1-naphthalen-1-yl-ethylamino)-hex-4-en-2-ynyl]-urea (Compound 243)

See GP5.

¹H NMR (DMSO-d₆): δ=8.27-7.40 (m, 11H), 7.78 (d, 1H), 7.77 (d, 1H), 6.95 (t, J=5.7, 1H), 6.14 (dt, J₁=15.7 Hz, J₂=5.4 Hz, 1H), 5.65 (dt, J₁=15.6 Hz, J₂=1.8 Hz, 1H), 4.57 (q, 1H), 4.05-3.95 (m, 2H), 3.80/3.70 (s, 6H), 3.20-100 (m, 2H), 1.36 (d, 3H).

### Example 135

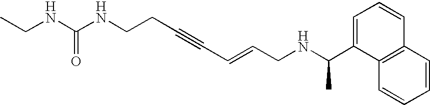

### (E)/(R)-1-Ethyl-3-[7-(1-naphthalen-1-yl-ethylamino)-hept-5-en-3-ynyl]-urea (Compound 244)

See GP5.

¹H NMR (DMSO-d₆): δ=8.22 (bd, 1H), 7.93 (m, 1H), 7.80 (d, 1H), 7.69 (d, 1H), 7.57-7.47 (m, 3H), 6.08 (dt, J₁=15.9 Hz, J₂=6.0 Hz, 1H), 5.93 (t, 1NH), 5.91 (t, 1NH), 5.63 (d, J=15.9 Hz, 1H), 4.62 (m, 1H), 3.14 (m, 2H), 3.10 (q, 2H), 2.99 (m, 2H), 2.37 (dt, 2H), 1.39 (d, 3H), 0.97 (t, 3H).

### Example 136

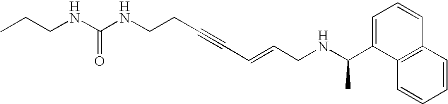

### (E)/(R)-1-[7-(1-Naphthalen-1-yl-ethylamino)-hept-5-en-3-ynyl]-3-propyl-urea (Compound 245)

See GP5.

¹H NMR (600 MHz, DMSO-d₆): δ=8.22 (bd, 1H), 7.93 (m, 1H), 7.80 (d, 1H), 7.69 (d, 1H), 7.56-7.46 (m, 3H), 6.08 (dt, J₁=15.9 Hz, J₂=5.9 Hz, 1H), 5.96 (t, NH), 5.92 (t, NH), 5.62 (dm, J=15.9 Hz, 1H), 4.61 (q, 1H), 3.18-3.06 (m, 4H), 2.93 (q, 2H), 2.37 (m, 2H), 1.38 (d, 3H), 1.36 (m, 2H), 0.82 (t, 3H).

### Example 137

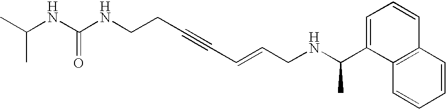

### (E)/(R)-1-Isopropyl-3-[7-(1-naphthalen-1-yl-ethylamino)-hept-5-en-3-ynyl]-urea (Compound 246)

See GP5.

¹H NMR (600 MHz, DMSO-d₆): δ=8.22 (bd, 1H), 7.93 (m, 1H), 7.80 (d, 1H), 7.68 (d, 1H), 7.56-7.46 (m, 3H), 6.09 (dt, J₁=15.9 Hz, J₂=6.0 Hz, 1H), 5.82 (t, 1NH), 5.80 (d, 1NH), 5.64 (d, J=15.9 Hz, 1H), 4.60 (q, 1H), 3.64 (m, 1H), 3.16-3.06 (m, 4H), 2.37 (m, 2H), 1.38 (d, 3H), 1.01 (d, 6H).

### Example 138

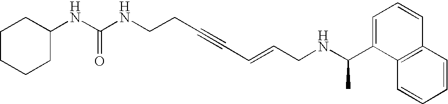

### (E)/(R)-1-Cyclohexyl-3-[7-(1-naphthalen-1-yl-ethylamino)-hept-5-en-3-ynyl]-urea (Compound 247)

See GP5.

¹H NMR (600 MHz, DMSO-d₆): δ=8.22 (bd, 1H), 7.92 (m, 1H), 7.79 (d, 1H), 7.68 (d, 1H), 7.56-7.46 (m, 3H), 6.09 (dt, J₁=15.9 Hz, J₂=5.9 Hz, 1H), 5.86 (d, NH), 5.83 (t, NH), 5.63 (dm, J=15.9 Hz, 1H), 4.58 (q, 1H), 3.33 (m, 1H), 3.15-3.04 (m, 4H), 2.37 (m, 2H), 1.72 (m, 2H), 1.62 (m, 2H), 1.51 (m, 1H), 1.37 (d, 3H), 1.24 (m, 2H), 1.16-1.01 (m, 3H).

### Example 139

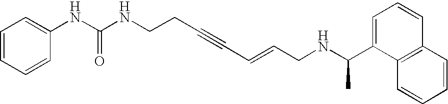

### (E)/(R)-1-[7-(1-Naphthalen-1-yl-ethylamino)-hept-5-en-3-ynyl]-3-phenyl-urea (Compound 248)

See GP5.

¹H NMR (600 MHz, DMSO-d₆): δ=8.58 (s, NH), 8.22 (bd, 1H), 7.92 (m, 1H), 7.79 (d, 1H), 7.68 (d, 1H), 7.55-7.47 (m, 3H), 7.38 (m, 2H), 7.21 (m, 2H), 6.88 (m, 1H), 6.28 (t, NH), 6.10 (dt, J₁=16.0 Hz, J₂=5.8 Hz, 1H), 5.63 (dm, J=16.0 Hz, 1H), 4.57 (q, 1H), 3.21 (q, 2H), 3.10 (m, 2H), 2.46 (m, 2H), 1.37 (d, 3H).

### Example 140

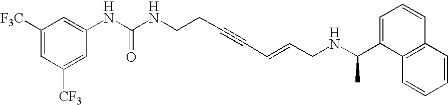

### (E)/(R)-1-(3,5-Bis-trifluoromethyl-phenyl)-3-[7-(1-naphthalen-1-yl-ethylamino)-hept-5-en-3-ynyl]-urea (Compound 249) See GP5.

¹H NMR (600 MHz, DMSO-d₆): δ=9.49 (bs, NH), 8.22 (bd, 1H), 8.09 (m, 2H), 7.93 (m, 1H), 7.81 (d, 1H), 7.68 (d, 1H), 7.57-7.47 (m, 4H), 6.70 (t, NH), 6.09 (dt, J₁=15.9 Hz, J₂=6.0 Hz, 1H), 5.65 (dm, J=15.9 Hz, 1H), 4.63 (q, 1H), 3.25 (q, 2H), 3.15 (m, 2H), 2.54-2.47 (m, 2H), 1.39 (d, 3H).

### Example 141

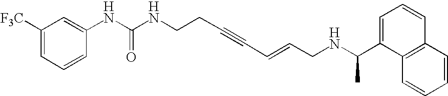

### (E)/(R)-1-[7-(1-Naphthalen-1-yl-ethylamino)-hept-5-en-3-ynyl]-3-(3-trifluoromethyl-phenyl)-urea (Compound 250)

See GP5.

¹H NMR (600 MHz, DMSO-d₆): δ=9.02 (s, NH), 8.22 (bd, 1H), 7.97 (m, 1H), 7.93 (m, 1H), 7.81 (d, 1H), 7.69 (d, 1H), 7.57-7.47 (m, 4H), 7.44 (t, 1H), 7.22 (bd, 1H), 6.44 (t, NH), 6.10 (dt, J₁=16.0 Hz, J₂=6.0 Hz, 1H), 5.65 (dm, J=16.0 Hz, 1H), 4.63 (q, 1H), 3.23 (q, 2H), 3.15 (m, 2H), 2.48 (m, 2H), 1.39 (d, 3H).

### Example 142

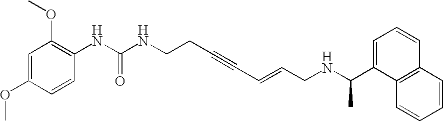

### (E)/(R)-1-(2,4-Dimethoxy-phenyl)-3-[7-(1-naphthalen-1-yl-ethylamino)-hept-5-en-3-ynyl]-urea (Compound 251)

See GP5.

¹H NMR (600 MHz, DMSO-d₆): δ=8.22 (bd, 1H), 7.92 (m, 1H), 7.85 (d, 1H), 7.79 (d, 1H), 7.75 (m, 1H), 7.68 (d, 1H), 7.56-7.47 (m, 3H), 6.83 (t, NH), 6.56 (d, 1H), 6.42 (dd, 1H), 6.10 (dt, J₁=15.9 Hz, J₂=5.9 Hz, 1H), 5.64 (dm, J=15.9 Hz, 1H), 4.59 (q, 1H), 3.80 (s, 3H), 3.70 (s, 3H), 3.19 (q, 2H), 3.12 (m, 2H), 2.44 (m, 2H), 1.38 (d, 3H).

### Example 143

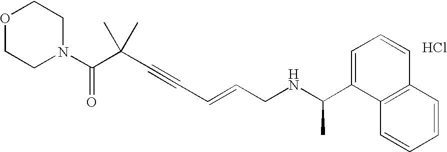

### (E)/(R)-2,2-Dimethyl-1-morpholin-4-yl-7-(1-naphthalen-1-yl-ethylamino)-hept-5-en-3-yn-1-one hydrochloride (Compound 252)

To a mixture of compound 283 (20 mg, 0.056 mmol) and morpholine hydrochloride (20 mg, 0.16 mmol) in Et₃N/DCM (1:10, 5 mL) was added pentafluorophenyl diphenylphosphate (40 mg, 0.10 mmol) at rt. After being stirred at this temperature for 1 h, the reaction mixture was concentrated in vacuo. Chromatography (EtOAc) gave a free amine, which was dissolved in Et₂O and treated with 4 N HCl in 1,4-dioxane, giving the title compound.

¹³C NMR (DMSO-d₆): δ=169.2, 133.8, 133.3, 130.1, 128.9, 128.8, 126.8, 126.1, 125.5, 124.3, 122.5, 116.0, 94.2, 80.8, 65.9, 51.5, 46.2, 36.2, 27.4, 19.7.

### Example 144

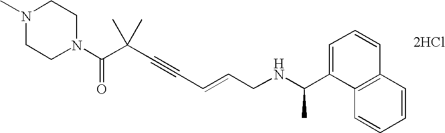

### (E)/(R)-2,2-Dimethyl-1-(4-methyl-piperazin-1-yl)-7-(1-naphthalen-1-yl-ethylamino)-hept-5-en-3-yn-1-one dihydrochloride (Compound 253)

Compound 283 (20 mg, 0.056 mmol) and 1-methylpiperazine (20 mg, 0.20 mmol) were treated as described for the preparation of compound 252. Chromatography (DCM/MeOH) gave a free amine, which was dissolved in Et₂O and treated with 4 N HCl in 1,4-dioxane, giving the title compound.

¹³C NMR (DMSO-d₆): δ=169.4, 133.8, 133.3, 130.1, 128.9, 128.8, 126.8, 126.1, 125.5, 124.4, 122.5, 115.7, 93.5, 81.1, 51.8, 51.8, 46.2, 41.9, 36.3, 27.4, 19.9.

### Example 145

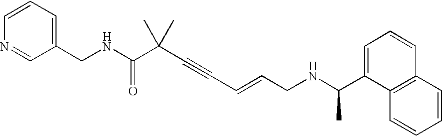

### (E)/(R)-2,2-Dimethyl-7-(1-naphthalen-1-yl-ethylamino)-hept-5-en-3-ynoic acid (pyridin-3-ylmethyl)-amide (Compound 254)

See GP3a.

LC/MS (METHOD A): (m/z) 412.2 (MH+); RT=3.96 min.

### Example 146

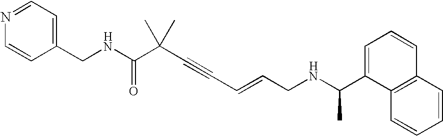

### (E)/(R)-2,2-Dimethyl-7-(1-naphthalen-1-yl-ethylamino)-hept-5-en-3-ynoic acid (pyridin-4-ylmethyl)-amide (Compound 255)

See GP3a.

LC/MS (METHOD A): (m/z) 412.2 (MH+); RT=3.82 min.

### Example 147

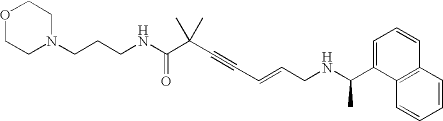

### (E)/(R)-2,2-Dimethyl-7-(1-naphthalen-1-yl-ethylamino)-hept-5-en-3-ynoic acid (3-morpholin-4-yl-propyl)-amide (Compound 256)

See GP3a.

LC/MS (METHOD A): (m/z) 448.3 (MH+); RT=3.76 min.

### Example 148

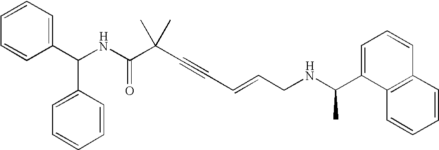

### (E)/(R)-2,2-Dimethyl-7-(1-naphthalen-1-yl-ethylamino)-hept-5-en-3-ynoic acid benzhydryl-amide (Compound 257)

See GP3a.

LC/MS (METHOD A): (m/z) 487.2 (MH+); RT=5.44 min.

### Example 149

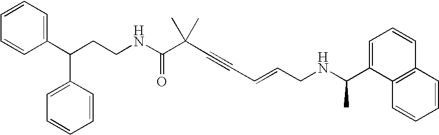

### (E)/(R)-2,2-Dimethyl-7-(1-naphthalen-1-yl-ethylamino)-hept-5-en-3-ynoic acid (3,3-diphenyl-propyl)-amide (Compound 258)

See GP3a.

LC/MS (METHOD A): (m/z) 515.2 (MH+); RT=5.56 min.

### Example 150

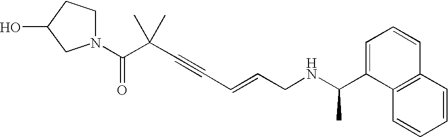

### (E)-1-[(R/S)]-3-Hydroxy-pyrrolidin-1-yl)-2,2-dimethyl-7-[(R)1-naphthalen-1-yl-ethylamino]-hept-5-en-3-yn-1-on (Compound 259)

See GP3a.

LC/MS (METHOD A): (m/z) 391.2 (MH+); RT=4.32 min.

### Example 151

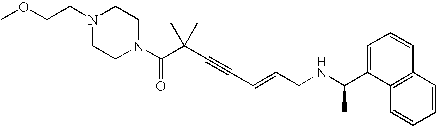

### (E)/(R)-1-[4-(2-Methoxy-ethyl)-piperazin-1-yl]-2,2-dimethyl-7-(1-naphthalen-1-yl-ethylamino)-hept-5-en-3-yn-1-one (Compound 260)

See GP3a.

LC/MS (METHOD A): (m/z) 448.2 (MH+); RT=3.79 min.

### Example 152

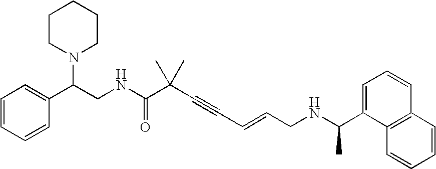

### (E)-2,2-Dimethyl-7-[(R)-1-naphthalen-1-yl-ethylamino)-hept-5-en-3-ynoic acid [(R/S)-2-phenyl-2-piperidin-1-yl-ethyl]-amide (Compound 261)

See GP3a.

¹³C NMR (DMSO-d₆): δ=163.3, 142.8, 140.8, 133.6, 130.9, 128.8, 128.5, 128.0, 127.4, 127.0, 125.9, 125.7, 125.5, 123.0, 122.8, 109.6, 92.2, 82.4, 67.2, 52.5, 50.1, 48.3, 40.5, 38.6, 27.4, 26.0, 24.3, 23.5.

### Example 153

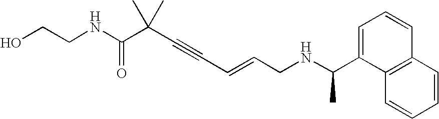

### (E)/(R)-2,2-Dimethyl-7-(1-naphthalen-1-yl-ethylamino)-hept-5-en-3-ynoic acid (2-hydroxy-ethyl)-amide (Compound 262)

See GP3a.

¹³C NMR (DMSO-d₆): δ=163.3, 142.6, 141.0, 133.6, 130.9, 128.8, 127.0, 125.9, 125.7, 125.4, 123.1, 122.8, 109.9, 92.6, 81.8, 59.6, 52.5, 48.4, 41.9, 38.4, 27.5, 23.6.

### Example 154

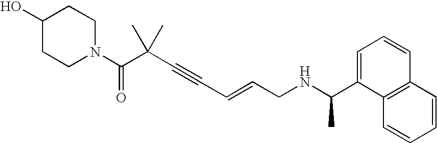

### (E)/(R)-1-(4-Hydroxy-piperidin-1-yl)-2,2-dimethyl-7-(1-naphthalen-1-yl-ethylamino)-hept-5-en-3-yn-1-one (Compound 263)

See GP3a.

¹³C NMR (DMSO-d₆): δ=163.4, 142.4, 133.6, 130.9, 128.8, 127.0, 125.9, 125.7, 125.4, 123.0, 122.9, 109.8, 92.1, 81.5, 65.6, 52.5, 48.5, 36.4, 34.2, 28.0, 23.6.

### Example 155

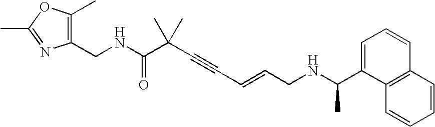

### (E)/(R)-2,2-Dimethyl-7-(1-naphthalen-1-yl-ethylamino)-hept-5-en-3-ynoic acid (2,5-dimethyl-oxazol-4-ylmethyl)-amide (Compound 264)

See GP3a.

LC/MS (METHOD A): (m/z) 444.2 (MH+); RT=4.77 min.

### Example 156

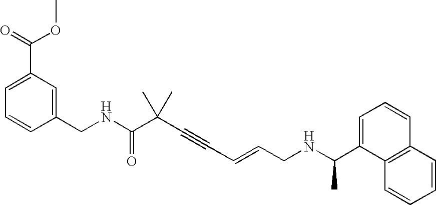

### (E)/(R)-3-{[2,2-Dimethyl-7-(1-naphthalen-1-yl-ethylamino)-hept-5-en-3-ynoylamino]-methyl}-benzoic acid methyl ester (Compound 265)

See GP3a.

LC/MS (METHOD A): (m/z) 469.2 (MH+); RT=5.02 min.

### Example 157

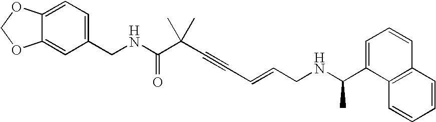

### (E)/(R)-2,2-Dimethyl-7-(1-naphthalen-1-yl-ethylamino)-hept-5-en-3-ynoic acid (benzo[1,3]dioxol-5-ylmethyl)-amide (Compound 266)

See GP3a.

LC/MS (METHOD A): (m/z) 455.2 (MH+); RT=4.99 min.

### Example 158

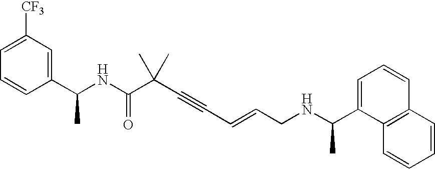

### (E)-2,2-Dimethyl-7-[(R)-1-naphthalen-1-yl-ethylamino)-hept-5-en-3-ynoic acid {1-[(S)-3-trifluoromethyl-phenyl)-ethyl}-amide (Compound 267)

See GP3a.

LC/MS (METHOD A): (m/z) 493.2 (MH+); RT=5.41 min.

### Example 159

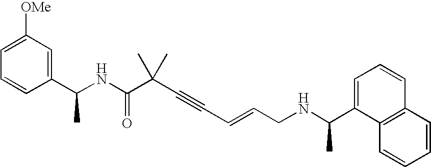

### (E)-2,2-Dimethyl-7-[(R)-1-naphthalen-1-yl-ethylamino)-hept-5-en-3-ynoic acid {1-[(S)-3-methoxy-phenyl)-ethyl}-amide (Compound 268)

See GP3a.

LC/MS (METHOD A): (m/z) 455.2 (MH+); RT=5.12 min.

### Example 160

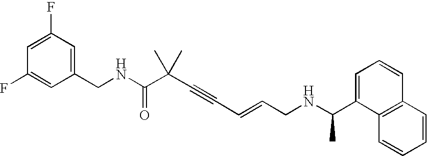

### (E)/(R)-2,2-Dimethyl-7-(1-naphthalen-1-yl-ethylamino)-hept-5-en-3-ynoic acid 3,5-difluoro-benzylamide (Compound 269)

See GP3a.

LC/MS (METHOD A): (m/z) 447.2 (MH+); RT=5.16 min.

### Example 161

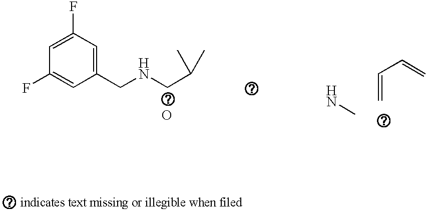

### (E)/(R)-2,2-Dimethyl-7-(1-naphthalen-1-yl-ethylamino)-hept-5-en-3-ynoic acid 4-t-butyl-benzylamide (Compound 270)

See GP3a.

LC/MS (METHOD A): (m/z) 467.2 (MH+); RT=5.57 min.

### Example 162

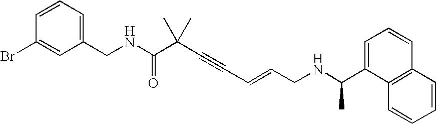

### (E)/(R)-2,2-Dimethyl-7-(1-naphthalen-1-yl-ethylamino)-hept-5-en-3-ynoic acid 3-bromo-benzylamide (Compound 271)

See GP3a.

LC/MS (METHOD A): (m/z) 489.1 (MH+); RT=5.24 min.

### Example 163

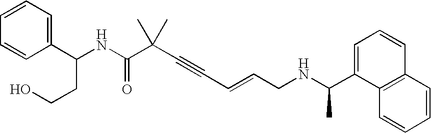

### (E)-2,2-Dimethyl-7-[(R)-1-naphthalen-1-yl-ethylamino]-hept-5-en-3-ynoic acid [(R/S)-3-hydroxy-1-phenyl-propyl)-amide (Compound 272)

See GP3a.

LC/MS (METHOD A): (m/z) 455.2 (MH+); RT=4.86 min.

### Example 164

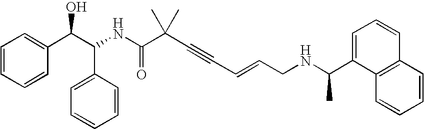

### (E)-2,2-Dimethyl-7-[(R)-1-naphthalen-1-yl-ethylamino)-hept-5-en-3-ynoic acid [(1R,2R)-2-hydroxy-1,2-diphenyl-ethyl]-amide (Compound 273)

See GP3a.

LC/MS (METHOD A): (m/z) 517.2 (MH+); RT=5.16 min.

### Example 165

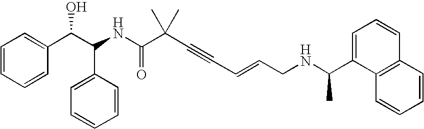

### (E)-2,2-Dimethyl-7-[(R)-1-naphthalen-1-yl-ethylamino)-hept-5-en-3-ynoic acid [(1S,2S)-2-hydroxy-1,2-diphenyl-ethyl]-amide (Compound 274)

See GP3a.

LC/MS (METHOD A): (m/z) 517.2 (MH+); RT=5.16 min.

### Example 166

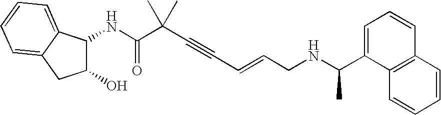

### (E)-2,2-Dimethyl-7-[(R)-1-naphthalen-1-yl-ethylamino]-hept-5-en-3-ynoic acid [(1S,2R)-2-hydroxy-indan-1-yl]-amide (Compound 275)

See GP3a.

LC/MS (METHOD A): (m/z) 453.2 (MH+); RT=4.94 min.

### Example 167

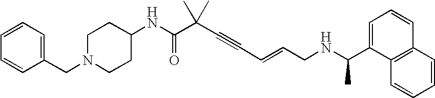

### (E)/(R)-2,2-Dimethyl-7-(1-naphthalen-1-yl-ethylamino)-hept-5-en-3-ynoic acid (1-benzyl-piperidin-4-yl)-amide (Compound 276)

See GP3a.

LC/MS (METHOD A): (m/z) 494.3 (MH+); RT=4.01 min.

### Example 168

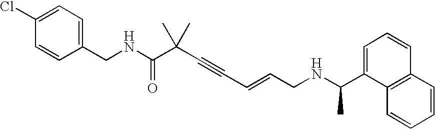

### (E)/(R)-2,2-Dimethyl-7-(1-naphthalen-1-yl-ethylamino)-hept-5-en-3-ynoic acid 4-chloro-benzylamide (Compound 277)

See GP3a.

LC/MS (METHOD A): (m/z) 445.2 (MH+); RT=5.21 min.

### Example 169

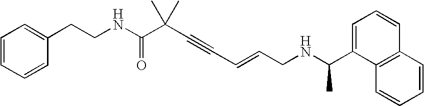

### (E)/(R)-2,2-Dimethyl-7-(1-naphthalen-1-yl-ethylamino)-hept-5-en-3-ynoic acid phenethyl-amide (Compound 278)

See GP3a.

LC/MS (METHOD A): (m/z) 425.2 (MH+); RT=5.09 min.

### Example 170

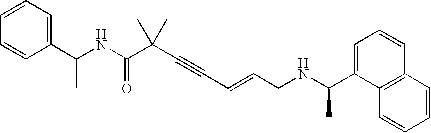

### (E)-2,2-Dimethyl-7-[(R)-1-naphthalen-1-yl-ethylamino]-hept-5-en-3-ynoic acid [(R/S)-1-phenyl-ethyl)-amide (Compound 279)

See GP3a.

LC/MS (METHOD A): (m/z) 425.2 (MH+); RT=5.12 min.

### Example 171

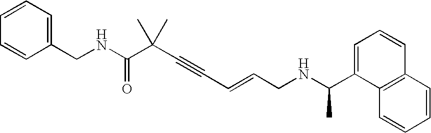

### (E)/(R)-2,2-Dimethyl-7-(1-naphthalen-1-yl-ethylamino)-hept-5-en-3-ynoic acid benzylamide (Compound 280)

See GP3a.

LC/MS (METHOD A): (m/z) 411.2 (MH+); RT=5.01 min.

### Example 172

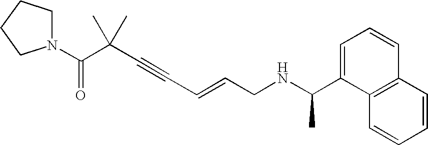

### (E)/(R)-2,2-Dimethyl-7-(1-naphthalen-1-yl-ethylamino)-1-pyrrolidin-1-yl-hept-5-en-3-yn-1-one (Compound 281)

See GP3a.

LC/MS (METHOD A): (m/z) 375.2 (MH+); RT=4.69 min.

### Example 173

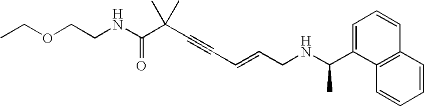

### (E)/(R)-2,2-Dimethyl-7-(1-naphthalen-1-yl-ethylamino)-hept-5-en-3-ynoic acid (2-ethoxy-ethyl)-amide (Compound 282)

See GP3a.

LC/MS (METHOD A): (m/z) 393.3 (MH+); RT=4.64 min.

### Example 174

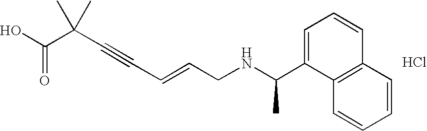

### (E)/(R)-2,2-Dimethyl-7-(1-naphthalen-1-yl-ethylamino)-hept-5-en-3-ynoic acid hydrochloride (Compound 283)

To a mixture of compound 168 (2.0 g, 4.46 mmol) in MeOH/H₂O (3:1, 50 mL) was added LiOH (1.0 g, 41.8 mmol) at rt. The obtained mixture was heated to reflux for 1 h. After the reaction was complete, MeOH was removed in vacuo. The aqueous residue was acidified by 4 N HCl. The precipitate was filtered and washed with H₂O and Et₂O, giving the title compound.

¹³C NMR (DMSO-d₅): δ=174.0, 133.8, 133.3, 133.0, 130.1, 128.9, 128.8, 126.8, 126.9, 125.5, 124.2, 122.5, 116.5, 95.0, 78.4, 51.6, 46.3, 37.8, 26.6, 19.7.

### Example 175

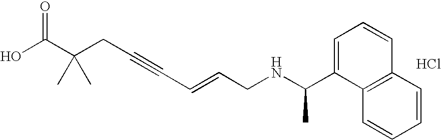

### (E)/(R)-2,2-Dimethyl-8-(1-naphthalen-1-yl-ethylamino)-oct-6-en-4-ynoic acid hydrochloride (Compound 284)

Compound 169 was treated as described for preparation of compound 283, giving the title compound.

¹³C NMR (CDCl₃): δ=177.3, 133.7, 133.3, 132.5, 130.1, 128.8, 126.8, 126.1, 125.5, 124.3, 122.5, 116.8, 89.9, 79.5, 51.6, 46.2, 41.3, 29.6, 24.0, 19.8.

### Example 176

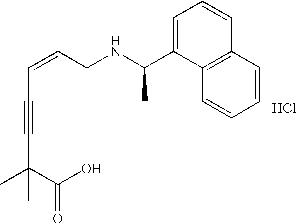

### (Z)/(R)-2,2-Dimethyl-7-(1-naphthalen-1-yl-ethylamino)-hept-5-en-3-ynoic acid hydrochloride (Compound 285)

Compound 170 was treated as described for preparation of compound 283, giving the title compound.

¹³C NMR (DMSO-d₆): δ=173.8, 133.5, 133.3, 132.4, 130.2, 129.0, 128.8, 126.9, 126.1, 125.4, 124.2, 122.4, 114.8, 100.1, 76.2, 51.3, 43.8, 37.9, 26.4, 19.6.

### Example 177

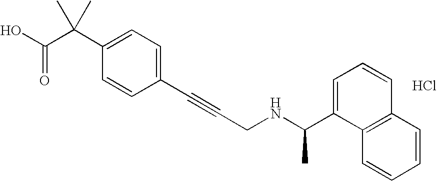

### (R)-2-Methyl-2-{4-[3-(1-naphthalen-1-yl-ethylamino)-prop-1-ynyl]-phenyl}-propionic acid hydrochloride (Compound 286)

A mixture of compound 174 (132 mg, 0.3 mmol) and LiOH (130 mg, 5.4 mmol) in MeOH/H₂O (3:1, 5 mL) was heated to reflux for 1 h. After the reaction was complete, MeOH was removed in vacuo. The aqueous residue was acidified by 4 N HCl. An oil product appeared. Removal of aqueous phase and wash with water gave the title compound as a foam.

¹³C NMR (DMSO-d₆): δ=177.0, 146.3, 133.6, 133.3, 131.4, 130.2, 129.0, 128.9, 126.8, 126.1, 125.5, 124.4, 122.5, 119.1, 86.8, 80.9, 51.1, 45.8, 34.8, 26.1, 19.7.

### Example 178

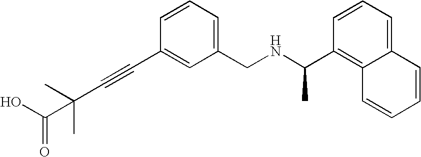

### (R)-2,2-Dimethyl-4-{3-[(1-naphthalen-1-yl-ethylamino)-methyl]-phenyl}-but-3-ynoic acid (Compound 287)

Compound 195 was treated as described for the preparation for compound 286. The aqueous residue was acidified to pH 5 by 4 N HCl. The precipitate was filtered off and washed with H₂O and small amount of Et₂O, giving the title compound as a solid.

¹³C NMR (DMSO-d₆): δ=174.3, 140.6, 133.5, 130.9, 130.8, 129.5, 128.6, 128.2, 128.1, 126.9, 125.7, 125.6, 125.3, 122.9, 122.9, 122.3, 92.8, 80.9, 52.3, 50.1, 37.9, 26.9, 23.3.

### Example 179

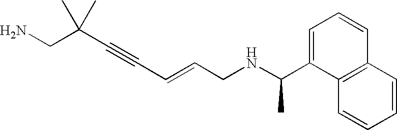

### (E)/(R)-6,6-Dimethyl-N*1*-(1-naphthalen-1-yl-ethyl)-hept-2-en-4-yne-1,7-diamine (Compound 288)

A solution of (E)/(R)-2-[2,2-Dimethyl-7-(1-naphthalen-1-yl-ethylamino)-hept-5-en-3-ynyl]-isoindole-1,3-dione (compound 404) from Preparation 3 (3.6 g, 8.25 mmol) and hydrazine monohydrate (8.0 mL, 160.0 mmol) in THF/EtOH (200 mL) was stirred at 60° C. for 1 h. After being cooled to it and filtered, the reaction mixture was concentrated in vacuo. The crude material was taken up in DCM (200 mL)/1 N NaOH (100 mL). The organic phase was dried over MgSO₄ and concentrated in vacuo, giving the title compound.

¹³C NMR (CDCl₃): δ=141.1, 140.9, 134.0, 131.3, 129.0, 127.3, 125.8, 125.7, 125.3, 123.0, 122.7, 111.0, 95.3, 79.7, 53.9, 53.0, 49.3, 34.7, 26.5, 23.7.

### Example 180

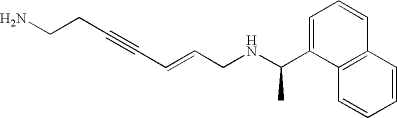

### (E)/(R)—N*1*-(1-naphthalen-1-yl-ethyl)-hept-2-en-4-yne-1,7-diamine (Compound 289)

(E)/(R)-2-[7-(1-Naphthalen-1-yl-ethylamino)-hept-5-en-3-ynyl]-isoindole-1,3-dione hydrochloride (compound 403) from Preparation 3 was treated as described for the preparation of compound 288, giving the title compound.

¹³C NMR (CDCl₃): δ=141.3, 140.9, 134.0, 131.3, 129.0, 127.3, 125.8, 125.7, 125.3, 122.9, 122.7, 111.0, 87.7, 80.2, 52.9, 49.2, 41.2, 24.5, 23.6.

### Example 181

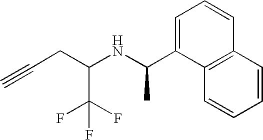

### [(R)-1-Naphthalen-1-yl-ethyl]-[(R/S)-1-trifluoromethyl-but-3-ynyl]-amine (Compound 290)

To a mixture of Zinc (85 mg, 1.3 mmol) in dry DMF (5 mL) was added dibromoethane under argon atmosphere. The reaction mixture was stirred at 75° C. for 30 min and cooled to 0° C. Chlorotrimethylsilane (11 mg, 0.1 mmol) was added, followed by addition of compound 224 (251 mg, 1 mmol) and propargyl bromide (0.16 mL, 1.4 mmol) in DMF (2 mL). The obtained reaction mixture was stirred at rt for 3 h. The reaction was quenched with saturated aq. solution of NH₄Cl. The mixture was extracted with EtOAc. The combined organic phases were concentrated in vacuo. The residue was purified by flash chromatography (heptane/EtOAc 1:0→1:1), giving the title compound as a colorless oil.

¹³C NMR (DMSO-d₆): δ=140.5, 133.4, 130.7, 128.7, 127.1, 126.5 (¹J_{CF}=286.1 Hz), 125.9, 125.6, 125.4, 123.3, 122.7, 79.7, 73.2, 54.9 (²J_{CF}=26.4 Hz), 50.8, 23.7, 19.3 (³J_{CF}=2.6 Hz).

### Example 182

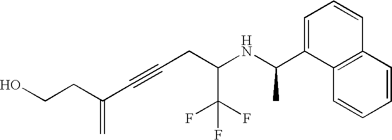

### [(R/S)-8,8,8-Trifluoro-3-methylene]-7-[(R)-1-naphthalen-1-yl-ethylamino]-oct-4-yn-1-ol (Compound 291)

3-Bromo-3-buten-1-ol and compound 290 was treated as described in GP2a. The crude was purified by flash chromatography (heptane/DCM 1:0→1:1) giving the title compound.

¹³C NMR (DMSO-d₅): δ=140.5, 133.4, 130.6, 128.7, 128.3, 127.1, 125.9, 125.6, 125.4, 123.1, 122.6, 122.1, 85.3, 82.6, 59.2, 55.1 (²J_{CF}=26.3 Hz), 50.6, 40.3, 23.8, 20.1 (³J_{CF}=2.6 Hz).

### Example 183

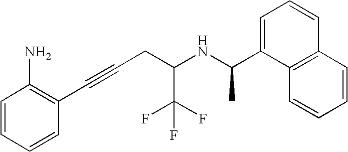

### 2-{[(R/S)-5,5,5-Trifluoro]-4-[(R)-1-naphthalen-1-yl-ethylamino]-pent-1-ynyl}-phenylamine (Compound 292)

2-Iodoaniline and compound 290 was treated as described in GP2a. The crude was purified by flash chromatography (heptane/DCM 1:0→1:1) giving the title compound.

¹H NMR (DMSO-d₅): δ=8.20-8.15 (m, 1H), 7.96-7.88 (m, 1H), 7.82 (d, 1H), 7.78 (d, 1H), 7.54-7.32 (m, 3H), 7.10-7.01 (m, 2H), 6.70 (d, 1H), 6.49 (dt, 1H), 5.36 (bs, 2NH), 4.99 (h, 1H), 3.40-3.28 (m, 1H), 3.05-2.73 (m, 2H), 1.41 (d, 3H).

### Example 184

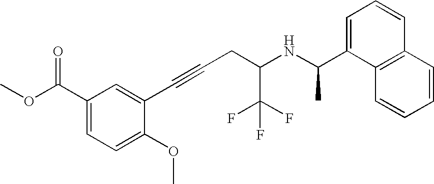

### 4-Methoxy-3-{[(R/S)-5,5,5-trifluoro]-4-[(R)-1-naphthalen-1-yl-ethylamino]-pent-1-ynyl}-benzoic acid methyl ester (Compound 293)

Methyl 3-Iodo-4-methoxy-benzoate and compound 290 was treated as described in GP2a. The crude was purified by flash chromatography (heptane/DCM 1:0→1:1) giving the title compound.

¹³C NMR (DMSO-d₆): δ=165.2, 163.1, 140.7, 133.9, 133.4, 131.1, 130.6, 128.6, 127.1, 126.5 (¹J_{CF}=285.0 Hz), 125.8, 125.5, 125.3, 123.2, 122.6, 121.7, 111.9, 111.2, 90.5, 77.5, 56.0, 55.3 (²J_{CF}=26.3 Hz), 51.9, 50.6, 23.8, 20.4 (³J_{CF}=1.5 Hz).

### Example 185

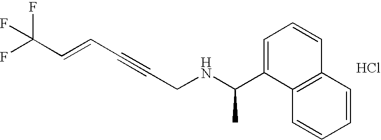

### (E)/(R)-(1-Naphthalen-1-yl-ethyl)-(6,6,6-trifluoro-hex-4-en-2-ynyl)-amine hydrochloride (Compound 294)

(R)-tert-Butyl ester (1-naphthalen-1-yl-ethyl)-prop-2-ynyl-carbamate (compound 434) from preparation 33 and 1-bromo-3,3,3-trifluoro-propene were treated as described in GP2a. The crude was purified by flash chromatography (heptane/EtOAc 1:0→1:1), giving a Boc-protected amine.

This Boc-protected amine was dissolved in DCM and silica gel was added. The mixture was concentrated to dryness. The residue was heated at 130° C. for 15 min and put into a column containing silica gel. Chromatography (heptane/EtOAc 1:0→1:1) gave the title compound.

¹³C NMR (DMSO-d₆): δ=133.3, 133.3, 130.2, 129.1, 128.8, 128.7 (²J_{CF}=33.2 Hz), 126.8, 126.1, 125.5, 124.5, 122.6, 122.5 (¹J_{CF}=269.6 Hz), 118.8 (³J_{CF}=7.9 Hz), 88.2, 82.5, 51.3, 34.5, 19.7.

### Preparation

### Preparation 1:

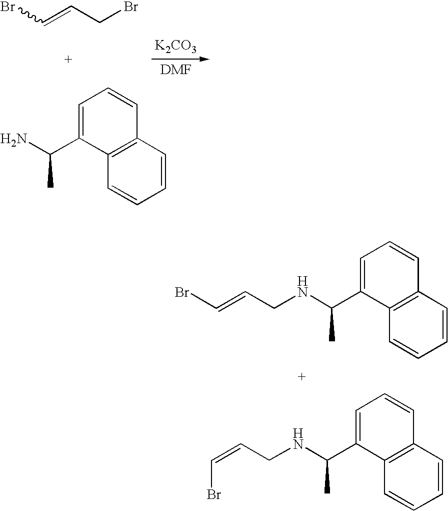

### (E)/(R)-(3-Bromo-allyl)-(1-naphthalen-1-yl-ethyl)-amine (Compound 201) and (Z)/(R)-(3-Bromo-allyl)-C1-naphthalen-1-yl-ethyl)-amine (Compound 402)

A mixture of (E/Z)-1,3-dibromo-propene (5.0 g, 25.0 mmol), (R)-1-naphthalen-1-yl-ethylamine (5.0 g, 29.2 g mmol), and K₂CO₃ (5.0 g) in DMF (30 mL) was treated as described in the General procedure 1. The crude was purified by chromatography (PE/EtOAc 3:1), giving firstly compound 201 and then compound 202.

### Compound 401:

¹³C NMR (CDCl₃): δ=140.6, 136.4, 134.0, 131.3, 129.0, 127.4, 125.8, 125.7, 125.4, 122.8, 122.8, 106.8, 52.7, 49.2, 23.6.

### Compound 402:

¹³C NMR (CDCl₃): δ=140.7, 134.0, 133.6, 131.3, 129.0, 127.3, 125.8, 125.7, 125.3, 122.9, 122.8, 109.0, 53.2, 46.8, 23.5.

### Preparation 2:

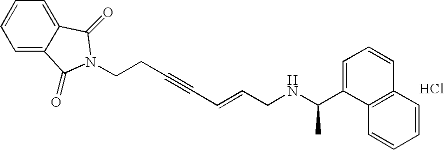

### (E)/(R)-2-[7-(1-Naphthalen-1-yl-ethylamino)-hept-5-en-3-ynyl]-isoindole-1,3-dione hydrochloride (Compound 403)

(E)/(R)-(3-Bromo-allyl)-(1-naphthalen-1-yl-ethyl)-amine (1 equiv) and 2-but-3-ynyl-isoindole-1,3-dione (3.5 equiv) were treated as described in GP2a. Chromatography (PE/EtOAc 1:1→0:1) gave a free amine. The free amine was redissolved in Et₂O. To the solution was added HCl in 1,4-dioxane (4N) until pH2 and the product was crystallized out. Filtration and wash of the crystals with small amount of Et₂O gave the title compound as a solid.

¹³C NMR (DMSO-d₆): δ=167.9, 134.9, 134.2, 133.7, 133.2, 131.8, 130.5, 129.3, 127.2, 126.5, 125.9, 124.8, 123.5, 122.9, 116.9, 89.5, 79.6, 51.9, 46.6, 36.5, 20.1, 18.8.

### Preparation 3:

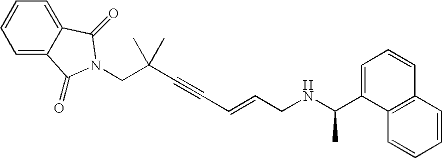

### (E)/(R)-2-[2,2-Dimethyl-7-(1-naphthalen-1-yl-ethylamino)-hept-5-en-3-ynyl]-isoindole-1,3-dione (Compound 404)

(E)/(R)-(3-Bromo-allyl)-(1-naphthalen-1-yl-ethyl)-amine (1 equiv) and 2-(2,2-dimethyl-but-3-ynyl)-isoindole-1,3-dione (3.5 equiv) were treated as described in GP2a. Chromatography (PE/EtOAc 1:1→0:1) gave the title compound.

¹³C NMR (CDCl₃): δ=168.5, 141.4, 140.9, 133.9, 132.1, 131.3, 128.9, 127.2, 125.8, 125.7, 125.3, 123.3, 123.0, 122.8, 110.8, 94.3, 80.0, 52.9, 49.2, 47.8, 34.2, 27.8, 23.6.

### Preparation 4:

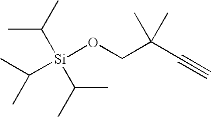

### (2,2-dimethyl-but-3-ynyloxy)-triisopropyl-silane (Compound 405)

To a suspension of 4-dodecyl-benzenesulfonyl azide (4.2 g, 12.0 mmol) and K₂CO₃ (4.1 g, 30.0 mmol) in MeCN (100 mL) was added dimethyl (2-oxopropyl)phosphate (2.0 g, 12 mmol) at rt. The mixture was stirred at this temperature for 0.5 h. A solution of 2,2-dimethyl-3-triisopropylsilanyloxy-propionaldehyde (2.60 g, 10 mmol) in MeOH (10 mL) was added. Stirring was continued for 3 days. The solvent was removed in vacuo. The residue was taken up in Et₂O and brine. The aqueous phase was extracted twice with Et₂O. The combined organic phases were dried and concentrated in vacuo. The residue was purified by chromatography (PE), giving the title compound as a colorless liquid.

¹H NMR (CDCl₃): δ=3.57 (s, 2H), 2.05 (s, 1H), 1.21 (s, 6H), 1.10-1.00 (m, 21H).

### Preparation 5:

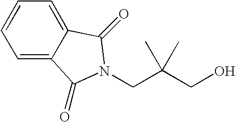

### 2-(3-hydroxy-2,2-dimethyl-propyl)-isoindole-1,3-dione (Compound 406)

A mixture of potassium phthalimide (20.0 g, 108.0 mmol) and 3-bromo-2,2-dimethyl-propan-1-ol (20 g, 119.7 mmol) in DMF (200 mL) was stirred at 135° C. (oil bath temperature) for 18 h.

The reaction mixture was poured into brine/H₂O (1/1) and extracted twice with Et₂O. The combined organic phases were dried over Na₂SO₄ and concentrated in vacuo, giving a yellowish solid. The solid was taken up in Et₂O. The insoluble solid was filtered off. To the filtrate was added PE. The suspension stood overnight. The crystals were filtered and dried, giving the title product as a white solid.

¹³C NMR (CDCl₃): δ=169.7, 134.3, 131.8, 123.5, 68.2, 43.9, 38.0, 22.9.

### Preparation 6:

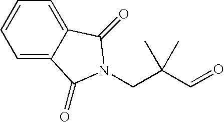

### 3-(1,3-Dioxo-1,3-dihydro-isoindol-2-yl)-2,2-dimethyl-propionaldehyde (compound 407)

To a mixture of compound 406 (14.0 g, 60.0 mmol), NMO (10.5 g, 90.0 mmol), and mol.sieve 4 Å (10 g) in DCM (150 mL) was added TPAP (0.42 g, 1.2 mmol) in portion. The obtained mixture was stirred at this temperature for two days. The mixture was filtered through a short column of silica gel (DCM). The solution was concentrated in vacuo. The residue was purified by chromatography (PE/DCM 3:1), giving title compound as an oil, which was used directly in the next step.

### Preparation 7:

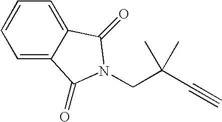

### 2-(2,2-dimethyl-but-3-ynyl)-isoindole-1,3-dione (Compound 408)

To a mixture of dodecylbenzenesulfonyl azide (12.0 g, 34.1 mmol) and K₂CO₃ (7.9 g, 57.2 mmol) was added dimethyl (2-oxopropyl)phosphonate (5.6 g, 33.7 mmol) at rt. The obtained mixture was stirred at this temperature for 5 days. The mixture was concentrated in vacuo. The residue was taken up in H₂O and extracted twice with Et₂O. The combined organic phases were concentrated in vacuo. The residue was purified by chromatography (PE/EA 6:1), giving an impure solid. The impure compound was washed with PE, giving the title compound as a white solid.

¹H NMR (CDCl₃): δ=7.93-7.80 (m, 2H), 7.79-7.68 (m, 2H), 3.77 (s, 2H), 2.13 (s, 1H), 1.01 (s, 6H).

### Preparation 8:

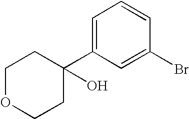

### 4-(3-bromo-phenyl)-tetrahydro-pyran-4-ol (Compound 409)

To a solution of 1-bromo-3-iodo-benzene (5.66 g, 20 mmol) in THF (50 mL) was added isopropylmagnesium chloride (24 mL, 1 M in Et₂O) at −10° C. After the solution was stirred at this temperature for 1 h, tetrahydro-pyran-4-one (2.0 mL, 20 mmol) was added. The reaction solution was stirred at rt for 18 h. the reaction was quenched with H₂O. The mixture was extracted twice with Et₂O. The combined organic phases were dried over MgSO₄ and concentrated in vacuo and the residue was purified by chromatography (PE/EtOAc 2:1), giving the title product as a white solid.

¹³C NMR (CDCl₃): δ=150.5, 130.3, 130.1, 128.0, 123.2, 122.8, 70.6, 63.7, 38.7.

### Preparation 9:

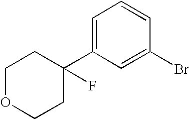

### 4-(3-bromo-phenyl)-4-fluoro-tetrahydro-pyran (Compound 410)

To a solution of compound 409 (1.00 g, 3.9 mmol) in DCM was added DAST (0.8 g, 5.0 mmol) at 0° C. The solution was stirred at this temperature for 0.5 h. The reaction was quenched with H₂O and extracted with DCM. The combined organic phases were concentrated in vacuo. The residue was purified by chromatography (PE/EtOAc 20:1), giving the title compound as an oil, which used directly in the next step without characterization.

### Preparation 10:

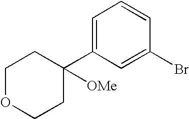

### 4-(3-bromo-phenyl)-4-methoxy-tetrahydro-pyran (Compound 411)

To a solution of compound 409 (1.00 g, 3.9 mmol) in DMF (10 mL) was added NaH (0.23 g, 60% in oil, 5.8 mmol). The mixture was stirred at this temperature for 0.5 h. MeI (0.8 mL, 12.8 mmol) was added. The mixture was stirred for another 0.5 h and poured into H₂O/brine 1:1 and extracted with Et₂O. The combined organic phases were dried and concentrated in vacuo. The residue was purified chromatography (PE/EA 2:1), giving the title product as an oil.

¹³C NMR (CDCl₃): δ=147.1, 130.5, 130.1, 129.2, 124.6, 122.9, 74.8, 63.5, 50.0, 35.2.

### Preparation 11:

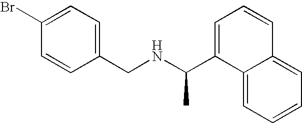

### (R)-4-Bromobenzyl-(1-naphthalen-1-yl-ethyl)-amine (Compound 412)

4-Bromobenzaldehyde and (R)-1-naphthalen-1-yl-ethylamine were treated as described in GP6. The crude was purified by flash chromatography (heptane/EtOAc 1:0→0:1).

¹³C NMR (DMSO-d₆): δ=141.7, 140.9, 133.9, 131.3, 131.2, 130.4, 129.0, 127.1, 126.1, 125.6, 123.3, 123.2, 119.7, 52.9, 50.5, 24.1.

### Preparation 12:

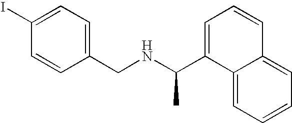

### (R)-(4-Iodo-benzyl)-(1-naphthalen-1-yl-ethyl)-amine (Compound 413)

4-Iodobenzaldehyde and (R)-1-naphthalen-1-yl-ethylamine were treated as described in GP6. The crude was purified by flash chromatography (heptane/EtOAc 1:0→0:1).

¹³C NMR (DMSO-d₆): δ=141.7, 141.3, 137.1, 133.9, 131.3, 130.7, 129.1, 127.2, 126.1, 125.7, 123.4, 123.2, 92.4, 52.9, 50.6, 24.1.

### Preparation 13:

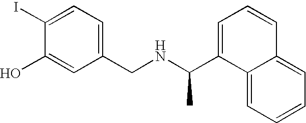

### (R)-(3-hydroxy-4-iodo-benzyl)-(1-naphthalen-1-yl-ethyl)-amine (Compound 414)

3-Hydroxy-4-iodobenzaldehyde and (R)-1-naphthalen-1-yl-ethylamine were treated as described in GP6. The crude was purified by flash chromatography (heptane/EtOAc 1:0→0:1).

¹³C NMR (DMSO-d₆): δ=156.3, 142.9, 141.3, 138.1, 133.5, 130.8, 128.6, 126.7, 125.7, 125.6, 125.2, 122.9, 122.6, 120.6, 114.5, 81.6, 52.4, 50.2, 23.6.

### Preparation 14:

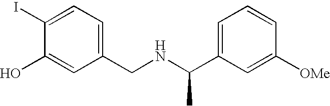

### (R)-(3-hydroxy-4-iodo-benzyl)-[1-(3-methoxy-phenyl)-ethyl]-amine (Compound 415)

3-Hydroxy-4-iodobenzaldehyde and (R)-1-naphthalen-1-yl-ethylamine were treated as described in GP6. The crude was purified by flash chromatography (heptane/EtOAc 1:0→0:1).

¹³C NMR (DMSO-d₆): δ=159.3, 156.3, 147.7, 142.9, 138.1, 129.1, 120.6, 118.7, 114.5, 111.9, 111.7, 81.5, 56.5, 54.8, 49.9, 24.3.

### Preparation 15:

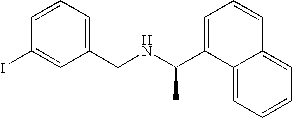

### (R)-(3-Iodo-benzyl)-(1-naphthalen-1-yl-ethyl)-amine (Compound 416)

3-Iodobenzaldehyde and (R)-1-naphthalen-1-yl-ethylamine were treated as described in GP6. The crude was purified by flash chromatography (heptane/EtOAc 1:0→0:1).

¹³C NMR (DMSO-d₆): δ=143.8, 141.2, 136.4, 135.1, 133.5, 130.9, 130.1, 128.6, 127.3, 126.7, 125.6, 125.2, 122.9, 122.8, 94.6, 52.4, 50.1, 23.6.

### Preparation 16:

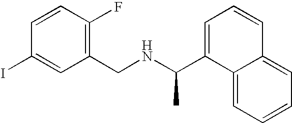

### (R)-(5-Fluoro-3-iodo-benzyl)-(1-naphthalen-1-yl-ethyl)-amine (Compound 417)

5-Fluoro-3-iodobenzaldehyde and (R)-1-naphthalen-1-yl-ethylamine were treated as described in GP6. The crude was purified by flash chromatography (heptane/EtOAc 1:0→0:1).

¹³C NMR (DMSO-d₆): δ=160.5 (¹J_{CF}=244.8 Hz), 141.5, 138.9 (³J_{CF}=4.6 Hz), 137.3 (³J_{CF}=7.8 Hz), 133.9, 131.3, 131.1 (²J_{CF}=16.6 Hz), 129.0, 127.2, 126.1, 126.0, 125.7, 123.3, 123.1, 117.8 (²J_{CF}=23.2 Hz), 88.5 (⁴J_{CF}=3.3 Hz), 53.1, 43.7 (³J_{CF}=2.7 Hz), 24.0.

### Preparation 17:

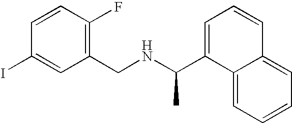

### (R)-(5-Fluoro-3-iodo-benzyl)-(1-phenyl-ethyl)-amine (Compound 418)

5-Fluoro-3-iodobenzaldehyde and (R)-1-phenyl-ethylamine were treated as described in GP6. The crude was purified by flash chromatography (heptane/EtOAc 1:0→0:1). ¹³C NMR (DMSO-d₆): δ=160.5 (¹J_{CF}=244.8 Hz), 146.1, 138.7 (³J_{CF}=4.6 Hz), 137.2 (³J_{CF}=8.0 Hz), 131.1 (²J_{CF}=15.9 Hz), 128.6, 127.0, 126.8, 117.8 (²J_{CF}=23.2 Hz), 88.5 (⁴J_{CF}=3.3 Hz), 57.4, 43.5 (⁴J_{CF}=2.7 Hz), 24.8.

### Preparation 18:

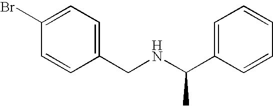

### (R)-Bromobenzyl-(1-phenyl-ethyl)-amine (Compound 419)

4-Bromobenzaldehyde and (R)-1-phenyl-ethylamine were treated as described in GP6. The crude was purified by flash chromatography (heptane/EtOAc 1:0→0:1).

¹³C NMR (DMSO-d₆): δ=146.3, 140.9, 131.2, 130.4, 128.6, 126.9, 119.7, 57.0, 50.2, 24.8.

### Preparation 19:

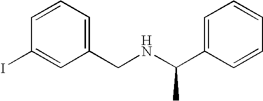

### (R)-(3-Iodo-benzyl)-(1-phenyl-ethyl)-amine (Compound 420)

3-Iodobenzaldehyde and (R)-1-phenyl-ethylamine were treated as described in GP6. The crude was purified by flash chromatography (heptane/EtOAc 1:0→0:1).

¹³C NMR (DMSO-d₆): δ=145.8, 143.8, 136.2, 135.0, 130.1, 128.1, 127.2, 126.5, 126.4, 94.6, 56.7, 49.9, 24.4.

### Preparation 20:

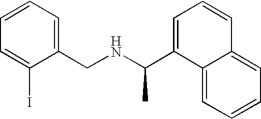

### (R)-(2-Iodo-benzyl)-(1-naphthalen-1-yl-ethyl)-amine (Compound 421)

2-Iodobenzaldehyde and (R)-1-naphthalen-1-yl-ethylamine were treated as described in GP6. The crude was purified by flash chromatography (heptane/EtOAc 1:0→0:1).

¹³C NMR (DMSO-d₆): δ=142.5, 141.1, 138.6, 133.5, 130.8, 129.2, 128.6, 128.1, 126.7, 125.7, 125.6, 125.2, 122.9, 122.7, 99.6, 55.5, 52.6, 23.7.

### Preparation 21:

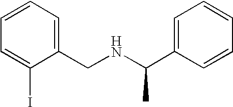

### (R)-(2-Iodo-benzyl)-(1-phenyl-ethyl)-amine (Compound 422)

2-Iodobenzaldehyde and (R)-1-phenyl-ethylamine were treated as described in GP6. The crude was purified by flash chromatography (heptane/EtOAc 1:0→0:1).

¹³C NMR (DMSO-d₆): δ=145.7, 142.5, 138.6, 129.1, 128.5, 128.1, 128.0, 126.5, 126.4, 99.6, 56.9, 55.3, 24.4.

### Preparation 22:

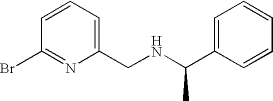

### (R)-(6-Bromo-pyridin-2-ylmethyl)-(1-phenyl-ethyl)-amine (Compound 423)

3-Iodobenzaldehyde and (R)-1-phenyl-ethylamine were treated as described in GP6. The crude was purified by flash chromatography (heptane/EtOAc 1:0→0:1), which was used directly in the next step.

### Preparation 23:

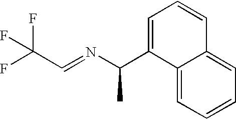

### (R)-(1-Naphthalen-1-yl-ethyl)-(2,2,2-trifluoro-ethylidene)-amine (Compound 424)

(R)-1-Naphthyl)ethylamine (10.0 g, 58.4 mmol) was mixed with trifluoracetaldehydethyl hemiacethal (10.7 g, 73.0 mmol) in toluene (150 mL) and the mixture turned cloudy. Equipped with Dean-Stark apparatus, the reaction was refluxed for 1 h. After additional trifluoracetaldehyd ethyl hemiacethal (2.10 g, 14.5 mmol) was added, the reaction was refluxed for further 2 h. Toluene was then removed under reduced pressure, furnishing a colorless oil. This crude product was further purified by flash chromatography (heptane/EtOAc 1:0→1:1), giving the title compound as a colorless oil

¹³C NMR (DMSO-d₆): δ=149.8 (²J_{CF}=36.4 Hz), 138.6, 133.5, 130.0, 128.8, 127.7, 126.2, 125.7, 125.6, 123.7, 123.0, 119.2 (¹J_{CF}=275.3 Hz), 62.7, 23.3

### Preparation 24:

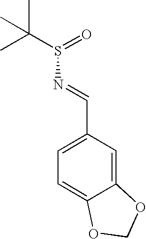

### Benzo[1,3]dioxol-5-ylmethylene 2-methyl-propane-2-sulfinamide (Compound 425)

3,4-Methylendioxy-benzaldehyde and (S)-(−)-2-methyl-2-propanesulfinamide were treated as described in GP7. The residue was purified by flash chromatography (heptane/EtOAc 1:0→1:1), giving the title compound.

¹³C NMR (DMSO-d₆): δ=161.6, 151.2, 148.1, 128.5, 126.7, 108.5, 106.8, 101.9, 57.0, 21.9.

### Preparation 25:

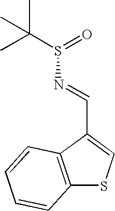

### Benzo[b]thiophen-3-ylmethylene 2-methyl-propane-2-sulfinamide (Compound 426)

1-Benzothiophene-3-carbaldehyde and (S)-(−)-2-methyl-2-propanesulfinamide were treated as described in GP7. The residue was purified by flash chromatography (heptane/EtOAc 1:0→1:1), giving the title compound.

¹³C NMR (DMSO-d₆): δ=157.2, 141.2, 140.1, 135.2, 131.4, 125.8, 125.7, 124.2, 123.2, 56.7, 21.9.

### Preparation 26:

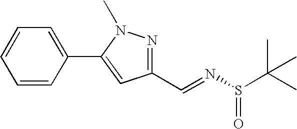

### 1-Methyl-5-phenyl-1H-pyrazol-3-ylmethylene 2-methyl-propane-2-sulfinamide (Compound 427)

1-Benzothiophene-3-carbaldehyde and (S)-(−)-2-methyl-2-propanesulfinamide were treated as described in GP7. The residue was purified by flash chromatography (heptane/EtOAc 1:0→1:1), giving the title compound.

¹³C NMR (DMSO-d₆): δ=155.7, 146.5, 145.1, 129.0, 128.9, 128.8, 128.6, 105.6, 57.0, 38.3, 21.9.

### Preparation 27:

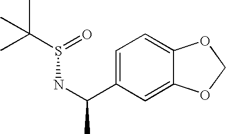

### (1-Benzo[1,3]dioxol-5-yl-ethyl) 2-methyl-propane-2-sulfinamide (Compound 428)

Compound 426 was treated as described in GP8, giving the title compound.

¹³C NMR (DMSO-d₆): δ=147.6, 146.4, 139.2, 120.2, 108.2, 107.4, 101.1, 55.2, 55.0, 25.4, 23.0

### Preparation 28:

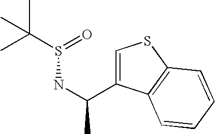

### (1-benzo[b]thiophen-3-yl-ethyl)-2-methyl-propane-2-sulfinamide (Compound 429)

Compound 426 was treated as described in GP8, giving the title compound.

¹³C NMR (DMSO-d₆): δ=140.4, 139.4, 137.7, 124.6, 124.1, 123.6, 123.3, 123.0, 55.4, 50.7, 23.2, 23.1.

### Preparation 29:

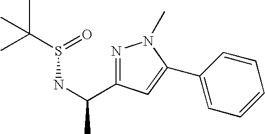

### [1-(1-Methyl-5-phenyl-1H-pyrazol-3-yl)-ethyl]2-methyl-propane-2-sulfinamide (Compound 430)

Compound 427 was treated as described in GP8, giving the title compound.

¹³C NMR (DMSO-d₆): δ=154.2, 143.7, 130.6, 129.2, 128.7, 128.6, 104.2, 55.4, 49.7, 37.7, 22.9, 22.8.

### Preparation 30:

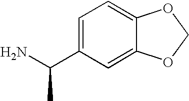

### (R)-1-Benzo[1,3]dioxol-5-yl-ethylamine (Compound 431)

Compound 428 was treated as described in GP9, giving the title compound as a colorless oil.

¹³C NMR (DMSO-d₆): δ=147.0, 145.3, 143.1, 118.5, 107.6, 106.2, 100.4, 50.4, 26.2.

### Preparation 31:

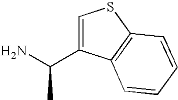

### (R)-1-Benzo[b]thiophen-3-yl-ethylamine (Compound 432)

Compound 429 was treated as described in GP9, giving the title compound as a colorless oil.

¹³C NMR (DMSO-d₆): δ=143.6, 140.2, 137.5, 124.0, 123.7, 122.8, 122.2, 120.5, 45.5, 24.5.

### Preparation 32:

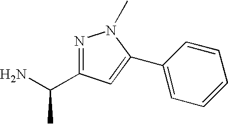

### (R)-1-(1-Methyl-5-phenyl-1H-pyrazol-3-yl)-ethylamine (Compound 433)

Compound 430 was treated as described in GP9, giving the title compound as a colorless oil.

¹³C NMR (DMSO-d₆): δ=157.2, 143.0, 130.5, 128.7, 128.2, 128.1, 102.2, 45.0, 37.1, 24.3.

### Preparation 33.

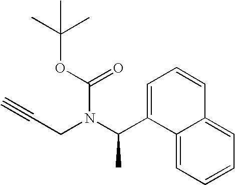

### (R)-tert-Butyl ester (1-naphthalen-1-yl-ethyl)-prop-2-ynyl-carbamate (Compound 434)

To (R)-(1-Naphthyl)ethylamine (1 equiv) and propargylbromide (1.1 eq) in H₂O (20 mL/g of the amine was added NaOH (1.5 equiv) and the reaction mixture was stirred at rt for 20 h.

(Boc)₂O (1.1 equiv) was added and the reaction mixture was stirred for additional 3 h before EtOAc was added. The organic phase was washed with 0.05 N HCl and water, dried (MgSO₄), and evaporated. The residue was purified by flash chromatography (heptane/EtOAc 1:0→1:1), giving the title compound.

¹³C NMR (DMSO-d₅): δ=153.8, 133.3, 131.3, 128.6, 128.4, 126.3, 125.7, 125.1, 124.5, 123.1, 81.3, 79.6, 72.3, 49.8, 31.3, 27.9, 17.2.

1-29. (canceled)

30. A compound of general formula Ia or Ib 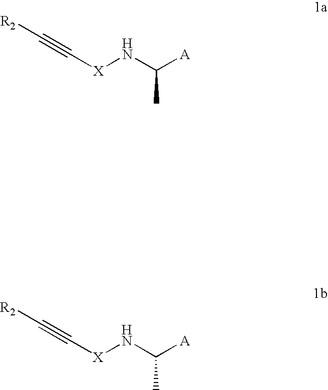
wherein
A represents C₁₋₁₀heteroaryl, C₆₋₁₄aryl or C₆₋₁₀heterocycloalkylaryl,
each of which are optionally substituted with one or more, same or different substituents selected from the group consisting of halogen, hydroxy, mercapto, trifluoromethyl, cyano, carboxy, CONH₂, nitro, oxo, —S(O)₂NH₂, C₂₋₃alkyl, C₂₋₄alkenyl, C₂₋₄alkynyl, C₁₋₄hydroxyalkyl, C₁₋₆halo alkyl, C₁₋₄alkoxy, C₁₋₄alkoxycarbonyl, C₁₋₄alkylcarbonyloxy, C₁₋₄alkoxycarbonyloxy, C₁₋₄alkoxysulfonyloxy, C₁₋₄alkoxycarbamoyl, C₁₋₄aminocarbonyl, C₁₋₄alkylthio, C₃₋₆cycloalkyl, C₃₋₆cycloalkenyl, C₁₋₆amino, —NH₂, C₁₋₆imino, C₁₋₄aminosulfonyl, C₁₋₄aminocarbonyloxy, C₁₋₄alkylsulfonyl, C₁₋₄alkoxyimino, C₁₋₄alkylcarbonylamino, C₁₋₄alkylsulfonyl, C₃₋₆heterocycloalkyl, C₃₋₆heterocycloalkenyl, C₁₋₄aminocarbonyloxy, C₁₋₁₀heteroaryl or C₆₋₁₄aryl,
wherein said C₂₋₃alkyl, C₂₋₄alkenyl, C₂₋₄alkynyl, C₁₋₄hydroxyalkyl, C₁₋₆haloalkyl, C₁₋₄alkoxy, C₁₋₄alkoxycarbonyl, C₁₋₄alkylcarbonyloxy, C₁₋₄alkoxycarbonyloxy, C₁₋₄alkoxysulfonyloxy, C₁₋₄alkoxycarbamoyl, C₁₋₄aminocarbonyl, C₁₋₄alkylthio, C₃₋₆cycloalkyl, C₃₋₆cycloalkenyl, C₁₋₆amino, C₁₋₆imino, C₁₋₄aminosulfonyl, C₁₋₄aminocarbonyloxy, C₁₋₄alkylsulfonylamino, C₁₋₄alkoxyimino, C₁₋₄alkylcarbonylamino, C₁₋₄alkylsulfonyl, C₃₋₆heterocycloalkyl, C₃₋₆heterocycloalkenyl, C₁₋₄aminocarbonyloxy, C₁₋₁₀heteroaryl or C₆₋₁₄aryl,
are optionally further substituted with one or more, same or different substituents selected from the group consisting of halogen, hydroxy, —NH₂, mercapto, trifluoromethyl, cyano, carboxy, CONH₂, nitro, oxo, —S(O)₂NH₂, C₁₋₄alkyl, C₁₋₆haloalkyl, C₁₋₃alkoxy or C₁₋₃hydroxyalkyl;
R₁ is C₁₋₆alkyl, C₂₋₆alkenyl, C₂₋₆alkynyl, C₁₋₆hydroxyalkyl, C₁₋₆haloalkyl, C₁₋₆amino, C₃₋₆cycloalkyl, or C₁₋₆heterocycloalkyl,
each of which are optionally substituted with one or more, same or different substituents selected from the group consisting of halogen, hydroxy, mercapto, trifluoromethyl, cyano, carboxy, CONH₂, nitro, oxo, C₁₋₃alkyl, C₂₋₄alkenyl, C₁₋₃hydroxyalkyl, C₁₋₃haloalkyl, C₁₋₄alkoxy, C₁₋₄alkoxycarbonyl, C₁₋₄amino, or —NH₂;
X represents —CR₃R₄—(CR₅R₆)ₙ—(CR₇═CR₈)ₘ—(C₆₋₁₄aryl)ᵣ-(C₁₋₁₀heteroaryl)ₛ-(CR₉R₁₀)ₚ—(CR₁₁═CR₁₂)_{q},
wherein n, m, p, q, r and s independently of each other is an integer of 0, 1, 2, 3, 4, 5, 6, 7, 8, 9, or 10,
R₃, R₄, R₅, R₆, R₇, R₈, R₉, R₁₀, R₁₁, R₁₂ independently of each other represent hydrogen, halogen, hydroxy, NH₂, C₁₋₆alkyl, C₁₋₆alkoxy, C₂₋₆alkenyl, C₂₋₆alkynyl, C₁₋₆hydroxyalkyl, C₁₋₆haloalkyl, C₃₋₆heterocycloalkyl, or C₃₋₆cycloalkyl,
the last eight of which are optionally substituted with one or more, same or different substituents selected from the group consisting of halogen, hydroxy, mercapto, trifluoromethyl, cyano, carboxy, CONH₂, nitro, oxo, C₁₋₃alkyl, C₂₋₄alkenyl, C₁₋₃hydroxyalkyl, C₁₋₃haloalkyl, C₁₋₄alkoxy, C₁₋₄alkoxycarbonyl, C₁₋₄amino, or —NH₂;
C₆₋₁₄aryl, C₁₋₁₀heteroaryl are optionally substituted with one or more, same or different substituents selected from the group consisting of halogen, hydroxy, mercapto, trifluoromethyl, cyano, carboxy, CONH₂, nitro, oxo, C₁₋₃alkyl, C₂₋₄alkenyl, C₁₋₃hydroxyalkyl, C₁₋₃haloalkyl, C₁₋₄alkoxy, C₁₋₄alkoxycarbonyl, C₁₋₄amino, or —NH₂;
R₂ represents C₁₋₆alkyl, C₂₋₆alkenyl, C₂₋₆alkynyl, C₁₋₆hydroxyalkyl, C₁₋₆haloalkyl, C₁₋₆amino, C₁₋₁₂alkylsilyl, C₆₋₃₀alkylarylsilyl, C₁₋₁₀hetero aryl, C₆₋₁₄aryl, C₁₋₁₀heterocyclo alkyl, C₁₋₁₀heterocycloalkenyl, C₁₋₈cycloalkyl, C₁₋₁₈cycloalkenyl,
each of which is optionally substituted with one or more, same or different substituents selected from the group consisting of
halogen, hydroxy, —NH₂, mercapto, trifluoromethyl, cyano, carboxy, CONH₂, nitro, oxo, —S(O)₂NH₂, —OSi(CH(CH₃)₂)₃, C₁₋₄alkyl, C₁₋₆alkylsilyl, C₂₋₄alkenyl, C₂₋₄alkynyl, C₁₋₄hydroxyalkyl, C₁₋₆haloalkyl, C₁₋₄alkoxy, C₁₋₄alkoxycarbonyl, C₁₋₆ureido, C₁₋₆thioureido, C₁₋₄alkylcarbonyloxy, C₁₋₄alkoxycarbonyloxy, C₁₋₄alkoxysulfonyloxy, C₁₋₄aminocarbonyl, C₁₋₄alkylthio, C₃₋₆cycloalkyl, C₃₋₆cycloalkenyl, C₁₋₁₀amino, C₁₋₆imino, C₁₋₄aminosulfonyl, C₁₋₄aminocarbonyloxy, C₁₋₄alkylsulfonylamino, C₆₋₁₀arylsulfonylamino, C₆₋₁₀arylamino, C₁₋₆heterocycloalkylamino, C₁₋₆heterocycloalkylcarbonyl, C₆₋₁₀arylcarbonylamino, C₁₋₄alkoxyimino, C₁₋₄alkylcarbonylamino, C₁₋₄alkenylcarbonylamino, C₃₋₆cycloalkenylcarbonylamino, C₃₋₆cycloalkylcarbonylamino, C₁₋₄alkoxycarbonylamino, C₁₋₁₀heterocycloalkylcarbonylamino, C₁₋₄alkylsulfonyl, C₆₋₁₄aryl, C₁₋₆heteroaryl, C₁₋₁₀heterocycloalkylaryl, C₁₋₆heterocycloalkyl, or C₂₋₆heterocycloalkenyl,
wherein said C₁₋₄alkyl, C₁₋₆alkylsilyl, C₂₋₄alkenyl, C₂₋₄alkynyl, C₁₋₄hydroxyalkyl, C₁₋₆haloalkyl, C₁₋₄alkoxy, C₁₋₄alkoxycarbonyl, C₁₋₆ureido, C₁₋₆thioureido, C₁₋₄alkylcarbonyloxy, C₁₋₄alkoxycarbonyloxy, C₁₋₄alkoxysulfonyloxy, C₁₋₄aminocarbonyl, C₆₋₁₀arylcarbonylamino, C₁₋₄alkylthio, C₃₋₆cycloalkyl, C₃₋₆cycloalkenyl, C₁₋₁₀amino, C₆₋₁₀arylamino, C₁₋₆heterocycloalkylamino, C₁₋₆heterocycloalkylcarbonyl, C₁₋₆imino, C₁₋₄aminosulfonyl, C₁₋₄aminocarbonyloxy, C₁₋₄alkylsulfonylamino, C₆₋₁₀arylsulfonylamino, C₁₋₄alkoxyimino, C₁₋₄alkylcarbonylamino, C₁₋₁₀heterocycloalkylcarbonylamino, C₁₋₄alkylsulfonyl, C₆₋₁₄aryl, C₁₋₆heteroaryl, C₁₋₆heterocycloalkyl, or C₂₋₆heterocycloalkenyl,
are optionally further substituted with one or more, same or different substituents selected from the group consisting of halogen, hydroxy, —NH₂, mercapto, trifluoromethyl, cyano, carboxy, CONH₂, nitro, oxo, —S(O)₂NH₂, C₁₋₄alkyl, C₁₋₆alkylsilyl, C₂₋₄alkenyl, C₂₋₄alkynyl, C₁₋₄hydroxyalkyl, C₁₋₆haloalkyl, C₁₋₄alkoxy, C₁₋₄alkoxycarbonyl, C₁₋₆ureido, C₁₋₆thioureido, C₁₋₄alkylcarbonyloxy, C₁₋₄alkoxycarbonyloxy, C₁₋₄alkoxysulfonyloxy, C₁₋₄alkoxycarbamoyl, C₁₋₄aminocarbonyl, C₁₋₄alkylthio, C₃₋₆cycloalkyl, C₃₋₆cycloalkenyl, C₁₋₆amino C₁₋₆imino, C₁₋₄aminosulfonyl, C₁₋₄aminocarbonyloxy, C₁₋₄alkylsulfonylamino, C₆₋₁₀arylsulfonylamino, C₁₋₄alkoxyimino, C₁₋₄alkylcarbonylamino, C₁₋₄alkylsulfonyl, C₆₋₁₄aryl, C₁₋₆heteroaryl, C₁₋₁₀heterocycloalkylaryl, C₁₋₆heterocycloalkyl, or C₂₋₆heterocycloalkenyl,
wherein said C₁₋₄alkyl, C₁₋₆alkylsilyl, C₂₋₄alkenyl, C₂₋₄alkynyl, C₁₋₄hydroxyalkyl, C₁₋₆haloalkyl, C₁₋₄alkoxy, C₁₋₄alkoxycarbonyl, C₁₋₆ureido, C₁₋₆thioureido, C₁₋₄alkylcarbonyloxy, C₁₋₄alkoxycarbonyloxy, C₁₋₄alkoxysulfonyloxy, C₁₋₄alkoxycarbamoyl, C₁₋₄aminocarbonyl, C₁₋₄alkylthio, C₃₋₆cycloalkyl, C₃₋₆cycloalkenyl, C₁₋₆amino, C₁₋₆imino, C₁₋₄aminosulfonyl, C₁₋₄aminocarbonyloxy, C₁₋₄alkylsulfonylamino, C₁₋₄alkoxyimino, C₁₋₄alkylcarbonylamino, C₆₋₁₀arylsulfonylamino, C₁₋₄alkylsulfonyl, C₆₋₁₄aryl, C₁₋₆heteroaryl, C₁₋₁₀heterocycloalkylaryl, C₁₋₆heterocycloalkyl, or C₂₋₆heterocycloalkenyl,
are optionally further substituted with one or more, same or different substituents selected from the group consisting of
halogen, hydroxy, —NH₂, mercapto, trifluoromethyl, cyano, carboxy, CONH₂, nitro, oxo, —S(O)₂NH₂, C₁₋₄alkyl, C₁₋₄hydroxyalkyl, C₁₋₃alkoxy, benzyl or C₁₋₄alkoxycarbonyl.
or R₂ represents hydrogen, carboxy, or hydroxy;
or a pharmaceutically acceptable salt, solvate, or ester thereof;
with the proviso that the compound of general formula I cannot be α-methyl-N-2-propyn-1-yl-benzenemethanamine, N-2-butynyl-α-methyl-benzenemethanamine, N-2-butynyl-α-methyl-1-naphthalenemethanamine and N-5-hexyn-1-yl-α-methyl-benzenemethanamine.

31. A compound according to claim 30, of formula Ia.

32. A compound according to claim 30, wherein X represents cis —CH═CH—CH₂—, trans —CH═CH—CH₂—, —CH₂—, —CH₂—CH₂—, —CH₂—CH₂—CH₂— or —CH₂—CH₂—CH₂—CH₂— optionally substituted with trifluoromethyl.

33. A compound according to claim 30, wherein X represents -phenylene-CH₂—, -thienyl-CH₂- or -pyridyl-CH₂—
wherein the phenylene, thienyl and pyridyl rings are optionally further substituted with one or more substituents selected from hydroxy or halogen.

34. A compound according to claim 33, wherein the ethynylene defined in formula Ia or Ib is attached to the phenylene ring in ortho-, meta- or para-position.

35. A compound according to claim 30, wherein A represents 1-naphthyl, 2-naphthyl or phenyl, each of which are optionally substituted as defined in claim 1 for the substitution of C₆₋₁₄aryl representing A.

36. A compound according to claim 30, wherein R₁ is methyl, ethyl, n-propyl, optionally substituted with halogen or hydroxy.

37. A compound according to claim 30, wherein R₂ represents hydrogen, carboxy, C₁₋₆alkyl, C₂₋₆alkenyl, C₃₋₈cylcoalkyl, C₆₋₁₀aryl, C₁₋₃hydroxyalkyl or C₁₋₃alkylsilyl,
the last six of which are optionally substituted with one or more, same or different substituents selected from the group consisting of NH₂, hydroxy, CF₃, C₁₋₃haloalkyl, fluoro, chloro, bromo, C₁₋₃alkylamino, phenyl, C₁₋₃alkylsilyl,
OSi(CH(CH₃)₂)₃, C₁₋₃alkoxy, C₁₋₃alkyl, cyano, or C₁₋₃hydroxyalkyl, C₃₋₆heterocycloalkyl, the latter further substituted with fluoro or methoxy.

38. A compound according to claim 37, wherein R₂ represents hydrogen, methyl, tert-butyl, cyclopropyl, aminopropyl, aminoethyl, hydroxymethyl, hydroxyethylvinyl, dimethylaminoethyl, dimethylaminoethyl, trifluoromethylphenyl, dimethylhydroxyethyl, trifluoromethylvinylene, hydroxylpropyl, hydroxyisopropyl, hydroxypropenyl, bromophenyl, aminophenyl, hydroxybutyl, phenyl, trimethylsilyl, hydroxyethylpropyl, hydroxymethylpropyl, phenylhydroxymethyl, trimethylsilylmethyl, methoxyphenyl, difluorophenyl, tolyl, hydroxyphenyl, chlorophenyl, cyanophenyl, cyanopropyl, fluorophenyl, hydroxymethylphenyl, hydroxyphenylmethyl, trimethylsilyl, methoxymethyl, methoxyisopropyl, hydroxyethyl, carboxy, fluorooxacyclohexanephenyl or methoxyoxacyclohexanephenylene or dimethylaminophenylene.

39. A compound according to claim 30, wherein R₂ represents C₁₋₄alkyl substituted with one or more, same or different substituents selected from C₁₋₄alkyl, C₁₋₆amino, C₁₋₄alkylcarbonylamino, C₁₋₄alkenylcarbonylamino, C₃₋₆cycloalkenylcarbonylamino, C₃₋₆cycloalkylcarbonylamino, C₆₋₁₀arylcarbonylamino, C₁₋₄alkoxycarbonylamino, C₁₋₁₀heterocycloalkylcarbonylamino, C₁₋₄alkylsulfonylamino, C₆₋₁₀arylsulfonylamino or C₁₋₆ureido, which are optionally substituted with one or more, same or different substituents selected from oxo, hydroxy, halogen, trifluoromethyl, C₁₋₄alkyl, C₂₋₄alkynyl, C₆₋₁₀aryl, C₁₋₄alkoxy, C₁₋₃haloalkyl, C₁₋₆heterocycloalkyl, trifluoromethylphenylene or trifluoromethylbenzyl.

40. A compound according to claim 39, wherein R₂ represents —CH₂—CH₂—, —CH₂— or —CH₂—C(CH₃)₂— substituted with formylamino, dimethylamino, diethylamino, dipropylamino, propenylcarbonylamino, butynecarbonylamino, benzylmethylamino, propylcarbonylamino, cyclohexenylcarbonylamino, cyclopropylcarbonylamino, dimethylpropylcarbonylamino, ethylpropylcarbonylamino, methoxymethylcarbonylamino, methylpiperidylcarbonylamino, isopropylcarbonylamino, dihydroxyphenylcarbonylamino, methylphenylsulfonylamino, ethylsulfonylamino, propylsulfonylamino, butylsulfonylamino, methoxyphenylsulfonylamino, trifluoromethylchlorophenylsulfonylamino, ethylureylene, isopropylureylene, cyclohexylureylene, phenylureylene, propylureylene, di-trifluoromethylphenylureylene, dimethoxyphenylureylene, trifluoromethylphenylureylene, di-trifluoromethylbenzylamino or tert-butoxycarbonylamino.

41. A compound according to claim 30, wherein R₂ represents C₁₋₄alkyl substituted with one or more same or different substituents selected from hydrogen or C₁₋₃alkyl,
and wherein the C₁₋₄alkyl is further substituted with one or more, same or different substituents selected from oxo, methoxy, carboxy, C₁₋₄alkyl, C₁₋₆alkoxycarbonyl or C₃₋₆heterocycloalkyl which are optionally substituted with one or more, same or different substituents selected from halogen, C₁₋₄alkyl, C₁₋₄alkoxy, C₁₋₃haloalkyl or C₆₋₁₀aryl.

42. A compound according to claim 41, wherein R₂ represents —CR₁₃R₁₄—(CH₂)ᵤ—,
wherein
u is an integer of 0, 1,
R₁₃, R₁₄ independently of each other represents hydrogen or methyl;
and wherein —CR₁₃R₁₄— is substituted with carboxy or C₁₋₆alkoxycarbonyl which are optionally substituted with one or more, same or different substituents selected from methyl, C₁₋₄alkyl, C₁₋₄alkoxy, C₁₋₃haloalkyl or C₆₋₁₀aryl.

43. A compound according to claim 42, wherein R₂ represents phenyl-CH₂—O—C(O)—C(CH₃)₂—, HO—C(O)—C(CH₃)₂— or R₁₅—O—C(O)—C(CH₃)₂—CH₂—.

44. A compound according to claim 30, wherein R₂ represents R₁₅—O—C(O)—(C(CH₃)₂)ₜ-phenylene- or R₁₅—O—C(O)-phenylene-,
wherein t is an integer of 0, 1,
R₁₅ represent hydrogen or methyl.
and the phenylene ring is optionally substituted with methoxy.

45. A compound according to claim 41, wherein A represents 1-naphthyl or phenyl,
wherein X represents —CH₂—, cis —CH═CH—CH₂— or trans —CH═CH—CH₂—,
R₁ is methyl.

46. A compound according to 41, wherein A represents 1-naphthyl, 3-methoxyphenylene or phenyl,
wherein X represents -phenylene-CH₂—, the phenylene being optionally substituted with halogen or hydroxy.
R₁ is methyl.

47. A compound according to claim 30, wherein R₂ represents —CR₁₃R₁₄—C(O)—NR₁₆R₁₇,
wherein R₁₃, R₁₄ independently of each other are as defined above;
and wherein R₁₆, R₁₇ independently represents hydrogen, C₁₋₃alkyl, C₃₋₆heterocycloalkyl or C₆₋₁₀aryl substituted with one or more, same or different substituents selected from hydroxy, C₁₋₃alkyl, C₁₋₃alkoxy, C₁₋₄hydroxyalkyl, C₃₋₆heterocyclo alkyl, C₁₋₁₀heterocycloalkylaryl, C₁₋₁₀heteroaryl, C₆₋₁₀aryl, whereas said C₁₋₃alkyl, C₁₋₃alkoxy, C₁₋₄hydroxyalkyl, C₃₋₆heterocycloalkyl, C₁₋₁₀heteroaryl or C₆₋₁₀aryl,
is optionally further substituted with methyl, tert-butyl, C₆₋₁₀aryl, halogen, methoxy, C₁₋₄alkoxycarbonyl or trifluoromethyl,
or R₁₆ and R₁₇ together with the N-atom to which they are attached form a C₁₋₆heterocycloalkyl ring optionally substituted with hydroxyl, C₁₋₃alkyl or phenyl the last two substituents optionally substituted with fluoro or methoxy.

48. A compound according to claim 47, wherein A represents 1-naphthyl,
wherein X represents —CH═CH—CH₂—,
R₁ is methyl.

49. A compound according to claim 47, wherein R₂ represents —C(CH₃)₂—C(O)—NR₁₈R₁₉,
wherein R₁₈ represents hydrogen or methyl,
and wherein R₁₉ represents pyridylmethylene, morpholinopropyl, diphenylmethylene, diphenylethylene, phenylpiperidinoethylene, hydroxyethylene, dimethyloxazolylmethylene, methoxycarbonylbenzyl, benzodioxolmethylene, trifluoromethylphenylethyl, methoxyphenylethyl, difluorobenzyl, tert-butylbenzyl, bromobenzyl phenylhydroxypropyl, diphenylhydroxyethyl, hydroxyindanyl, benzylpiperidyl, chlorobenzyl, phenylethylene, phenylethyl, benzyl, pyrrolidyloxodimethylethyl or ethoxyethylene.

50. A compound according to claim 47, wherein R₂ represents
—C(O)—C(CH₃)₂— substituted with pyrrolidinyl, hydroxypyrrolidinyl, methoxyethylpiperazinyl or hydroxypiperidino.

51. A compound according to claim 30, of formula Ia,
wherein X represents —CH═CH—CH₂—, —CH₂—, —CH₂—CH₂—, or —CH₂—CH₂—CH₂—;
wherein A represents 1-naphthyl or phenyl, optionally substituted with hydroxy, halogen or methoxy;
R₁ is methyl;
and R₂ is as defined in claim 30.

52. A compound according to claim 30, of formula Ia wherein
X represents -phenylene-CH₂—, -thienyl-CH₂—, -pyridyl-CH₂— wherein the phenylene, thienyl and pyridyl rings are optionally substituted with one or more substituents selected from hydroxy, fluoro or bromo;
wherein A represents 1-naphthyl or phenyl optionally substituted with methoxy; and
R₁ is methyl.

53. A compound according to claim 30, wherein A represents indolyl, benzothienyl, benzodioxol, methylphenylpyrazolyl,
R₁ is methyl;
X is cis or trans —CH═CH—CH₂— and
R₂ is tert-butyl.

54. A compound according to claim 30, selected from the group consisting of
(R)-(1-Naphthalen-1-yl-ethyl)-pent-4-ynyl-amine (compound 102),
(E)/(R)-(6,6-Dimethyl-hept-2-en-4-ynyl)-(1-naphthalen-1-yl-ethyl)-amine (compound 103),
(Z)/(R)-(6,6-Dimethyl-hept-2-en-4-ynyl)-(1-naphthalen-1-yl-ethyl)-amine (compound 104),
(E)/(R)-(5-Cyclopropyl-pent-2-en-4-ynyl)-(1-naphthalen-1-yl-ethyl)-amine (compound 105),
(E)/(R)-6-Methyl-(1-naphthalen-1-yl-ethyl)-hept-2-en-4-yne-1,6-diamine (compound 106),
(E)/(R)-6-(1-Naphthalen-1-yl-ethylamino)-hex-4-en-2-yn-1-ol (compound 107),
(E)/(R)-2-Methyl-7-(1-naphthalen-1-yl-ethylamino)-hept-5-en-3-yn-2-ol (compound 108),
(E)/(3R/S)-3-Methyl-8-[(R)-1-naphthalen-1-yl-ethylamino]-oct-6-en-4-yn-3-ol (compound 109),
(E)/(1R/S)-6-[(R)-1-naphthalen-1-yl-ethylamino]-1-phenyl-hex-4-en-2-yn-1-ol (compound 110),
(Z)/(R)-6-(1-Naphthalen-1-yl-ethylamino)-hex-4-en-2-ynoic acid (compound 111),
(E)/(R)-(1-Naphthalen-1-yl-ethyl)-(5-trimethylsilanyl-pent-2-en-4-ynyl)-amine (compound 112),
(Z)/(R)-(1-Naphthalen-1-yl-ethyl)-(5-trimethylsilanyl-pent-2-en-4-ynyl)-amine (compound 113),
(E)/(R)-(1-Naphthalen-1-yl-ethyl)-(6-trimethylsilanyl-hex-2-en-4-ynyl)-amine (compound 114),
(E)/(R)-(1-Naphthalen-1-yl-ethyl)-(5-phenyl-pent-2-en-4-ynyl)-amine (compound 115),
(E)/(R)-(1-Naphthalen-1-yl-ethyl)-(5-p-tolyl-pent-2-en-4-ynyl)-amine (compound 116),
(E)/(R)-(1-Naphthalen-1-yl-ethyl)-[5-(4-trifluoromethyl-phenyl)-pent-2-en-4-ynyl]-amine (compound 117),
(E)/(R)-(1-Naphthalen-1-yl-ethyl)-[5-(3-trifluoromethyl-phenyl)-pent-2-en-4-ynyl]-amine (compound 118),
(E)/(R)-[5-(2-Fluoro-phenyl)-pent-2-en-4-ynyl]-(1-naphthalen-1-yl-ethyl)-amine (compound 119),
(E)/(R)-[5-(2,4-Difluoro-phenyl)-pent-2-en-4-ynyl]-(1-naphthalen-1-yl-ethyl)-amine (compound 120),
(E)/(R)-[5-(4-Chloro-phenyl)-pent-2-en-4-ynyl]-(1-naphthalen-1-yl-ethyl)-amine (compound 121),
(E)/(R)-[5-(4-Bromo-phenyl)-pent-2-en-4-ynyl]-(1-naphthalen-1-yl-ethyl)-amine (compound 122),
(E)/(R)-4-[5-(1-Naphthalen-1-yl-ethylamino)-pent-3-en-1-ynyl]-benzonitrile (compound 123),
(E)/(R)-4-[5-(1-Naphthalen-1-yl-ethylamino)-pent-3-en-1-ynyl]-aniline (compound 124),
(E)/(R)-2-[5-(1-Naphthalen-1-yl-ethylamino)-pent-3-en-1-ynyl]-aniline (compound 125),
(E)/(R)-Dimethyl-{4-[5-(1-naphthalen-1-yl-ethylamino)-pent-3-en-1-ynyl]}-aniline (compound 126),
(E)/(R)-3-[5-(1-Naphthalen-1-yl-ethylamino)-pent-3-en-1-ynyl]-phenol (compound 127),
(E)/(R)-[5-(3-Methoxy-phenyl)-pent-2-en-4-ynyl]-(1-naphthalen-1-yl-ethyl)-amine (compound 128),
(E)/(R)-{4-[5-(1-Naphthalen-1-yl-ethylamino)-pent-3-en-1-ynyl]-phenyl}-methanol (compound 129),
(E)/(R)-(6,6-Dimethyl-hept-2-en-4-ynyl)-[1-(3-methoxy-phenyl)-ethyl]-amine, and
(Z)/(R)-(6,6-Dimethyl-hept-2-en-4-ynyl)-[1-(3-methoxy-phenyl)-ethyl]-amine,
or a pharmaceutically acceptable salt, solvate, or ester thereof, such as
(R)-But-2-ynyl-(1-naphthalen-1-yl-ethyl)-amine hydrochloride (compound 101),
(E)/(R)-(6,6-Dimethyl-hept-2-en-4-ynyl)-[1-(3-methoxy-phenyl)-ethyl]-amine hydro chloride (compound 130),
(Z)/(R)-(6,6-Dimethyl-hept-2-en-4-ynyl)-[1-(3-methoxy-phenyl)-ethyl]-amine hydrochloride (compound 131),
(R)-(1-Naphthalen-1-yl-ethyl)-prop-2-ynyl-amine (compound 132),
(R)-[1-(3-Methoxy-phenyl)-ethyl]-prop-2-ynyl-amine (compound 133),
(R)-But-3-ynyl-(1-naphthalen-1-yl-ethyl)-amine (compound 134),
(R)-Hex-5-ynyl-(1-naphthalen-1-yl-ethyl)-amine (compound 135),
(E)/(R)-(6,6-Dimethyl-hept-2-en-4-ynyl)-[1-(4-fluoro-3-methoxy-phenyl)-ethyl]-amine (compound 136),
(Z)/(R)-(6,6-Dimethyl-kept-2-en-4-ynyl)-[1-(4-fluoro-3-methoxy-phenyl)-ethyl]-amine (compound 137),
(E)/(R)-(6,6-Dimethyl-hept-2-en-4-ynyl)-[1-(1H-indol-7-yl)-ethyl]-amine (compound 138),
(Z)/(R)-(6,6-Dimethyl-hept-2-en-4-ynyl)-[1-(1H-indol-7-yl)-ethyl]-amine (compound 139),
(E)/(R)-(6,6-Dimethyl-hept-2-en-4-ynyl)-[1-(1H-indol-4-yl)-ethyl]-amine (compound 140),
(Z)/(R)-(6,6-Dimethyl-hept-2-en-4-ynyl)-[1-(1H-indol-4-yl)-ethyl]-amine (compound 141),
(E)/(R)-(1-Benzo[1,3]dioxol-5-yl-ethyl)-(6,6-dimethyl-hept-2-en-4-ynyl)-amine (compound 142),
(Z)/(R)-(1-Benzo[1,3]dioxol-5-yl-ethyl)-(6,6-dimethyl-hept-2-en-4-ynyl)-amine (compound 143),
(E)/(R)-(1-Benzo[b]thiophen-3-yl-ethyl)-(6,6-dimethyl-hept-2-en-4-ynyl)-amine (compound 144),
(E)/(R)-(1-Benzo[b]thiophen-3-yl-ethyl)-(6,6-dimethyl-hept-2-en-4-ynyl)-amine (compound 145),
(E)/(R)-(6,6-Dimethyl-hept-2-en-4-ynyl)-[1-(1-methyl-5-phenyl-1H-pyrazol-3-yl)-ethyl]-amine (compound 146),
(Z)/(R)-(6,6-Dimethyl-hept-2-en-4-ynyl)-[1-(1-methyl-5-phenyl-1H-pyrazol-3-yl)-ethyl]-amine (compound 147),
(Z)/(R)-2-Methyl-7-(1-naphthalen-1-yl-ethylamino)-hept-5-en-3-yn-2-ol (compound 148),
(E)/(R)-9-(1-Naphthalen-1-yl-ethylamino)-non-7-en-5-yn-1-ol (compound 149),
(E)/(R)-9-(1-Naphthalen-1-yl-ethylamino)-non-7-en-5-ynenitrile (compound 150),
(E)/(R)-{2-[5-(1-Naphthalen-1-yl-ethylamino)-pent-3-en-1-ynyl]-phenyl}-methanol (compound 151),
(E,E)/((R)-8-(1-Naphthalen-1-yl-ethylamino)-octa-2,6-dien-4-yn-1-ol (compound 152),
(E)/(R)-3-Ethyl-8-(1-naphthalen-1-yl-ethylamino)-oct-6-en-4-yn-3-ol (compound 153),
(E)/(R)-(6-Methoxy-hex-2-en-4-ynyl)-(1-naphthalen-1-yl-ethyl)-amine hydrochloride (compound 154),
(E)/(R)-(6-Methoxy-6-methyl-hept-2-en-4-ynyl)-(1-naphthalen-1-yl-ethyl)-amine hydrochloride (compound 155),
(E)/(R)-7-(1-Naphthalen-1-yl-ethylamino)-hept-5-en-3-yn-1-ol hydrochloride (compound 156),
(E)/(R)-(6,6-Dimethyl-7-triisopropylsilanyloxy-hept-2-en-4-ynyl)-(1-naphthalen-1-yl-ethyl)-amine (compound 157),
(E)/(R)-2,2-Dimethyl-7-(1-naphthalen-1-yl-ethylamino)-hept-5-en-3-yn-1-ol hydrochloride (compound 158),
(E)/(2R/S)-7-[(R)-1-Naphthalen-1-yl-ethylamino]-hept-5-en-3-yn-2-ol hydrochloride (compound 159),
(E)/(R)-8-(1-Naphthalen-1-yl-ethylamino)-oct-6-en-4-yn-1-ol hydrochloride (compound 160),
(E)/(R)—N*6*,N*6*-Dimethyl-N*1*-(1-naphthalen-1-yl-ethyl)-hex-2-en-4-yne-1,6-diamine dihydrochloride (compound 161),
(E)/(R)—N*6*-Benzyl-N*6*-methyl-N*1*-(1-naphthalen-1-yl-ethyl)-hex-2-en-4-yne-1,6-diamine dihydrochloride (compound 162),
(E)/(R)—N*6*,N*6*-Diethyl-N*1*-(1-naphthalen-1-yl-ethyl)-hex-2-en-4-yne-1,6-diamine dihydrochloride (compound 163),
(E)/(R)—N*1*-(1-Naphthalen-1-yl-ethyl)-N*6*,N*6*-dipropyl-hex-2-en-4-yne-1,6-diamine dihydrochloride (compound 164),
(E)/(R)-(4-{5-[1-(3-Methoxy-phenyl)-ethylamino]-pent-3-en-1-ynyl}-phenyl)-methanol (compound 165),
(E)/(R)-7-[1-(3-Methoxy-phenyl)-ethylamino]-2-methyl-hept-5-en-3-yn-2-ol (compound 166),
(E)/(4R/S)-9-[(R)-1-(3-Methoxy-phenyl)-ethylamino]-non-7-en-5-yn-4-ol (compound 167),
(E)/(R)-Benzyl 2,2-dimethyl-7-(1-naphthalen-1-yl-ethylamino)-hept-5-en-3-ynoate (compound 168),
(E)/(R)-Methyl 2,2-dimethyl-8-(1-naphthalen-1-yl-ethylamino)-oct-6-en-4-ynoate (compound 169),
(Z)/(R)Benzyl 2,2-dimethyl-7-(1-naphthalen-1-yl-ethylamino)-hept-5-en-3-ynoate (compound 170),
(E)/(R)-tert-Butyl [6-(1-naphthalen-1-yl-ethylamino)-hex-4-en-2-ynyl]-carbamate (compound 171),
(R)-(1-Naphthalen-1-yl-ethyl)-[3-(3-trifluoromethyl-phenyl)-prop-2-ynyl]-amine hydrochloride (compound 172),
(R)-(1-Naphthalen-1-yl-ethyl)-[3-(4-trifluoromethyl-phenyl)-prop-2-ynyl]-amine hydrochloride (compound 173),
(R)-Methyl 2-methyl-2-{4-[3-(1-naphthalen-1-yl-ethylamino)-prop-1-ynyl]-phenyl}-propanoate hydrochloride (compound 174),
(R)-[1-(3-Methoxy-phenyl)-ethyl]-{3-[3-(4-methoxy-tetrahydro-pyran-4-yl)-phenyl]-prop-2-ynyl}-amine (compound 175),
(R)-[1-(3-Methoxy-phenyl)-ethyl]-{3-[3-(4-fluoro-tetrahydro-pyran-4-yl)-phenyl]-prop-2-ynyl}-amine (compound 176),
(R)-(1-Naphthalen-1-yl-ethyl)-(3-trimethylsilanylethynyl-benzyl)-amine (compound 177),
(R)-(1-Phenyl-ethyl)-(3-trimethylsilanylethynyl-benzyl)-amine (compound 178),
(R)-(1-Naphthalen-1-yl-ethyl)-(4-trimethylsilanylethynyl-benzyl)-amine (compound 179),
(R)-(1-Phenyl-ethyl)-(4-trimethylsilanylethynyl-benzyl)-amine (compound 180),
(R)-3-{3-[(1-Naphthalen-1-yl-ethylamino)-methyl]-phenyl}-prop-2-yn-1-ol (compound 181),
(R)-2-Methyl-4-{3-[(1-naphthalen-1-yl-ethylamino)-methyl]-phenyl}-but-3-yn-2-ol (compound 182),
(R)-2-Methyl-4-{2-[(1-phenyl-ethylamino)-methyl]-phenyl}-but-3-yn-2-ol (compound 183),
(R)-1,1-Dimethyl-3-{3-[(1-naphthalen-1-yl-ethylamino)-methyl]-phenyl}-prop-2-ynylamine (compound 184),
(R)-(1-Phenyl-ethyl)-(4-phenylethynyl-benzyl)-amine (compound 185),
(R)-(1-Naphthalen-1-yl-ethyl)-(4-phenylethynyl-benzyl)-amine (compound 186),
(R)-3-{4-[(1-Naphthalen-1-yl-ethylamino)-methyl]-phenyl}-prop-2-yn-1-ol (compound 187),
(R)-3-{4-[(1-Phenyl-ethylamino)-methyl]-phenyl}-prop-2-yn-1-ol (compound 188),
(R)-4-{4-[(1-Naphthalen-1-yl-ethylamino)-methyl]-phenyl}-but-3-yn-1-ol (compound 189),
(R)-4-{4-[(1-Phenyl-ethylamino)-methyl]-phenyl}-but-3-yn-1-ol (compound 190),
(R)-(1-Naphthalen-1-yl-ethyl)-(3-phenylethynyl-benzyl)-amine (compound 191),
(R)-(1-Phenyl-ethyl)-(3-phenylethynyl-benzyl)amine (compound 192),
(R)-Benzyl 2,2-dimethyl-4-{3-[(1-phenyl-ethylamino)-methyl]-phenyl}-but-3-ynoate (compound 193),
(R)-Benzyl 4-{4-fluoro-3-[(1-phenyl-ethylamino)-methyl]-phenyl}-2,2-dimethyl-but-3-ynoate (compound 194),
(R)-Benzyl 2,2-dimethyl-4-{3-[(1-naphthalen-1-yl-ethylamino)-methyl]-phenyl}-but-3-ynoate (compound 195),
(R)-2-Methyl-4-{3-[(1-phenyl-ethylamino)-methyl]-phenyl}-but-3-yn-2-ol (compound 196),
(R)-4-{4-Fluoro-3 [(1-phenyl-ethylamino)-methyl]-phenyl}-2-methyl-but-3-yn-2-ol (compound 197),
(R)-4-{4-Fluoro-3-[(1-naphthalen-1-yl-ethylamino)-methyl]-phenyl}-2-methyl-but-3-yn-2-ol (compound 198),
(R)-2-Methyl-4-{6-[(1-naphthalen-1-yl-ethylamino)-methyl]-pyridin-2-yl}-but-3-yn-2-ol (compound 199),
(R)-(3-{3-[(1-Phenyl-ethylamino)-methyl]-phenyl}-prop-2-ynyl)-bis-(4-trifluoromethyl-benzyl)-amine (compound 200),
(R)-Diethyl-(3-{3-[(1-naphthalen-1-yl-ethylamino)-methyl]-phenyl}-prop-2-ynyl)-amine (compound 201),
(R)-Benzyl 2,2-dimethyl-4-{4-[(1-naphthalen-1-yl-ethylamino)-methyl]-phenyl}-but-3-ynoate (compound 202),
(R)-Benzyl 4-(4-{[1-(3-methoxy-phenyl)-ethylamino]-methyl}-phenyl)-2,2-dimethyl-but-3-ynoate (compound 203),
(R)-(4-Ethynyl-benzyl)-(1-naphthalen-1-yl-ethyl)-amine (compound 204),
(R)-(4-Ethynyl-benzyl)-(1-phenyl-ethyl)-amine (compound 205),
(R)-2-(3-Hydroxy-3-methyl-but-1-ynyl)-5-[(1-naphthalen-1-yl-ethylamino)-methyl]-phenol (compound 206),
(R)-2-(3-Diethylamino-prop-1-ynyl)-5-[(1-naphthalen-1-yl-ethylamino)-methyl]-phenol (compound 207),
(R)-Benzyl 4-{2-Hydroxy-4-[(1-naphthalen-1-yl-ethylamino)-methyl]-phenyl}-2,2-dimethyl-but-3-ynoate (compound 208),
(R)-(2-Ethynyl-benzyl)-(1-naphthalen-1-yl-ethyl)-amine (compound 209),
(R)-(2-Ethynyl-benzyl)-(1-phenyl-ethyl)-amine (compound 210),
(R)-2-Methyl-4-{5-[(1-naphthalen-1-yl-ethylamino)-methyl]-thiophen-2-yl}-but-3-yn-2-ol (compound 211),
(R)-(1-Naphthalen-1-yl-ethyl)-(4-phenylethynyl-thiophen-2-ylmethyl)-amine (compound 212),
(R)-(1-Naphthalen-1-yl-ethyl)-(5-phenylethynyl-thiophen-2-ylmethyl)-amine (compound 213),
(R)-3-{5-[(1-Naphthalen-1-yl-ethylamino)-methyl]-thiophen-3-yl}-prop-2-yn-1-ol (compound 214),
(R)-3-{5-[(1-Phenyl-ethylamino)-methyl]-thiophen-3-yl}-prop-2-yn-1-ol (compound 215),
(R)-(1-Phenyl-ethyl)-(5-phenylethynyl-thiophen-2-ylmethyl)-amine (compound 216),
(R)-(1-Phenyl-ethyl)-(4-phenylethynyl-thiophen-2-ylmethyl)-amine (compound 217),
(E)/(R)-2-Ethyl-N-[6-(1-naphthalen-1-yl-ethylamino)-hex-4-en-2-ynyl]-butyramide (compound 218),
(E)/(R)-1-Methyl-piperidine-4-carboxylic acid [6-(1-naphthalen-1-yl-ethylamino)-hex-4-en-2-ynyl]-amide (compound 219),
(E)/(R)-3,3-Dimethyl-N-[6-(1-naphthalen-1-yl-ethylamino)-hex-4-en-2-ynyl]-butyramide (compound 220),
(E)/(R)—N-[7-(1-Naphthalen-1-yl-ethylamino)-hept-5-en-3-ynyl]-butyramide (compound 221),
(E)/(R)-3,3-Dimethyl-N-[7-(1-naphthalen-1-yl-ethylamino)-hept-5-en-3-ynyl]-butyramide (compound 222),
(E)/(R)-2-Ethyl-N-[7-(1-naphthalen-1-yl-ethylamino)-hept-5-en-3-ynyl]-butyramide (compound 223),
(E)/(R)-2-Methoxy-N-[7-(1-naphthalen-1-yl-ethylamino)-hept-5-en-3-ynyl]-acetamide (compound 224),
(E)/(R)-1-Methyl-piperidine-4-carboxylic acid [7-(1-naphthalen-1-yl-ethylamino)-hept-5-en-3-ynyl]-amide (compound 225),
(E)-/(R)—N-[7-(1-Naphthalen-1-yl-ethylamino)-hept-5-en-3-ynyl]-formamide (compound 226),
(R) —N-[7-(1-Naphthalen-1-yl-ethylamino)-hept-5-en-3-ynyl]-isobutyramide (compound 227),
(E)/(R)-But-2-enoic acid [2,2-dimethyl-7-(1-naphthalen-1-yl-ethylamino)-hept-5-en-3-ynyl]-amide (compound 228),
(E)/(R)-Cyclohex-3-enecarboxylic acid [2,2-dimethyl-7-(1-naphthalen-1-yl-ethylamino)-hept-5-en-3-ynyl]-amide hydrogen hexafluorophosphate (compound 229),
(E)/(R)-Pent-4-ynoic acid [2,2-dimethyl-7-(1-naphthalen-1-yl-ethylamino)-hept-5-en-3-ynyl]-amide hydrogen hexafluorophosphate (compound 230),
(E)/(R)—N-[2,2-Dimethyl-7-(1-naphthalen-1-yl-ethylamino)-hept-5-en-3-ynyl]-2,4-dihydroxy-benzamide (compound 231),
(E)/(R)-Cyclopropanecarboxylic acid [2,2-dimethyl-7-(1-naphthalen-1-yl-ethylamino)-hept-5-en-3-ynyl]-amide hydrogen hexafluorophosphate (compound 232),
(E)/(R)-Ethanesulfonic acid [6-(1-naphthalen-1-yl-ethylamino)-hex-4-en-2-ynyl]-amide (compound 233),
(E)/(R)-Propane-1-sulfonic acid [6-(1-naphthalen-1-yl-ethylamino)-hex-4-en-2-ynyl]-amide (compound 234),
(E)/(R)-Butane-1-sulfonic acid [6-(1-naphthalen-1-yl-ethylamino)-hex-4-en-2-ynyl]-amide (compound 235),
(E)/(R)-4-Methyl-N-[7-(1-naphthalen-1-yl-ethylamino)-hept-5-en-3-ynyl]-benzenesulfonamide (compound 236),
(E)/(R)-2-Chloro-N-[7-(1-naphthalen-1-yl-ethylamino)-hept-5-en-3-ynyl]-5-trifluoromethyl-benzenesulfonamide (compound 237),
(E)/(R)-4-Methoxy-N-[7-(1-naphthalen-1-yl-ethylamino)-hept-5-en-3-ynyl]-benzenesulfonamide (compound 238),
(E)/(R)-Ethanesulfonic acid [7-(1-naphthalen-1-yl-ethylamino)-hept-5-en-3-ynyl]-amide (compound 239),
(E)/(R)-Propane-1-sulfonic acid [7-(1-naphthalen-1-yl-ethylamino)-hept-5-en-3-ynyl]-amide (compound 240),
(E)/(R)-Butane-1-sulfonic acid [7-(1-naphthalen-1-yl-ethylamino)-hept-5-en-3-ynyl]-amide (compound 241),
(E)/(R)-1-Ethyl-3-[6-(1-naphthalen-1-yl-ethylamino)-hex-4-en-2-ynyl]-urea (compound 242),
(E)/(R)-1-(2,6-Dimethoxy-phenyl)-3-[6-(1-naphthalen-1-yl-ethylamino)-hex-4-en-2-ynyl]-urea (compound 243),
(E)/(R)-1-Ethyl-3-[7-(1-naphthalen-1-yl-ethylamino)-hept-5-en-3-ynyl]-urea (compound 244),
(E)/(R)-1-[7-(1-Naphthalen-1-yl-ethylamino)-hept-5-en-3-ynyl]-3-propyl-urea (compound 245),
(E)/(R)-1-Isopropyl-3-[7-(1-naphthalen-1-yl-ethylamino)-hept-5-en-3-ynyl]-urea (compound 246),
(E)/(R)-1-Cyclohexyl-3-[7-(1-naphthalen-1-yl-ethylamino)-hept-5-en-3-ynyl]-urea (compound 247),
(E)/(R)-1-[7-(1-Naphthalen-1-yl-ethylamino)-hept-5-en-3-ynyl]-3-phenyl-urea (compound 248),
(E)/(R)-1-(3,5-Bis-trifluoromethyl-phenyl)-3-[7-(1-naphthalen-1-yl-ethylamino)-hept-5-en-3-ynyl]-urea (compound 249),
(E)/(R)-1-[7-(1-Naphthalen-1-yl-ethylamino)-hept-5-en-3-ynyl]-3-(3-trifluoromethyl-phenyl)-urea (compound 250),
(E)/(R)-1-(2,4-Dimethoxy-phenyl)-3-[7-(1-naphthalen-1-yl-ethylamino)-hept-5-en-3-ynyl]-urea (compound 251),
(E)/(R)-2,2-Dimethyl-1-morpholin-4-yl-7-(1-naphthalen-1-yl-ethylamino)-hept-5-en-3-yn-1-one hydrochloride (compound 252),
(E)/(R)-2,2-Dimethyl-1-(4-methyl-piperazin-1-yl)-7-(1-naphthalen-1-yl-ethylamino)-hept-5-en-3-yn-1-one dihydrochloride (compound 253),
(E)/(R)-2,2-Dimethyl-7-(1-naphthalen-1-yl-ethylamino)-hept-5-en-3-ynoic acid (pyridin-3-ylmethyl)-amide (compound 254),
(E)/(R)-2,2-Dimethyl-7-(1-naphthalen-1-yl-ethylamino)-hept-5-en-3-ynoic acid (pyridin-4-ylmethyl)-amide (compound 255),
(E)/(R)-2,2-Dimethyl-7-(1-naphthalen-1-yl-ethylamino)-hept-5-en-3-ynoic acid (3-morpholin-4-yl-propyl)-amide (compound 256),
(E)/(R)-2,2-Dimethyl-7-(1-naphthalen-1-yl-ethylamino)-hept-5-en-3-ynoic acid benzhydryl-amide (compound 257),
(E)/(R)-2,2-Dimethyl-7-(1-naphthalen-1-yl-ethylamino)-hept-5-en-3-ynoic acid (3,3-diphenyl-propyl)-amide (compound 258),
(E)-1-[(R/S)]-3-Hydroxy-pyrrolidin-1-yl)-2,2-dimethyl-7-[(R)1-naphthalen-1-yl-ethylamino]-hept-5-en-3-yn-1-on (compound 259),
(E)/(R)-1-[4-(2-Methoxy-ethyl)-piperazin-1-yl]-2,2-dimethyl-7-(1-naphthalen-1-yl-ethylamino)-hept-5-en-3-yn-1-one (compound 260),
(E)-2,2-Dimethyl-7-[(R)-1-naphthalen-1-yl-ethylamino)-hept-5-en-3-ynoic acid [(R/S)-2-phenyl-2-piperidin-1-yl-ethyl]-amide (compound 261),
(E)/(R)-2,2-Dimethyl-7-(1-naphthalen-1-yl-ethylamino)-hept-5-en-3-ynoic acid (2-hydroxy-ethyl)-amide (compound 262),
(E)/(R)-1-(4-Hydroxy-piperidin-1-yl)-2,2-dimethyl-7-(1-naphthalen-1-yl-ethylamino)-hept-5-en-3-yn-1-one (compound 263),
(E)/(R)-2,2-Dimethyl-7-(1-naphthalen-1-yl-ethylamino)-hept-5-en-3-ynoic acid (2,5-dimethyl-oxazol-4-ylmethyl)-amide (compound 264),
(E)/(R)-3-{[2,2-Dimethyl-7-(1-naphthalen-1-yl-ethylamino)-hept-5-en-3-ynoylamino]-methyl}-benzoic acid methyl ester (compound 265),
(E)/(R)-2,2-Dimethyl-7-(1-naphthalen-1-yl-ethylamino)-hept-5-en-3-ynoic acid (benzo[1,3]dioxol-5-ylmethyl)-amide (compound 266),
(E)-2,2-Dimethyl-7-[(R)-1-naphthalen-1-yl-ethylamino)-hept-5-en-3-ynoic acid {1-[(S)-3-trifluoromethyl-phenyl)-ethyl}-amide (compound 267),
(E)-2,2-Dimethyl-7-[(R)-1-naphthalen-1-yl-ethylamino)-hept-5-en-3-ynoic acid {1-[(S)-3-methoxy-phenyl)-ethyl}-amide (compound 268),
(E)/(R)-2,2-Dimethyl-7-(1-naphthalen-1-yl-ethylamino)-hept-5-en-3-ynoic acid 3,5-difluoro-benzylamide (compound 269),
(E)/(R)-2,2-Dimethyl-7-(1-naphthalen-1-yl-ethylamino)-hept-5-en-3-ynoic acid 4-t-butyl-benzylamide (compound 270),
(E)/(R)-2,2-Dimethyl-7-(1-naphthalen-1-yl-ethylamino)-hept-5-en-3-ynoic acid 3-bromo-benzylamide (compound 271),
(E)-2,2-Dimethyl-7-[(R)-1-naphthalen-1-yl-ethylamino]-hept-5-en-3-ynoic acid [(R/S)-3-hydroxy-1-phenyl-propyl)-amide (compound 272),
(E)-2,2-Dimethyl-7-[(R)-1-naphthalen-1-yl-ethylamino)-hept-5-en-3-ynoic acid [(1R,2R)-2-hydroxy-1,2-diphenyl-ethyl]-amide (compound 273),
(E)-2,2-Dimethyl-7-[(R)-1-naphthalen-1-yl-ethylamino)-hept-5-en-3-ynoic acid [(1S,2S)-2-hydroxy-1,2-diphenyl-ethyl]-amide (compound 274),
(E)-2,2-Dimethyl-7-[(R)-1-naphthalen-1-yl-ethylamino]-hept-5-en-3-ynoic acid [(1S,2R)-2-hydroxy-indan-1-yl]-amide (compound 275),
(E)/(R)-2,2-Dimethyl-7-(1-naphthalen-1-yl-ethylamino)-hept-5-en-3-ynoic acid (1-benzyl-piperidin-4-yl)-amide (compound 276),
(E)/(R)-2,2-Dimethyl-7-(1-naphthalen-1-yl-ethylamino)-hept-5-en-3-ynoic acid 4-chloro-benzylamide (compound 277),
(E)/(R)-2,2-Dimethyl-7-(1-naphthalen-1-yl-ethylamino)-hept-5-en-3-ynoic acid phenethyl-amide (Compound 278),
(E)-2,2-Dimethyl-7-[(R)-1-naphthalen-1-yl-ethylamino]-hept-5-en-3-ynoic acid [(R/S)-1-phenyl-ethyl)-amide (Compound 279),
(E)/(R)-2,2-Dimethyl-7-(1-naphthalen-1-yl-ethylamino)-hept-5-en-3-ynoic acid benzylamide (compound 280),
(E)/(R)-2,2-Dimethyl-7-(1-naphthalen-1-yl-ethylamino)-1-pyrrolidin-1-yl-hept-5-en-3-yn-1 one (compound 281),
(E)/(R)-2,2-Dimethyl-7-(1-naphthalen-1-yl-ethylamino)-hept-5-en-3-ynoic acid (2-ethoxy-ethyl)-amide (compound 282),
(E)/(R)-2,2-Dimethyl-7-(1-naphthalen-1-yl-ethylamino)-hept-5-en-3-ynoic acid hydrochloride (compound 283),
(E)/(R)-2,2-Dimethyl-8-(1-naphthalen-1-yl-ethylamino)-oct-6-en-4-ynoic acid hydro chloride (compound 284),
(Z)/(R)-2,2-Dimethyl-7-(1-naphthalen-1-yl-ethylamino)-hept-5-en-3-ynoic acid hydro chloride (compound 285),
(R)-2-Methyl-2-{4-[3-(1-naphthalen-1-yl-ethylamino)-prop-1-ynyl]-phenyl}-propionic acid hydrochloride (compound 286),
(R)-2,2-Dimethyl-4-{3-[(1-naphthalen-1-yl-ethylamino)-methyl]-phenyl}-but-3-ynoic acid (compound 287),
(E)/(R)-6,6-Dimethyl-N*1*-(1-naphthalen-1-yl-ethyl)-hept-2-en-4-yne-1,7-diamine (compound 288),
(E)/(R)—N*1*-(1-naphthalen-1-yl-ethyl)-hept-2-en-4-yne-1,7-diamine (compound 289),
[(R)-1-Naphthalen-1-yl-ethyl]-[(R/S)-1-trifluoromethyl-but-3-ynyl]-amine (compound 290),
[(R/S)-8,8,8-Trifluoro-3-methylene]-7-[(R)-1-naphthalen-1-yl-ethylamino]-oct-4-yn-1-ol (compound 291),
2-{[(R/S)-5,5,5-Trifluoro]-4-[(R)-1-naphthalen-1-yl-ethylamino]-pent-1-ynyl}-phenylamine (compound 292),
4-Methoxy-3-{[(R/S)-5,5,5-trifluoro]-4-[(R)-1-naphthalen-1-yl-ethylamino]-pent-1-ynyl}-benzoic acid methyl ester (compound 293) or
(E)/(R)-(1-Naphthalen-1-yl-ethyl)-(6,6,6-trifluoro-hex-4-en-2-ynyl)-amine hydrochloride (compound 294).

54. A compound according to claim 30, or (R)-But-2-ynyl-(1-naphthalen-1-yl-ethyl)-amine, α-methyl-N-2-propyn-1-yl-benzenemethanamine, N-2-butynyl-α-methyl-benzenemethanamine, N-2-butynyl-α-methyl-1-naphthalenemethanamine or N-5-hexyn-1-yl-α-methyl-benzenemethanamine for use as a medicament.

55. Use of a compound according to claim 30, or (R)-But-2-ynyl-(1-naphthalen-1-yl-ethyl)-amine, α-methyl-N-2-propyn-1-yl-benzenemethanamine, N-2-butynyl-α-methyl-benzenemethanamine, N-2-butynyl-α-methyl-1-naphthalenemethanamine or N-5-hexyn-1-yl-α-methyl-benzenemethanamine for the manufacture of a medicament for the prophylaxis, treatment or amelioration of physiological disorders or diseases associated with disturbances of CaSR activity, such as hyperparathyroidism.

56. A pharmaceutical composition comprising a compound according to claim 30, or a pharmaceutically acceptable salt, solvate, or ester thereof together with a pharmaceutically acceptable vehicle or excipient.

57. A method of preventing, treating or ameliorating parathyroid carcinoma, parathyroid adenoma, primary parathyroid hyperplasia, cardiac, renal or intestinal disfunction, diseases of the central nervous system, chronic renal failure, chronic kidney disease, primary hyperparathyroidism, secondary hyperparathyroidism, tertiary hyperparathyroidism, anemia, cardiovascular diseases, renal osteodystrophy, osteitis fibrosa, adynamic bone disease, osteoporosis, steroid induced osteoporosis, senile osteoporosis, post menopausal osteoporosis, osteomalacia and related bone disorders, bone loss post renal transplantation, cardiovascular diseases, gastrointestinal diseases, endocrine and neurodegenerative diseases, cancer, neurodegenerative diseases, Alzheimer's disease, anemia, hypercalcemia, or renal bone diseases, the method comprising administering to a patient in need thereof an effective amount of a compound according to claim 30,
optionally in combination or as supplement with an active vitamin-D sterol or vitamin-D derivative, such as 1-α-hydroxycholecalciferol, ergocalciferol, cholecalciferol, 25-hydroxycholecalciferol, 1-α-25-dihydroxycholecalciferol, or in combination or as supplement with phosphate binders, estrogens, calcitonin or biphosphonates.
